(19) United States
(12) Patent Application Publication
(10) Pub. No.: US 2024/0191212 A1
(43) Pub. Date: Jun. 13, 2024

(54) Nucleic Acid Molecules Encoding Indoleamine 2,3-Dioxygenase-2

(71) Applicant: LANKENAU INSTITUTE FOR MEDICAL RESEARCH, WYNNEWOOD, PA (US)

(72) Inventors: George C. Prendergast, Penn Valley, PA (US); Richard Metz, Pennington, NJ (US)

(21) Appl. No.: 18/491,078

(22) Filed: Oct. 20, 2023

Related U.S. Application Data
(63) Continuation of application No. 17/145,002, filed on Jan. 8, 2021, now Pat. No. RE49708.
Application No. 17/145,002 is a reissue of application No. 12/273,296, filed on Nov. 18, 2008, now Pat. No. 8,058,416.
Application No. 12/273,296 is a continuation-in-part of application No. PCT/US07/69271, filed on May 18, 2007.
(60) Provisional application No. 60/801,255, filed on May 18, 2006.
(60) Provisional application No. 60/886,815, filed on Jan. 26, 2007.
(60) Provisional application No. 60/914,472, filed on Apr. 27, 2007.

(51) Int. Cl. C12N 9/02; A01K 67/0276; C12Q 1/26

(52) U.S. Cl. CPC C12N 9/0069; A01K 67/0276; C12Q 1/26; C07K 2319/21; C07K 2319/40; C12N 2830/006

(57) ABSTRACT

The nucleotide and amino acid sequences of indoleamine 2,3-dioxygenase-2 (IDO2) and methods of use thereof are provided.

This application is a continuation application of U.S. application Ser. No. 17/145,002, filed Jan. 8, 2021, which is a reissue application of U.S. Pat. No. 8,058,416, issued Nov. 15, 2011, previously U.S. patent application Ser. No. 12/273,296, filed Nov. 18, 2008, which is a continuation-in-part of PCT/US2007/069271, filed on May 18, 2007, which claims priority under 35 U.S.C. § 119(e) to U.S. Provisional Patent Application No. 60/801,255, filed on May 18, 2006, U.S. Provisional Patent Application No. 60/886,815, filed on Jan. 26, 2007, and U.S. Provisional Patent Application No. 60/914,472, filed on Apr. 27, 2007. The foregoing applications are incorporated by reference herein.

This invention was made with government support under CA109542 awarded by the National Institutes of Health. The government has certain rights in the invention.

### FIELD OF THE INVENTION

This invention relates generally to the fields of cellular metabolism, oncology, and immunology. Specifically, the invention provides the nucleotide and amino acid sequence of indoleamine 2,3-dioxygenase-2 (IDO2) and methods of use thereof.

### BACKGROUND OF THE INVENTION

Tumors, virus-infected cells, and diseased cells characteristically express atypical, potentially immunoreactive antigens. Accumulating evidence suggests that the failure of the immune system to mount an effective response against progressively growing tumors or virally infected cells is not due to a lack of recognizable antigens. Immunosuppression is poorly understood and mechanisms by which cells escape immune surveillance have been poorly explored. Recently, it has been shown that cytotoxic T cells become tolerized by a reduction in local concentrations of tryptophan that are elicited by indoleamine 2,3-dioxygenase-1 (IDO1) activity.

IDO1 is an oxidoreductase that catalyzes the rate-limiting step in tryptophan catabolism. This enzyme is structurally distinct from tryptophan dioxygenase (TDO), which is responsible for dietary tryptophan catabolism in the liver. IDO1 is an IFN-γ target gene that has been suggested to play a role in immunomodulation (Mellor and Munn (1999) Immunol. Today, 20:469-473). Elevation of IDO1 activity depletes the levels of tryptophan in local cellular environments. Induction of IDO1 in antigen-presenting cells, where IDO1 is regulated by IFN-γ, blocks the activation of T cells, which are especially sensitive to tryptophan depletion. T cells must undergo 1-2 rounds of cell division to become activated, but in response to tryptophan depletion they arrest in G1 instead. In this way, IDO1 has been proposed to inhibit the T_{H}1 responses that promote cytotoxic T cell development.

IDO1 has been proposed to modulate gene expression. This modulation is proposed to occur through a pathway involving GCN2, whose activation has been shown to lead to altered gene expression. The proposed pathway involves the following steps. First, IDO1 activity results in the metabolism of tryptophan. Second, the deprivation of tryptophan leads to tRNAs being uncharged. The presence of uncharged tRNAs results in the activation of GCN2 kinase and a general response pathway for amino acid starvation. Third, the active GCN2 kinase phosphorylates serine 52 of the alpha subunit of eukaryotic initiation factor 2 (eIF2α), which is known to be an important translation control mechanism. The regulation of eIF2α activity is governed by the phosphorylation of serine 52. Currently, there are at least three known kinases, i.e., IFN-inducible dsRNA-dependent protein kinase, heme-regulated repressor, and general control (GCN2), which can phosphorylate serine 52 in eIF2α. The phosphorylation of serine 52 in eIF2α prevents the GDP-GTP exchange activity of eIF2α resulting in the suppression of protein synthesis.

GCN2 has been shown to be important for IDO1-dependent responses since a GCN2 knock-out animal phenocopies the IDO1 knock-out animal.

The role of IDO1 in immunosuppression has been demonstrated by the ability of 1-methyl-tryptophan (1MT), a specific and bioactive IDO1 inhibitor (Cady and Sono (1991) Arch. Biochem. Biophys. 291:326-333), to elicit MHC-restricted and T cell-mediated rejection of allogeneic mouse concepti (Mellor et al. (2001) Nat. Immunol. 2:64-68; Munn et al. (1998) Science. 281: 1191-93). This effect is consistent with the high levels of IDO1 expression in placental trophoblast cells (Sedlmayr et al. (2002) Mol. Hum. Reprod. 8:385-391).

Significantly, IDO1 activity has been shown to be elevated frequently in human tumors and/or in cancer patients (Yasui et al. (1986) Proc. Natl. Acad. Sci. USA. 83:6622-26; Taylor and Feng 11991) FASEB J. 5:2516-22). Since IDO1 can modulate immune responses, one logical implication is that IDO1 elevation in cancer may promote tumor immunosuppression (Mellor and Munn (1999) Immunol. Today, 20:469-473; Munn et al. (1999) J. Exp. Med. 189:1363-72; Munn et al. (1998) Science. 281:1191-93). This possibility is supported by the observation that many cancers, including breast cancer, are characterized by a loss of beneficial immune functions that can limit malignant development. For example, T_{H}1 responses (of which IFN-γ production is a hallmark) that promote the production of cytotoxic T cells are suppressed during cancer progression. A resultant hypothesis from this data was that if IDO1 drives cancer progression by blunting T cell activation, then IDO1 inhibition in animals should blunt tumor growth by reversing IDO1-mediated immunosuppression.

Notably, there are two stereoisomers of the IDO1 inhibitor 1MT, e.g., D-1MT and L-1MT. L-1MT inhibits IDO1 and exhibits a characteristic pattern of antitumor activities. In contrast, D-1MT shares the same in vivo properties, but does not inhibit IDO1 itself. One explanation for these results is that D-1MT inhibits an enzyme that is related to, but distinct from IDO1. However, no enzymes related to IDO1 have been identified.

### SUMMARY OF THE INVENTION

In accordance with the present invention nucleic acid molecules encoding indoleamine 2,3-dioxygenase-2 (IDO2) and isoforms, splice variants, and mutants thereof are provided. In another embodiment of the instant invention, primers, probes, antisense molecules, and siRNAs for detection and regulation of IDO2 are provided.

Another aspect of the invention includes amino acid sequences of IDO2. Additionally, antibodies immunologically specific for IDO2 are encompassed by this invention.

In yet another embodiment of the instant invention, methods are provided for the screening of compounds for their ability to the modulate activities of IDO2. The screening methods can be performed in vitro or in host cells, or in animals transgenic for IDO2 expression.

### BRIEF DESCRIPTION OF THE DRAWINGS

FIGS. 1A and 1B provide amino acid sequences of isoforms of IDO2 (SEQ ID NO: 1 and SEQ ID NO: 2, respectively). FIG. 1C provides a schematic of the IDO2 genome organization based on GenBank Accession No. NW_923907.1 when the IDO2 isoform comprises 10 coding exons and about 1227 coding bases.

FIGS. 2A and 2B provide amino acid sequences of mouse IDO2 isoforms (SEQ ID NO: 3 and SEQ ID NO: 4, respectively).

FIG. 3A provides a sequence alignment of the amino acid sequences of a human IDO2 (top strand; amino acids 30-416 of SEQ ID NO: 1 shown) and a human IDO1 (bottom strand; amino acids 13-400 of SEQ ID NO: 5). The underlined residues indicate the conserved histidine and aspartic acid residues necessary for heme binding. # indicates identical and + indicates similar amino acids. FIG. 3B provides the amino acid sequence of human IDO1 (GenBank Accession No AAH27882.1; SEQ ID NO: 5). FIG. 3C provides a sequence alignment of the amino acid sequence of a human IDO2 (SEQ ID NO: 2) and the amino acid sequence of a mouse IDO2 (SEQ ID NO: 4). + indicates similar amino acids. FIGS. 3D-3G provide an amino acid and nucleotide alignment of a murine IDO2 (amino acid, SEQ ID NO: 4; nucleotide, SEQ ID NO: 13) and a murine IDO1 (amino acid, SEQ ID NO: 6; nucleotide, SEQ ID NO: 7). The underlined region indicate exon boundaries.

FIG. 4A is a nucleotide sequence of human IDO2 (SEQ ID NO: 8) which encodes SEQ ID NO: 1. The nucleotide sequence in capital letters (SEQ ID NO: 9) is the sequence encoding the human IDO2 protein. FIG. 4B is a nucleotide sequence of mouse IDO2 (SEQ ID NO: 10) which encodes SEQ ID NO: 3. The nucleotide sequence in capital letters (SEQ ID NO. 11) is the sequence encoding the mouse IDO2 protein. FIGS. 4C and 4D are nucleotide sequences (SEQ ID NO: 12 and SEQ ID NO: 13, respectively) encoding human and murine IDO2 proteins, respectively. Underlined nucleotides indicate the first nucleotide of a new exon.

FIG. 5 provides a Western blot of murine IDO1 and IDO02 expression in T-REx™-293 cells. U is untreated cells, D is doxycycline treated cells, and DT refers to doxycycline and tryptophan treated cells.

FIG. 6 provides graphs of the growth of T-Rex™ cells with no vector (center panel), pcDNA4TO-muIDO1 (left panel) or pcDNA4TO-muIDO2 (right panel). Cells were grown in media alone or in the presence of doxycycline, tryptophan, or both doxycycline and tryptophan.

FIG. 7 provides graphs demonstrating the ability of IDO1 and IDO2 expressing 293 cells to metabolize tryptophan. A kynurenine assay was conducted to determine the ability of IDO1 and IDO2 expressing cells to metabolize tryptophan. The expression of IDO1 or IDO2 was induced by the presence of doxycycline. The cells were also exposed to increasing amounts of tryptophan.

FIG. 8A provides a graph demonstrating the inhibition of IDO1 by methyl thiohydantione (MTH). T-Rex cells or IDO1 expressing T-Rex cells (C10) were treated with doxycycline in the presence of increasing concentrations of DMSO (carrier) or a known IDO1 inhibitor (MTH). The cells were maintained in growth media plus supplements for 5 days, after which the media was harvested and subjected to analysis for the presence of kynurenine. FIG. 8B provides a graph demonstrating that IDO2 metabolizes tryptophan and can be inhibited by 1-methyl-D-tryptophan (1M-D-T). IDO2 expressing T-Rex cells were treated with doxycycline in the presence of increasing concentrations of DMSO (carrier) or the D, L and mixed DL racemic isomers of 1-methyl-tryptophan, (1M-D-T, 1M-L-T and 1M-LD-T, respectively). The cells were maintained in growth media plus supplements for 5 days after which the media was harvested and subjected to analysis for the presence of kynurenine.

FIG. 9A is a Western blot demonstrating that IDO1 and tryptophan deprivation stimulates the expression of the LIP isoforms of NFIL-6. T-REx™ cells (control) and T-REx™ cells expressing IDO1 were either uninduced or induced with doxycycline in the presence or absence of added tryptophan (100 μM). FIG. 9B is a Western blot demonstrating that IDO2, regardless of tryptophan levels, stimulates the expression of the LIP isoforms of NFIL-6. T-REx™ cells (control) and T-REx™ cells expressing IDO2 were either uninduced or induced with doxycycline in the presence or absence of added tryptophan. Cells were also optionally treated with the D, L and mixed DL racemic isomers of 1-methyl-tryptophan, (1M-D-T, 1M-L-T and 1M-LD-T, respectively). FIG. 9C is a Western blot demonstrating that IDO2 expression results in the phosphorylation of elF2α. T-REx™ cells expressing IDO2 were either uninduced or induced with doxycycline in the presence or absence of added tryptophan.

FIGS. 10A-10Q provide a genomic sequence of murine IDO2 (SEQ ID NO: 14) with select restriction enzyme sites and intron/exon boundaries included.

FIGS. 11A-11U provide a genomic sequence of human IDO2 (SEQ ID NO: 15) with select restriction enzyme sites and intron/exon boundaries included.

FIGS. 12A (SEQ ID NOs: 30-43, top to bottom) and 12B (SEQ ID NOs: 44-59, top to bottom) provide a schematic of IDO2 nucleic acid molecules and IDO2 primers for murine and human IDO2, respectively. FIG. 12C provides IDO1 primers and cloning primers (SEQ ID NOs: 60-82, top to bottom).

FIG. 13 provides a schematic of the locations of the introns and exons of IDO2 including exon 1a.

FIG. 14 provides a nucleotide sequence (SEQ ID NO: 20) of IDO2 which comprises 11 exons and encodes SEQ ID NO: 1. Asterisks depict the exon junctions.

FIGS. 15A-15Y is a genomic sequence of human IDO2 (SEQ ID NO: 21) with select restriction enzyme sites and intron/exon boundaries included.

FIGS. 16A-16G provide a nucleotide sequence alignment of a human IDO02 (SEQ ID NO: 12) with IDO2 splice variants and mutants: IDO2 Δ3/4/6 (SEQ ID NO: 22), IDO2 Δ8 (SEQ ID NO: 23), IDO2 Δ6/8 (SEQ ID NO: 24), IDO2 Δ4/5 (SEQ ID NO: 25), IDO2 R235W (SEQ ID NO: 26), IDO2 Y332stop (SEQ ID NO: 27), and a splice variant yielding an alternative exon 8 (SEQ ID NO: 29). SEQ ID NO: 28 is the amino acid sequence. Exon boundaries are indicated and the encoded amino acids are also provided.

FIG. 17 is a graph of the kynurenine activity of various IDO2 splice variants or mutants alone or co-expressed with IDO2, IDO1, or empty vector (Puc).

FIGS. 18A and 18B are images of Western blots of cellular lysates of 293 cells expressing the following: 1: no IDOs, 2: mouse IDO2 (WT), 3: human IDO2 (WT), 4: mouse IDO1/IDO2, 5: mouse IDO2, 6: mouse IDO2/1 chimera, 7: human IDO1/mouse IDO2, 8: human IDO2 (R-W), 9: mouse IDO2 (R-W), 10: hu8man IDO2 (Y-*), 11; human IDO1. The Western blot in FIG. 18A was probed with an anti-IDO1 polyclonal sera (1:200) and the Western blot in FIG. 18B was probed with an anti-muIDO2 monoclonal antibody (MAb 7.4.6; 1:100).

FIG. 19A is a schematic of human IDO2 mRNA and the location of the primers used for RT-PCR. FIG. 19B provides images of the RT-PCR assays performed on total RNA from the indicated tissues. The primers used in the RT-PCR are indicated at the right of the panels and GAPDH is provided as a control. FIG. 19C is an image of an agarose gel demonstrating the presence of various IDO2 isoforms in human placenta.

FIGS. 20A (SEQ ID NOs: 83-122, top to bottom) and 20B (SEQ ID NOs: 124-147, top to bottom) provide target sequences and siRNA sequences for human and mouse IDO2, respectively.

FIG. 21 is a schematic of a target vector strategy for the construction of a conditional IDO2 knockout mice strain. Removal of exons 9 and 10 in the IDO2 gene happens upon crossing with a transgenic Cre mouse expressing Cre recombinase under the control of tissue specific promoters.

FIG. 22 is a schematic of a screening strategy for IDO-2 knockin mice upon removal of the selection marker NeoR.

FIG. 23A is a schematic of vector pVLmuIDO2.

FIGS. 23B-23I provide the sequence of vector pVLmuIDO2 (SEQ ID NO: 162).

FIG. 24A is a schematic of vector pVLhuIDO2.

FIGS. 245-241 provide the sequence of vector pVLhuIDO2 (SEQ ID NO: 163).

FIGS. 25A and 25B are graphs of the inhibition studies of varying amounts of D-1-methyl-tryptophan and L-1-methyl-tryptophan on IDO1 and IDO2 whole cell lysates, respectively.

### DETAILED DESCRIPTION OF THE INVENTION

The involvement of IDO1 in immune suppression, particularly the suppression of T cell immunity, has been well documented. As stated hereinabove, there is evidence to suggest that a previously unidentified enzyme related to IDO1 is also involved in immune suppression. Indeed, L-1MT has been shown to be effective at inhibiting IDO1 activity and tumor progression, but the stereoisomer D-1MT does not significantly inhibit IDO1 activity yet retains antitumor properties. Further, at least one IDO1 antisera has been generated which recognizes, in addition to IDO1, a differently regulated protein with a slightly larger apparent molecular weight as determined by Western blot.

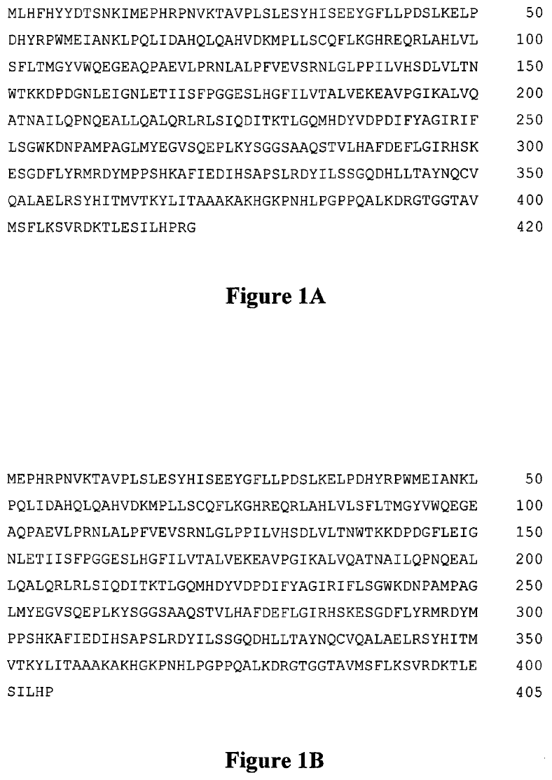

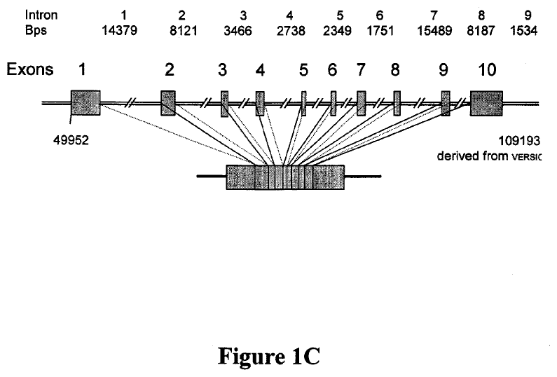

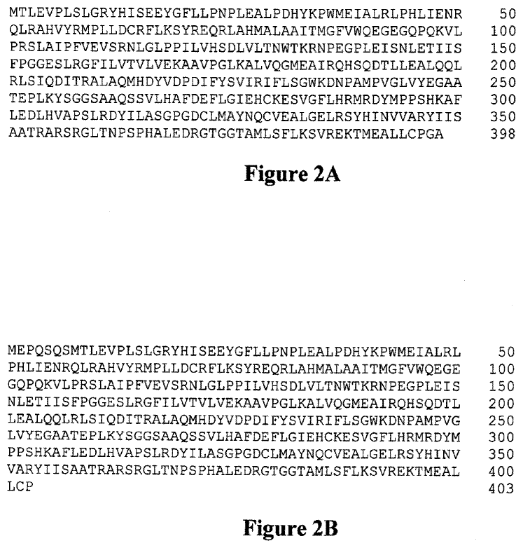

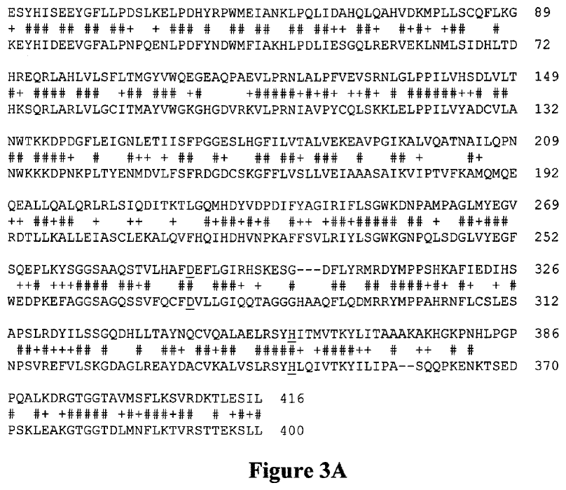

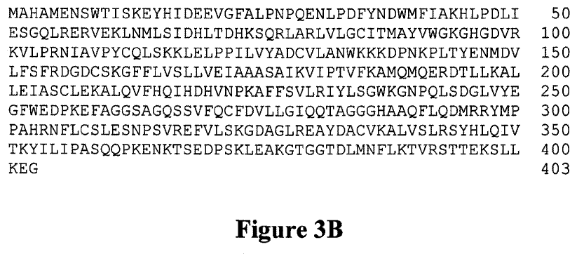

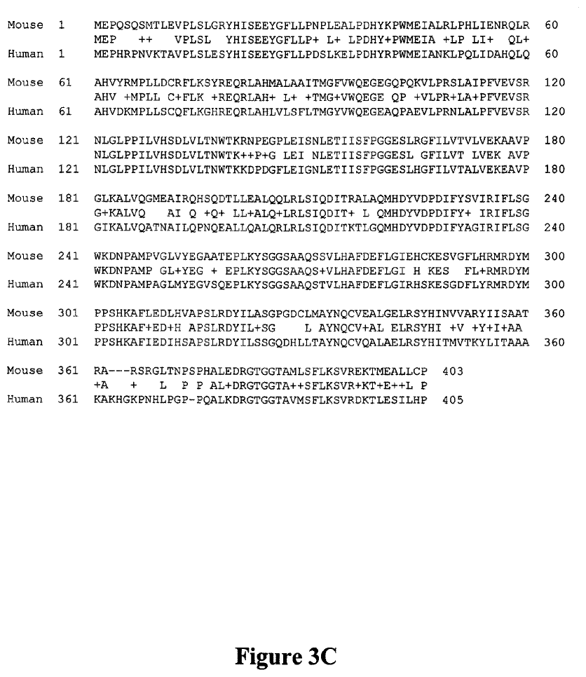

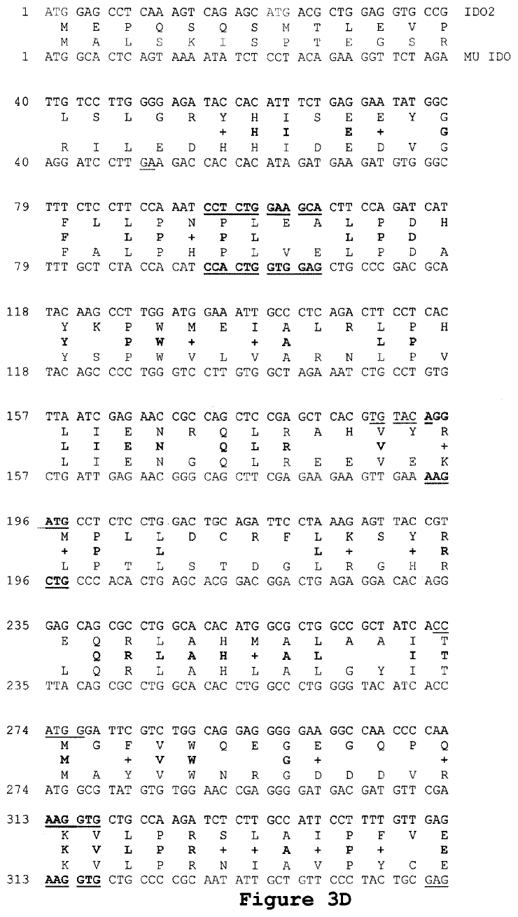

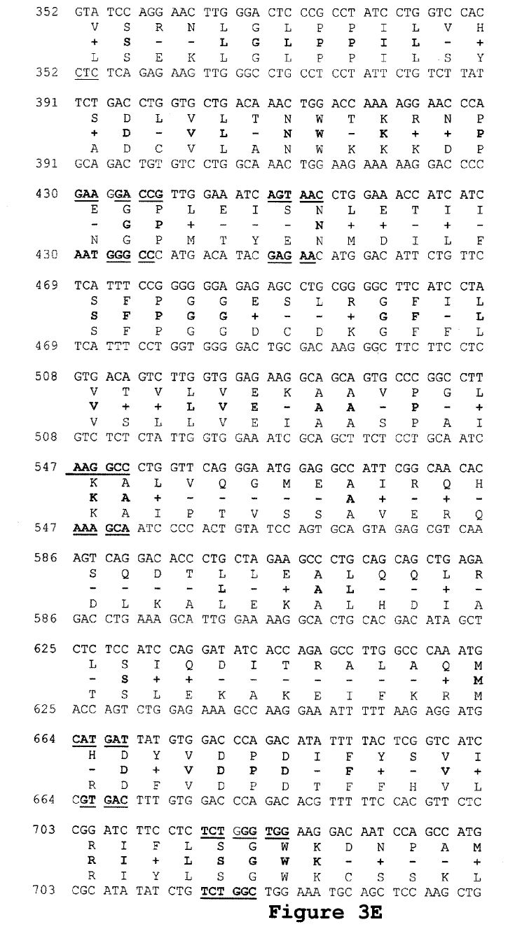

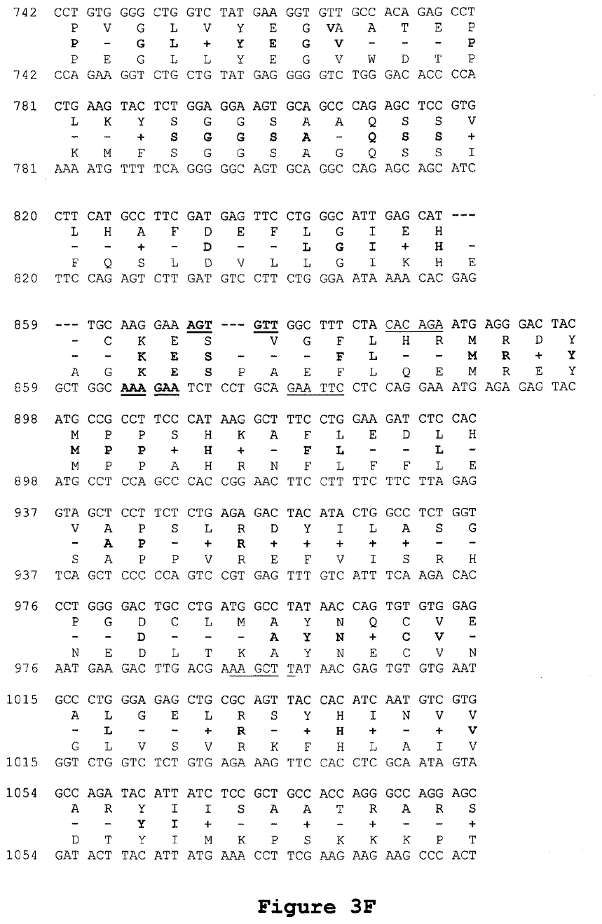

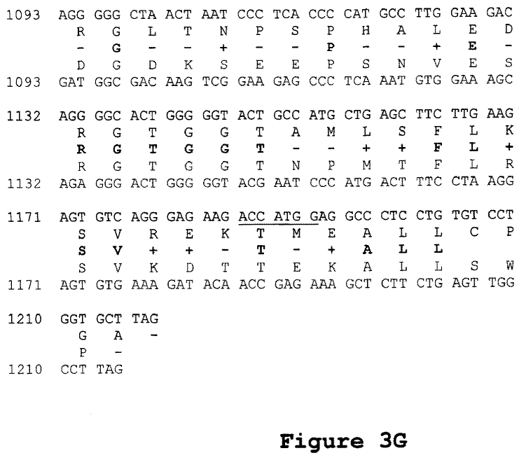

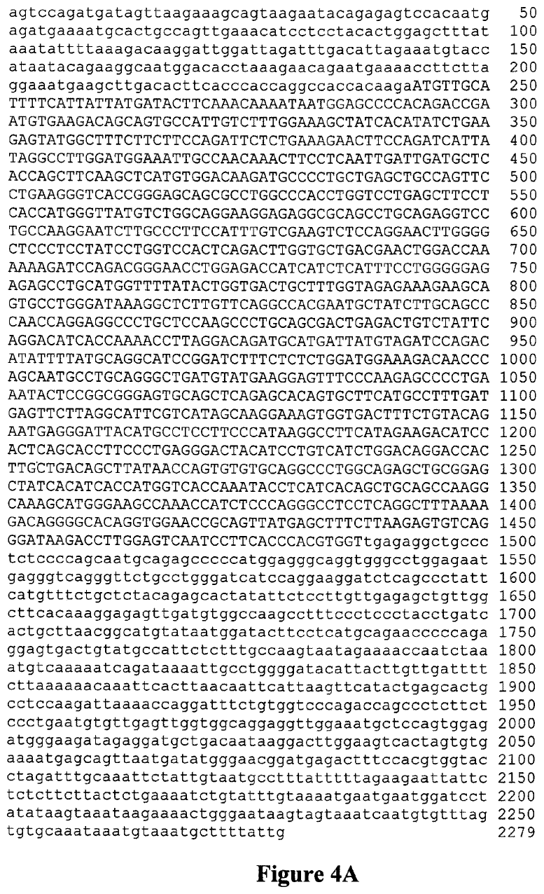

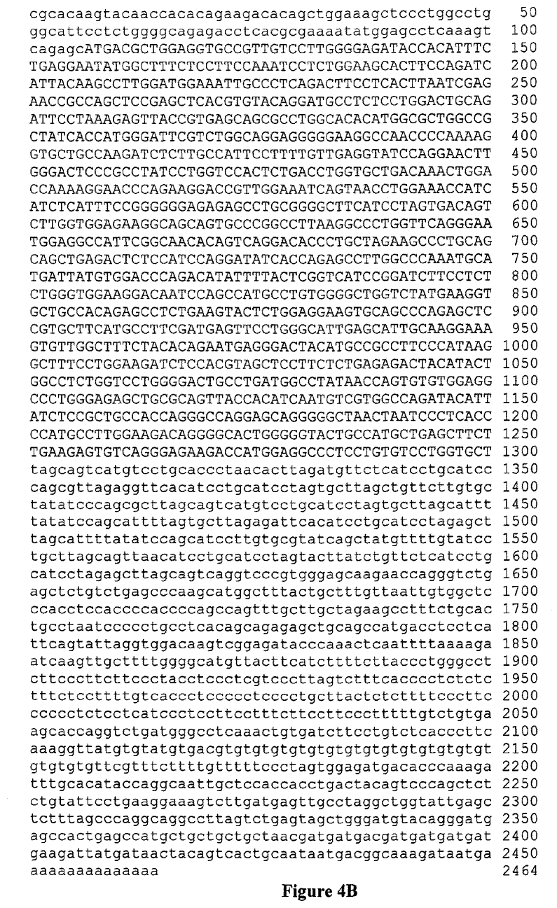

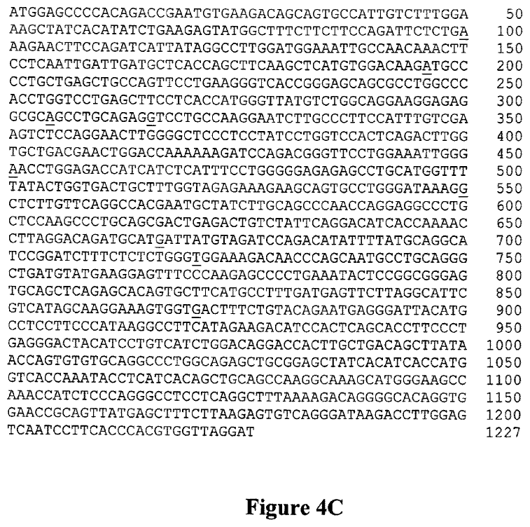

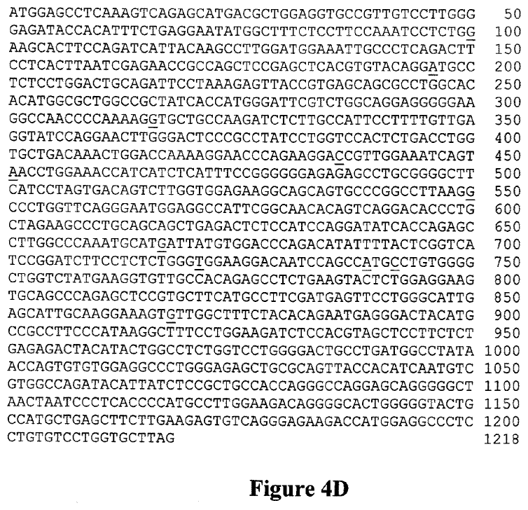

In accordance with the instant invention, a novel homolog of IDO has been identified and named indoleamine 2,3-dioxygenase-2 (IDO2, also referred to as indoleamine 2,3-dioxygenase-like-1 (INDOL1) and IDO-NT). Amino acid sequences of human and mouse IDO2 are provided herein (see, e.g., FIGS. 1 and 2, respectively). Nucleotide sequences of human and mouse IDO2 are also provided (see, e.g., FIGS. 4A and 4B, respectively). Notably, IDO2 has approximately 45% sequence identity with human IDO1 (FIG. 3A).

Significantly, IDO2 shares the structural features of IDO1 that are known to be essential for heme binding and tryptophan catabolic activity. Indeed, the histidine and aspartic acid residues required for heme binding are conserved between IDO1 and IDO2 (see FIG. 3A).

Various isoforms, splice variants, and mutant IDO2 nucleic acids and proteins are provided. One isoform of IDO2 is also slightly larger than IDO1 (420 amino acids versus 403 amino acids). This size difference may account for the larger species sometimes seen in Western blots with antisera directed to IDO1. Indeed, portions of the sequence of the IDO1 peptide used to generate the antisera are conserved in IDO2. Based on these facts, it appears that certain IDO1 antisera are capable of cross-reacting with IDO2.

The 5′ untranslated region of the IDO2 gene comprising the promoter elements, has a high degree of homology with the consensus sequences for the key immune regulatory transcription factors STAT1 and IRF-7. Significantly, IRF-7 is the master regulator of dendritic cells which present antigen to T cells and control T cell activation versus anergy. This similarity in the 5′ regulatory region to the consensus sequence of these immune regulatory transcription factors suggests that IDO2 plays a role in the regulation of the immune response. Indeed, in silico analysis (e.g., an electronic Northern based on serial analysis of gene expression (SAGE)) determined that the highest expression of IDO2 would be in dendritic cells, where IDO1 is also known to be expressed, and pre-dendritic cells (such as JAWS II cells).

IDO2 nucleic acid molecules and proteins may be useful in the diagnosis and prognosis of immune modulation, like IDO1. IDO2 may also be used in the methods described by U.S. Pat. Nos. 6,451,840 and 6,482,416 for IDO1. Furthermore, as described hereinbelow, IDO2 is a candidate therapeutic target for diseases and disorders associated with immune modulation and/or protein misfolding such as cancer, viral infections, and other pathological conditions.

The activation of GCN2 translation control pathway alters the expression of the transcription factor Nuclear Factor IL6 (NFIL6; also known in the literature as C/EBPβ/IL6DBP/TCF5). The CCAAT-enhancer binding protein beta (C/EBPβ) is a basic leucine zipper (bZIP) transcription factor selectively expressed during differentiation of liver, adipose tissue, blood cells, and endocrine pancreas. C/EBPβ encodes four isoforms arising from the differential initiation of translation at four different in-frame AUGs, thereby producing the 40 kDa, 35 kDa, 20 kDa and 8.5 kDa C/EBPβ isoforms. The Lap and LIP isoforms of NF-IL6 have a variety of pleiotropic activities and are involved in regulation cell function, cell growth, and immune response. Lap-1 and Lap-2 have an antiproliferative effect, regulate cell cycle control and cytokine expression, and are pro-inflammatory. LIP is a dominant negative isoform. Therefore, the altered expression of LIP is a relevant biomarker for IDO1 and IDO2 activity in any cell expressing NF-IL6.

IDO1 expression leads to the increase in LIP (also called liver inhibitory protein) which is a dominant negative regulator of Lap1/2 activities, since it lacks certain activation and regulatory domains on LAPs (Liver activation proteins) and can form heterodimers with the LAP proteins, thereby altering their activity. By inference, it may be concluded that the activation of LIP is dependent on the deprivation of tryptophan, the activation of the GCN2 pathway and the increase in LIP production, which in turn results in altered gene expression and cell function. The data described herein support this model since the addition of exogenous tryptophan, even when IDO1 is highly expressed, blocks the production of LIP and activation of the GCN2 pathway.

IDO2 expression also leads to the activation of GCN2 kinase and increased LIP production. However the activation of the GCN2-LIP pathway is not inhibited by the addition of exogenous tryptophan. Therefore, it may be concluded that the mechanism of action for IDO2 is not tryptophan deprivation but rather the direct activation of the GCN2 kinase-LIP pathway. This is may be due to the generation of uncharged tryptophan tRNAs directly, the charging of tRNAs with kynurenine, or some other effect on the GCN2-LIP pathway.

It is also demonstrated herein that IDO2 activity can be inhibited by 1-methyl-D-tryptophan (1M-D-T) which blocks the activation of GCN2 kinase and subsequent altered LIP expression.

The activation of IDO2, like IDO1, has distinct and important properties for the regulation of immune responses. It is known that IDO1 exerts its activity locally by depriving the local cell environment of tryptophan, activating the GCN2 pathway, and suppressing T-cell activation and other immune activities. IDO2 directly activates the GCN2 pathway and exerts its activity globally and systemically in any cell in which it is expressed, including immune T cells and antigen presenting cells. IDO2 may also impact cell activities at distal locations as the altered expression of LIP/LAP is known to affect the expression of soluble growth factors and cytokines that can impact cell responses, like immune activation. One such scenario would be the activation of IDO2 in an antigen presenting cell (APC), such as a dendritic cell, which converts the APC in the presence of antigen to a tolerogenic cell that suppresses the immune response to a particular antigen.

### I. Definitions

The term “IDO inhibitor” refers to an agent capable of inhibiting the activity of indoleamine 2,3-dioxygenase (IDO) (such as IDO1 and IDO2) and thereby reversing IDO-mediated immunosuppression. An IDO inhibitor may be a competitive, noncompetitive, or irreversible IDO inhibitor. “A competitive IDO inhibitor” is a compound that reversibly inhibits IDO enzyme activity at the catalytic site (including, without limitation, 1-methyl-tryptophan); “a noncompetitive IDO Inhibitor” is a compound that reversibly inhibits IDO enzyme activity at a non-catalytic site (including, without limitation, norharman); and “an irreversible IDO inhibitor” is a compound that irreversibly destroys IDO enzyme activity by forming a covalent bond with the enzyme (including, without limitation, cyclopropyl/aziridinyl tryptophan derivatives). IDO inhibitors include, without limitation, 1-methyl-DL-tryptophan (1MT; Sigma-Aldrich; St. Louis, MO), β-(3-benzofuranyl)-DL-alanine (Sigma-Aldrich), beta-(3-benzo(b)thienyl)-DL-alanine (Sigma-Aldrich), 6-nitro-L-tryptophan (Sigma-Aldrich), indole 3-carbinol (LKT Laboratories; St. Paul, MN), 3,3′-diindolylmethane (LRT Laboratories), epigallocatechin gallate (LKT Laboratories), 5-Br-4-Cl-indoxyl 1,3-diacetate (Sigma-Aldrich), 9-vinylcarbazole (Sigma-Aldrich), acemetacin (Sigma-Aldrich), 5-bromo-DL-tryptophan (Sigma-Aldrich), 5-bromoindoxyl diacetate (Sigma-Aldrich), phenyl-TH-DL-trp (3-(N-phenyl-thiohydantoin)-indole) (Sigma-Aldrich), propenyl-TH-DL-trp (3-(N-allyl-thiohydantoin)-indole) (Asinex; Moscow, Russia), methyl-TH-DL-trp (3-(N-methyl-thiohydantoin)-indole) (Sigma-Aldrich), brassinin (LKT Laboratories), 5-methyl-brassinin (Mehta, et. al. (1994) Anticancer Res., 14:1209-1213); 3,3′-diindolylmethane (DIM; LKT Laboratories), indole-3-carbinol (I3C; LKT Laboratories), and the IDO inhibitors provided in U.S. patent application Ser. No. 10/550,444 and U.S. Provisional Application 60/730,706. IDO inhibitors may selectively or preferentially inhibit IDO1 (an “IDO1 inhibitor”) and/or IDO2 (an “IDO2 inhibitor”). IDO inhibitors include, without limitation, nucleic acid molecules (e.g., siRNA, antisense oligonucleotides), peptides, chemical compounds, and antibodies, or biologically active fragments thereof.

“Pharmaceutically acceptable” indicates approval by a regulatory agency of the Federal government or a state government. “Pharmaceutically acceptable” agents may be listed in the U.S. Pharmacopeia or other generally recognized pharmacopeia for use in animals, and more particularly in humans.

A “carrier” refers to, for example, a diluent, adjuvant, excipient, auxiliary agent or vehicle with which an active agent of the present invention is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous saline solutions and aqueous dextrose and glycerol solutions are preferably employed as carriers, particularly for injectable solutions. Suitable pharmaceutical carriers are described in “Remington's Pharmaceutical Sciences” by E. W. Martin.

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism.

When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An “isolated nucleic acid” (either DNA or RNA) may further represent a molecule produced directly by biological or synthetic means and separated from other components present during its production.

A “replicon” is any genetic element, for example, a plasmid, cosmid, bacmid, plastid, phage or virus, which is capable of replication largely under its own control. A replicon may be either RNA or DNA and may be single or double stranded. Generally, a “viral replicon” is a replicon which contains the complete genome of the virus. A “sub-genomic replicon” refers to a viral replicon that contains something less than the full viral genome, but is still capable of replicating itself. For example, a sub-genomic replicon may contain most of the genes encoding for the non-structural proteins of the virus, but not most of the genes encoding for the structural proteins.

A “vector” is a replicon, such as a plasmid, cosmid, bacmid, phage or virus, to which another genetic sequence or element (either DNA or RNA) may be attached so as to bring about the replication of the attached sequence or element.

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

The terms “percent similarity,” “percent identity” and “percent homology,” when referring to a particular sequence, are used as set forth in the University of Wisconsin GCG software program.

The term “substantially pure” refers to a preparation comprising at least 50-60% by weight of a given material (e.g., nucleic acid, oligonucleotide, protein, etc.). More preferably, the preparation comprises at least 75% by weight, and most preferably 90-95% by weight of the given compound. Purity is measured by methods appropriate for the given compound (e.g. chromatographic methods, agarose or polyacrylamide gel electrophoresis, HPLC analysis, and the like).

The term “oligonucleotides” as used herein refers to sequences, primers and probes of the present invention, and is defined as a nucleic acid molecule comprised of two or more ribo- or deoxyribonucleotides, preferably more than three. The exact size of the oligonucleotide will depend on various factors and on the particular application and use of the oligonucleotide.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as appropriate temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able to anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and use of the method. For example, for diagnostic applications, depending on the complexity of the target sequence, the oligonucleotide probe typically contains 15-25 or more nucleotides, although it may contain fewer nucleotides. The probes herein are selected to be complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

Polymerase chain reaction (PCR) has been described in U.S. Pat. Nos. 4,683,195, 4,800,195, and 4,965,188, the entire disclosures of which are incorporated by reference herein.

With respect to single stranded nucleic acids, particularly oligonucleotides, the term “specifically hybridizing” refers to the association between two single-stranded nucleotide molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA molecule of the invention, to the substantial exclusion of hybridization of the oligonucleotide with single-stranded nucleic acids of non-complementary sequence. Appropriate conditions enabling specific hybridization of single stranded nucleic acid molecules of varying complementarity are well known in the art.

For instance, one common formula for calculating the stringency conditions required to achieve hybridization between nucleic acid molecules of a specified sequence homology is set forth below (Sambrook et al., 1989, Molecular Cloning; A Laboratory Manual, Cold Spring Harbor Laboratory Press):

Tₘ=81.5° C.+16.6 Log [Na+]+0.41(% G+C)−0.63(% formamide)−600/#bp in duplex

As an illustration of the above formula, using [Na+]=[0.368] and 50% formamide, with GC content of 42% and an average probe size of 200 bases, the Tₘ is 57° C. The Ta of a DNA duplex decreases by 1-1.5° C. with every 1% decrease in homology. Thus, targets with greater than about 75% sequence identity would be observed using a hybridization temperature of 42° C.

The stringency of the hybridization and wash depend primarily on the salt concentration and temperature of the solutions. In general, to maximize the rate of annealing of the probe with its target, the hybridization is usually carried out at salt and temperature conditions that are 20-25° C. below the calculated Tₘ of the hybrid. Wash conditions should be as stringent as possible for the degree of identity of the probe for the target. In general, wash conditions are selected to be approximately 12-20° C. below the Tₘ of the hybrid. In regards to the nucleic acids of the current invention, a moderate stringency hybridization is defined as hybridization in 6×SSC, 5×Denhardt's solution, 0.5% SDS and 100 μg/ml denatured salmon sperm DNA at 42° C., and washed in 2×SSC and 0.5% SDS at 55° C. for 15 minutes. A high stringency hybridization is defined as hybridization in 6×SSC, 5×Denhardt's solution, 0.5% SDS and 100 μg/ml denatured salmon sperm DNA at 42° C., and washed in 1×SSC and 0.5% SDS at 65° C. for 15 minutes. A very high stringency hybridization is defined as hybridization in 6×SSC, 5×Denhardt's solution, 0.5% SDS and 100 μg/ml denatured salmon sperm DNA at 42° C., and washed in 0.1×SSC and 0.5% SDS at 65° C. for 15 minutes.

The term “isolated protein” or “isolated and purified protein” is sometimes used herein. This term refers primarily to a protein produced by expression of an isolated nucleic acid molecule of the invention. Alternatively, this term may refer to a protein that has been sufficiently separated from other proteins with which it would naturally be associated, so as to exist in “substantially pure” form. “Isolated” is not meant to exclude artificial or synthetic mixtures with other compounds or materials, or the presence of impurities that do not interfere with the fundamental activity, and that may be present, for example, due to incomplete purification, or the addition of stabilizers.

The term “gene” refers to a nucleic acid comprising an open reading frame encoding a polypeptide, including both exon and (optionally) intron sequences. The nucleic acid may also optionally include non-coding sequences such as promoter or enhancer sequences. The term “intron” refers to a DNA sequence present in a given gene that is not translated into protein, is generally found between exons, and is “spliced out” during processing of the mRNA transcript. As used herein, the term “exon” refers to a nucleic acid sequence found in genomic DNA that is predicted and/or experimentally confirmed to contribute contiguous sequence to a mature (e.g., spliced) mRNA transcript and/or is translated into protein.

As used herein, the phrase “splice variants” refers to RNA molecules initially transcribed from the same genomic DNA sequence but which have undergone alternative RNA splicing. Alternative RNA splicing occurs when a primary RNA transcript undergoes splicing, generally for the removal of introns, which results in the production of more than one mRNA molecule, which may encode different amino acid sequences. The term splice variant may also refer to the proteins encoded by the above RNA molecules. As used herein, the phrase “alternative splicing” includes all types of RNA processing that lead to expression of plural protein isoforms from a single gene. As such, the phrase “splice variant” embraces mRNAs transcribed from a given gene that, however processed, collectively encode plural protein isoforms. For example, and by way of illustration only, splice variants can include exon insertions, exon extensions, exon truncations, exon deletions, alternatives in the 5′ untranslated region and alternatives in the 3′ untranslated region.

The phrase “consisting essentially of” when referring to a particular nucleotide or amino acid means a sequence having the properties of a given SEQ ID NO. For example, when used in reference to an amino acid sequence, the phrase includes the sequence per se and molecular modifications that would not affect the basic and novel characteristics of the sequence.

The term “promoters” or “promoter” as used herein can refer to a DNA sequence that is located adjacent to a DNA sequence that encodes a recombinant product. A promoter is preferably linked operatively to an adjacent DNA sequence. A promoter typically increases an amount of recombinant product expressed from a DNA sequence as compared to an amount of the expressed recombinant product when no promoter exists. A promoter from one organism can be utilized to enhance recombinant product expression from a DNA sequence that originates from another organism. For example, a vertebrate promoter may be used for the expression of jellyfish GFP in vertebrates. In addition, one promoter element can increase an amount of recombinant products expressed for multiple DNA sequences attached in tandem. Hence, one promoter element can enhance the expression of one or more recombinant products. Multiple promoter elements are well-known to persons of ordinary skill in the art.

The term “enhancers” or “enhancer” as used herein can refer to a DNA sequence that is located adjacent to the DNA sequence that encodes a recombinant product. Enhancer elements are typically located upstream of a promoter element or can be located downstream of or within a coding DNA sequence (e.g., a DNA sequence transcribed or translated into a recombinant product or products). Hence, an enhancer element can be located 100 base pairs, 200 base pairs, or 300 or more base pairs upstream or downstream of a DNA sequence that encodes recombinant product. Enhancer elements can increase an amount of recombinant product expressed from a DNA sequence above increased expression afforded by a promoter element. Multiple enhancer elements are readily available to persons of ordinary skill in the art.

The terms “transfected” and “transfection” as used herein refer to methods of delivering exogenous DNA into a cell. These methods involve a variety of techniques, such as treating cells with high concentrations of salt, an electric field, liposomes, polycationic micelles, or detergent, to render a host cell outer membrane or wall permeable to nucleic acid molecules of interest. These specified methods are not limiting and the invention relates to any transformation technique well known to a person of ordinary skill in the art.

An “antibody” or “antibody molecule” is any immunoglobulin, including antibodies and fragments thereof, that binds to a specific antigen. The term includes polyclonal, monoclonal, chimeric, single domain (Dab) and bispecific antibodies. As used herein, antibody or antibody molecule contemplates recombinantly generated intact immunoglobulin molecules and immunologically active portions of an immunoglobulin molecule such as, without limitation: Fab, Fab′, F(ab′)₂, F(v), scFv, scFv₂, scFv-Fc, minibody, diabody, tetrabody, single variable domain (e.g., variable heavy domain, variable light domain), bispecific, Affibody® molecules (Affibody, Bromma, Sweden), and peptabodies (Terskikh et al. (1997) PNAS 94:1663-1668).

Chemotherapeutic agents are compounds that exhibit anticancer activity and/or are detrimental to a cell (e.g., a toxin). Suitable chemotherapeutic agents include, but are not limited to: toxins (e.g., saporin, ricin, abrin, ethidium bromide, diptheria toxin, Pseudomonas exotoxin, and others listed above); alkylating agents (e.g., nitrogen mustards such as chlorambucil, cyclophosphamide, isofamide, mechlorethamine, melphalan, and uracil mustard; aziridines such as thiotepa; methanesulphonate esters such as busulfan; nitroso ureas such as carmustine, lomustine, and streptozocin; platinum complexes such as cisplatin and carboplatin; bioreductive alkylators such as mitomycin, procarbazine, dacarbazine and altretamine); DNA strand-breakage agents (e.g., bleomycin); topoisomerase II inhibitors (e.g., amsacrine, dactinomycin, daunorubicin, idarubicin, mitoxantrone, doxorubicin, etoposide, and teniposide); DNA minor groove binding agents (e.g., plicamydin); antimetabolites (e.g., folate antagonists such as methotrexate and trimetrexate; pyrimidine antagonists such as fluorouracil, fluorodeoxyuridine, CB3717, azacitidine, cytarabine, and floxuridine; purine antagonists such as mercaptopurine, 6-thioguanine, fludarabine, pentostatin; asparginase; and ribonucleotide reductase inhibitors such as hydroxyurea); tubulin interactive agents (e.g., vincristine, vinblastine, and paclitaxel (Taxol)); hormonal agents (e.g., estrogens; conjugated estrogens; ethinyl estradiol; diethylstilbesterol; chlortrianisen; idenestrol; progestins such as hydroxyprogesterone caproate, medroxyprogesterone, and megestrol; and androgens such as testosterone, testosterone propionate, fluoxymesterone, and methyltestosterone); adrenal corticosteroids (e.g., prednisone, dexamethasone, methylprednisolone, and prednisolone); leutinizing hormone releasing agents or gonadotropin-releasing hormone antagonists (e.g., leuprolide acetate and goserelin acetate); and antihormonal antigens (e.g., tamoxifen, antiandrogen agents such as flutamide; and antiadrenal agents such as mitotane and aminoglutethimide). In a particular embodiment, the chemotherapeutic agent is selected from the group consisting of: placitaxel (Taxol®), cisplatin, docetaxol, carboplatin, vincristine, vinblastine, methotrexate, cyclophosphamide, CPT-11, 5-fluorouracil (5-FU), gemcitabine, estramustine, carmustine, adriamycin (doxorubicin), etoposide, arsenic trioxide, irinotecan, and epothilone derivatives.

“Natural allelic variants”, “mutants” and “derivatives” of particular sequences of nucleic acids refer to nucleic acid sequences that are closely related to a particular sequence but which may possess, either naturally or by design, changes in sequence or structure. By closely related, it is meant that at least about 75%, but often, more than 90%, of the nucleotides of the sequence match over the defined length of the nucleic acid sequence referred to using a specific SEQ ID NO. Changes or differences in nucleotide sequence between closely related nucleic acid sequences may represent nucleotide changes in the sequence that arise during the course of normal replication or duplication in nature of the particular nucleic acid sequence. Other changes may be specifically designed and introduced into the sequence for specific purposes, such as to change an amino acid codon or sequence in a regulatory region of the nucleic acid. Such specific changes may be made in vitro using a variety of mutagenesis techniques or produced in a host organism placed under particular selection conditions that induce or select for the changes. Such sequence variants generated specifically may be referred to as “mutants” or “derivatives” of the original sequence.

### II. Nucleic Acid Molecules

Nucleic acid molecules encoding the IDO2 proteins of the invention may be prepared by two general methods: (1) synthesis from appropriate nucleotide triphosphates and (2) isolation and/or amplification from biological sources. These methods utilize protocols well known in the art. The availability of nucleotide sequence information, such as the sequences provided herein, enables preparation of an isolated nucleic acid molecule of the invention by oligonucleotide synthesis. Synthetic oligonucleotides may be prepared by the phosphoramidite method employed in the Applied Biosystems 38A DNA Synthesizer or similar devices. The resultant construct may be purified according to methods known in the art, such as high performance liquid chromatography (HPLC). Long, double-stranded polynucleotides may be synthesized in stages, due to any size limitations inherent in the oligonucleotide synthetic methods.

Nucleic acid sequences encoding the IDO2 proteins of the invention may be isolated from appropriate biological sources using methods known in the art. In one embodiment, a cDNA clone is isolated from a cDNA expression library, preferably of human origin. In an alternative embodiment, utilizing the sequence Information provided by the cDNA sequence, genomic clones encoding altered IDO2 proteins may be isolated. Additionally, cDNA or genomic clones having homology with human and mouse IDO2 may be isolated from other species using oligonucleotide probes corresponding to predetermined sequences within the human and mouse IDO2 encoding nucleic acids.

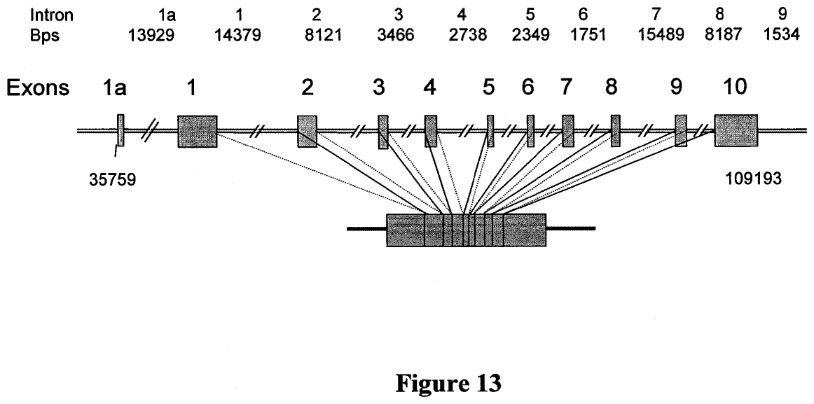

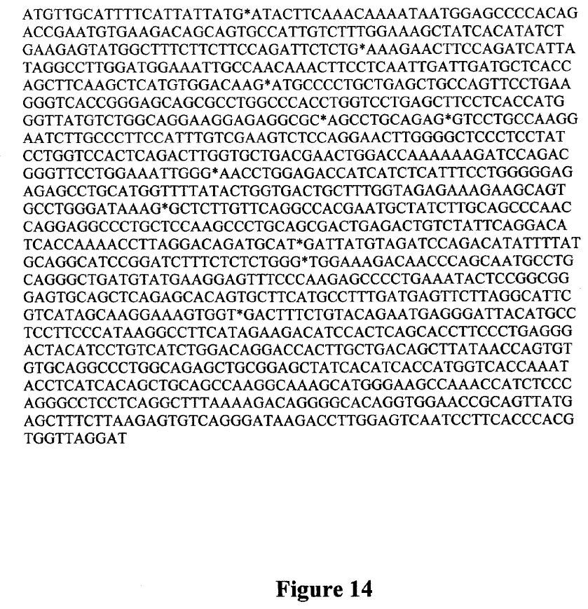

The present invention also encompasses splice variants of IDO2. FIGS. 1C and 13 provide a schematic of the locations of the introns and exons of IDO2. Generally, IDO2 comprises 10 or 11 exons depending on whether exon 1a is included. The genomic region is about 73234 basepairs from the start codon ATG to the stop codon TAG with about 1266 coding bases. FIG. 14 provides a nucleotide sequence of IDO2 comprising all exons.

Exemplary nucleotide sequences encoding human IDO2 are SEQ ID NOs: 8, 9, 12, 15, 20, and 21 and exemplary nucleotide sequences encoding mouse IDO2 are SEQ ID NOs: 10, 11, 13, and 14. The nucleotide sequence of certain splice variants and mutants are also provided in SEQ ID NOs: 22-25. An IDO2 nucleotide sequence may have 75%, 80%, 85%, 90%, 95%, 97%, or 99% homology with any of these nucleic acid molecules.

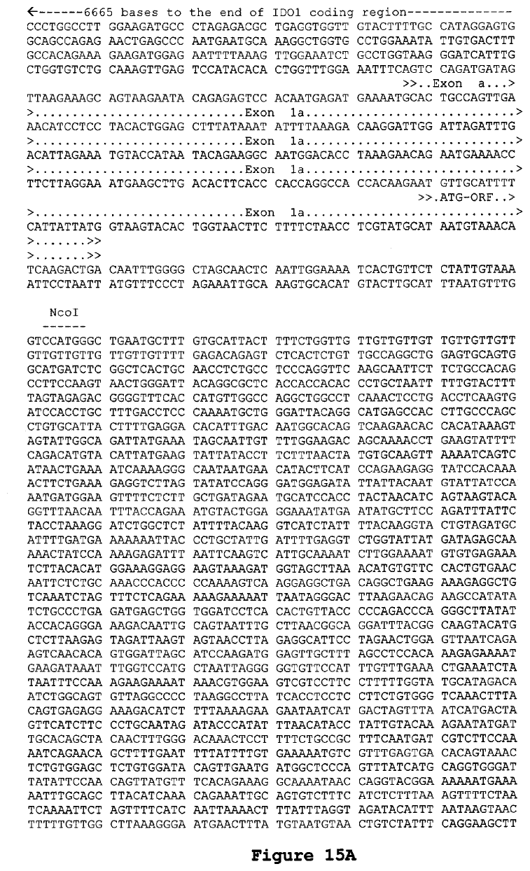

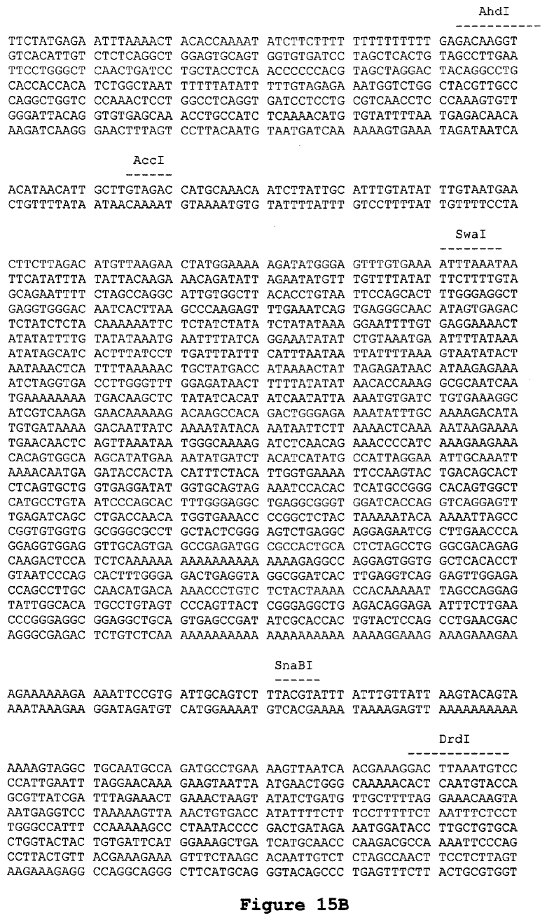

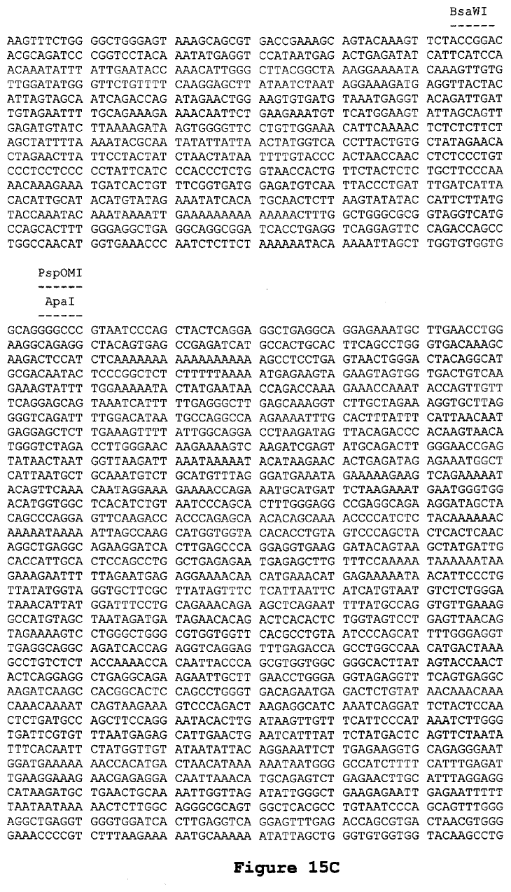

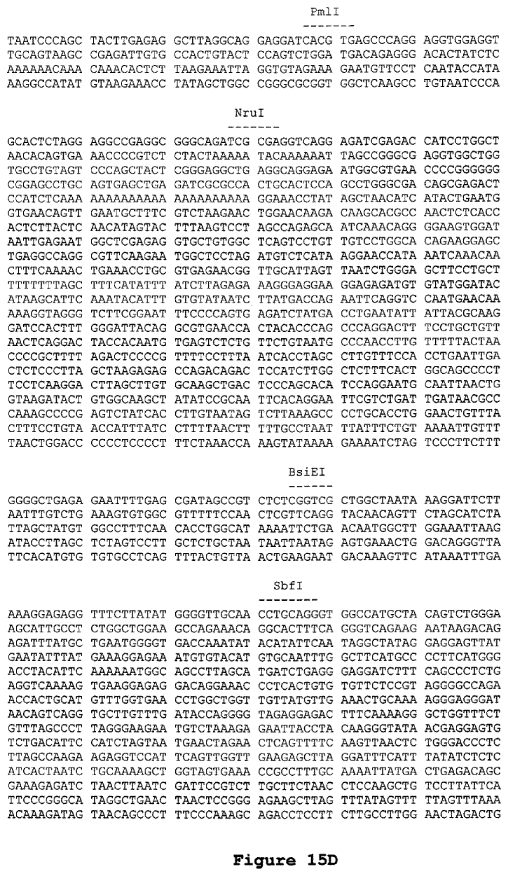

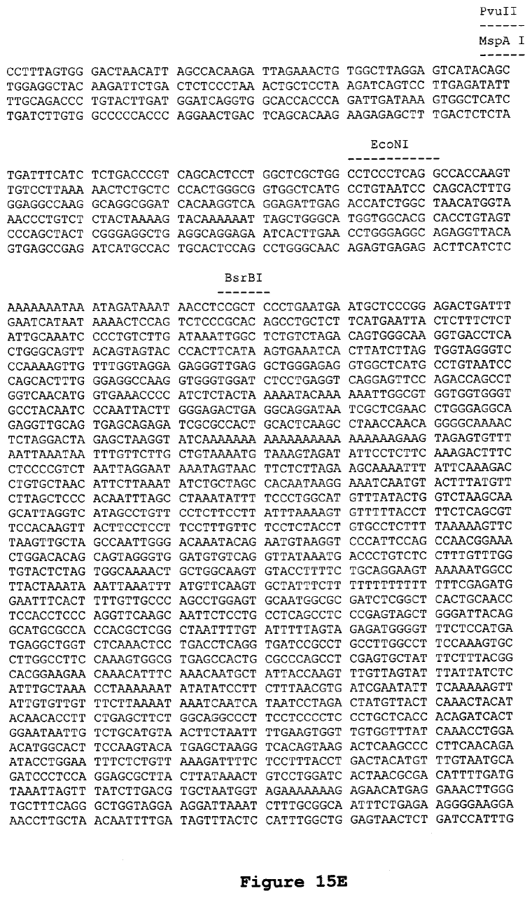

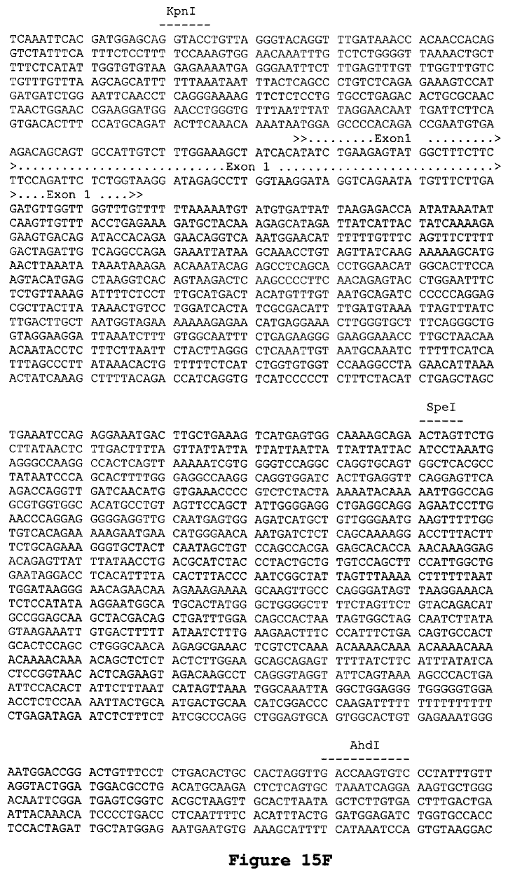

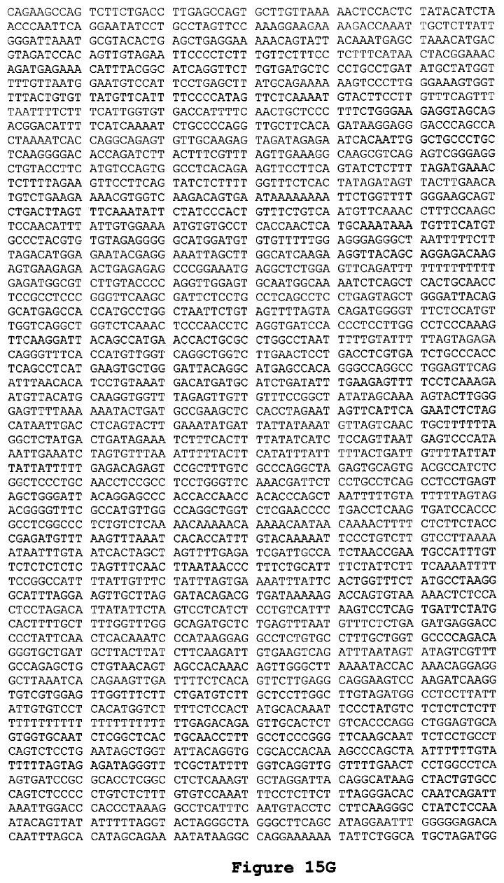

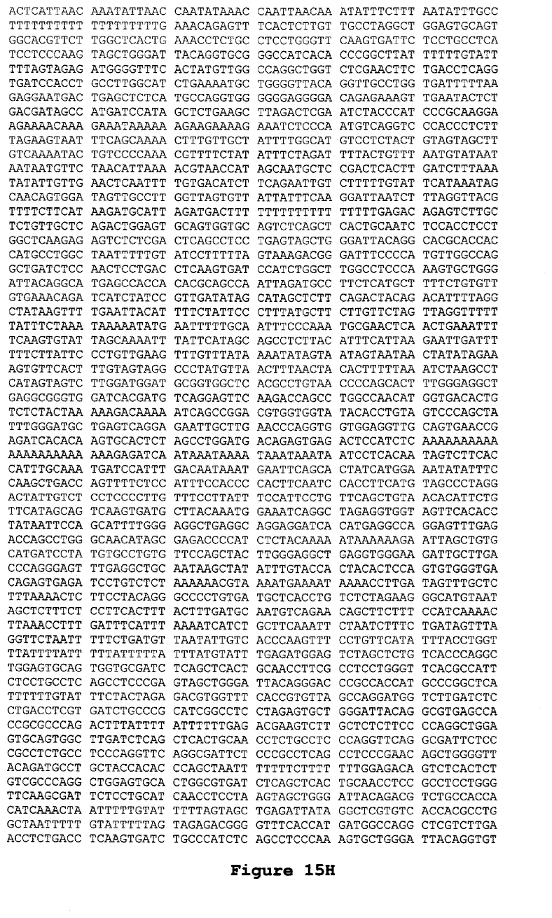

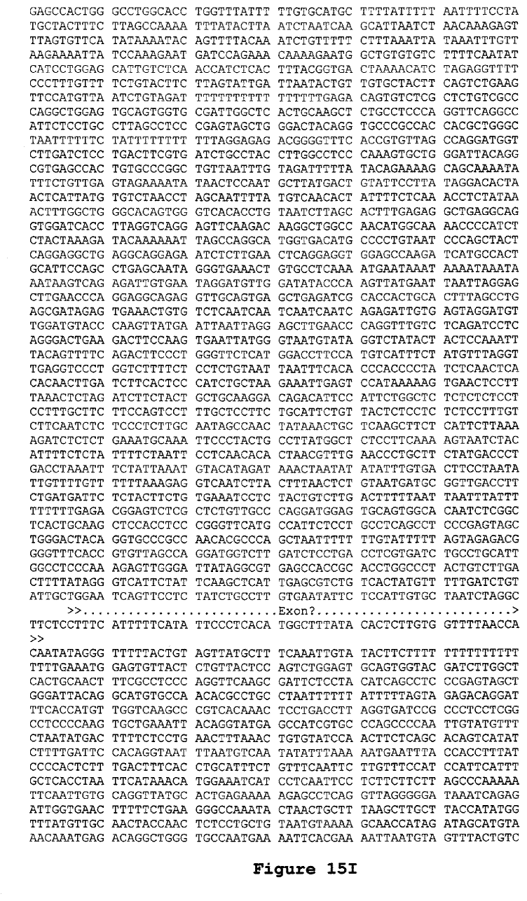

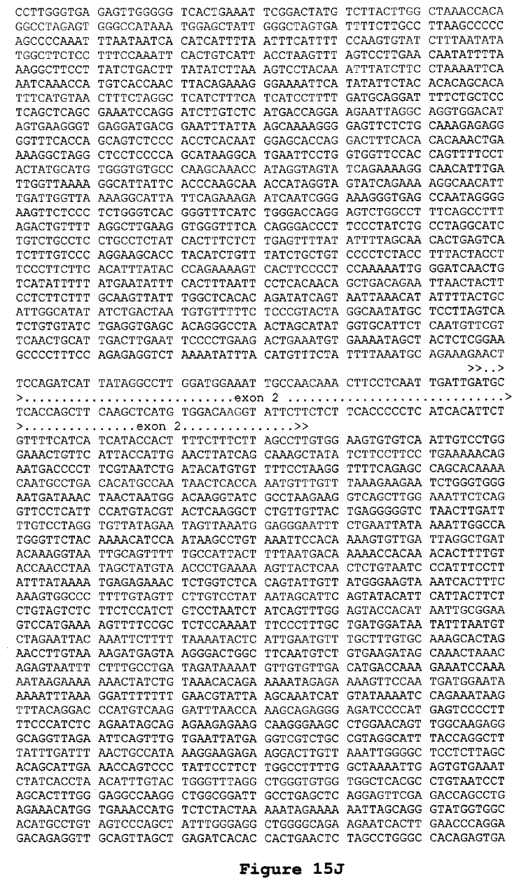

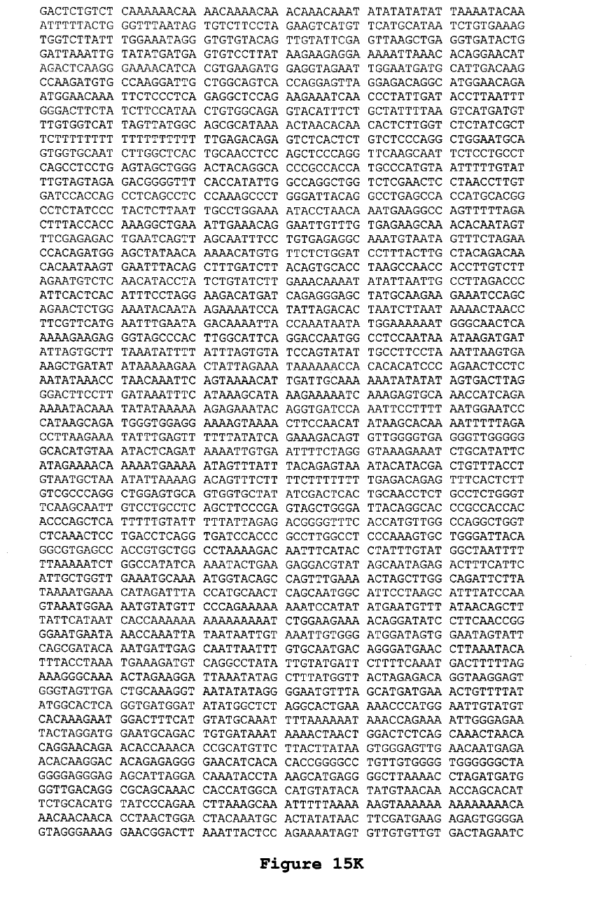

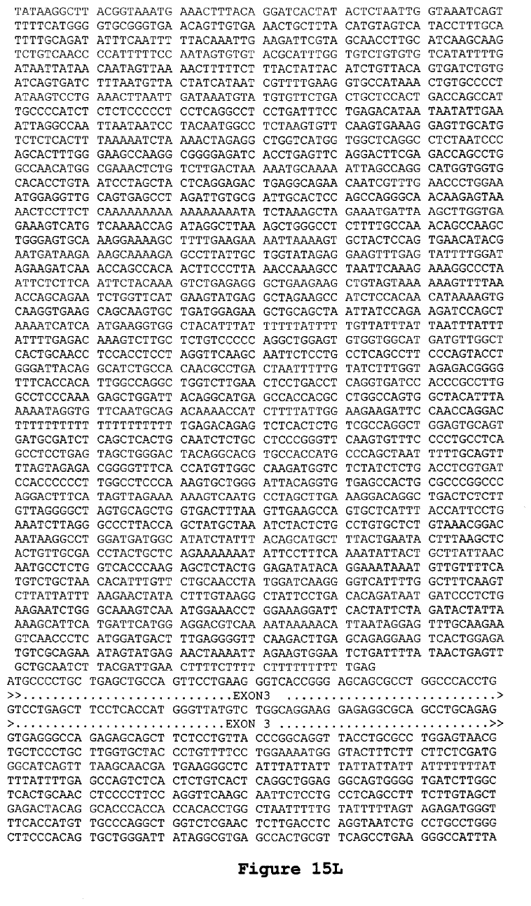

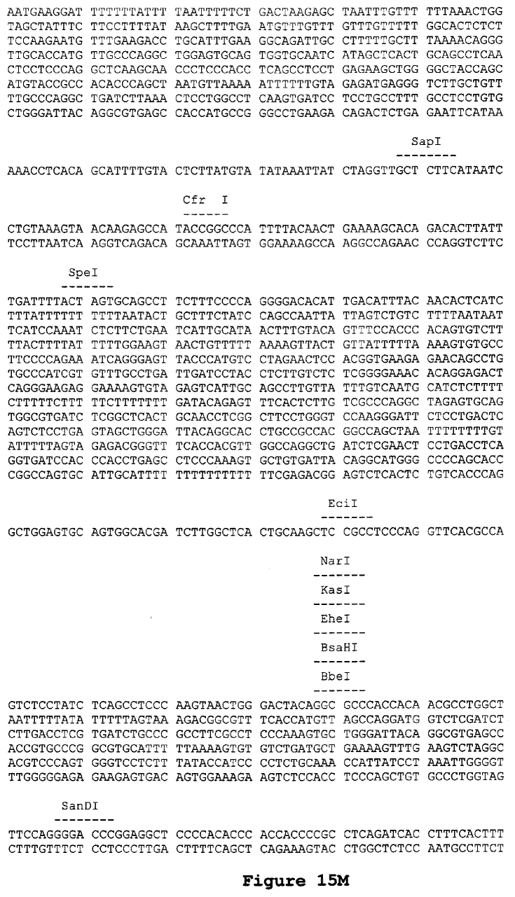

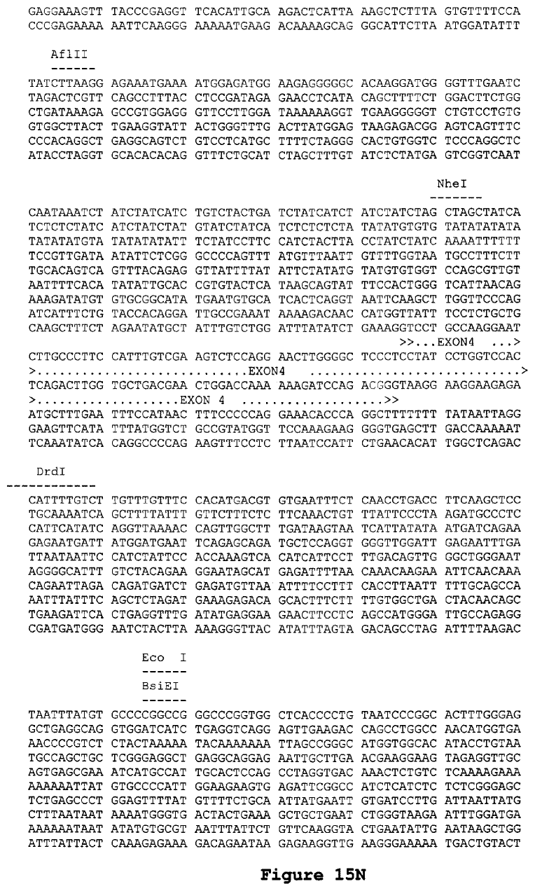

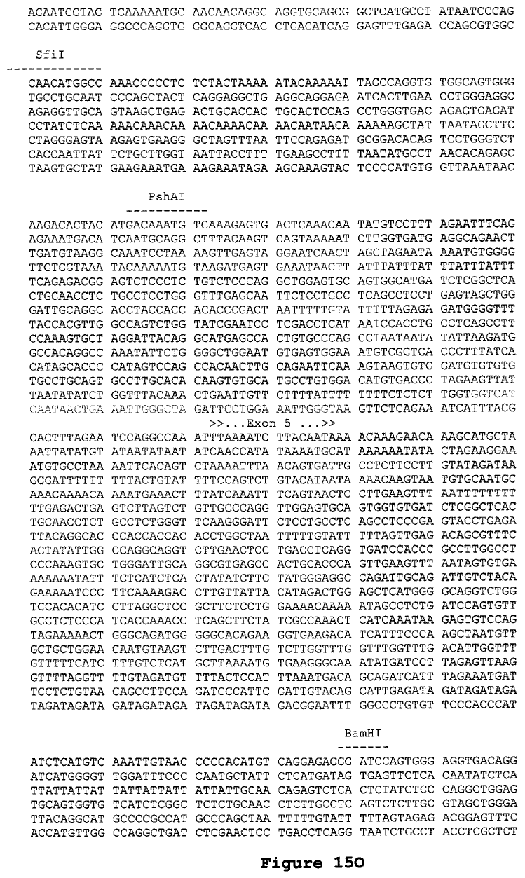

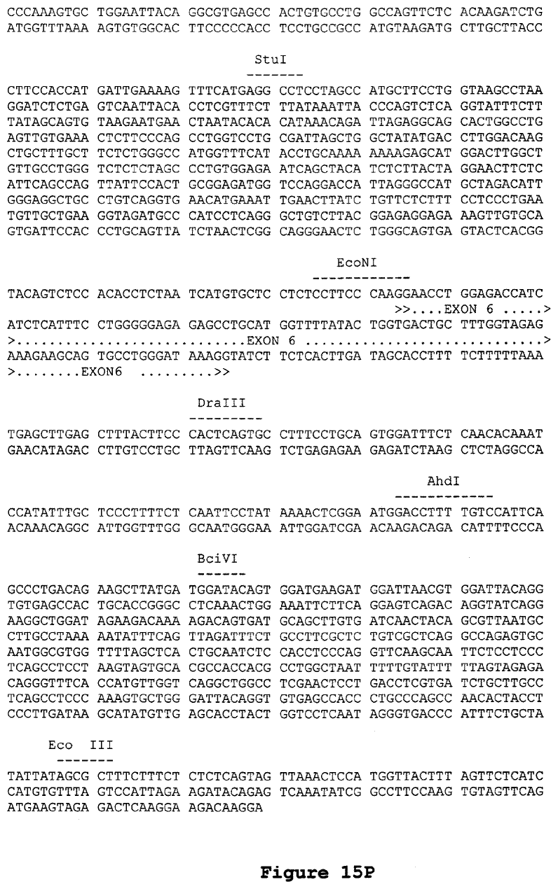

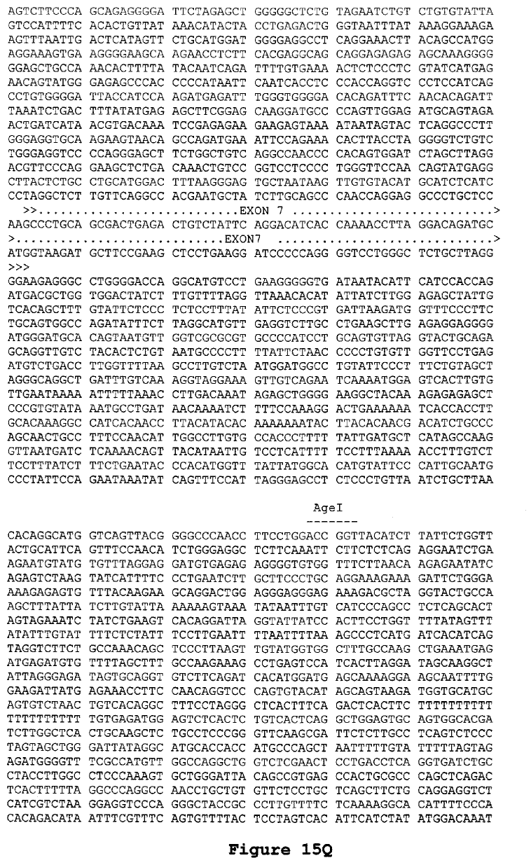

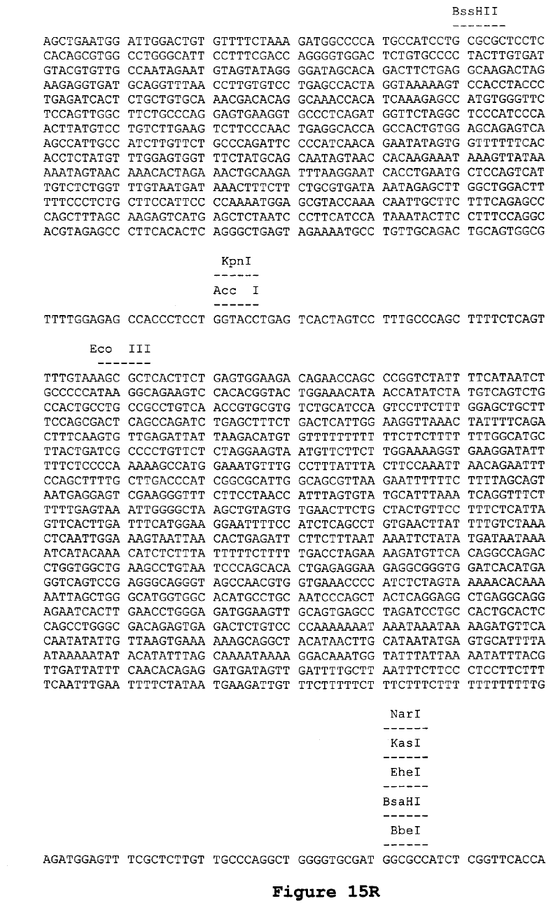

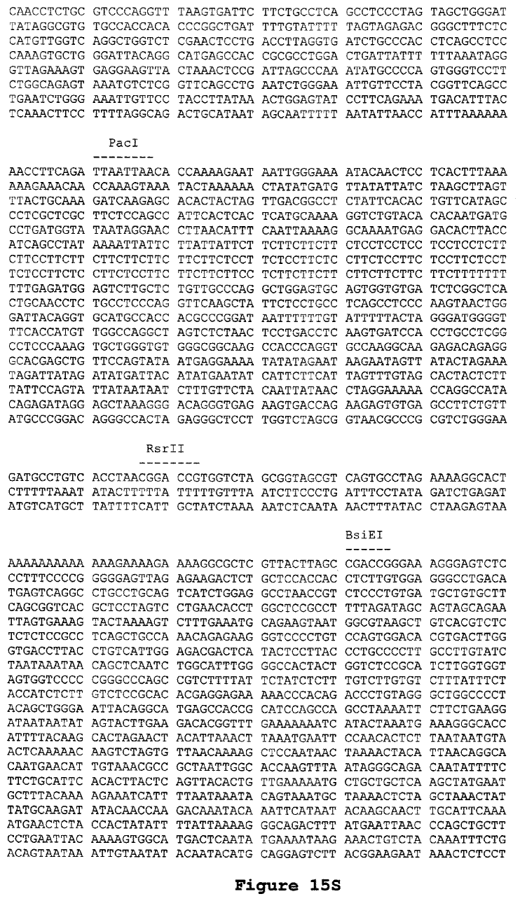

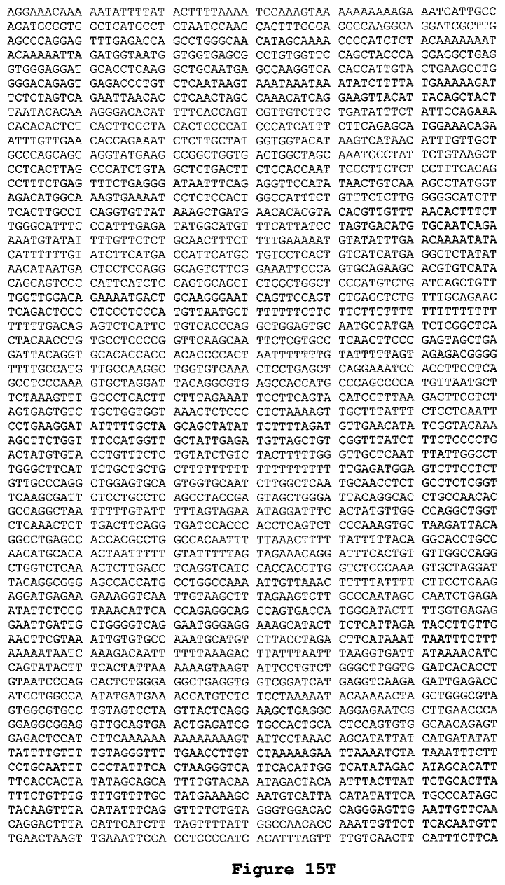

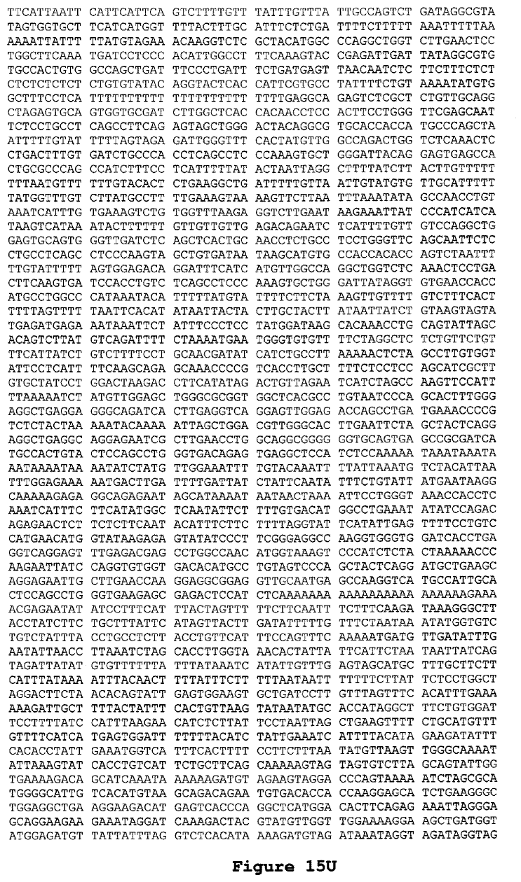

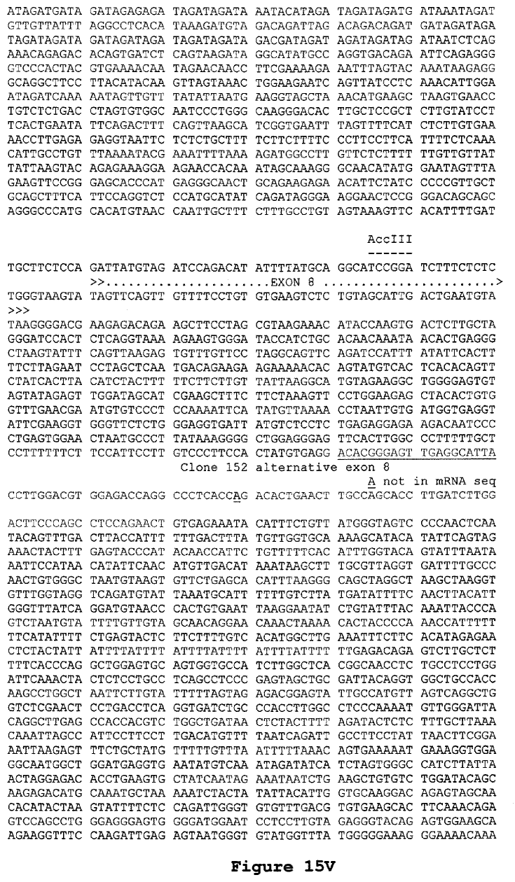

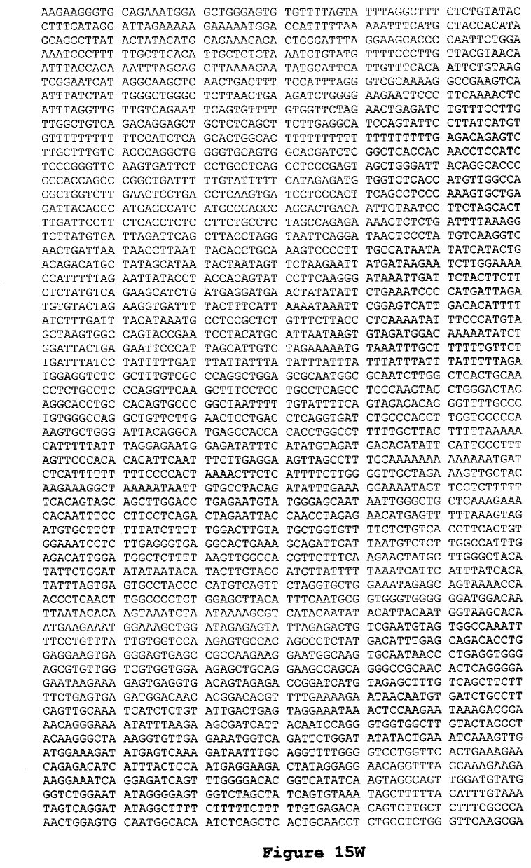

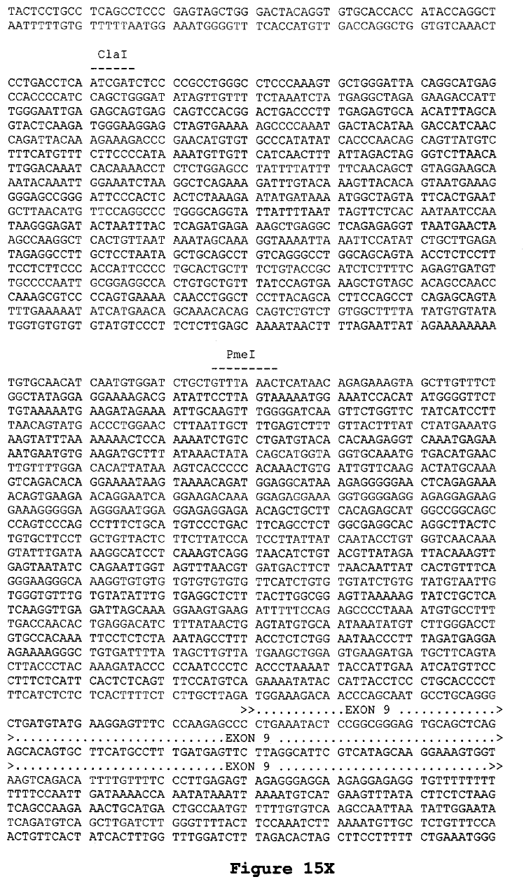

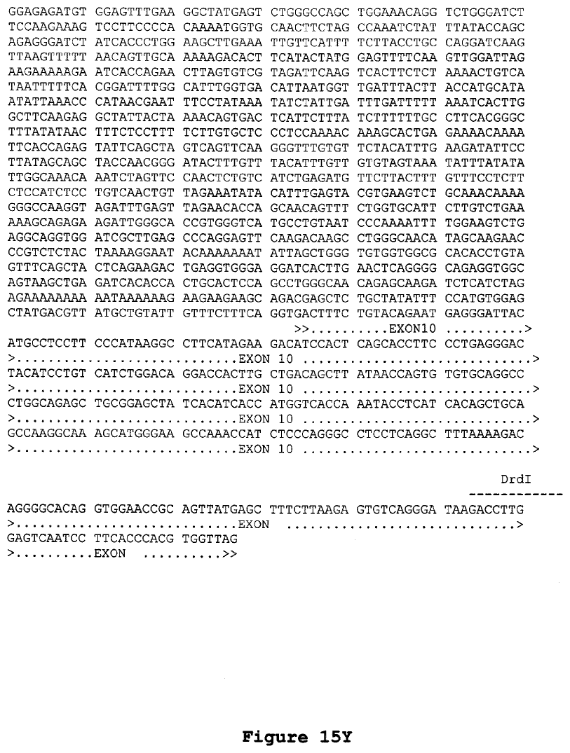

The instant invention also encompasses IDO2 nucleic acid molecules which lack at least one of the 11 exons depicted in FIGS. 13 and 15A-15Y. For example, the IDO2 nucleic acid molecule may lack any one of the eleven exons (e.g., IDO2 Δ1a, IDO2 Δ1, IDO2 Δ2, IDO2 Δ3, IDO2 Δ4, IDO2 Δ5, IDO2 Δ6, IDO2 Δ7, IDO2 Δ8, IDO2 Δ9, and IDO2 Δ10). The nucleic acid molecule may also lack 2, 3, 4, 5, 6, 7, 8, 9, or 10 exons, wherein the exons are either consecutive or nonconsecutive. In a particular embodiment, the IDO2 splice variants are selected from the group consisting of; IDO2 Δ1a, IDO2 Δ3/4/6, IDO2 Δ8, IDO2 Δ6/8, and IDO2 Δ4/5.

The instant invention also encompasses allelic variants and mutants of IDO2. Natural allelic variants of the instant invention include, without limitation, IDO2 R235W and IDO2 Y332stop.

In accordance with the present invention, nucleic acids having the appropriate level of sequence homology with a nucleic acid molecule encoding IDO2 may be identified by using hybridization and washing conditions of appropriate stringency.

Nucleic acids of the present invention may be maintained as DNA in any convenient vector. IDO2 encoding nucleic acid molecules of the invention include cDNA, genomic DNA, RNA, and fragments thereof which may be single- or double-stranded. Thus, this invention provides oligonucleotides having sequences capable of hybridizing with at least one sequence of a nucleic acid molecule of the present invention.

Also encompassed in the scope of the present invention are oligonucleotide probes which specifically hybridize with the IDO2 nucleic acid molecules of the invention. Primers capable of specifically amplifying IDO2 encoding nucleic acids described herein are also contemplated herein. As mentioned previously, such oligonucleotides are useful as probes and primers for detecting, isolating or amplifying altered IDO2 genes.

It will be appreciated by persons skilled in the art that variants (e.g., allelic variants) of IDO2 sequences exist, for example, in the human population, and must be taken into account when designing and/or utilizing oligonucleotides of the invention. Accordingly, it is within the scope of the present invention to encompass such variants, with respect to the IDO2 sequences disclosed herein or the oligonucleotides targeted to specific locations on the respective genes or RNA transcripts. Accordingly, the term “natural allelic variants” is used herein to refer to various specific nucleotide sequences of the invention and variants thereof that would occur in a human population. The usage of different wobble codons and genetic polymorphisms which give rise to conservative or neutral amino acid substitutions in the encoded protein are examples of such variants. Such variants would not demonstrate altered IDO2 activity or protein levels. Additionally, the term “substantially complementary” refers to oligonucleotide sequences that may not be perfectly matched to a target sequence, but such mismatches do not materially affect the ability of the oligonucleotide to hybridize with its target sequence under the conditions described.

The present invention also encompasses antisense nucleic acid molecules which may be targeted to translation initiation sites and/or splice sites to inhibit the expression of IDO2. Such antisense molecules are typically between about 15 and about 30 nucleotides in length and often span the translational start site of mRNA molecules. Antisense constructs may also be generated which contain the entire IDO2 sequence in reverse orientation. Antisense oligonucleotides targeted to any known nucleotide sequence can be prepared by oligonucleotide synthesis according to standard methods.

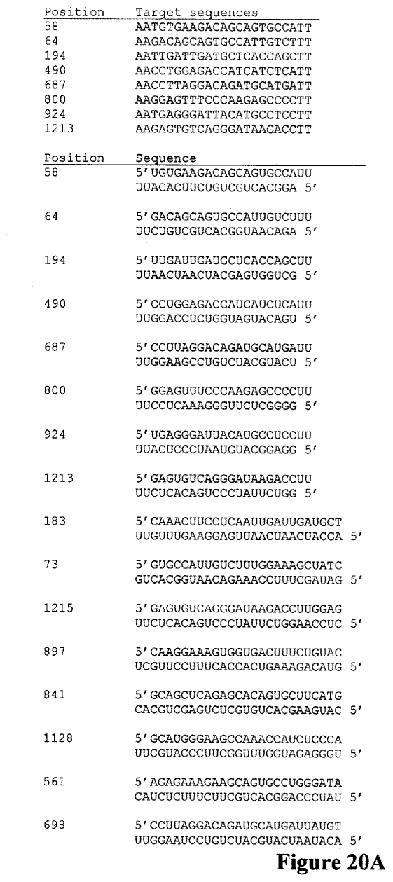

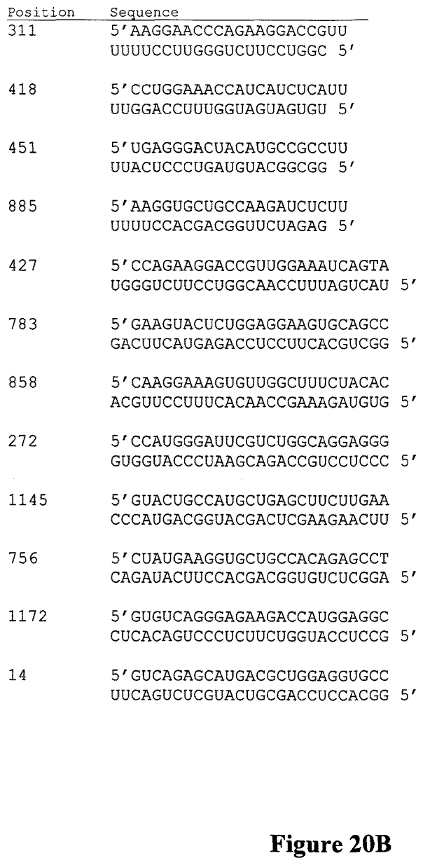

Small interfering RNA (siRNA) molecules designed to inhibit expression of IDO2 are also encompassed in the instant invention. Typically, siRNA molecules are double-stranded RNA molecules between about 12 and 30 nucleotides in length, more typically about 21 nucleotides in length (see Ausubel et al.). Exemplary siRNAs for human and mouse IDO2 are provided in FIGS. 20A and 20B, respectively.

Several methods of modifying oligodeoxyribonucleotides are known in the art. For example, methylphosphonate oligonucleotide analogs may be synthesized wherein the negative charge on the inter-nucleotide phosphate bridge is eliminated by replacing the negatively charged phosphate oxygen with a methyl group. See Uhlmann et al., Chemical Review, 90: 544-584 (1990). Another common modification, which is utilized in a preferred embodiment of the present invention, is the synthesis of oligodeoxyribonucleotide phosphorothioates. In these analogs, one of the phosphate oxygen atoms not involved in the phosphate bridge is replaced by a sulphur atom, resulting in the negative charge being distributed asymmetrically and located mainly on the sulphur atoms. When compared to unmodified oligonucleotides, oligonucleotide phosphorothioates are improved with respect to stability to nucleases, retention of solubility in water and stability to base-catalyzed hydrolysis. See Uhlmann et al., supra at 548-50; Cohen, J. S. (ed.) Oligodeoxynucleotides: Antisense Inhibitors of Gene Expression, CRC Press, Inc., Boca Raton, FL (1989).

Other modifications of oligodeoxyribonucleotides to produce stable, membrane permeable oligonucleotide analogs are commonly known in the art. For a review of such methods, see generally, Uhlmann et al., supra, and Cohen, supra which also describe methods for synthesis of such molecules. In addition, modified oligoribonucleotides may be utilized in the present invention. However, oligodeoxyribonucleotides are preferred due to their enhanced stability, ease of manufacture and the variety of methods available for analog synthesis.

Still other modifications of the oligonucleotides may include coupling sequences that code for RNase H to the antisense oligonucleotide. This enzyme (RNase H) will then hydrolyze the hybrid formed by the oligonucleotide and the specific targeted mRNA. Alkylating derivatives of oligonucleotides and derivatives containing lipophilic groups can also be used. Alkylating derivatives form covalent bonds with the mRNA, thereby inhibiting their ability to translate proteins. Lipophilic derivatives of oligonucleotides will increase their membrane permeability, thus enhancing penetration into tissue. Besides targeting the mRNAs, other antisense molecules can target the DNA, forming triple DNA helixes (DNA triplexes). Another strategy is to administer sense DNA strands which will bind to specific regulator cis or trans active protein elements on the DNA molecule.

Deoxynucleotide dithioates (phosphorodithioate DNA) may also be utilized in this invention. These compounds which have nucleoside-OPS₂O nucleoside linkages, are phosphorus achiral, anionic and are similar to natural DNA. They form duplexes with unmodified complementary DNA. They also activate RNase H and are resistant to nucleases, making them potentially useful as therapeutic agents. One such compound has been shown to inhibit HIV-1 reverse transcriptase (Caruthers et al., INSERM/NIH Conference on Antisense Oligonucleotides and Ribonuclease H, Arcachon, France 1992).

In accordance with the present invention, antisense oligonucleotides and siRNA may be produced by expression of DNA sequences cloned into plasmid or retroviral vectors. Using standard methodology known to those skilled in the art, it is possible to maintain the antisense RNA-encoding DNA in any convenient cloning vector (see Ausubel et al., eds. Current Protocols in Molecular Biology, John Wiley and Sons, Inc., (2005)).

Various genetic regulatory control elements may be incorporated into antisense RNA-encoding expression vectors to facilitate propagation in both eukaryotic and prokaryotic cells. Different promoters may be utilized to drive expression of the antisense sequences, the cytomegalovirus immediate early promoter being preferred as it promotes a high level of expression of downstream sequences. Polyadenylation signal sequences are also utilized to promote mRNA stability. Sequences preferred for use in the invention include, but are not limited to, bovine growth hormone polyadenylation signal sequences or thymidine kinase polyadenylation signal sequences. Antibiotic resistance markers are also included in these vectors to enable selection of transformed cells. These may include, for example, genes that confer hygromycin, neomycin or ampicillin resistance.

Cells and transgenic animals comprising a nucleic acid encoding IDO2 are also encompassed by the instant invention. The term “transgenic animal” is intended to include any non-human animal, preferably vertebrate, in which one or more of the cells of the animal contain heterologous/exogenous nucleic acid encoding IDO2, optionally from a different species tool encoding nucleic acids may also be inserted. Non-human animals include without limitation, rodents, mice, rats, non-human primates, sheep, dog, cow, amphibians, zebrafish, reptiles, and the like. In a preferred embodiment, the animal is a mouse. In another embodiment, the transgenic animal comprising a heterologous nucleic acid encoding IDO2 has modulated (e.g., increased) susceptibility to cancer and/or infection by microbes (e.g., viruses, bacteria, pathogens) compared to a wild-type mouse. In another embodiment, these transgenic animals exhibit at least one of increased LIP production, decrease in LAPs, decreased activation of GCN2, decreased elf2-α phosphorylation, and modulation of production of pro-inflammatory cytokines.

IDO2 knockout animals are also encompassed by the instant invention. Modifications and/or deletions may render the naturally occurring gene nonfunctional, thereby producing a “knock out” transgenic animal (e.g., IDO2^{-/-}). In other words, the transgenic mice comprise homozygous null mutations in the endogenous IDO2 gene. The transgenic animal of the instant invention may comprise a genome comprising a disruption of an endogenous IDO2 gene, wherein the IDO2 gene disruption leads to the failure to express IDO2 and/or a functional IDO2 (e.g., an IDO2 which lacks tryptophan catabolism activity and/or heme binding). The disruption of the endogenous IDO2 can be obtained through the disruption (e.g., insertion of a nucleic acid sequence (e.g., selectable marker) or deletion (e.g., all or part)) of at least one exon. In a particular embodiment, exon 1 is disrupted. In another embodiment, exon 9 and/or 10 is disrupted. Knock-in animals are also encompassed in the present invention as transgenic IDO2 animals. In one preferred embodiment, the IDO2 gene or exons of IDO2 gene are flanked by site specific recombinase recognition sequences such as Cre/LoxP. These mice are conditional is knock-out of IDO2 since the portion of the IDO2 gene flanked by the loxP sequences can be excised in vivo upon exposure to the site-specific recombinase Cre, provided into the genome of the offspring by crossing the knock-in IDO2 mouse with a transgenic mouse carrying the Cre recombinase under the control of a tissue-specific or inducible promoter.

The transgenic mouse may also comprise a knockout of the endogenous IDO1 gene. In a particular embodiment, the transgenic animal is devoid of IDO2 activity. In another embodiment, the transgenic animal has undetectable levels of IDO2. In yet another embodiment, the transgenic animal is fertile and transmits the homozygous null mutations to its offspring. In still another embodiment, the IDO2 knockout animal has modulated (e.g., decreased) susceptibility to cancer and/or infection by microbes (e.g., virus, bacteria, fungus, parasite, pathogen) compared to a wild-type animal. In another embodiment, the IDO2 knockout animals exhibit at least one of decreased LIP production, increase in LAPs, increased activation of GCN2, increased elf2-α phosphorylation, and modulation of production of pro-inflammatory cytokines.

Transgenic animals of the instant invention may be useful for the establishment of a nonhuman model for diseases involving improper expression and/or regulation of IDO2. The transgenic animals may also be useful as in vivo models for drug screening studies for certain human diseases, and for eventual treatment of disorders or diseases associated with IDO2, such as, without limitation, diseases that have an autoimmune component, diseases associated with improper IDO1 and/or IDO2 expression and/or regulation, and the like. In a particular embodiment, the course of microbial infection in mice devoid of IDO2 activity (i.e., knockout) or comprising a heterologous/exogenous nucleic acid encoding IDO2 may be assessed by a) inoculating the transgenic mouse with a microbe; b) inoculating a wild type mouse with the same microbe; and c) determining whether the course of infection in the transgenic mice is altered to that observed in the wild-type mice, thereby determining whether mice devoid of IDO2 or expressing heterologous/exogenous IDO2 are more/less/equally susceptible to infection by the microbe. In yet another embodiment, the susceptibility to cancer instead of a microbe can be determined by the above method.

### III. Proteins

IDO2 proteins of the present invention may be prepared in a variety of ways, according to known methods. The proteins may be purified from appropriate sources, e.g., transformed bacterial or animal cultured cells or tissues, by immunoaffinity purification. The availability of nucleic acid molecules encoding IDO2 protein enables production of the protein using in vitro expression methods and cell-free expression systems known in the art. In vitro transcription and translation systems are commercially available, e.g., from Promega Biotech (Madison, WI) or Gibco-BRL (Gaithersburg, MD).

Alternatively, larger quantities of IDO2 protein may be produced by expression in a suitable prokaryotic or eukaryotic system. For example, part or all of a DNA molecule encoding for IDO2 may be inserted into a plasmid vector adapted for expression in a bacterial cell, such as E. coli. Such vectors comprise the regulatory elements necessary for expression of the DNA in the host cell positioned in such a manner as to permit expression of the DNA in the host cell. Such regulatory elements required for expression include promoter sequences, transcription initiation sequences and, optionally, enhancer sequences.

IDO2 protein produced by gene expression in a recombinant prokaryotic or eukaryotic system may be purified according to methods known in the art. A commercially available expression/secretion system can be used, whereby the recombinant protein is expressed and thereafter secreted from the host cell, and readily purified from the surrounding medium. If expression/secretion vectors are not used, an alternative approach involves purifying the recombinant protein by affinity separation, such as by immunological interaction with antibodies that bind specifically to the recombinant protein or nickel columns for isolation of recombinant proteins tagged with 6-8 histidine residues at their N-terminus or C-terminus. Alternative tags may comprise the FLAG epitope or the hemagglutinin epitope. Such methods are commonly used by skilled practitioners.

IDO2 protein of the invention, prepared by the aforementioned methods, may be analyzed according to standard procedures. For example, such protein may be subjected to amino acid sequence analysis, according to known methods.

The instant invention encompasses the amino acid sequences encoded by the nucleic acid molecules described herein. Exemplary amino acid sequences of human IDO2 are SEQ ID NO; 1, 2, and 29 and exemplary amino acid sequences of murine IDO2 are SEQ ID NO: 3 and 4. An IDO2 amino acid sequence may have 75%, 80%, 85%, 90%, 95%, 97%, or 99% homology with these sequences.

### IV. Antibodies

The present invention also encompasses antibodies capable of immunospecifically binding to proteins of the invention. Specifically, the antibodies may specifically bind to IDO2 to the exclusion of IDO1. Alternatively, antibodies can be generated which recognize both IDO2 and IDO1. The antibodies may also be generated to bind IDO2 splice variants and mutants of the instant invention either in addition to IDO2 and/or IDO1 or to the exclusion of other IDO2 proteins. Polyclonal antibodies directed toward IDO2 protein and variants thereof may be prepared according to standard methods. In a particular embodiment, monoclonal antibodies are prepared, which react immunospecifically with the various epitopes of the IDO2 protein. Monoclonal antibodies may be prepared according to general methods known in the art. For example, polyclonal and monoclonal antibodies may be prepared as described in Current Protocols in Molecular Biology, Ausubel et al. eds. Antibodies may be prepared by chemical cross-linking, hybrid hybridoma techniques and/or by expression of recombinant antibody fragments expressed in host cells, such as bacteria or yeast cells. The antibody molecules may then be isolated and purified from the expression system. The antibodies optionally comprise a purification tag by which the antibody can be purified.

The purity of the antibody molecules of the invention may be assessed using standard methods known to those of skill in the art, including, but not limited to, ELISA, immunohistochemistry, ion-exchange chromatography, affinity chromatography, immobilized metal affinity chromatography (IMAC), size exclusion chromatography, polyacrylamide gel electrophoresis (PAGE), western blotting, surface plasmon resonance and mass spectroscopy.

Polyclonal or monoclonal antibodies that immunospecifically interact with IDO2 proteins can be utilized for identifying and purifying such proteins. For example, antibodies may be utilized for affinity separation of proteins with which they immunospecifically interact. Antibodies may also be used to immunoprecipitate proteins from a sample containing a mixture of proteins and other biological molecules.

Additionally, target-specific antibodies, optionally selected by a functional assay, may be isolated. The crystal structure may then be obtained for the antibody. This structure yields a pharmacore upon which subsequent drug design can be based. Alternatively, the protein crystallography may be bypassed altogether by generating anti-idiotypic antibodies (anti-ids) to a functional, pharmacologically active antibody. As a mirror image of a mirror image, the binding site of the anti-ids would be expected to be an analog of the original molecule. The anti-id could then be used to identify and isolate peptides from banks of chemically or biologically produced banks of peptides. Selected peptides would then act as the pharmacore.

### V. Therapeutic Uses

The present invention also encompasses methods for tumor suppression. Specifically, a therapeutically effective amount of at least one IDO2 inhibitor (optionally one which does not inhibit IDO1) can be administered to a patient, in need thereof, for the treatment of cancer. Cancers that may be treated using the present protocol include, but are not limited to: prostate cancers, colorectum, pancreas, cervix, stomach, endometrium, brain, liver, bladder, ovary, testis, head, neck, skin (including melanoma and basal carcinoma), mesothelial lining, white blood cell (including lymphoma and leukemia) esophagus, breast, muscle, connective tissue, lung (including small-cell lung carcinoma and non-small-cell carcinoma), adrenal gland, thyroid, kidney, or bone; glioblastoma, mesothelioma, renal cell carcinoma, gastric carcinoma, sarcoma, choriocarcinoma, cutaneous basocellular carcinoma, and testicular seminoma.

The at least one IDO2 inhibitor (optionally one which does not inhibit IDO1) may also be administered in combination with at least one signal transduction inhibitor (STI), as described, in the context of IDO inhibitors, in PCT/US04/05155 and PCT/US04/05154 and U.S. patent application Ser. Nos. 10/550,444 and 10/551,151, with at least one chemotherapeutic agent, and/or at least one immunomodulator, as described in U.S. patent application Ser. Nos. 10/550,444 and 10/551,151.

The present invention also encompasses pharmaceutical compositions comprising at least one of the IDO2 inhibitor(s) (optionally one which does not inhibit IDO1) in a pharmaceutically acceptable carrier. Such a pharmaceutical composition may be administered, in a therapeutically effective amount, to a patient in need thereof for the treatment of cancer. The pharmaceutical compositions may further comprise at least one STI, at least one chemotherapeutic agent, and/or at least one immunomodulator.

The present invention also encompasses methods for the treatment of chronic viral infections (see U.S. patent application Ser. Nos. 10/550,444 and 10/551,151). Specifically, a therapeutically effective amount of at least one IDO2 inhibitor (optionally one which does not inhibit IDO1) can be administered to a patient having a chronic viral infection. Additionally, at least one chemotherapeutic agent and/or at least one antiviral agent may be co-administered with the at least one IDO2 inhibitor.

Chronic viral infections that may be treated using the present combinatorial treatment include, but are not limited to, diseases caused by: hepatitis C virus (HCV), human papilloma virus (HPV), cytomegalovirus (CMV), herpes simplex virus (HSV), Epstein-Barr virus (EBV), varicella zoster virus, coxsackie virus, and human immunodeficiency virus (HIV). Notably, parasitic infections (e.g. malaria) may also be treated by the above methods wherein compounds known to treat the parasitic conditions may also be co-administered.

Suitable antiviral agents include, without limitation: acyclovir; gangcyclovir; foscarnet; ribavirin, and antiretrovirals such as, for example, nucleoside analogue reverse transcriptase inhibitors (e.g., azidothymidine (AZT), ddl, ddC, 3TC, d4T), non-nucleoside reverse transcriptase inhibitors (e.g., efavirenz, nevirapine), nucleotide analogue reverse transcriptase inhibitors, and protease inhibitors.

The present invention also encompasses pharmaceutical compositions comprising at least one of the IDO2 inhibitor (optionally one which does not inhibit IDO1) in a pharmaceutically acceptable carrier for the treatment of chronic viral infections. Such a pharmaceutical composition may be administered, in a therapeutically effective amount, to a patient in need thereof for the treatment of a chronic viral infection. The pharmaceutical compositions may further comprise at least one chemotherapeutic agent and/or at least one antiviral agent.

The pharmaceutical compositions of the present invention can be administered by any suitable route, for example, by injection, by oral, pulmonary, nasal or other modes of administration. In general, pharmaceutical compositions of the present invention, comprise, among other things, pharmaceutically acceptable diluents, preservatives, solubilizers, emulsifiers, adjuvants and/or carriers. Such compositions can include diluents of various buffer content (e.g., Tris-HCl, acetate, phosphate), pH and ionic strength; and additives such as detergents and solubilizing agents (e.g., Tween 80, Polysorbate 80), anti-oxidants (e.g., ascorbic acid, sodium metabisulfite), preservatives (e.g., Thimersol, benzyl alcohol) and bulking substances (e.g., lactose, mannitol). The compositions can be incorporated into particulate preparations of polymeric compounds such as polylactic acid, polyglycolic acid, etc., or into liposomes. Such compositions may influence the physical state, stability, rate of in vivo release, and rate of in vivo clearance of components of a pharmaceutical composition of the present invention. See, e.g., Remington's Pharmaceutical Sciences, 18th Ed. (1990, Mack Publishing Co., Easton, PA 18042) pages 1435-1712 which are herein incorporated by reference. The pharmaceutical composition of the present invention can be prepared, for example, in liquid form, or can be in dried powder form (e.g., lyophilized).

In yet another embodiment, the pharmaceutical compositions of the present invention can be delivered in a controlled release system, such as using an intravenous infusion, an implantable osmotic pump, a transdermal patch, liposomes, or other modes of administration. In a particular embodiment, a pump may be used (see Langer, supra: Sefton, CRC Crit. Ref. Biomed. Eng. (1987) 14:201; Buchwald et al., Surgery (1980) 88:507; Saudek et al., N. Engl. J. Med. (1989) 321:574). In another embodiment, polymeric materials may be employed (see Medical Applications of Controlled Release, Langer and Wise (ads.), CRC Press: Boca Raton, Florida (1974); Controlled Drug Bioavailability, Drug Product Design and Performance, Smolen and Ball (eds.), Wiley: New York (1984); Ranger and Peppas, J. Macromol. Sci. Rev. Macromol. Chem. (1983) 23:61: see also Levy et al., Science (1985) 228:190; During et al., Ann. Neurol. (1989) 25:351; Howard et al., J. Neurosurg. (1989) 71:105). In yet another embodiment, a controlled release system can be placed in proximity of the target tissues of the animal, thus requiring only a fraction of the systemic dose (see, e.g., Goodson, in Medical Applications of Controlled Release, supra, (1984) vol. 2, pp. 115-138). In particular, a controlled release device can be introduced into an animal in proximity to the site of inappropriate immune activation or a tumor. Other controlled release systems are discussed in the review by Langer (Science (1990) 249:1527-1533).

IDO2 may also be used to tolerize the immune system for a desired antigen. The IDO2 may be delivered as a nucleic acid molecule and/or a protein, optionally in a pharmaceutically acceptable carrier. The IDO2 may be administered to antigen presenting cells with the desired antigen in vivo or in vitro. For example, the antigen presenting cells, optionally obtained from the patient to be treated, can be treated ex vivo and then administered to the patient. These methods may be used to treat, for example, diabetes, lupus, Myastenia Gravis, Crohn's disease, and other autoimmune disorders.

IDO2 inhibitors as described hereinabove may also be used to alter the immune system to become active against a particular antigen. The antigen may be “foreign”, e.g., viral, bacterial, and tumor specific antigens. Alternatively, the antigen may be a “self” antigen, e.g., amyloid, scrappie prions, and other misfolded proteins. The administration of the IDO2 inhibitors to patients would helpful in treating disorders/diseases associated with the particular antigen. The IDO2 inhibitors may be administered to APCs in vivo, in vitro, or ex vivo. The APCs may be loaded with the antigen of interest.

IDO2 and IDO2 inhibitors may also be administered to patients in need thereof to modulate the production of pro-inflammatory cytokines (e.g., IL6, TNFα, CD14, IL-1β, IL-S, IL-12, MCP1, m-CSF, G-CSF, and iNOS). IDO2 inhibitors would decrease LIP production, thereby leading to an apparent increase in LAPs, which regulate pro-inflammatory cytokines. IDO2, administered as a nucleic acid molecule and/or protein, or agents which augment IDO2 expression levels or activity would increase LIP activity, thereby causing a decrease in LAP activities.

IDO2 may also be involved in the production of proteins possessing a modified tryptophan amino acid. Specifically, IDO2 may generate kynurenine tRNAs in place of tryptophan tRNAs. As such, kynurenine may be incorporated into a peptide. The ability to incorporate a modified amino acid into a protein implicates IDO2 as an epigenetic post transcriptional modifier of gene function. The modifications by IDO2 may relate not only to normal biological function but also encompass modifications which affect disease states. For example, modified or misfolded proteins due to the incorporation of IDO2 altered amino acids may be causative factors in several diseases or disorders including, without limitation, cancer, immune disease, Alzheimer's, prion disorders, metabolic disorders, cardiovascular disease, Parkinson's, Huntington's, age-related disorders, and neurological disorders. Indeed, it has been previously demonstrated that the nitrosylation of tryptophan in beta amyloid results in the misfolding of the protein and abnormal amyloid production. Similarly, the incorporation of kynurenine into a protein may provide a “normal” biological role in altering protein function. However, under chronic conditions brought about by over-expression or over-activity of IDO2 (such as a chronic inflammation), the incorporation kynurenine may lead to “abnormal” gene products and/or expression which could alter critical factors controlling cell growth, differentiation, and function. Accordingly, the administration of IDO2 inhibitors to patients having disorders or diseases associated with proteins altered by IDO2 may be therapeutic. In order to determine if IDO2 inhibition would be therapeutic for a particular disease, the proteins associated with the disease may be screened for the presence of kynurenine and/or screened to determine if the protein is misfolded.

In addition to the IDO2 inhibitors described hereinabove, the splice variants and mutants of the instant invention may be employed to inhibit IDO2 and/or IDO1 activity in a subject. As described hereinbelow in Example 2, IDO2 splice variants and mutants (e.g., natural allelic variants R235W and Y332stop) can inhibit the activity of IDO2. Accordingly, IDO2 splice variants and mutants and/or nucleic acid molecules encoding the IDO2 splice variants and mutants may be administered to a subject in accordance with the methods described hereinabove as an IDO2 inhibitor.

### VI. Screening Methods

The biochemistry of IDO has been well established, the enzyme having first been isolated in 1963 (Higuchi, K., et al. (1963) Federation Proc. 22:243 (abstr.); Shimizu, T., et al. (1978) J. Biol. Chem. 253:4700-6). IDO is a monomeric, heme-containing oxidoreductase with a molecular weight of approximately 41 kDa. To maintain the active ferrous form during in vitro catalysis, the enzyme requires methylene blue in combination with either superoxide or a reductant such as ascorbic acid. In vivo, it is suggested that a flavin or tetrahydrobiopterin may fulfill the role of the methylene blue dye and that there is likely to be a specific site for noncompetitive IDO inhibitors. Active enzyme can be produced by expressing the cloned, His-tagged version of the mammalian gene in bacteria (Littlejohn, T. K., et al. (2000) Prot. Exp. Purif. 19:22-29). This provides a convenient source of enzyme for biochemical analysis. A conventional biochemical assay for IDO activity based on spectaphotometric measurement of the production of kynurenine (the hydrolysis product of N-formyl-kynurenine) from tryptophan (Daubener, W., et al. (1994) J. Immunol. Methods 168:39-47) may be used as the read-out for both the enzymatic and cell-based assays. An enzymatic assay provides a facile, high-throughput screen for identifying compounds with IDO inhibitory activity, This assay may also be used to determine Ki values for specific compounds, which is important for the development of SAR (structure activity relationship) around the different compound series. A cell-based assay can confirm the IDO inhibitory activity of identified compounds and address the initial issue of bioavailability—the ability of compounds to inhibit intracellular IDO. Specificity for IDO inhibition may be examined in the cell-based assay by comparing against the other known tryptophan catabolizing enzyme tryptophan dioxygenase (IDO, also referred to in the literature as IDO2).

Because of the homology between IDO1 and IDO2, methods employed for screening for inhibitors of IDO1 activity may be employed for screening compounds which modulate IDO2 activity of the IDO2 proteins described herein. Indeed, as demonstrated hereinbelow, IDO2 can produce kynurenine from tryptophan, thereby allowing the above enzyme assays to be employed with IDO2. Inhibitors may be specific for IDO2 or may effectively inhibit both IDO1 and IDO2. For example, D-1MT does not inhibit IDO1, but is an inhibitor of IDO2 activity. IDO inhibitors, as described hereinabove, may also be screened for their ability to inhibit IDO2. While the above describes the screening of compounds to identify inhibitors of IDO2, the screening assays can be employed to screen for modulators of IDO2 activity, e.g., for compounds that increase IDO2 expression levels or activity and/or compounds which decrease IDO2 activity.

In accordance with the present invention, the nucleotide sequences of both human and mouse IDO2 have been determined. For biochemical studies, IDO2 protein, optionally tagged with a purification tag which allows the isolation of the protein, may be expressed and isolated from an expression system. For example, C-terminal His-tagged IDO2 protein may be produced in E. coli using the IPTG-inducible pET5a vector system and isolated over a nickel column. The yield of the partially purified protein can be verified by gel electrophoresis and the concentration estimated by comparison to protein standards. To assay IDO2 enzymatic activity, a 96-well plate spectraphotometric assay for kynurenine production may be run following published procedures (Littlejohn, T. K., et al. (2000) Prot. Exp. Purif. 19:22-29; Takikawa, O., et al. (1988) J. Biol. Chem. 263:2041-8; see also U.S. patent application Ser. Nos. 10/550,444 and 10/551,151).

With regard to cell-based assays, cells, e.g., COS-1 cells and 293 cells, may be transfected (e.g., transiently transfected) with a plasmid expressing IDO2 (see Munn et al. (1999) J. Exp. Med. 189:1363-1372 for an exemplary method). Optionally, a companion set of cells is transfected with a TDO or IDO expressing plasmid. After transfection, the cells are contacted with the test compound. Subsequently, the supernatant can be spectrophotometrically assayed for kynurenine as described for the enzyme assay.

The following examples are provided to illustrate various embodiments of the present invention. The examples are illustrative and are not intended to limit the invention in any way.

### Example 1

### Reagents

Blasticidin (Invitrogen; Carlsbad, CA) was prepared as a 1000× solution at 5 mg/mL in sterile water and stored in 500 μL aliquots frozen at −20° C. Zeocin (Zeo; Invitrogen) was prepared as 100 mg/mL aliquots and stored frozen at −20° C.

Doxycycline (Dox; Sigma, St. Louis, MO) was prepared as a 20 mg/mL master stock in ethanol and stored frozen at −20° C. The working 100× stock (2 μg/mL) was prepared by diluting the master stock 1:10,000 in growth media. The working stock was stored in 500 μL aliquots and stored frozen and in the dark at −20° C.

1-Methyl-D-Tryptophan (1M-D-T; Sigma-Aldrich Cat #45283) was solubilized in DMSO/0.1 N HCL as a 100 mM stock solution and stored in 100 μL aliquots at −20° C. 1-Methyl-L-Tryptophan (1M-L-T; Sigma-Aldrich Cat #447439) was solubilized in DMSO/0.1 N HCL as a 100 mM stock solution and stored in 100 μL aliquots at −20° C. 1-Methyl-DL-Tryptophan (1M-DL-T; Sigma-Aldrich Cat #860646) was solubilized in DMSO/0.1. N HCL as a 100 mM stock solution and stored in 100 μL aliquots at −20° C. 5-(indol-3-methyl)-3-methyl-2-thio-Hydantoin (MTH; Sigma-Aldrich Cat #M6006) was solubilized in DMSO as a 100 mM stock solution and stored in 100 μL aliquots at −20° C. L-Tryptophan (Tryp; Sigma-Aldrich Cat #T0254) was solubilized in DMSO as a 100 mM stock solution and stored in 100 μL aliquots frozen at −20° C.

Dulbecco's Modified Eagle Medium (DMEM) was obtained from Mediatech, Inc. (Herndon, VA; Cat #10-013-CV). Alpha minimum essential medium (α-MEM) was obtained from Invitrogen and contains ribonucleosides and deoxynucleosides/4 mM glutamax/1 mM sodium pyruvate and was stored at 4° C. Trypsin EDTA (1×) was obtained from Mediatech (Cat #25-053-CI) 10 mL aliquots were stored frozen at −20° C. until use. Fetal bovine serum (FBS) was obtained from Hyclone (Logan, UT; Cat 4 SV30014.03) and 50 mL aliquots were stored frozen at −20° C. until use. Penicillin/streptomycin 100×(Pen/Strep) was obtained from Mediatech (Cat #30-002-C) which contained 10,000 I.U. Penicillin/mL and 10,000 μg/mL Streptomycin. L-Glutamine (L-glut) was obtained from Mediatech (Cat #25-005-CV) as a 200 mM solution (29.23 mg/mL with 8.5 g/L NaCl). Optimem was obtained from Invitrogen (Cat #31985-062) and stored in 10 mL aliquots at 4° C.

Antibodies to the phosporylated eIF2α (anti-eIF2α-P (ser52)) were affinity purified. Rabbit polyclonal antibodies raised against the short amino acid sequence containing the phosphorylated Ser-52 (Santa Cruz Biotechnology, Santa Cruz, CA, sc-12412). For Western blotting, anti-eIF2α-P was diluted at 1:200 in blocking buffer (PBS/0.14 tween-20, 3% w/v non-fat dry milk, phosphatase inhibitor cocktail (Calbiochem, La Jolla, CA, Cat #524625)).

Antibodies to the c-terminal region of C/EBPβ were affinity purified rabbit polyclonal antibodies raised against a peptide corresponding to the C-terminus of C/EBPβ of rat origin (Santa Cruz Biotechnology, sc-150). For Western blotting anti-C/EBPβ was diluted at 1:200 in blocking buffer (PBS/0.1% tween-20, 3% w/v non-fat dry milk).

Anti-V5-Horse radish peroxidase (HRP) conjugated antibody was purchased from Invitrogen and stored at 4° C. For Western blotting anti-V5-HRP was diluted at 1:5000 in blocking buffer (PBS/0.1% tween-20, 0.3% w/v non-fat dry milk). Affinity purified goat anti-rabbit IgG antibody and affinity purified horse anti-mouse IgG antibody conjugated to HRP (Cell Signaling technologies, Danvers, MA, cat #7074 and #7076, respectively) were typically diluted 1:1000 in blocking buffer (PBS/0.1% tween-20, 3% w/v non-fat dry milk).

GM-CSF was stored frozen at −20° C. as a 1000× solution (5 mg/mL). Interferon-γ (IFN-γ), IL-10, and lipopolysaccharide (LPS) were stored frozen at −20° C. as a 1000× solution.

### Methods

Western Analysis: Cell lysates were prepared as follows: Cells were removed from the dish by scrapping or pipetting in PBS and collected by centrifugation (2000 rpm/5 minutes). The supernatant was discarded and the cell pellet was lysed in RIPA buffer [50 mM Tris-HCl pH 7.4/150 mM NaCl/l % Triton x-100/1% Sodium deoxycholate/0.1% SDS] plus a protease inhibitor cocktail (Calbiochem Cat #539134). Typically 5×10⁵ cells were resuspended in 200 μL of RIPA buffer. Cells lysates were vortexed and incubated on ice or frozen at −20° C. until use. Prior to use lysates were clarified by centrifugation (10,000 rpm/15 minutes) and heat denatured at 95° C. in SDS/sample buffer (100 mM Tris/25% glycerol/2% SDS/0.01% bromophenol blue/25 mM DTT). Proteins were separated by PAGE using NuPAGE® Gels (Invitrogen) and transferred at 100 Volts on to Immobilon-NC Nitrocellulose membranes (Millipore, Billerica, MA, Cat #HAHY00010) in transfer buffer (25 mM Tris pH 8.3/192 mM glycine/20% methanol). The protein containing membranes were typically blocked at room temperature for 1 hour in blocking buffer (PBS/0.1% tween-20, 3% w/v non-fat dry milk). Primary antibody was added to the blot in blocking buffer at the suggested dilution, incubated by shaking at room temperature for 1 hour or at 4° C. for 12-18 hours and washed 5-6 times in PBS/0.1% Tween. For secondary antibody applications, the secondary antibody containing HRP was added in blocking buffer at the appropriate dilution, incubated by shaking at room temperature for 1 hour, and washed 5-6 times in PBS/0.1% Tween. The western membranes were developed by Chemiluminescence using SuperSignal® West Pico substrate (Pierce, Rockford, IL, Cat #34078), exposed to autography film for an appropriate period of time (3 seconds to 18 hours), and developed.

Kynurenine detection assay: This assay measures the product of the indoleamine 2,3-dioxygenase conversion of L-tryptophan to N-formyl-kynurenine by acid hydrolysis to kynurenine, which is measured indirectly though the absorbance (480 nm) of imine produced by the reaction of the aromatic amino group of klynurenine with p-dimethylaminobenzaldehyde (Ehrlich's reagent) as described by Takikawa et al. (J. Biol. Chem. (1988) 263:2041-2048). Typically, 200 μL of media from the test sample is combined with 12.5 μL of 30% TCA and incubated at 50° C. for 30 minutes. Evaporation was avoided by sealing the rim of the plate or tubes with parafilm. Samples are clarified by centrifugation for 10 minutes at 3-10K rpm. 100 μL of the supernatant was removed to a fresh well in an assay plate and mixed with 100 μL of fresh Ehrlich's reagent (2% p-dimethylaminobenzaldehyde w/v with glacial acetic acid). The plates were incubated for 10-30 minutes at room temperature and the absorbance was read at 490 nm. Typically, assays were performed in triplicate and the data analyzed using Excel software program (Microsoft). Background/control samples were typically derived from media from uninduced cells or cells that lack IDO1/2 expression vectors. Typically, control values were averaged and subtracted from the sample values.

Surforhodamine B colorimeter assay: This assay is used to assess cell density and is based on the measurement of cellular protein content and performed as described Vichai and Kirtikara (Nature Protocols (2006) 1:1112). For a 96 well dish, sample wells were maintained in 200 μL of media and fixed by adding 100 μL of cold 10% (wt/v) TCA to each well and the plates were incubated at 4° C. for at least 1 hour. The supernatant may be removed and saved for Kynurenine detection assay. The plates are then washed four times by dipping the plate slowly into a 2 liter beaker of tap water. The wash is removed by gently flicking or blotting the plate. The water in the 2 liter beaker is changed after each wash. Any excess water was blotted and the plates were air dried. Plates can be stored indefinitely at room temperature at this stage. For analysis, 100 μL of 0.057% (wt/v) Surforhodamine B (SRB)/1% acetic acid (v/v) solution was added and the plates incubated at room temperature for 30 minutes. Plates were then rinsed four times in 1% acetic acid by dipping in a 2 L beaker of wash solution, as performed with the first wash. The plates were then allowed to air dry in an exhaust hood. Sample wells were then developed by adding 200 μL of 10 mM Tris base (pH 10.5), incubating at room temperature for 30 minutes with gentle shaking, and measuring the optical density (O.D.) at 510 nm. Typically samples were performed in triplicate and the data analyzed using Excel software program (Microsoft).

Cell Culture: 293-T-REx™ cells (Invitrogen) constitutively express a tet operator repressor protein and were maintained in DMEM/10% FBS/1×Pen/Strep/2 mM L-glut/blasticidin (5 ng/mL) at 37° C. with a 5% CO₂ in air atmosphere and typically split prior to confluency. Cells were passaged by splitting the culture 1/10—by removing media by aspiration, washing 1× with PBS, incubating with 0.254 trypsin/EDTA until the cells detach, disbursing the cells in fresh growth media, and plating at 1/10 dilutions in fresh growth media. For long term cryopreservation, cells are detached from the plate as described above, collected by centrifugation, resuspended in freeze medium (growth medium/10% DMSO), stored in 1.8 mL cyropreservation vials (˜2-5×10⁶ cells per vial) in liquid nitrogen vapor storage tanks.

JawsII (ATCC CRL-11904) is an immortalized mouse dendritic cell line derived from the bone marrow of p53 knock-out Blk/6 mouse (U.S. Pat. No. 5,648,219). Cells were maintained in DMEM/10% FBS supplemented with GM-CSF at 5 ng/mL and grown at 37° C. with a 5% CO₂ in air atmosphere. For maintenance floating cells are removed by pipetting. Detached cells were removed by rinsing in 0.25% trypsin/EDTA and pooled with the floating cells. Cells were collected by centrifugation (1000 rpm 1 minute) and resuspended in fresh media, and subcultured at a 1:2 ratio. For long term cryopreservation, floating and adherent cells were detached and pooled from the plate as described above, collected by centrifugation, resuspended in freeze medium (growth medium/5% DMSO), stored in 1.8 mL cyropreservation vials (˜2-5×10⁶ cells per vial) in liquid nitrogen vapor storage tanks.

Transfection. Typically, T-REx™ cells were transfected by lipofection whereby complexes are formed by mixing 1.6 μg of circular plasmid and 4 μL Lipofectamine 2000 (Invitrogen) in a final volume of 400 μL Optimem, as per protocol. These complexes were then added directly to expanding cultures of cells for a total of 6 to 20 hours. After 24-48 hours, cells were either split and placed in media and selection antibiotic (typically 100 ug/mL Zeo) or induced with Dox for transient expression.

RNA preparation: Total RNA was prepared from tissue and cultured cells using PureLink™ Micro-to-Midi Total RNA Purification System (Invitrogen, Cat #12183-018). For RNA preparation from tissue, the tissue (˜10 mg) was cut into small pieces and mixed with 500 μL of kit lysis buffer and passaged through a 21 G needle in lysis buffer several times until the lysate was well homogenized. Then the lystae was passaged over the spin homogenizer column (Invitrogen). The supernatants were then processed as standard lysates. Briefly 1 volume of 70% Ethanol was added to the lysates, mixed well by vortexing, and loaded on to the RNA spin column, as described. Following the first wash, the samples were treated with RNase-free Dnase-I on the column as described in the protocol. The RNA was eluted in nuclease-free water and immediately stored at −80° C. The concentration of RNA was determined spectrophotometrically (O.D. at 260 nm).

Reverse Transcription: c-DNA was prepared from total RNA that was reverse transcribed using ThermoScript™ RT-PCR system (Invitrogen) according to the manufacturer's protocol. Briefly, 1-2 μL of RNA ranging from 0.01 to 1 μg of total RNA was mixed with either a specific reverse strand primer (10 pm) or a random hexamer (50 ng), 1 mM dUTP, 1×cDNA synthesis buffer, 20 mM DTT/40 U RNaseOUT™, and 15 units of ThermoScript™ RT in a 20 μL volume. Reaction conditions varied, but typically the reaction was heated to 55° C. for 2 minutes and incubated at 52° C. for the specific primer for 60 minutes and terminated by heating at 85° C. for 5 minutes. For random priming the reactions were incubated at 25° C. for 20 minutes and then 50 minutes at 50° C.

PCR: Double stranded molecules were generated by PCR amplification under the appropriate cycling conditions (typically, 2 minute denaturation at 95° C.; followed by 30 cycles of 30 seconds at 95° C. (denaturation); 30 seconds at 55° C. (annealing); and 30 seconds at 72° C. (extension); and a final 2 minute extension at 72° C.). Typically, 50 μL reaction mixes contained template DNA (0.2-100 ng), 10 pmoles of each primer flanking the target sequence, 0.2 mM of each dNTPs, appropriate MgCl₂ concentrations (1-2.5 mM), and 3-4 units of Tag Polymerase. For cloning, PCR products were typically purified using a commercially available PCR product clean-up procedure (Promega or Qiagen) and eluted in sterile dH₂O.

### Results

The complete open reading frames (ORF) of the murine and human IDO2 mRNA have been delineated and confirmed by sequence analysis by RT-PCR. The exons corresponding to the ORF have been mapped by comparison to the genomic sequence in the GenBank and Celera Databases.

Both IDO1 and IDO2 are present on chromosome 8. The distance between the coding regions varies between the sequences available for human and mouse. In the mouse, the distance between the last exon of IDO1 and the first exon of IDO2 is about 4500 bases. The distance between the two genes, as determined from the available human genomic region, is about 20,000 bases.

Amino acid sequences of human and murine IDO2 are provided in FIGS. 1 and 2, respectively. Coding regions of human and murine IDO2 are provided in FIGS. 4A and 4B, respectively. FIGS. 3A, 3C, and 3D provide sequence alignments of human IDO1 and IDO2 amino acid sequences, human and murine IDO2 amino acid sequences, and murine IDO1 and IDO2 nucleotide and amino acid sequences, respectively.

The complete coding regions of murine IDO1 and IDO2 were cloned in-frame with the V5/His TAG coding region in pcDNA4/TO (Invitrogen) which contains a regulatory region that is repressed by the tetracyclin regulated repressor (TetR). The complete coding regions of IDO1 and IDO2 were generated using gene specific primers [IDO2 5′KpnATG (TCCGGTACCATGGAGCCTCAAAGTCAG, SEQ ID NO: 16) and IDO1 5′KpnATG (ATCCGGTACCATGGCACTCAGTAAAATA, SEQ ID NO: 17)] and 3′ primers that were gene specific and removed the termination codon [IDO2minstop (TGTCCTGGTGCTAAGGGTCAAGACAATTCT, SEQ ID NO: 18) and IDO1 minus stop (TGAGTTGGCCTAAGGGTCAAGACAATTCT, SEQ ID NO: 19)]. The IDO1 and 2 coding regions where then cloned in-frame with the V5/His carboxyl terminal Tag in the pcDNA4 TO vector (Invitrogen).

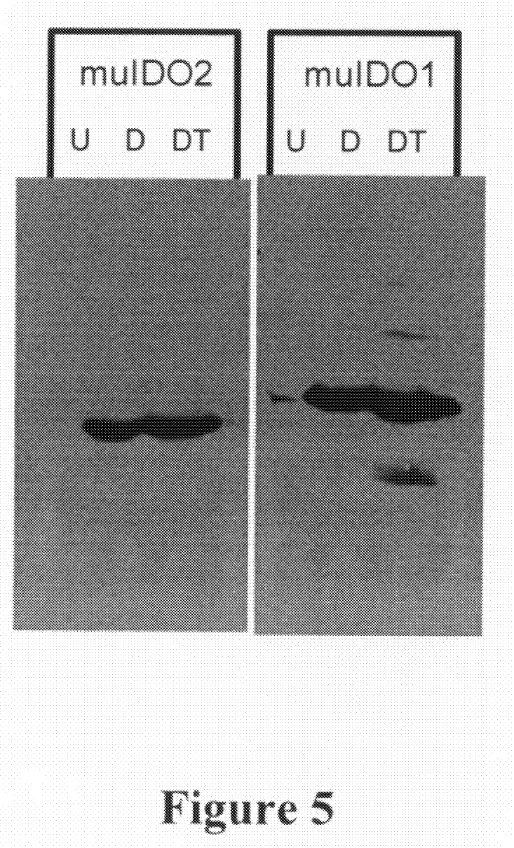

Murine IDO1 and IDO2 were expressed as fusions with a C-terminal V5 epitope. Both were detected by Western blot analysis in T-REx™-293 cells following the addition of doxycycline (2 μg/mL) or doxycycline combined with tryptophan (100 μM) (FIG. 5). Little or no IDO1 or IDO2 was detected in the absence of doxycycline. IDO2 migrates slightly faster (apparent lower molecule weight) than IDO1. Both proteins were detected using an HRP-conjugated antibody to the V5 C-terminal tag (Invitrogen). Bound antibody was detected by chemiluminescence (SuperSignal (West Pico, Pierce).

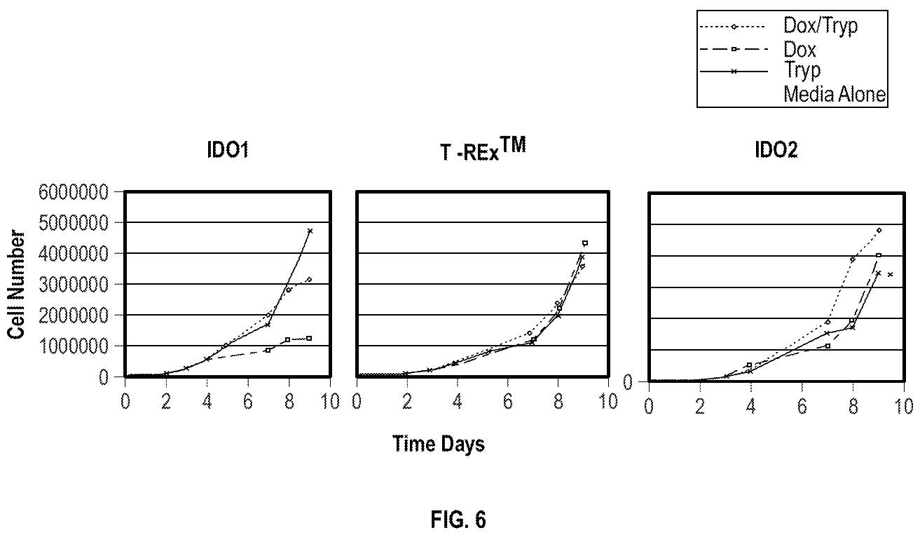

The growth characteristics of T-REx™ expressing IDO1 and IDO2 were also examined. FIG. 6 shows that growth curves of the T-REx™ cells with no vector (center panel), pcDNA4TO-muIDO1 (left panel) or pcDNA4TO-muIDO2 (right panel). Cells were grown unsupplemented (media alone), or in the presence of doxycycline (Dox-20 ng/mL), tryptophan (Tryp-100 μM) or both doxycycline and tryptophan (Dox/Tryp). All cells grew normally in the absence of doxycycline (media alone) or in the presence of additional Tryptophan (100 μM). In the presence of doxycycline (20 ng/mL) the IDO1 expressing cells grow less well due to deprivation of tryptophan from the media. IDO1 expressing cells in the presence of doxycycline and tryptophan grew well until day 8-10. IDO2 expressing cells consistently grew better in the presence of doxycycline and tryptophan. For IDO2 containing cells, no significant changes in cell growth was observed following doxycycline or tryptophan additions alone. Similarly the growth characteristic of T-REx™ cells with no vector was unaffected by the doxycycline, tryptophan or dox/tryp treatments.

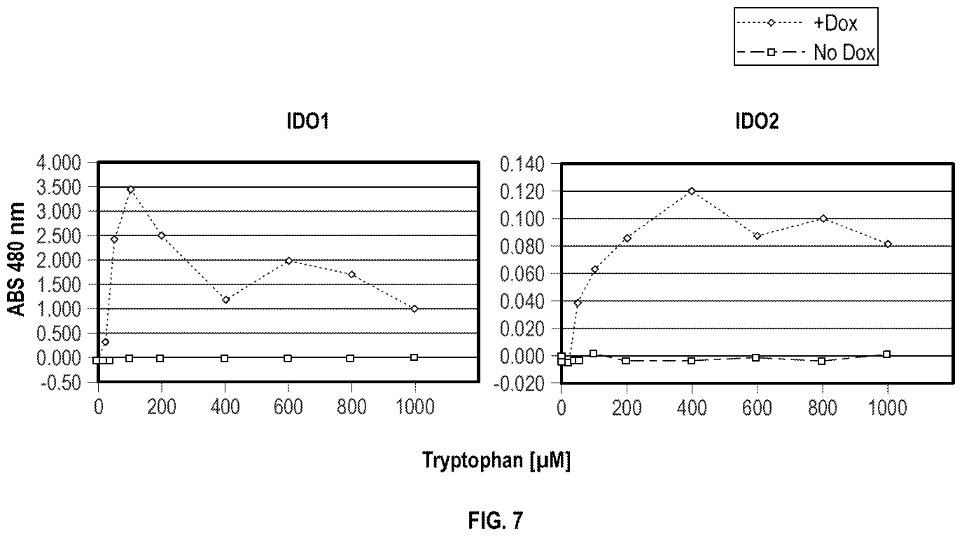

The ability of IDO1 and IDO2 expressing 293 cells to metabolize tryptophan was also studied (FIG. 7). The kynurenine assay indicates that tryptophan is metabolized in IDO1 and IDO2 expressing cells. The assay was performed in triplicate on media harvested 72 hours in the absence of induction (no doxycycline) or following induction with doxycycline (20 ng/mL). Cells were also exposed to increasing amounts of supplemental tryptophan to the media (0 to 1000 μM). Significantly higher levels of kynurenine were detected in both IDO1 and IDO2 expressing cells when the tryptophan levels were higher than media alone. No significant kynurenine was detected in IDO2 expressing cells in the absence of supplemental tryptophan. These data suggest that IDO1 and IDO2 possess indoleamine dioxygenase activity. However the activity of IDO2 is far lower than that of IDO1 (note the abs scale is different between IDO1 and IDO2 experiments). Taken together these data suggest the IDO2, although possessing weak indoleamine dioxygenase activity, may have a different and preferred substrate than IDO1. Potential substrates of IDO2 include, without limitation, hydroxyl tryptophan, serotonin, melatonin, indoleacrylate, methyl-tryptamine, and indole-3-propanoate. Alternatively, IDO2 may require a co-factor for its full activation that is different that that present for IDO1.

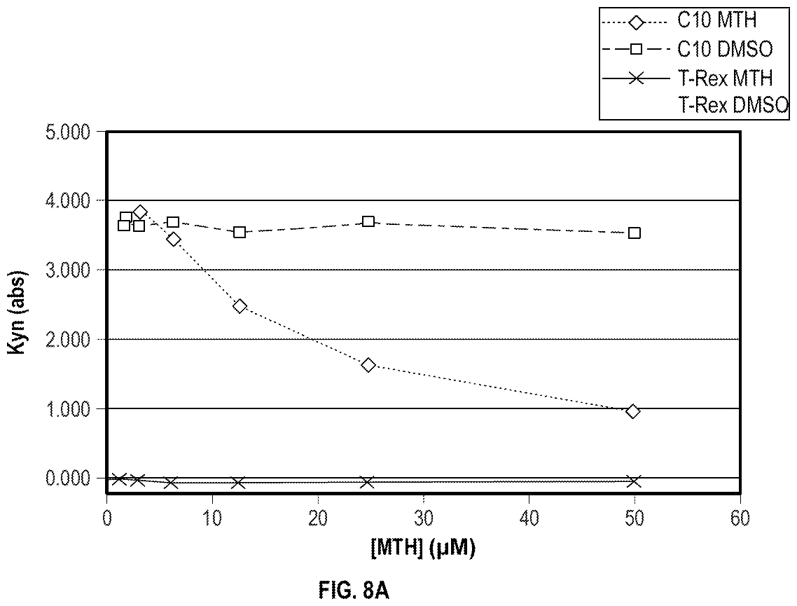

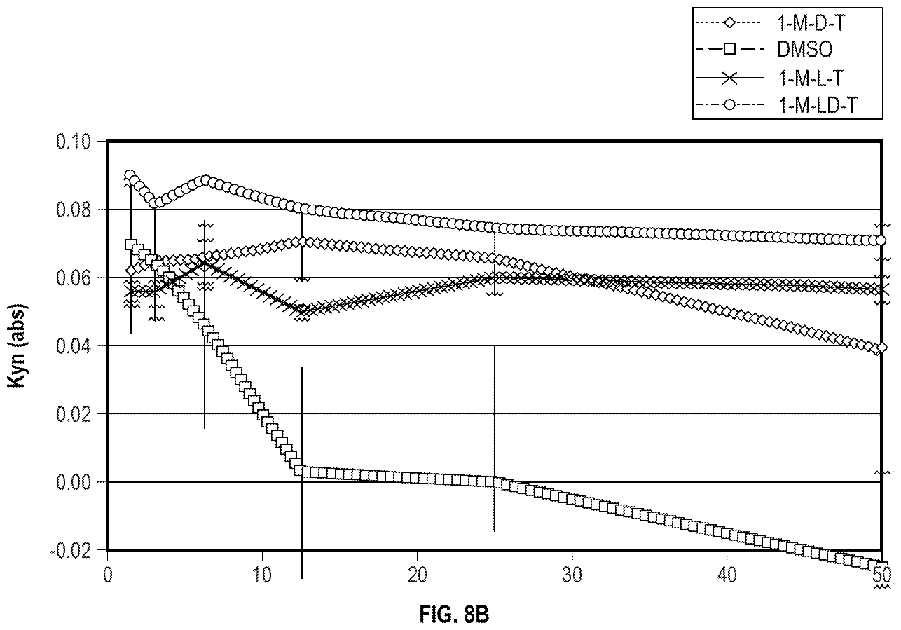

IDO1 expressing 293 cells metabolize tryptophan and can be inhibited by methyl thiohydantione (MTH). T-REx™ cells or IDO1 expressing T-REx™ cells were treated with doxycycline in the presence of increasing concentrations of DMSO (carrier) or known IDO1 inhibitor (MTH). The cells were maintained in growth media plus supplements for 5 days, after which the media was harvested and subjected to Analysis for the presence of kynurenine. The decrease in kynurenine production in the IDO1 expressing cells was observed in a dose dependent manner while no changes in kynurenne were observed in DMSO only treated cells (FIG. 8A). The control T-REx™ cells (no vector) do not produce any detectable kynurenine. The presence of inhibitor was not toxic to the cells. These data demonstrate the ability to use this cell based assay to assess indoleamine dioxygenase activity of IDO1 by using a known IDO1 inhibitor.

IDO2 expressing 293 cells metabolize tryptophan and can be inhibited by 1-methyl-D-tryptophan (1M-D-T). IDO2 expressing T-REx™ cells were treated with doxycycline in the presence of increasing concentrations of DMSO (carrier) or a variety of potential inhibitors, including the D, L and mixed DL racemic isomers of 1 methyl-tryptophan, (1M-D-T, 1M-L-T and 1M-LD-T, respectively) (FIG. 8B). The cells were maintained in growth media plus supplements for 5 days, after which the media was harvested and subjected to analysis for the presence of kynurenine. The decrease in kynurenine production in the IDO2 expressing cells was observed only with 1M-D-T in a dose dependent manner while no changes in kynurenine levels was observed in DMSO only treated cells or the other inhibitors. The control T-REx™ cells (no vector) do not produce any detectable kynurenine and were used as background absorbance values which were subtracted from the experimentally obtained values. The presence of inhibitor was not toxic to the cells.

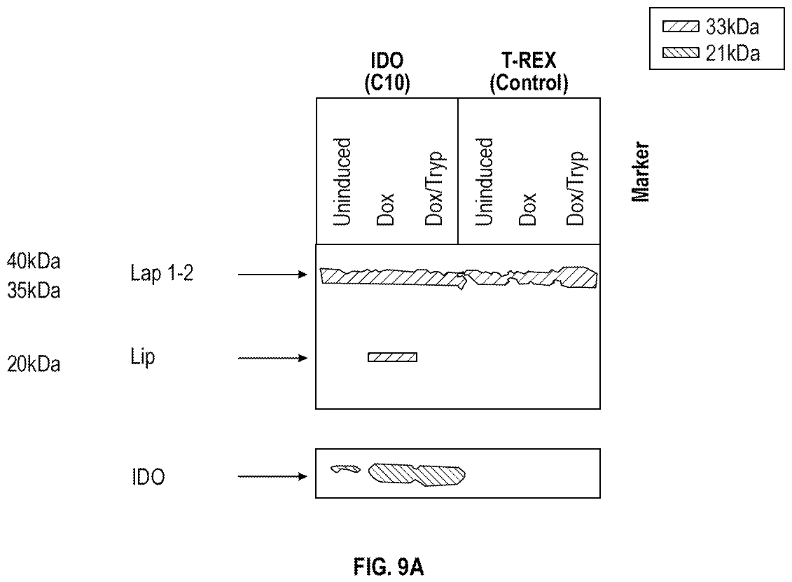

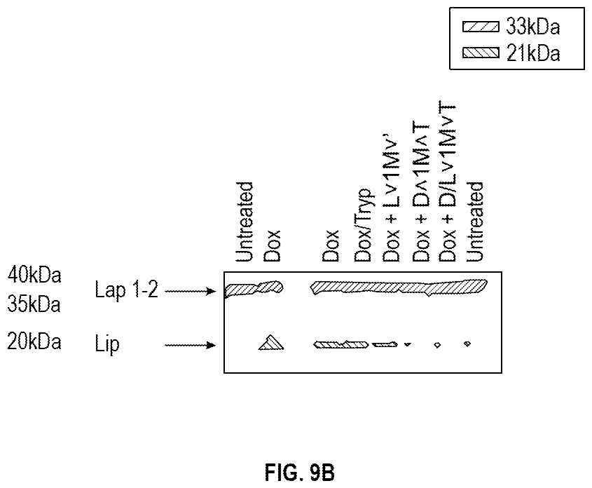

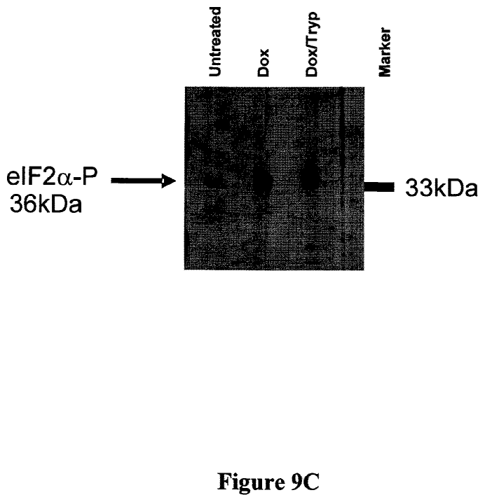

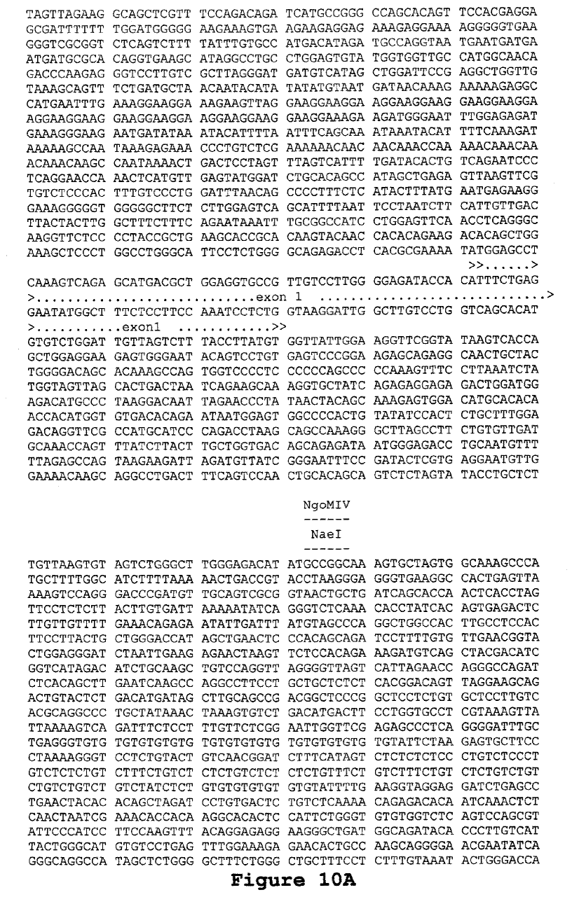

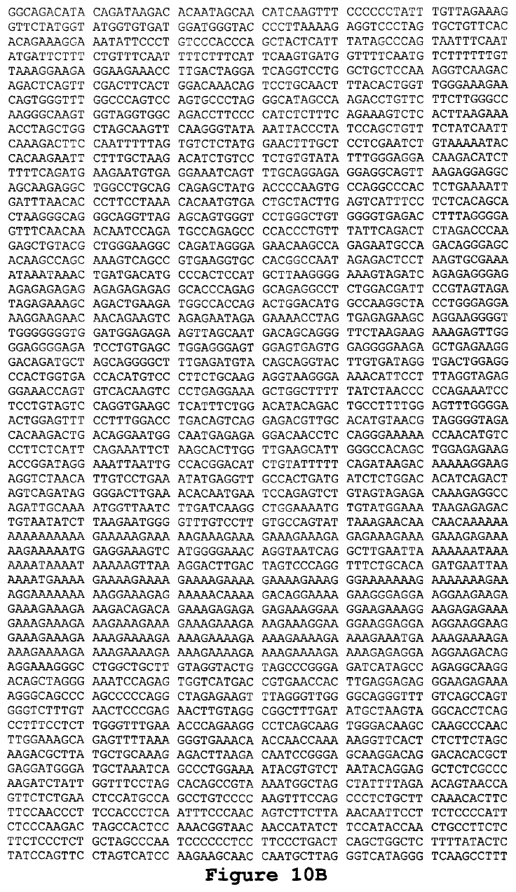

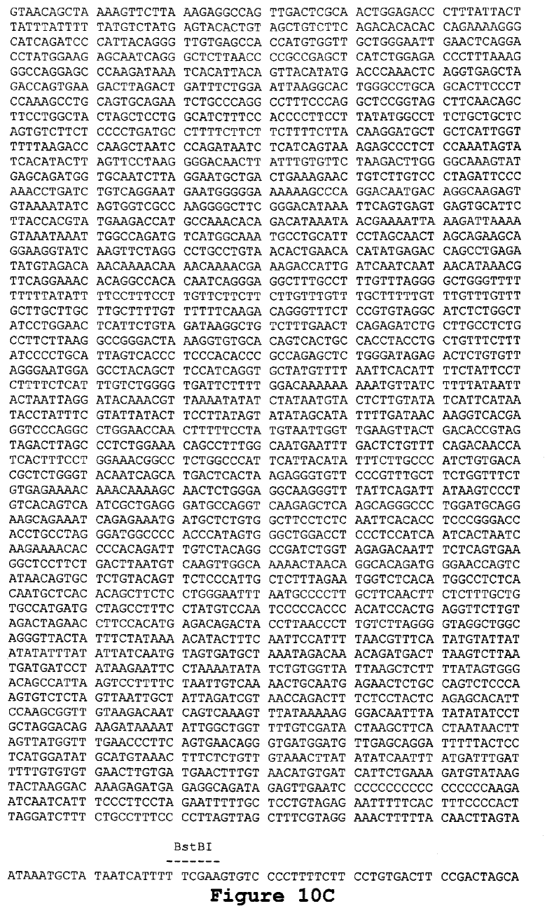

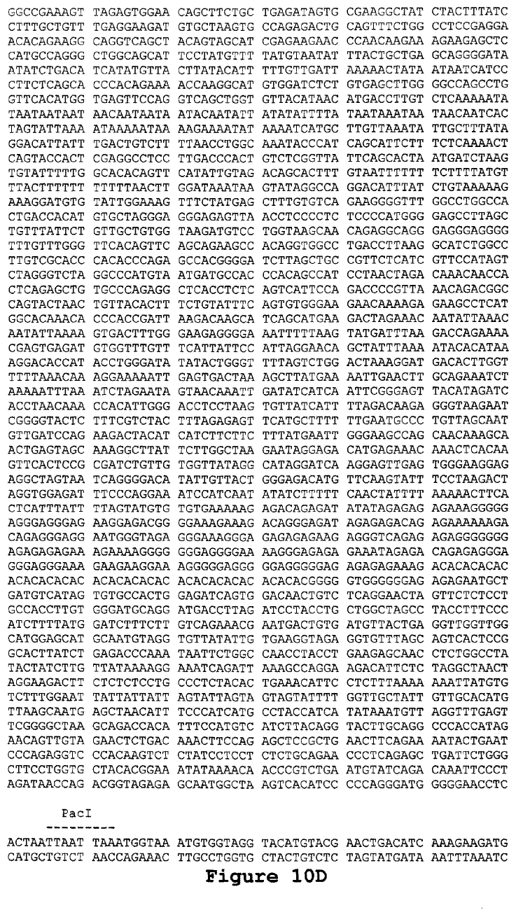

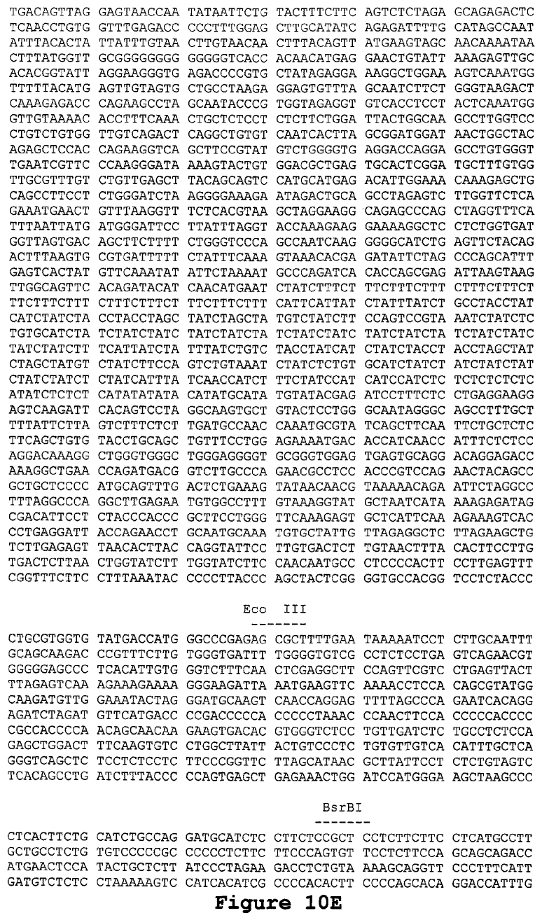

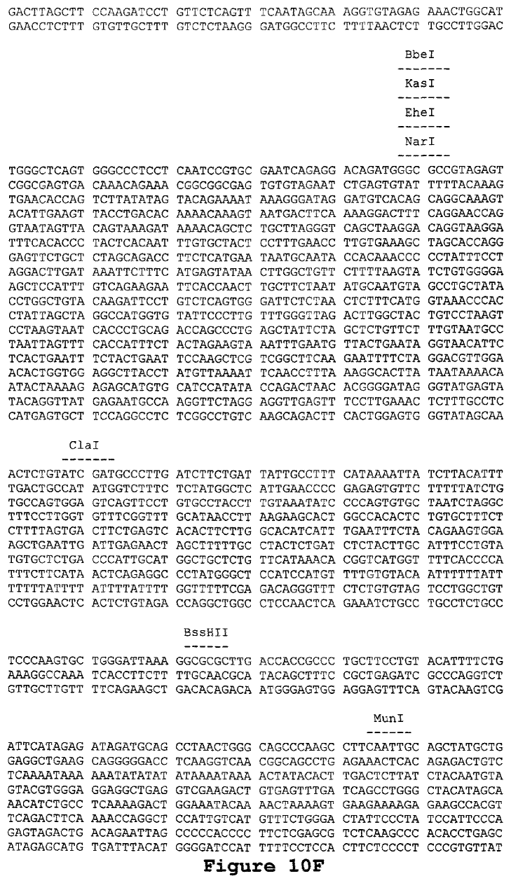

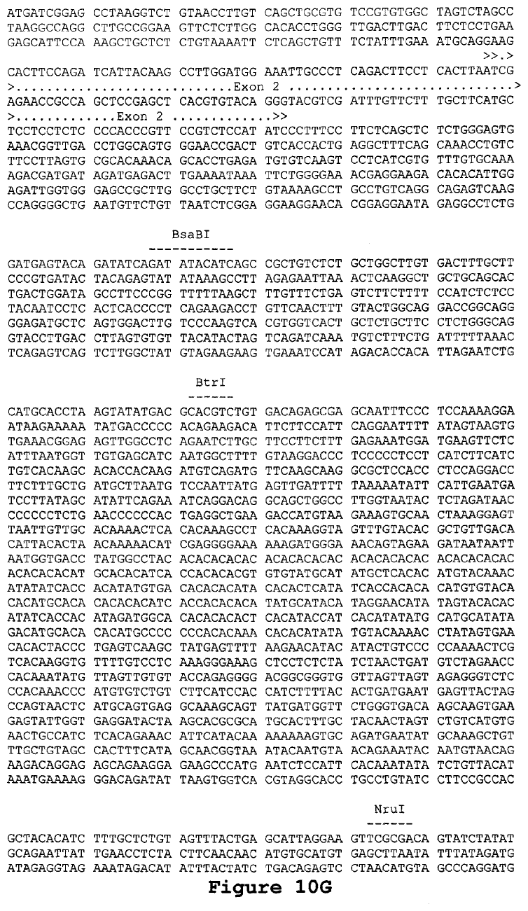

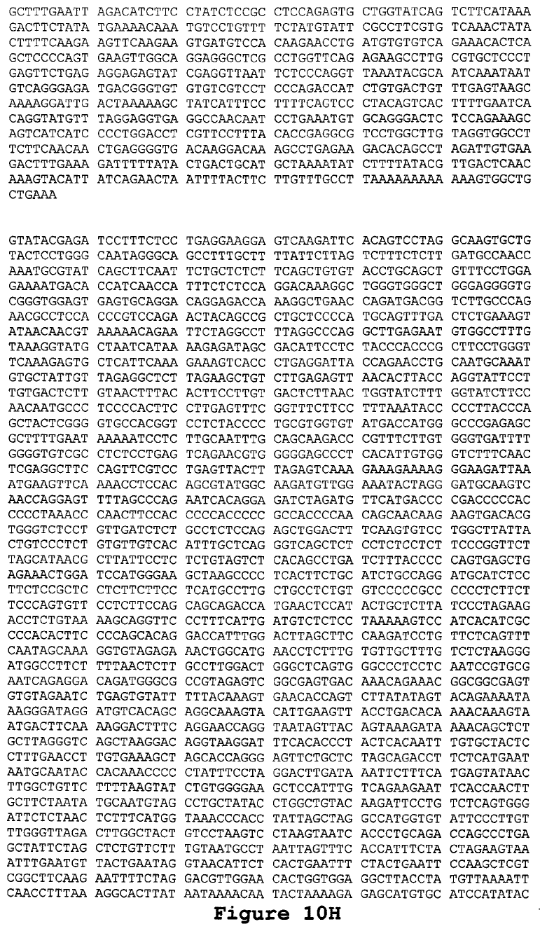

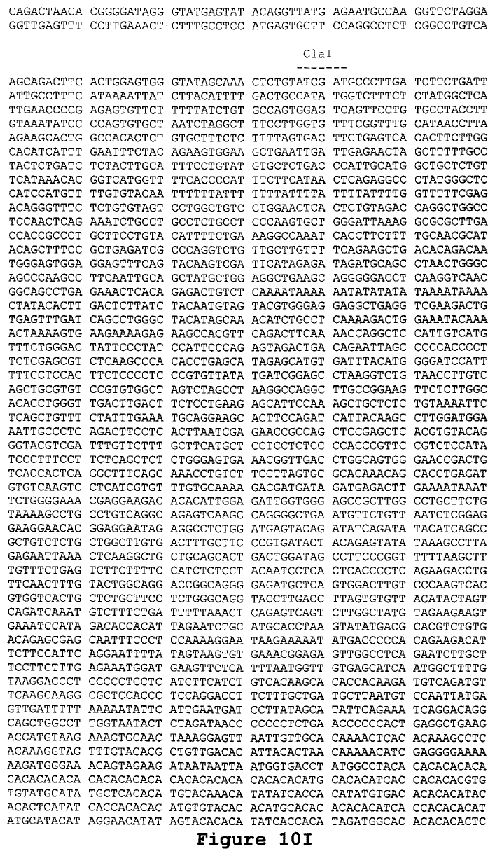

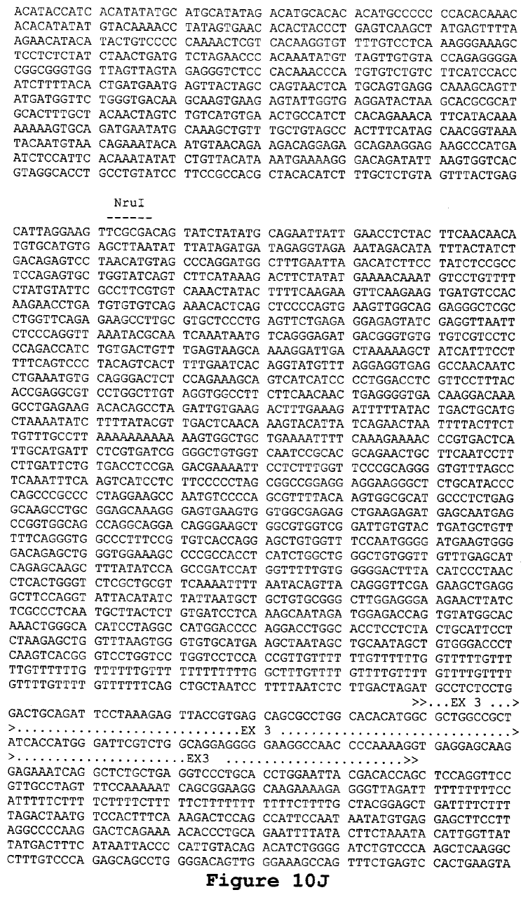

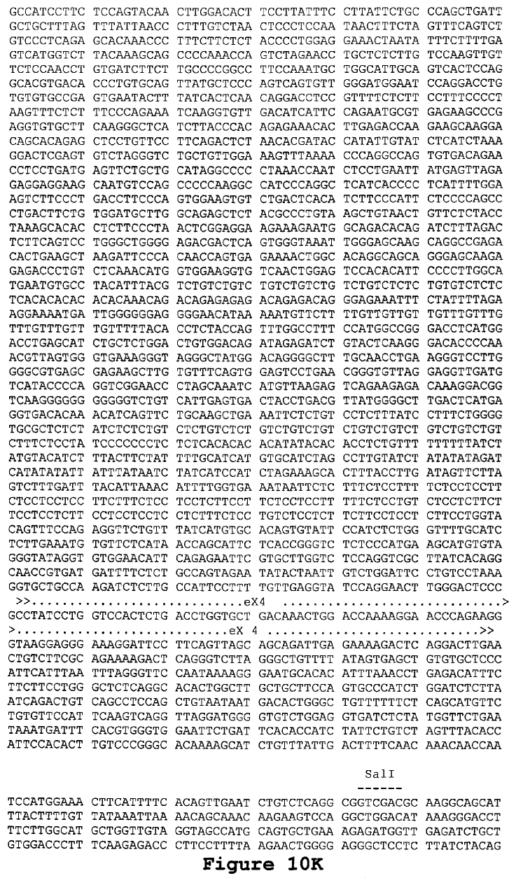

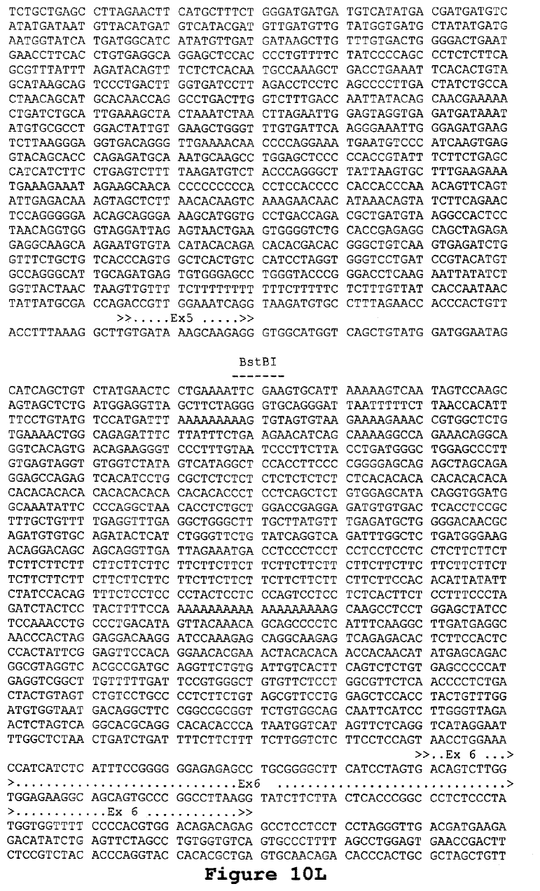

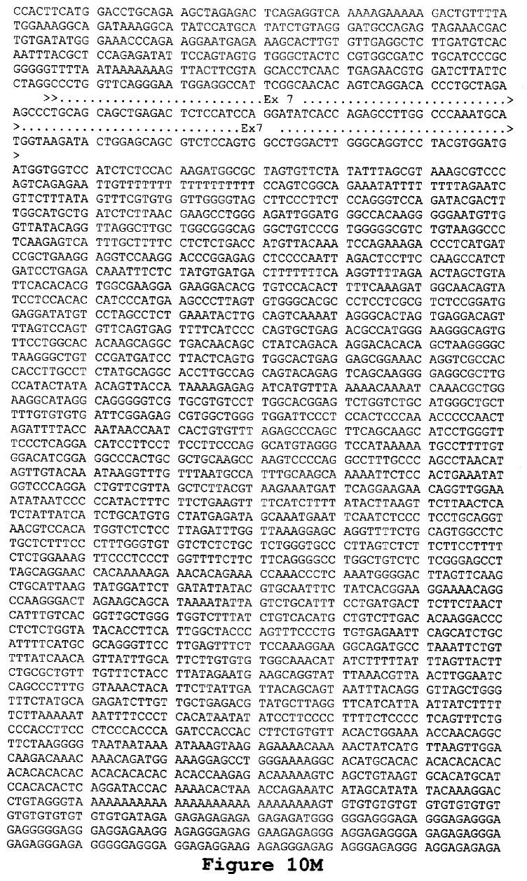

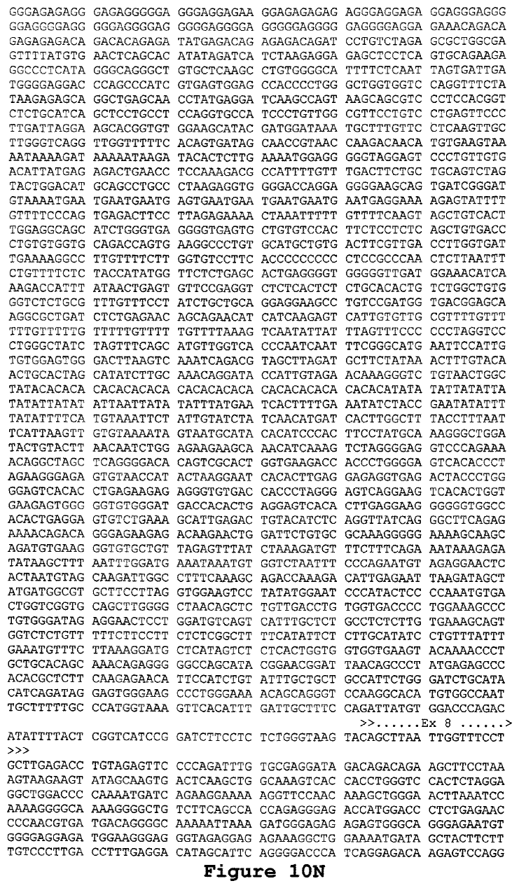

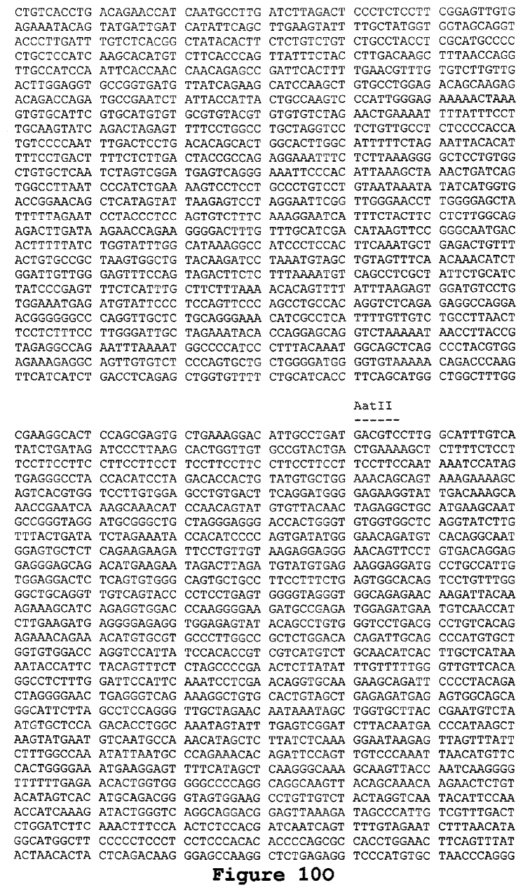

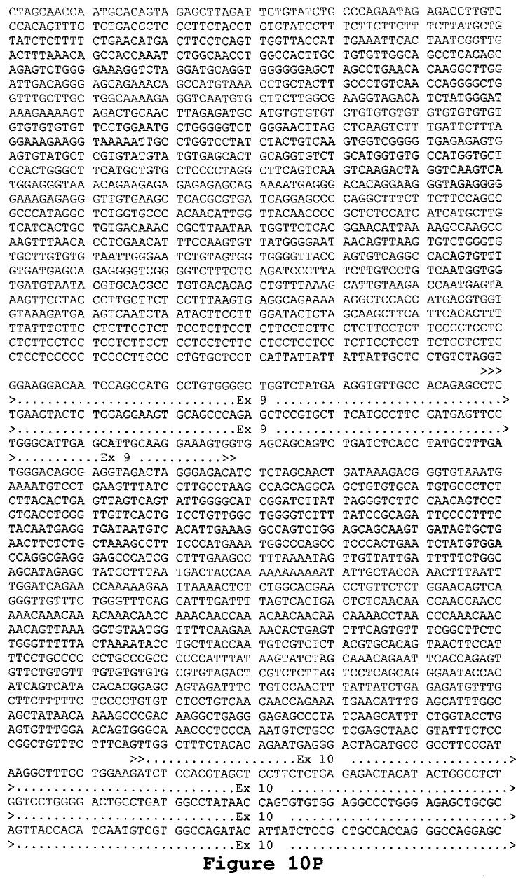

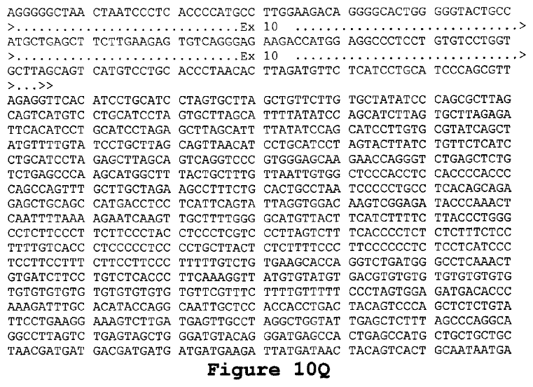

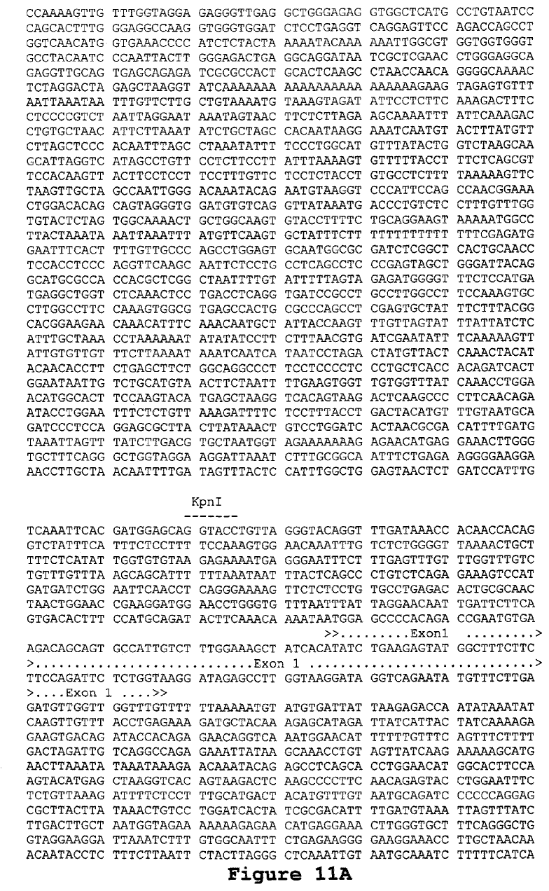

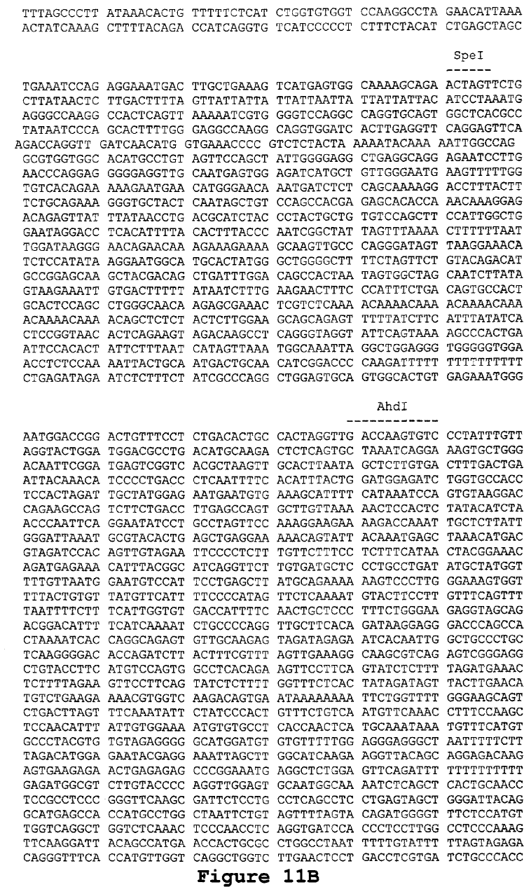

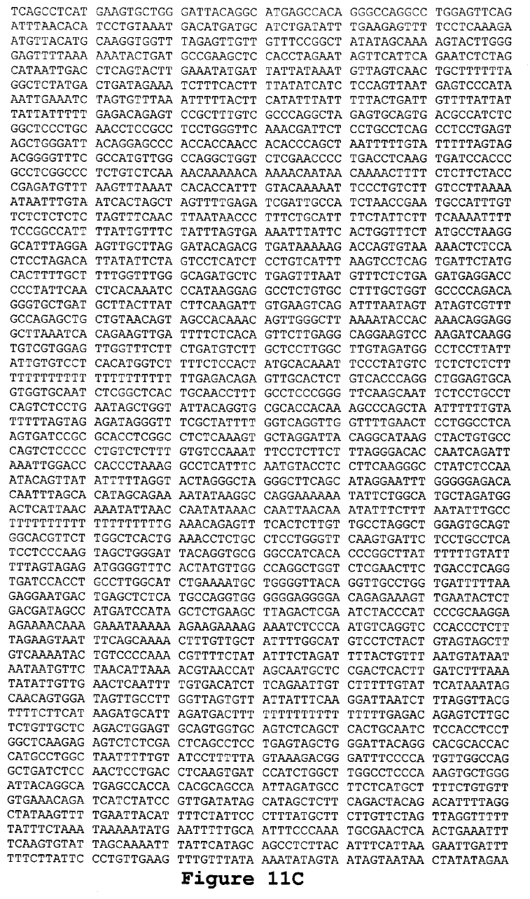

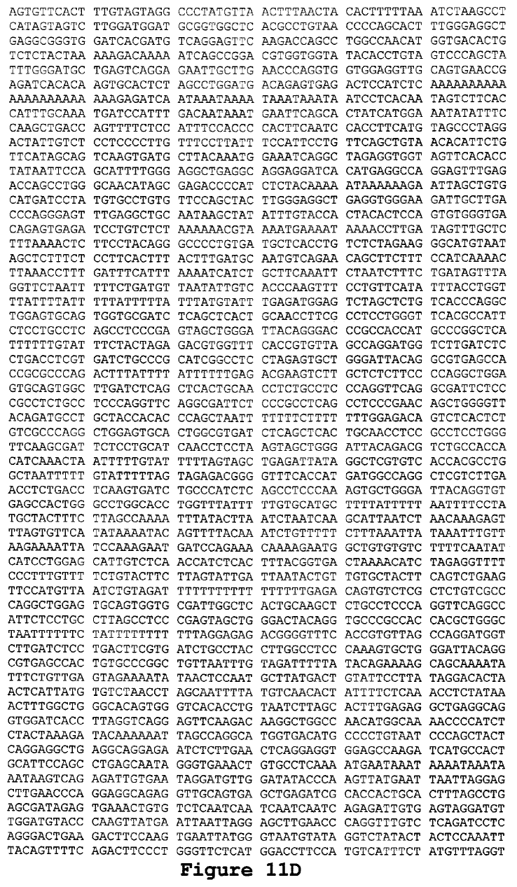

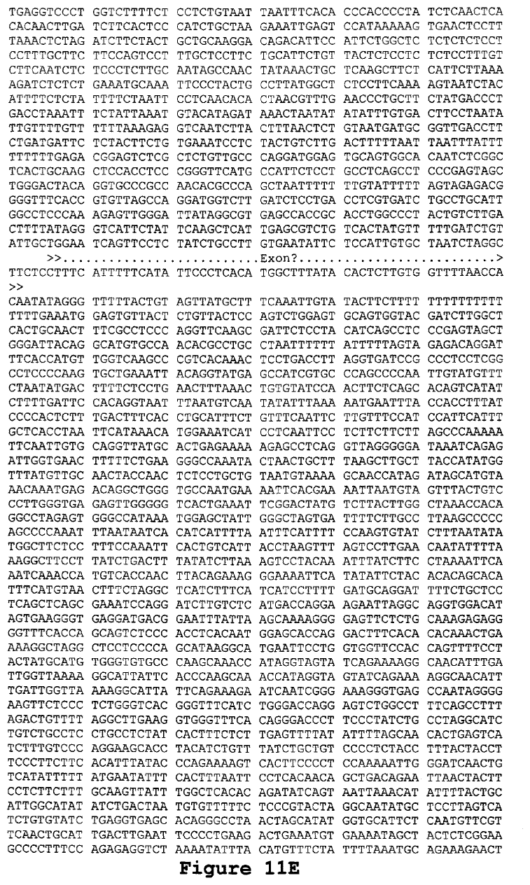

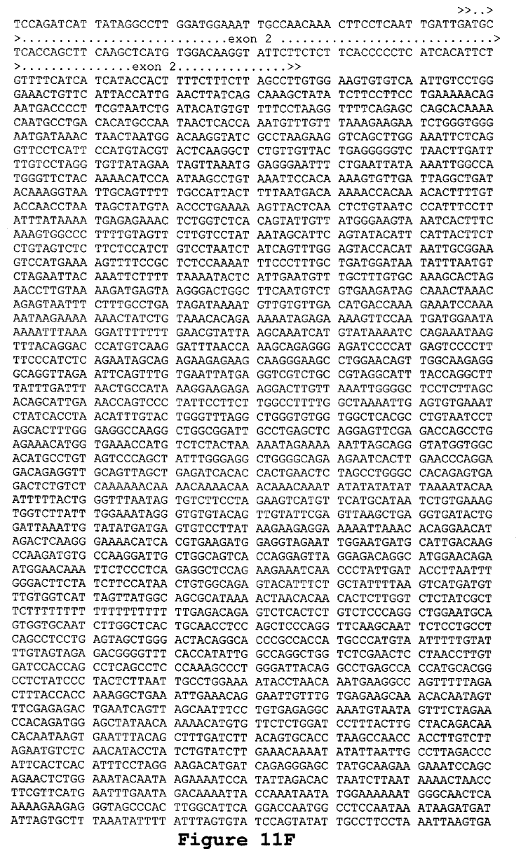

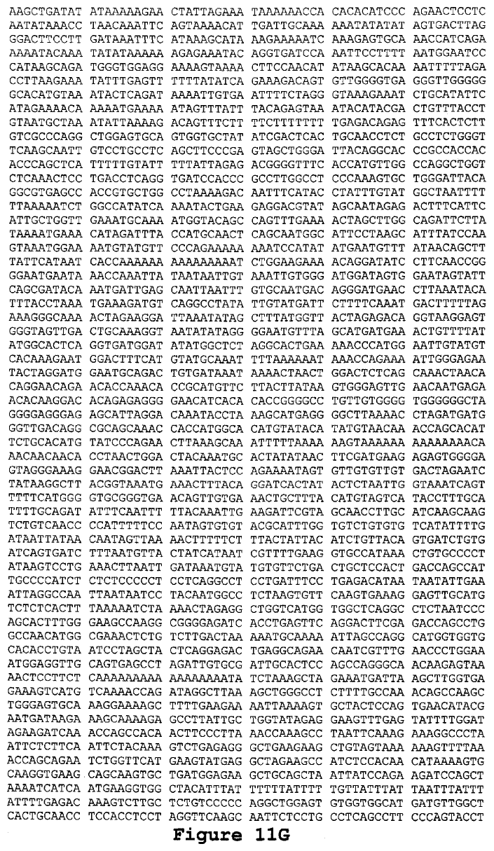

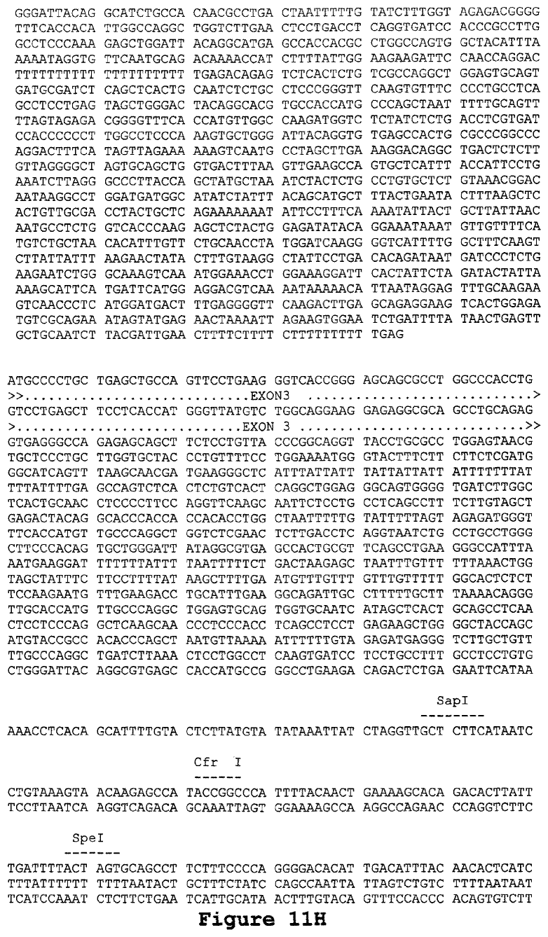

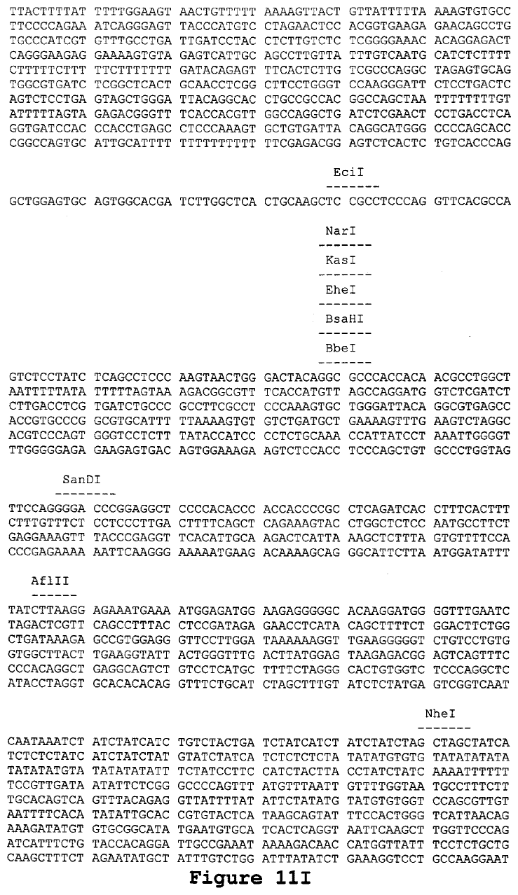

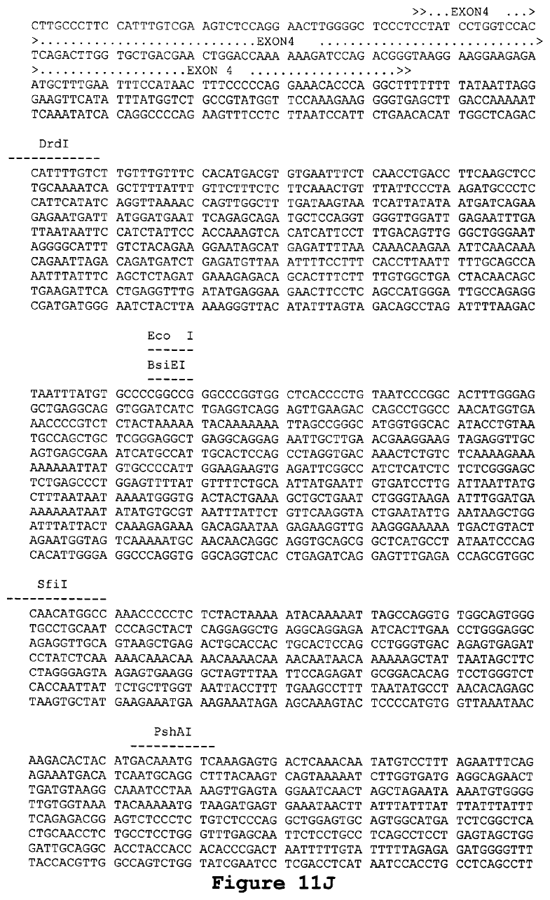

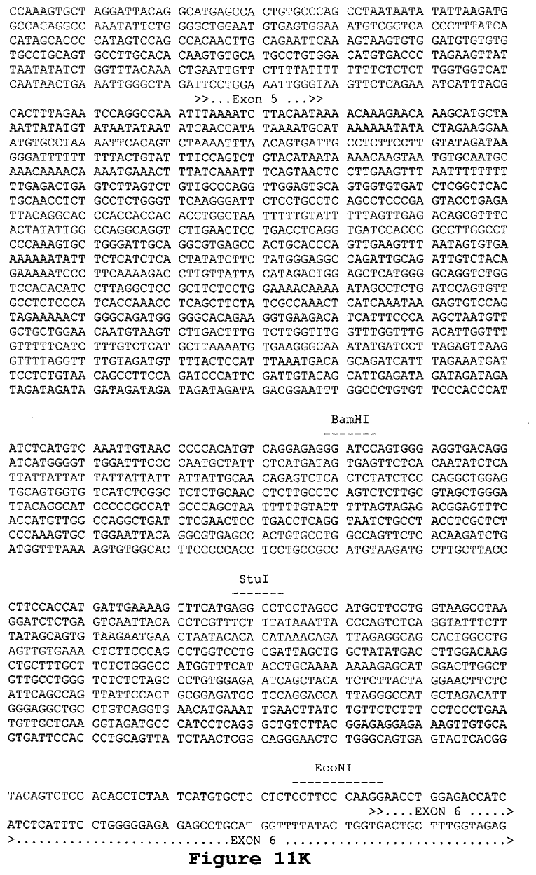

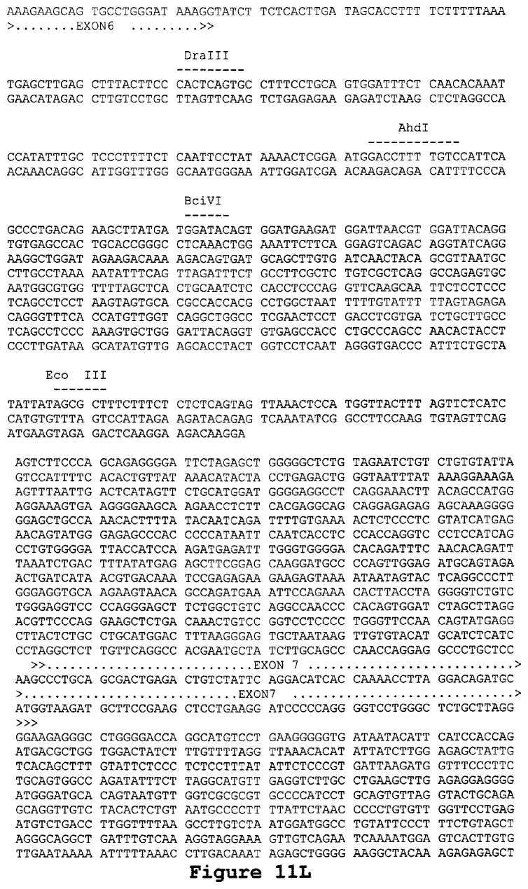

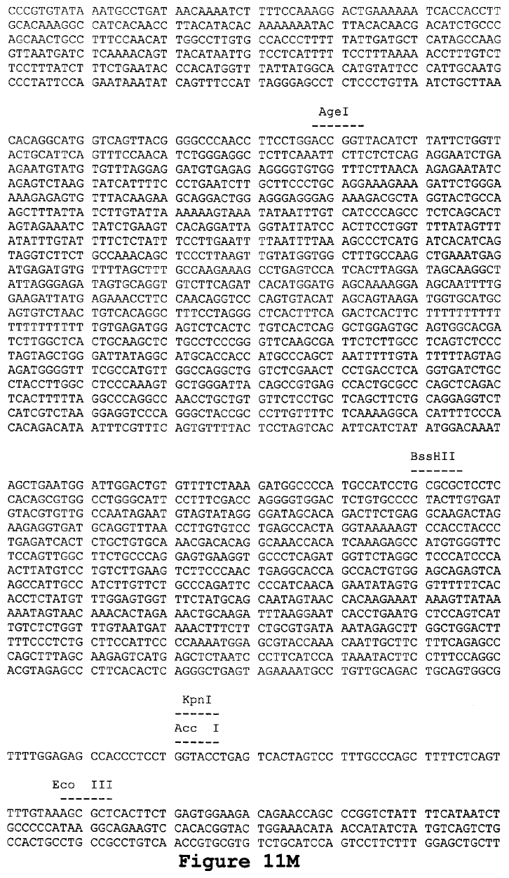

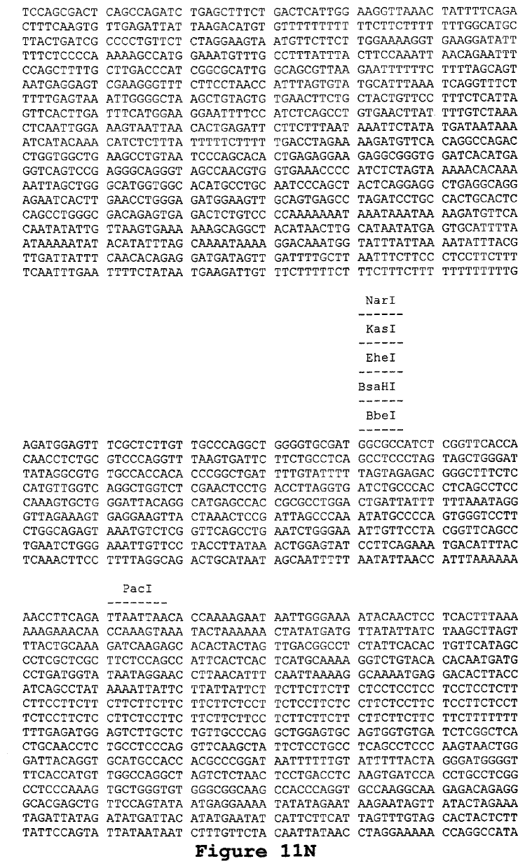

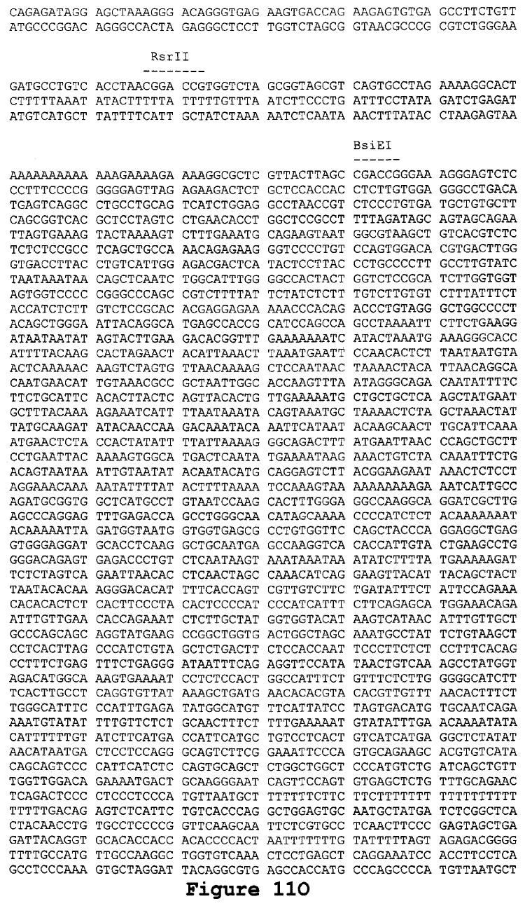

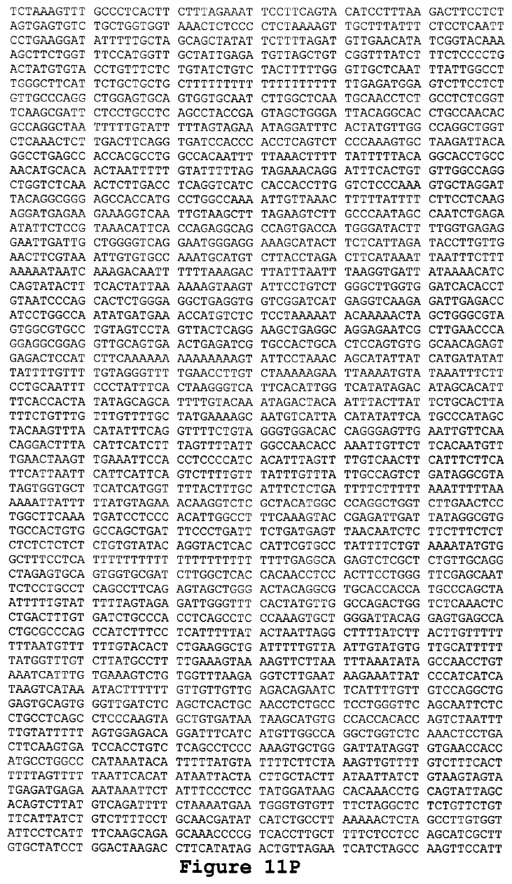

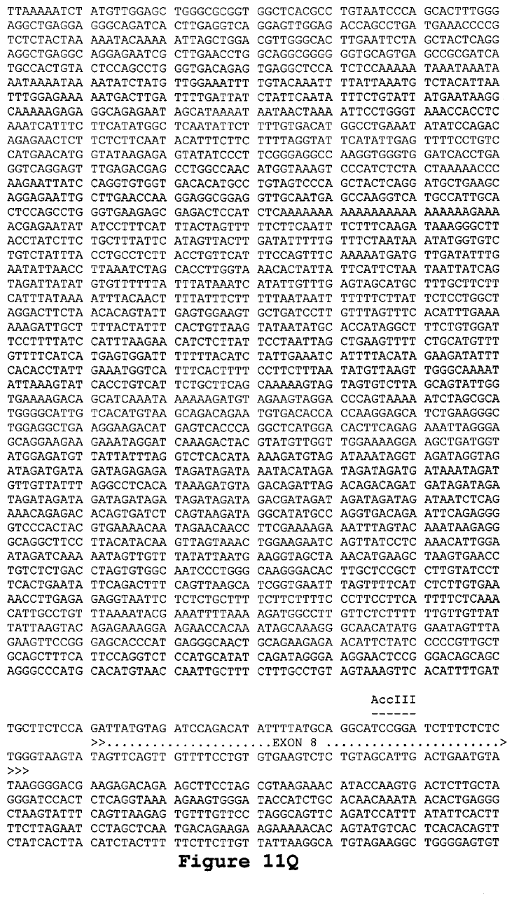

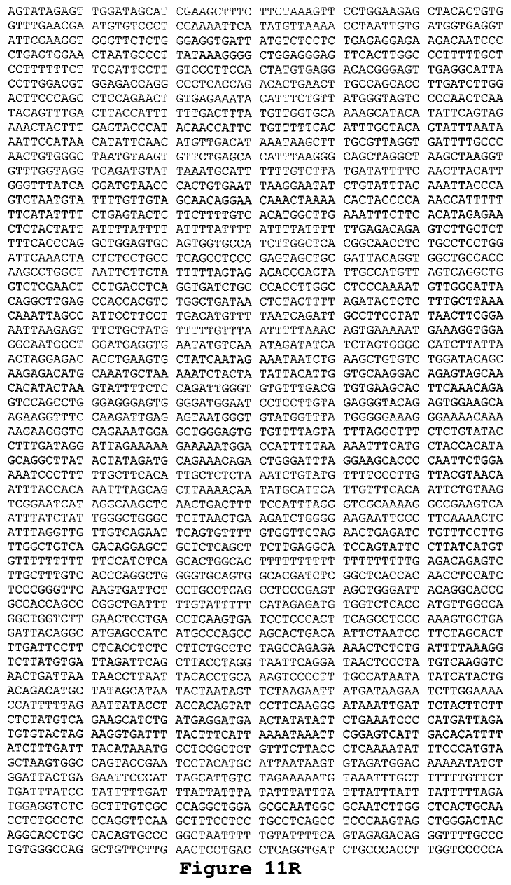

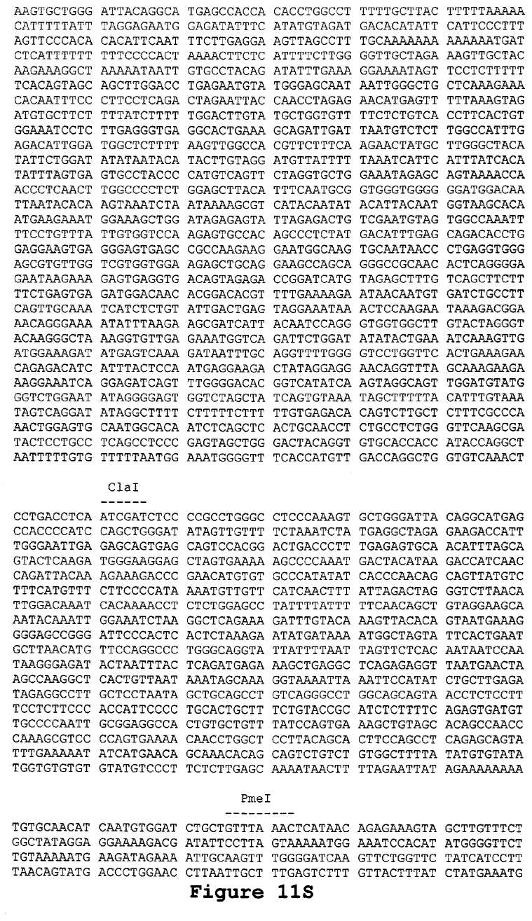

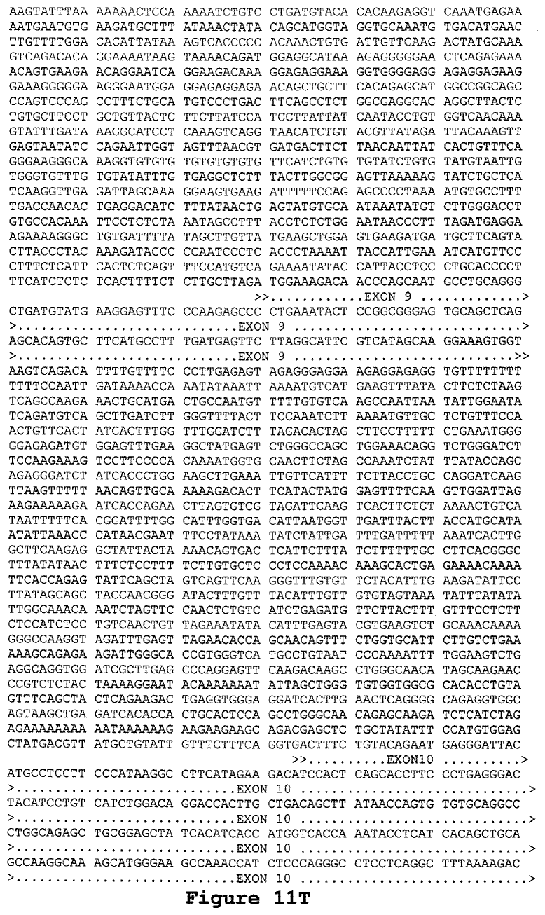

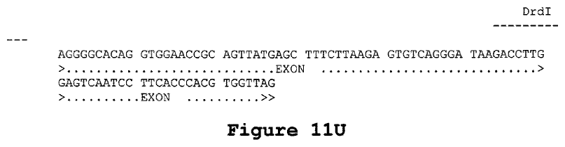

FIG. 9A demonstrates that IDO1 induces LIP expression. IDO1 is expressed in these cells when stimulated by doxycycline. When doxycycline is added to these cells, LIP is expressed (72 hours after stimulation). However, in the presence of exogenously added tryptophan (100 μM) the expression of LIP is eliminated; even when IDO1 is expressed. These data demonstrate that the LIP activation occurs via tryptophan deprivation, GCN2 activation, and differential LIP synthesis.

FIG. 9B demonstrates that the same activation of LIP occurs following IDO2 activation, but it is not inhibited by excess tryptophan. These data show that IDO2 activates the same pathway leading to LIP activation but that this pathway is not dependent on tryptophan deprivation. Also this data shows that the IDO2 activation of LIP is inhibited by the D isoform and, to a lesser extent, the L isoform of 1 methyl tryptophan. The selectivity for the D isoform to inhibit IDO2's activation of LIP demonstrates that IDO2 and IDO1 activities can be distinguished pharmacologically.

FIG. 9C provides a Western blot demonstrating that, like IDO2's activation of LIP expression, IDO2 also activates the GCN2 kinase leading to elf2-α phosphorylation (the protein target of GCN2). This activity is dependent of IDO2 expression which is not inhibited by excess tryptophan. These data suggest that IDO2 functions within this pathway but the activation of GCN2 kinase, phosphorylation of elf2-alpha, and LIP expression is not dependent on tryptophan deprivation. Unlike IDO1, which activates GCN2 kinase pathway through the deprivation of tryptophan and can be relieved by exogenous tryptophan, the IDO2 mechanism of action is different and can not be relieved by exogenous tryptophan. Therefore, IDO2 functions more directly in activating the GCN2 pathway, perhaps by acting on the tryptophanyl-tRNA directly.

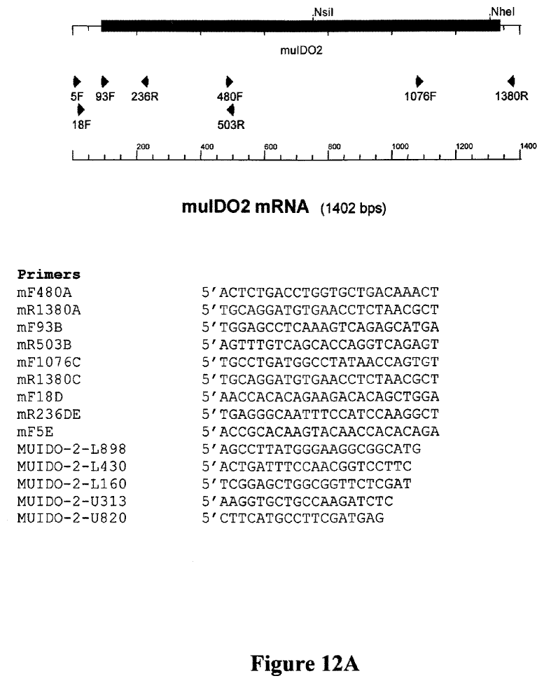

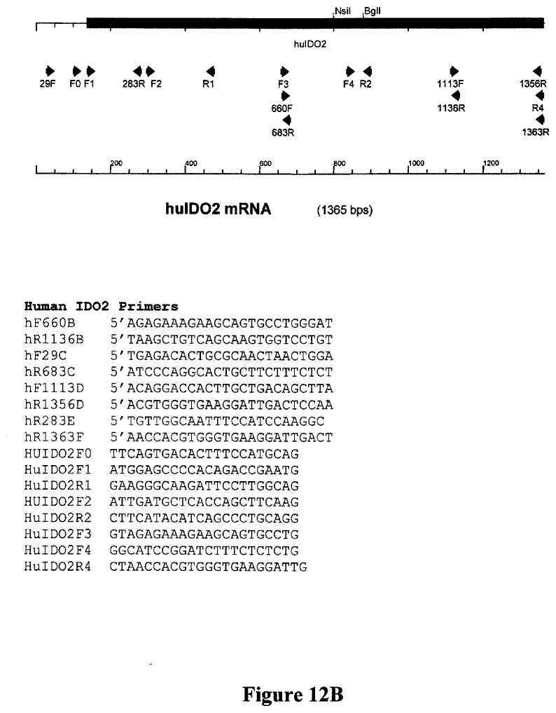

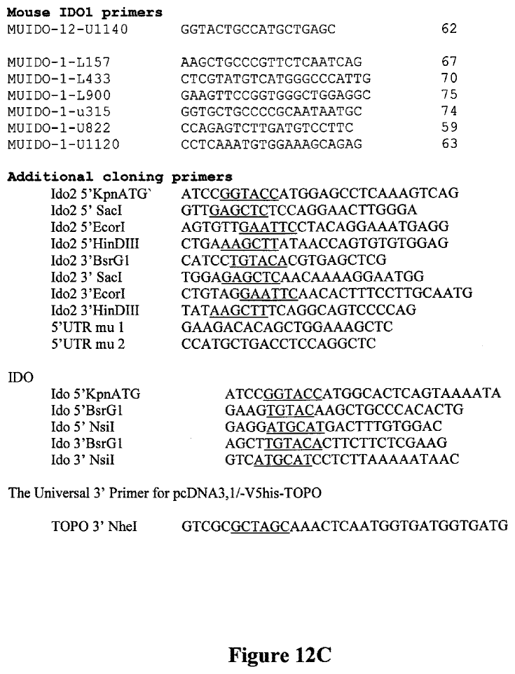

FIGS. 12A-12C provide schematics of murine and human IDO2 as well as primers which can be employed to amplify regions of IDO2.

### Example 2

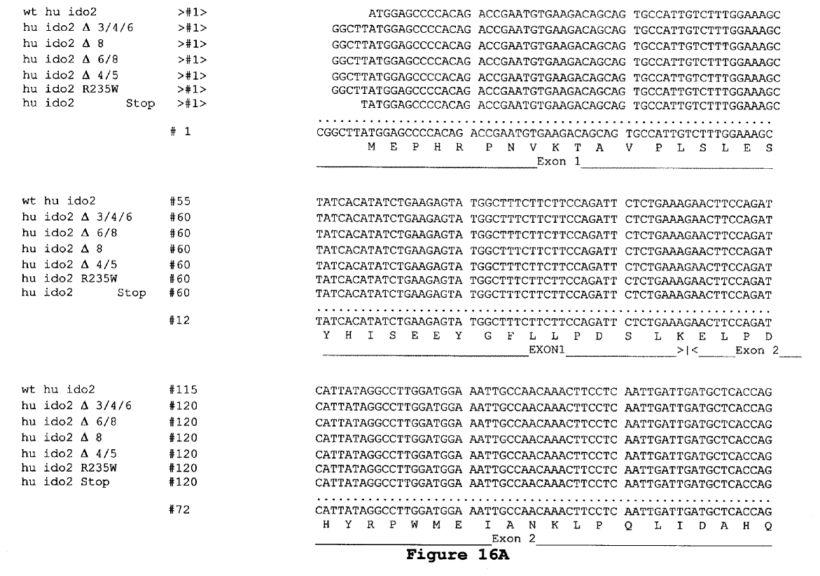

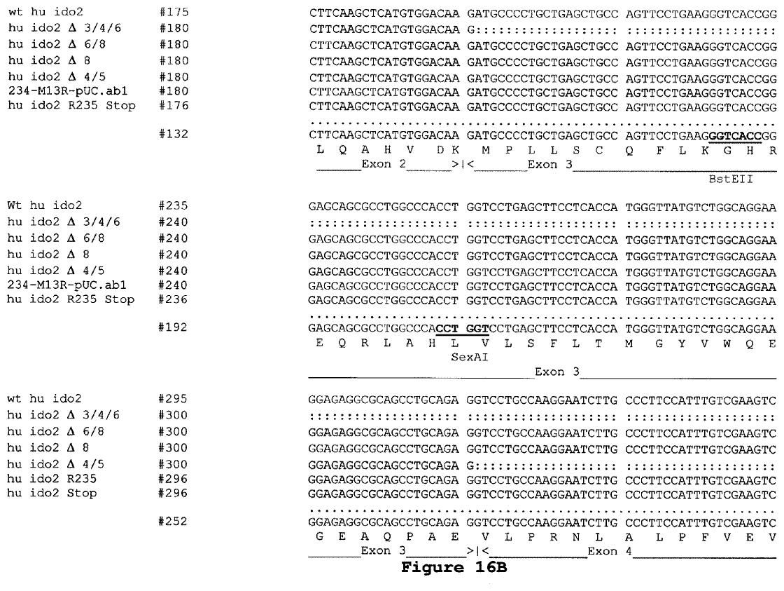

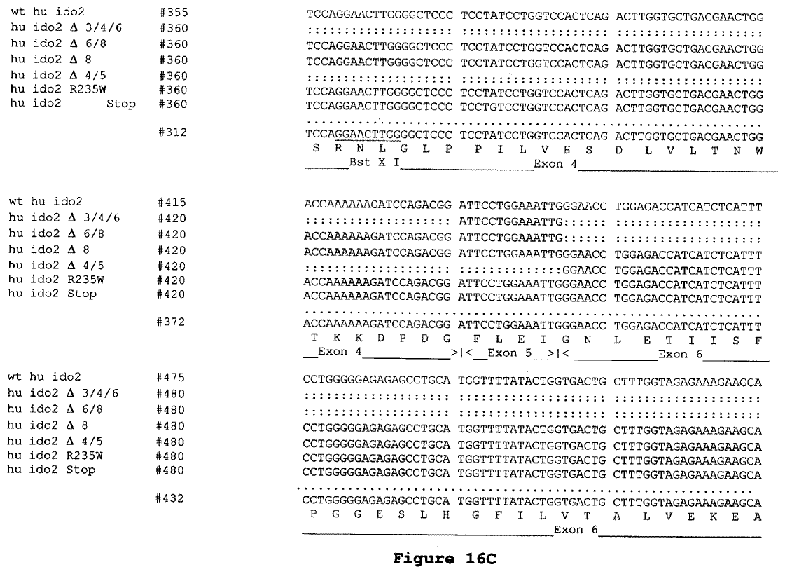

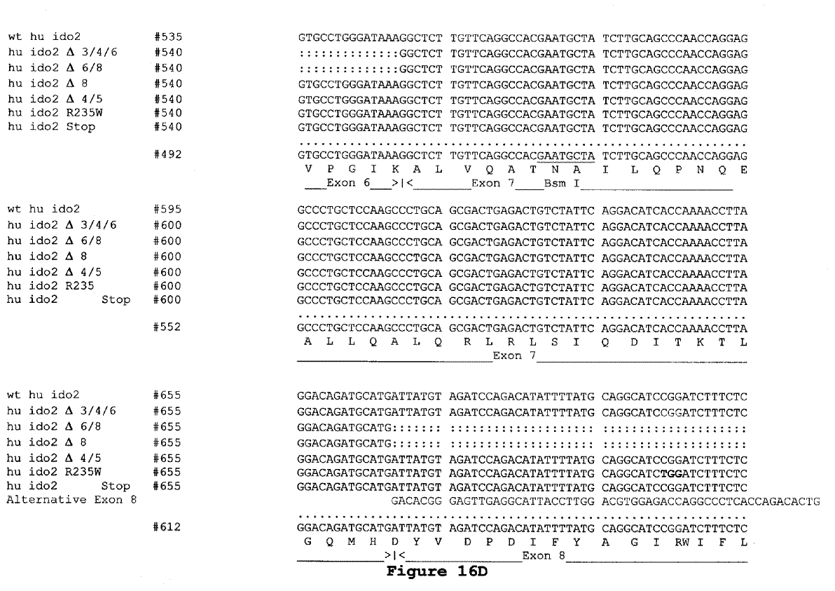

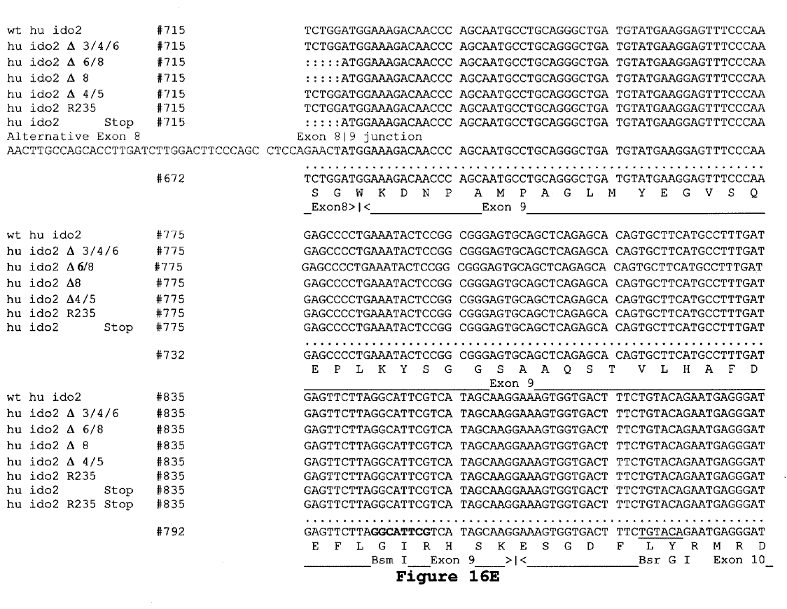

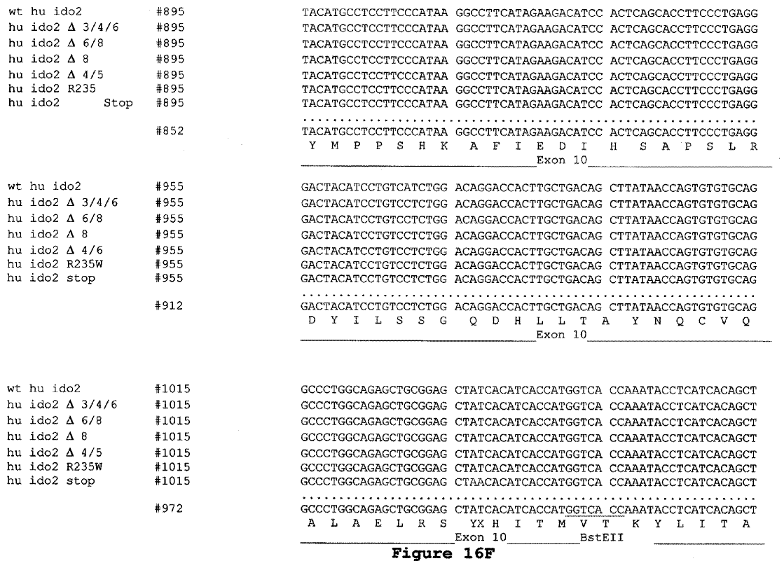

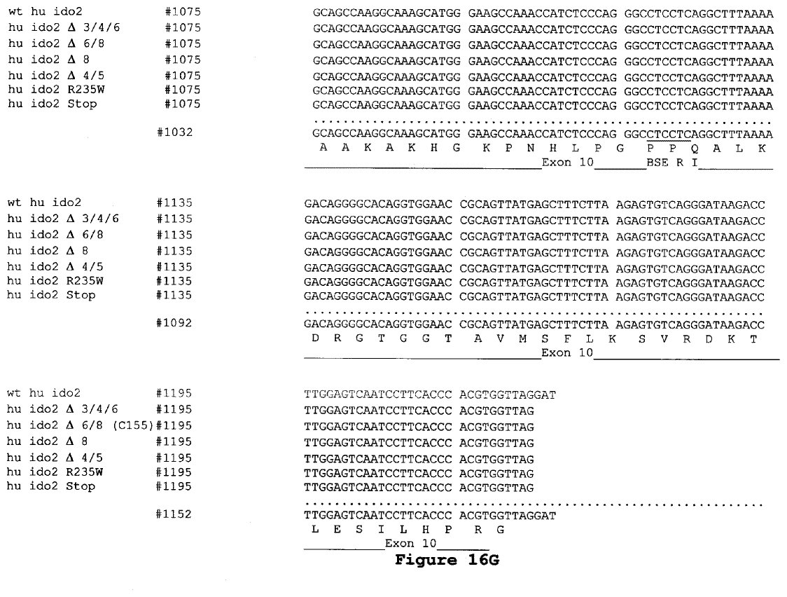

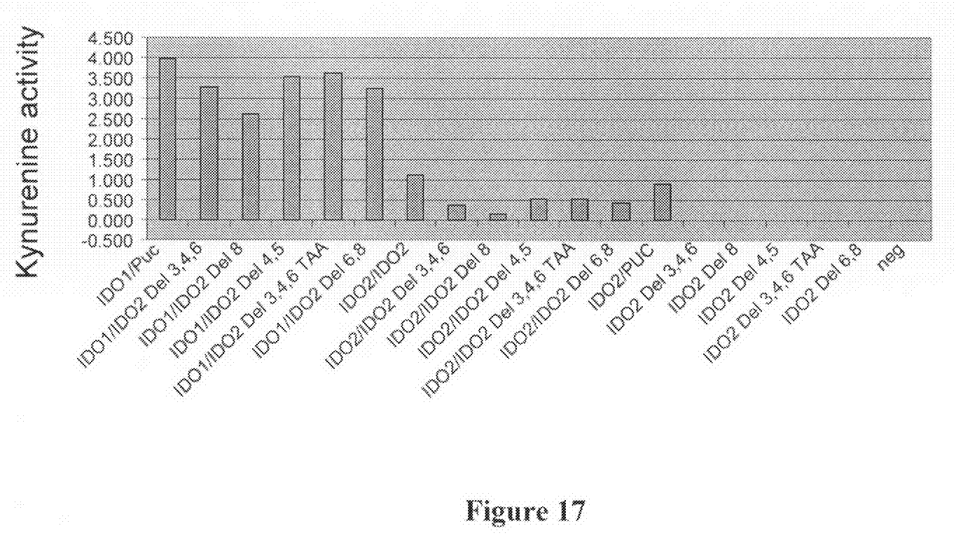

Human IDO2 CDNAs were cloned from RNA isolated from normal human liver. Splice variants were identified which lead to alternative reading frames and/or early termination of the primary sequence of IDO2. These splice variants are IDO2 Δ 3/4/6, IDO2 Δ 8, IDO2 Δ 6/9, and IDO2 Δ 4/5. FIG. 16 provides the nucleotide sequences of these splice variants. Vectors comprising the splice variants were transiently transfected into 293 (T-Rex™) cells and a kynurenine detection assay was performed as described hereinabove to determine the presence of indole dioxygenase activity with the expressed IDO2 splice variants. As seen in FIG. 17, the tested splice variants did not possess detectable indole dioxygenase activity.

Two prominent single nucleotide polymorphisms (SNP) were also cloned. These two SNPs are not linked (i.e., they are not on the same allele) and are differentially represented in various populations as indicated by the SNP database. The R235W SNP encoded in exon 8 alters the arginine (R) present at amino acid position 235 to a tryptophan (W). The SNP at amino acid position 332 introduces a stop codon at Tyrosine 332 (Y332stop). Both SNPs render the IDO2 molecule inactive when assayed for the production of kynurenine following a transient transfection assay as described hereinabove. As such these SNPs may be important indicators/markers for the propensity or resistance to certain disease states.

Additionally, as seen in FIGS. 15A-15Y and 16, an alternative splice site exists in exon 8 of IDO2, leading to an alternate sequence for exon 8.

When the alternatively spliced variants are co-expressed with either wild-type IDO1 or IDO2 lower, the indole dioxygenase activity, as assessed by the kynurenine detection assay, is reduced compared to wild-type IDO1 or IDO2 alone, even though IDO1 or IDO2 protein levels are the same in each reaction (see FIG. 17). These data indicate that an RNA or peptide product of the truncated IDO2 and/or alternatively spliced IDO2 may function to inhibit IDO1 and IDO2 activity. Such interactions may be dependent on direct or indirect protein-protein interactions between the truncated product and the full length IDO1 and IDO2 peptides. Alternatively the spliced variants may inhibit co-factor activity and the normal activation of IDO1/2 enzyme via changes in the oxidative state of the Fe component of coordinated Heme group. Nevertheless the negative interaction of these splice variants suggests the utility of a compound or peptide whose interaction with the splice variants or full length IDO1 or IDO2 will block the negative and inhibitory interactions resulting in increased IDO1 or IDO2 activity. Applications for such a compound or peptide include diseases that have an autoimmune component.

### Example 3

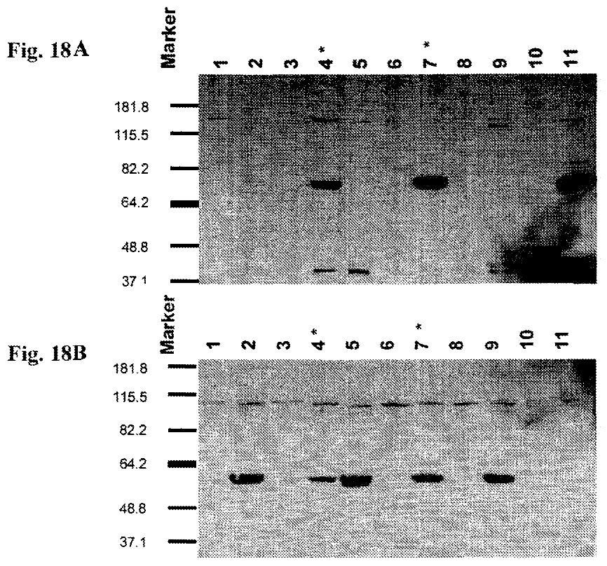

Monoclonal antibodies specific to mouse IDO2 were generated using standard techniques. FIG. 18 provides images of Western blots which demonstrate the species and IDO-isoform specificity of the generated monoclonal antibodies. Mouse or human IDO1 and/or IDO2 were expressed in 293 (T-REX) cells. Additionally, the R235W (R-W) and Y332stop (Y-*) isoforms of human and mouse IDO2 were also expressed in 293 (T-REX) cells. As seen in FIG. 18B, monoclonal antibodies derived from hybridoma clones (7.4.6, 7.4.9 and 8.8.6) are specific to mouse IDO2 protein and do not recognize the human or mouse IDO1 proteins (shown reacting with rabbit polyclonal sera directed to both human and mouse IDO1). FIG. 18A demonstrates the reactivity of an IDO1 polyclonal sera which recognizes both human and mouse IDO1.

### Example 4

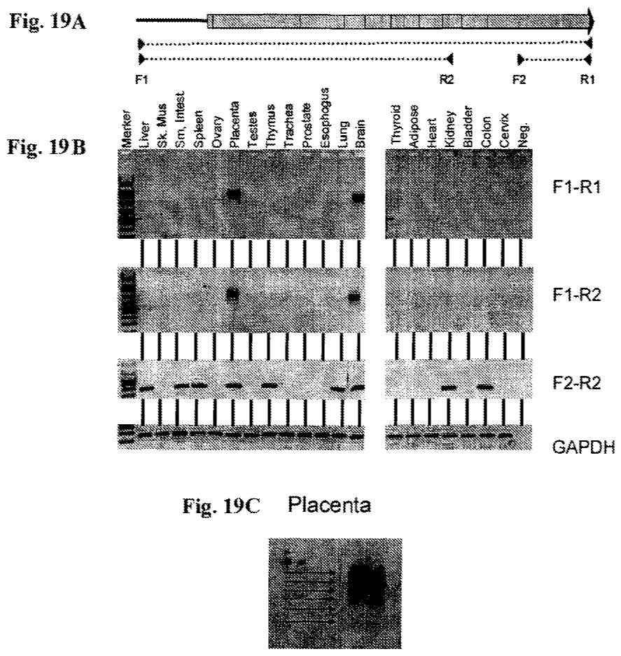

The tissue specific expression of human IDO2 was determined using T-PCR. A human tissue panel of total RNA (Ambion, Austin, TX) was analyzed by RT-PCR. The RT reaction utilized oligo-dt priming followed by the specific amplification using primers specific to different regions of the IDO2 mRNA (FIG. 19A), Primer F1 recognized a region in the 5′ untranslated region of the mRNA. Transcripts containing this region of the gene (exon 1a) are detected only in the placenta and brain (FIG. 19B). In contrast, primers specific to the 3′ end of the gene (primers spanning exons 9-10) demonstrated a broader pattern of expression. The presence of splice variants can be detected in both the placenta (FIG. 19C) and brain tissue. Without being bound by theory, this data suggests that there may be alternative promoters initiating at exon 1a and at exon 1, as exon 1 is more widely expressed than exon 1a. Primers F2-R2 were used because exons 9 and 10 are present in all splice variants detected so far.

### Example 5

The present example describes the generation of an indoleamine 2,3-dioxygenase-2 (IDO-2) gene conditional knockout allele in mice using homologous recombination in mouse embryonic stem cells and subsequent blastocyst injection of the appropriate targeted ES cells.

The mouse IDO-2 gene is located on chromosome 8, only 6 kb downstream from the IDO gene. The fact that the two genes are so close together makes it less desirable to attempt to knock out IDO-2 function by eliminating the first exons. As described herein, IDO-2 is organized into ten exons. Exons 9 and 10 play a crucial role for the enzyme function. As such, a conditional allele that upon recombination will eliminate only these two exons was created.

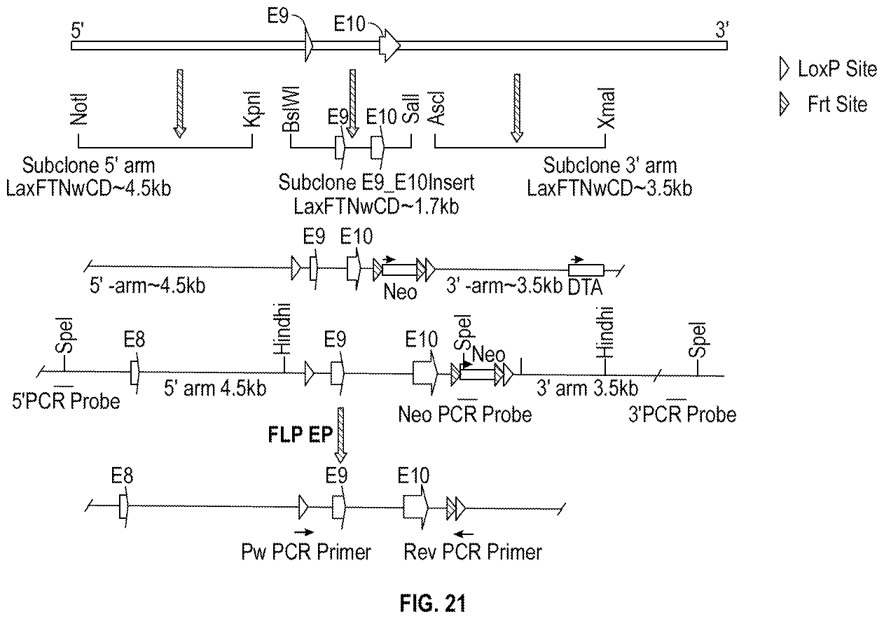

RP23-339B16 BAC clone (which contains both IDO and IDO2 genes) was used for generating the homologous arms and the conditional KO region for the gene targeting vector, as well as the southern probes for screening targeted events. The recombination targeting vector (FIG. 21) will result in the Cre-dependent deletion of exons 9 and 10. Exons 9 and 10 were chosen because the analogous structural domain of IDO1 constitutes the major catalytic domain of the enzyme. Similarly in vitro mutagenesis studies on IDO2 demonstrate that this region is required for enzymatic activity.

As demonstrated in FIG. 21, the unconditional knock-out was generated using a positive (Neo) and negative (dipteria toxin A; DTA) selection approach, whereby the DNA construct possesses a DTA expression cassette distal from the homologous arms to negatively select for non-homologous recombination and a neomycin positive selection cassette flanked by the sites for the FLP recombinase. In addition, the targeting vector has two Lox-P sites for the Cre recombinase each flanking exons 9 and 10, which allows for the Cre-dependent excision of exons 9 and 10. Embryonic stem (ES) cells (C57/Bl6) were electroporated with the targeting construct and the surviving Neo^{R} transfected clones were screened by Southern analysis to identify ES cells carrying targeted IDO2 alleles. Six of the ES cell clones containing the recombinant targeted conditional allele were electroporated with Flp recombinase-expressing plasmid to eliminate the neo cassette. Two positive FLP treated Neo⁽⁻⁾ clones expanded for further analysis using long-range PCR and Southern analysis. Selected clones with normal karyotypes were either injected into blastocysts (to generate a conditional IDO2-knockout chimeric embryo) or electroporated with CRE recombinase to remove exons 9 and 10. Four CRE treated ES cell clones were identified to possess the proper exon 9/10 deletion using PCR analysis, expanded, and injected into blastocysts (to generate an IDO2 knockout chimeric mouse embryo).

The 5′ homologous arm (4.5 kb), 3′ homologous arm (3.5 kb) and conditional, KO region (1.7 kb) were generated by PCR. The fragments were cloned in the LoxFtNwCD or (for sequencing purposes) PCR 4.0 vectors and confirmed by restriction digestion and end-sequencing.

The final vector was obtained by standard molecular cloning. Aside from the homologous arms, the final vector also contains loxP sequences flanking the conditional KO region (0.25 kb), Frt sequences flanking the Neo expression cassette (for positive selection of the ES cells), and a DTA expression cassette (for negative expression of the ES cells). The final vector was confirmed by both restriction digestion and end sequencing analysis. NotI was used for linearizing the final vector prior to electroporation.

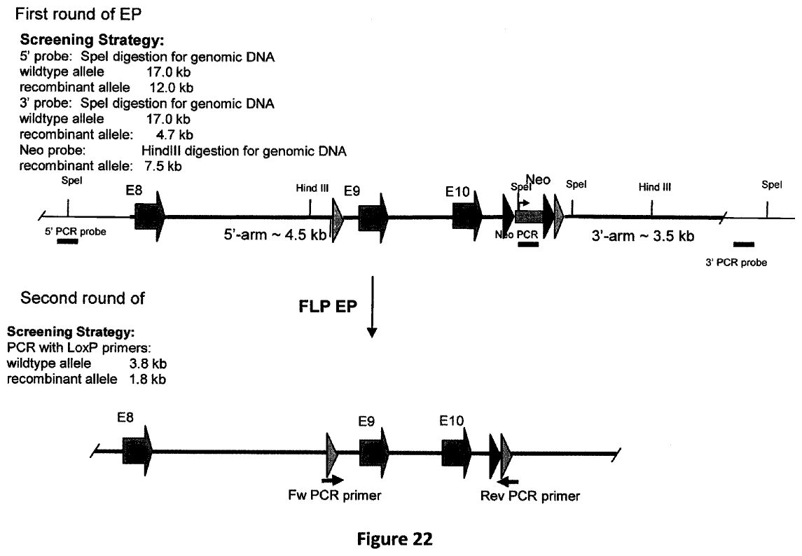

The 5′ and 3′ external probes were generated by PCR and were tested by genomic Southern analysis for screening of the ES cells. The probes were cloned in the pCR4.0 backbone and confirmed by sequencing. A screening strategy for mIDO-2 knockouts is provided in FIG. 22.

The offspring of the chimera pups for the conditional and unconditional IDO2 knockout animals were born, analyzed for germline transmission of the modified IDO2 alleles, and subsequently bred to homozygosity.

As indicated hereinabove, the homologous arms were generated by PCR. The 5′-arm was cloned into LoxFtNwCD in NotI/KpnI sites, upstream from loxP site. The template was BAC clone RP23-339B16 and the primers used were:

LDI_ID02_5′F: Tm 57.8
(SEQ ID NO: 148)
actggcggccgcATGCCGAATCTATTACCATTACTGCC

LDI_IDO2_5′R: Tm 60.4 (SEQ ID NO: 149)
actgggtaccACACCTTCATAGACCAGCCCCACA

The E9/E10 CKO region was cloned in BsiWI/SalI sites, upstream from FRT site. The template was BAC clone RP23-339B16 and the primers used were:

LDI_IDO2_INSF: Tm 60.4
(SEQ ID NO: 150)
actgcgtacgcatatgTGTGGGGCTGGTCTATGAAGGTGT

LDI_ID02_INSR: Tm 52.8 (SEQ ID NO: 151)
actggtcgacGGGATATAGCACAAGAACAGCTAAG

The 3′ arm was cloned in LoxFtNwCD in AscI/XmaI sites, BAC clone RP23-339B16 was used as the template and the primers used were:

LDI_IDO2_3′F: Tm 52.8
(SEQ ID NO: 152)
actgggcgcgccCTTAGCTGTTCTTGTGCTATATCCC

LDI_ID02_3′R: Tm 56.8 (SE ID NO: 153)
actgcccgggGGTGTCTGTGAGATTTTGAGAATAGTCC

The 5′ and 3′ arms and the CKO region were end sequenced. The sequence of the 5′ end of the 5′ arm was determined to be:

(SEQ ID NO: 154)
ATGCCGAATCTATTACCATTACTGCCAAGTCCCATTGGGAGAAAAACTAA
AGTGTGCATTCGTGCATGTGTGCGTGTACGTGTGTGTCTAGAACTGAAAA
TTTTATTTCCTTGCAAGTATCAGACTAGAGTTTTCCTGGCCTGCTAGGTC
CTCTGTTGCCTCTCCCCACCATGTCCCCAATTTGACTCCTGACACAGCAC
TGGCACTTGGCATTTTTCTAGAATTACACATTTTCCTGACTTTTCTCTTG
ACTACCGCCAGAGGAAATTTCTCTTAAAGGGGCTCCTGTGGCTGTGCTCA
ATCTAGTCGGATGAGTCAGGGAAATTCCCACATTAAAGCTAAACTGATCA
GTGGCCTTAATCCCATCTGAAAAGTCCTCCTGCCCTGTCCTGTAATAAAT
ATATCATGGTGACCGGAACAGCTCATAGTATTAAGAGTCCTAGGAATTCG
GTTGGGAACCTTGGGGAGCTATTTTTAGAATCCTACCCTCCAGTGTCTTT
CAAAGGAATCATTTCTACTTCCTCTTGGCAGAGACTTGATAAGAACCAGA
AGGGGACTTTGTTTGCATCGACATAAGTTCCGGGCAATGACACTTTTTAT
CTGGTATTTGGCATAAAGGCCATCCCTCCACTTCAAATGCTGAGACTGTT
TACTGTGCCGCTAAGTGGCTGTACAAGATCCTAAATGTAGCTGTAGTTTC
AACAAACATCTGGATTGTTGGGAGTTTCCAGTAGACTTCTCTTTAAAATC
TCAGCCTCGCTATTCTGCATCTATCCCGAGTTTCTCATTTGCTTCTTTAA
AACACAGTTTTATTTAAGAGTGGATGTCCTGTGGAAATGAGATGTATTCC
CTCCAGTTCCCAGCC.

The sequence of the 3′ end of 5′ arm was determined to be.

(SEQ ID NO: 155)
TGAGTGTATGCTCGTGTATGTATGTGAGCACTGCAGGTGTCTGCATGGTG
TGCCATGGTGCTCCACTGGGCTTCATGCTGTGCTCCCCTAGGCTTCAGTC
AAGTCAAGACTAGGTCAAGTCATGGAGGGTAAACAGAAGAGAGAGAGAGC
AGAAAATGAGGGACACAGGAAGGGTAGAGGGGGAAAGAGAGGGTTGTGAA
GCTCACGCGTCATCAGGAGCCCCAGGCTTTCTTCTTCCAGCCGCCCATAG
GCTCTGGTGCCCACAACATTGGTTACAACCCCGCTCTCCATCATCATGCT
TGTCATCACTGCTGTGACAAACCGCTTAATAATGGTTCTCACGGAACATT
AAAAGCCAAGCCAAGTTTAACACCTCGAACATTTCCAAGTGTTATGGGGA
ATAACAGTTAAGTGTCTGGGTGTGCTTGTGTGTAATTGGGAATCTGTAGT
GGTGGGGTTACCAGTGTCAGGCCACAGTGTTTGTGATGAGCAGAGGGGTC
GGGGTCTTTCTCAGATCCCTTATCTTGTCCTGTCAATGGTGGTGATGTAA
TAGGTGCACGCCTGTGACAGAGCTGTTTAAAGCATTGTAAGACCAATGAG
TAAAGTTCCTACCCTTGCTTCTCCTTTAAGTGAGGCAGAAAAAGGCTCCA
CCATGACGTGGTGTAAAGATGAAGTCAATCTAATACTTCCTTGGATACTC
TAGCAAGCTTCATTCACACTTTTTATTTCTTCCTCTTCCTCTTCCTCTTC
CTCTTCCTCTTCCTCTTCCTCTTCCCCTCCTCCTCTTCCTCCTCCTCTTC
CTCCTCCTCTTCCTCCTCCTCCTCTTCCTCCTTCTCCTCTTCCTCCTCCC
CCTCCCCTTCCCCTGTGCTCCTCATTATTATTATTATTGCTCCTGTCTAG
GTGGAAGGACAATCCAGCCATGCCTGTGGGGCTGGTCTATGAAGGTGT.

The sequence of the 5′ end of CKO region (E9) was determined to be:

(SEQ ID NO: 156)
GGTACCAAAGGCCGCAATGGCCAAACCTGCAGGAAGTACCAGAGCTCCCT
AGGTTCTAGAACCGGTGACGTCAAGCTCGAATAACTTCGTATAATGTATG
CTATACGAAGTTATCCTGCAGGTCGATCGAGACCGTACGCTATGTGTGGG
GCTGGTCTATGAAGGTGTTGCCACAGAGCCTCTGAAGTACTCTGGAGGAA
GTGCAGCCCAGAGCTCCGTGCTTCATGCCTTCGATGAGTTCCTGGGCATT
GAGCATTGCAAGGAAAGTGGTGAGCAGCAGTCTGATCTCACCTATGCTTT
GATGGGACAGCGAGGTAGACTAGGGAGACATCTCTAGCAACTGATAAAGA
CGGGTGTAAATGAAAATGTCCTGAAGTTTATCCTTGCCTAAGCCAGCAGG
CAGCTGTGTGCATGTGCCCTCTCTTACACTGAGTTAGTCAGTATTGGGGC
ATCGGATCTTATTAGGGTCTTCCAACAGTCCTGTGACCTGGGTTGTTCAC
TGTCCTGTTGGCTGGGGTCTTTTATCCGCAGATTCCCCTTTCTACAATGA
GGTGATAATGTCACATTGAAAGGCCAGTCTGGAGCAGCAAGTGATAGTGC
TGAACTTCTCTGCTAAAGCCTTTCCCATGAAATGCCCCAGCCTCCCACTG
AATCTATGTGGACCAGGCGAGGGAGCCCATCGCTTTGAAGCCTTTAAAA
T.

The sequence of the CKO region (E10) was determined to be:

(SEQ ID NO: 157)
CAGAAATGAACATTTGAGCATTTGGCAGCTATAACAAAAGCCCGACAAGG
CTGAGGGAGAGCCCTATCAAGCATTTCTGGTACCTGAGTGTTTGGAACAG
TGGGCAAACCCTCCCAAATGTCTGCCTCGAGCTAACGTATTTCTCCCGGC
TGTTTCTTTCAGTTGGCTTTCTACACAGAATGAGGGACTACATGCCGCCT
TCCCATAAGGCTTTCCTGGAAGATCTCCACGTAGCTCCTTCTCTGAGAGA
CTACATACTGGCCTCTGGTCCTGGGGACTGCCTGATGGCCTATAACCAGT
GTGTGGAGGCCCTGGGAGAGCTGCGCAGTTACCACATCAATGTCGTGGCC
AGATACATTATCTCCGCTGCCACCAGGGCCAGGAGCAGGGGGCTAACTAA
TCCCTCACCCCATGCCTTGGAAGACAGGGGCACTGGGGGTACTGCCATGC
TGAGCTTCTTGAAGAGTGTCAGGGAGAAGACCATGGAGGCCCTCCTGTGT
CCTGGTGCTTAGCAGTCATGTCCTGCACCCTAACACTTAGATGTTCTCAT
CCTGCATCCCAGCGTTAGAGGTTCACATCCTGCATCCTAGTGCTTAGCTG
TTCTTGTGCTATATCCCGTCGACCAAGTTCCTATACTTTCTAGAGAATAG
GAACTTCGGATCCACGATTCGAGGGCCCCTGCAGGTCAATTCTACCGGGT
AGGGGAGGCGCTTTTCCCAAGGCAGTCTGGAGCATGCGCTTTANCAGCCC
CGCTGGGCACTTGGCGCTACACAAGTGGCCTCTGGCCTCGCACACATTCC
ACA.

The sequence of the 5′ end of 3′ arm was determined to be:

(SEQ ID NO: 158)
ATCCCAGCGCTTAGCAGTCATCTCCTGCATCCTAGTCCTTAGCATTTTAT
ATCCAGCATCTTAGTGCTTAGAGATTCACATCCTGCATCCTAGAGCTTAG
CATTTTATATCCAGCATCCTTGTGCGTATCAGCTATGTTTTGTATCCTGC
TTAGCAGTTAACATCCTGCATCCTAGTACTTATCTGTTCTCATCCTGCAT
CCTAGAGCTTAGCAGTCAGGTCCCGTGGGAGCAAGAACCAGGGTCTGAGC
TCTGTCTGAGCCCAAGCATGGCTTTACTGCTTTGTTAATTGTGGCTCCCA
CCTCCACCCCACCCCAGCCAGTTTGCTTGCTAGAAGCCTTTCTGCACTGC
CTAATCCCCCTGCCTCACAGCAGAGAGCTGCAGCCATGACCTCCTCATTC
AGTATTAGGTGGACAAGTCGGAGATACCCAAACTCAATTTTAAAAGAATC
AAGTTGCTTTTGGGGCATGTTACTTCATCTTTTCTTACCCTGGGCCTCTA
TGACCTCCTCATTCAGTATTAGGTGGACAAGTCGGAGATACCCAAACTCA
ATTTTAAAAGAATCAAGTTGCTTTT.

The sequence of the 3′ end of 3′ arm was determined to be:

(SEQ ID NO: 159)
AGCTCTAAGAGAAGGAATACAGCTTGGGACAGAGTTTGTTTTGAAGTGGG
GTTCAGGTGCATTTCCTGGGTGTGTCCCTCATTTGGGGGTGTTAGGAGGC
AGACAATGCTAAGGGCATGGTTTGATATGGTAGACTGACCATCCTGGGGT
CCCTTCAGCTTGTGACACTCGCTGTACTGCTGGACTCTGCTGAGCCCTTT
GAAGCCAGGACTCCTCCTCTGCTGCAGGAGTGCAGTGTCCTTCTTGCTGT
ATGAAGCTGGGACAATGCTCTTTGGCCTTCATACTGGACATCCCATTGAG
AAGCTTGTCACTCTGTAGAGAATAGACATCGCCCCCTTGTGGTTGTGAGG
CTGCCCAGGACTTACTGCGGGGGGGGGGGGGGCATGTTGTCCAGCATAAG
GAGAGAAGACCCCACTGCATGCTGCTGGGAAAAGGAAAGTAACGTTCAGA
GTAGTTTCTACTGGCTGCCTGCGCTCTCACGCCTGTAAGAACAAACGTCC
TAATGTCTGCATGTGGAGGAAGGAGCCAGGGGTGCTTAGGGTGCTGTTGG
TCCCACCAATGTACTACTCATCTGGAAGACCTTGTCTTGGTTTTCTTGCC
ACTGGGACAAAGTGGGATTGGGTATCAGTCTCCCCATGTCGGGCAAATGT
ACTTGAACAAGCAGCATGGTGGACCAGACATGGGACTATTCTCAAAATCT
CACAGACACCCCCGGGGGGCATGTTACTTCATCTTTTCTTACCCTGGGCC
TCT.

The sequence of the 5′ probe (SEQ ID NO: 160) is:

CTAGGGAGTCAGGAAGTCACACTGGTGAAGAGTGGGGGTGTGGGATGACC
ACACTGAGGAGTCACACTTGAGGAAGGGGGGTGGCCACACTGAGGAGTGT
CTGAAAGCATTGAGACTGTACATCTCAGGTTATCAGGGCTTCAGAGAAAA
CAGACAGGGAGAAGAGACAAGAACTGGATTCTGTGCGCAAAGGGGGAAAA
GCAAGCAGATGTGAAGGGTGTGCTGTTAGAGTTTATCTAAAGATGTTTCT
TTCAGAAATAAAGAGATATAAGCTTTAATTTGGATGAAATAAATGTGGTC
TAATTTCCCAGAATGTAGAGGAACTCACTAATGTAGCAAGATTGGCCTTT
CAAAGCAGACCAAAGACATTGAGAATTAAGATAGCTATGATGGCGTGCTT
CCTTAGGTGGAAGTCCTATATGGAATCCCATACTCCCCAAATGTGACTGG
TCG.

The sequence of the 3′ probe (SEQ ID NO: 161) is:

AGGCAGGAGGGTCAGGAGTTCAAGGCCAGCCTAGTATACATGCAACAGTG
TCTCAAAAATCAAAACAGAGAGGAGGGCAGGAAAGGAGAAGGGAGCCAGG
GAGAGAAGGGAAGAGGAAGGGAGGGAGGGGGAAATGGAGGGAGGGGGAGA
TGGAGGGAGGGGGAGATGGAGGGAGGGGGAGATGGAGGGAGGGGGAGATG
GAGGGAGGGGGAGATGGAGGGAGGGGGATGGGAAAGAGGGTTTACAGCCC
TGGTTTATCTTGAACAGAATCCTTACTTTGTCCCTCAGAGTGACAGGACT
GAAAAGATTGTCCCAGGATTTTGGCTGCAAAGCAAGGTCCACTCAGAGAC
CACAGAGCTCG.

### Example 6

### Production of Recombinant IDO2 Using Baculovirus Expression Vectors

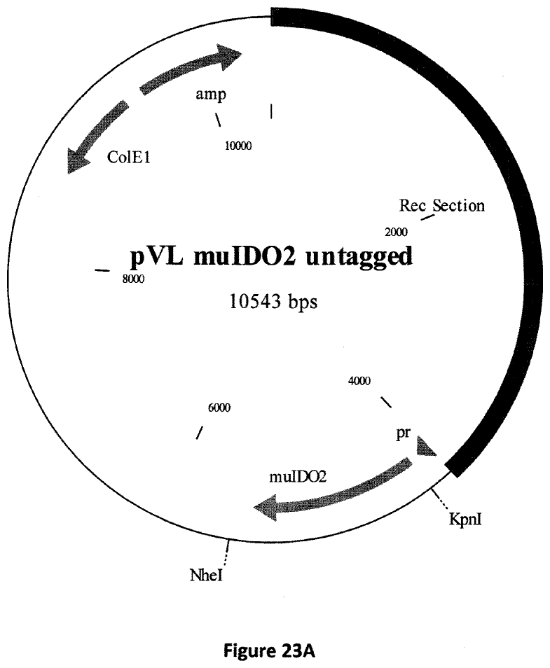

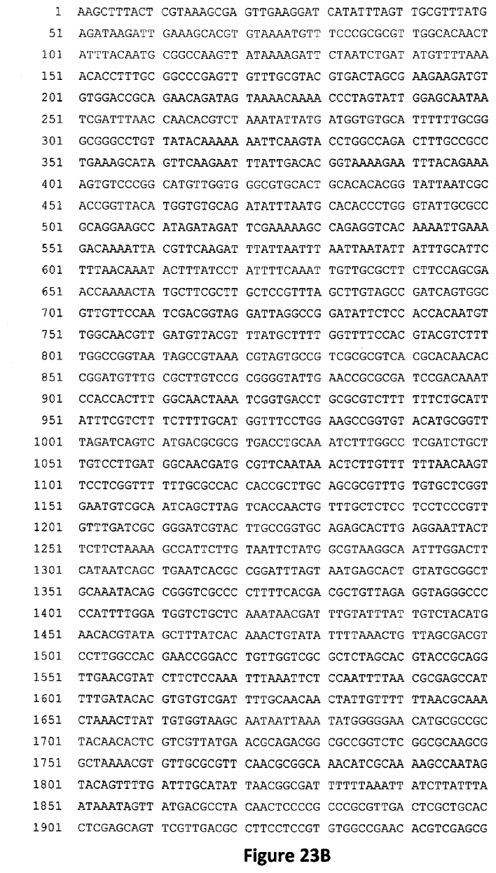

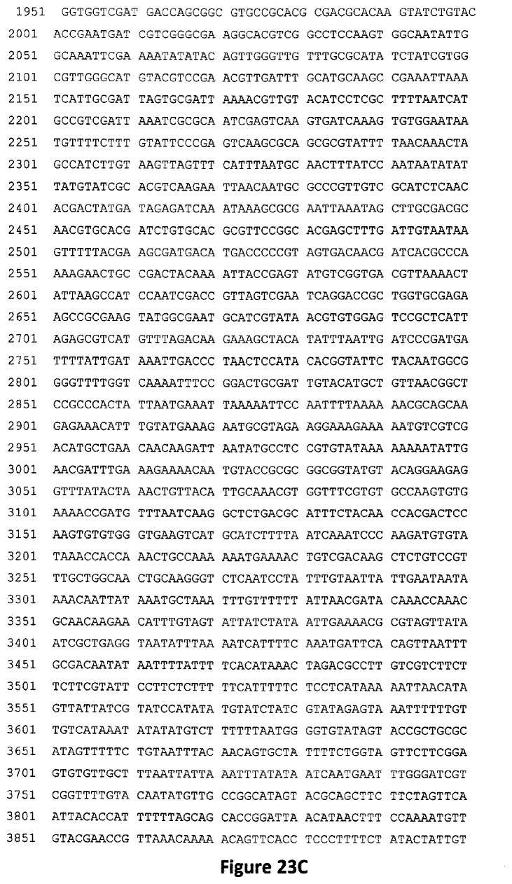

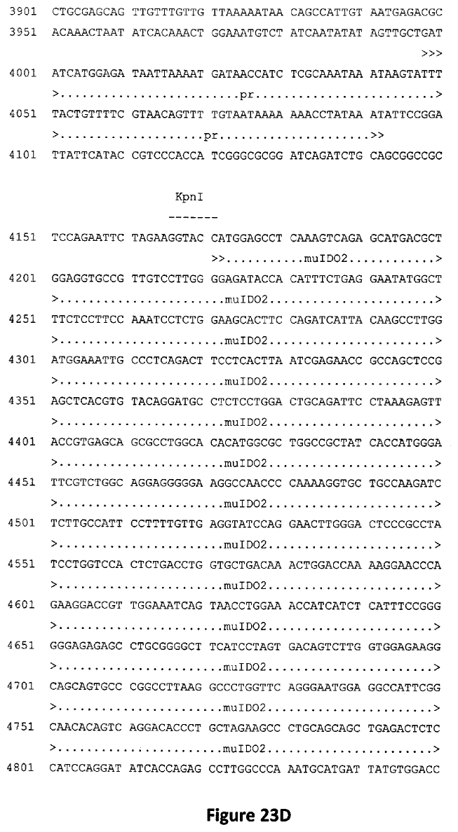

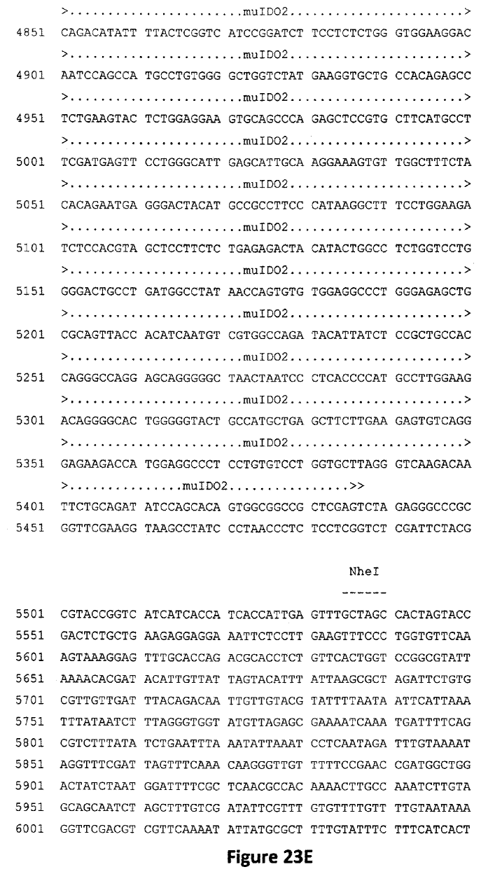

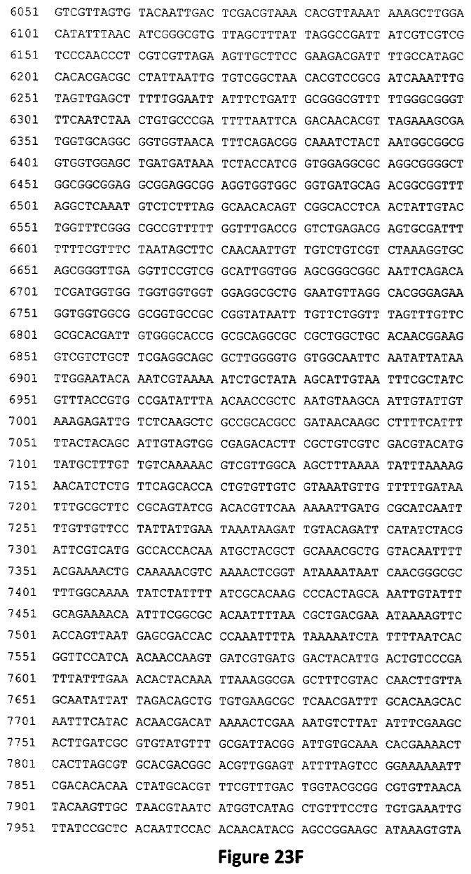

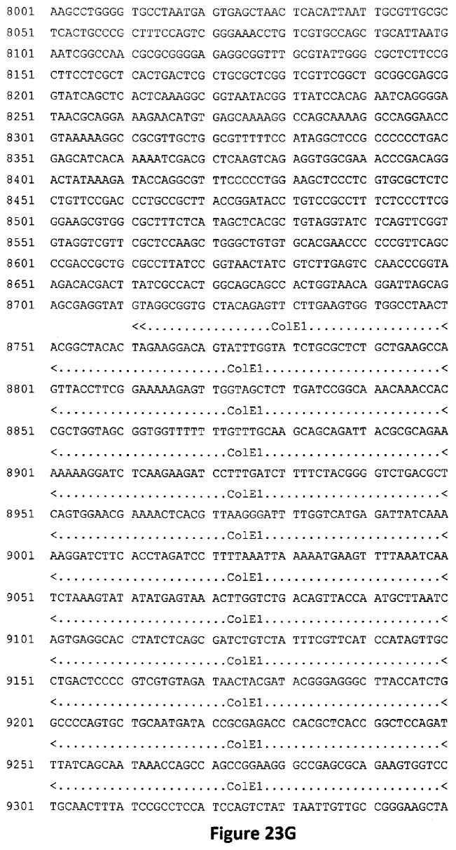

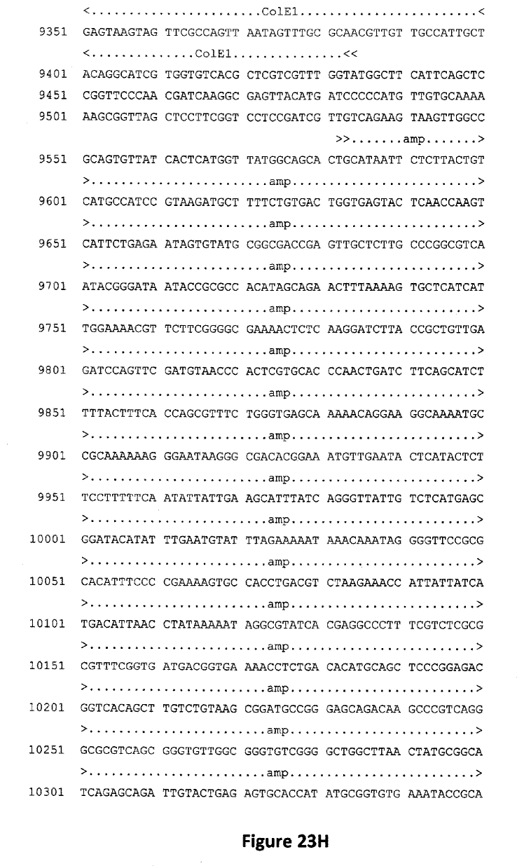

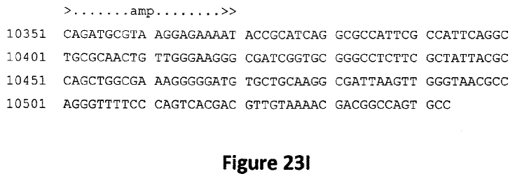

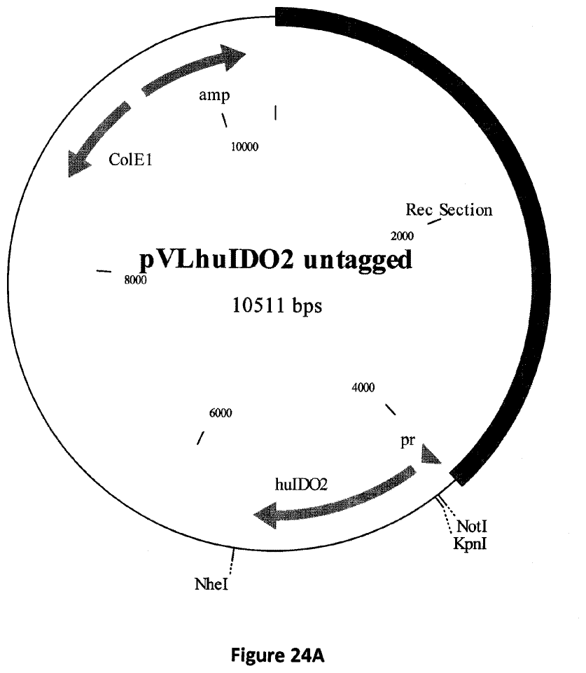

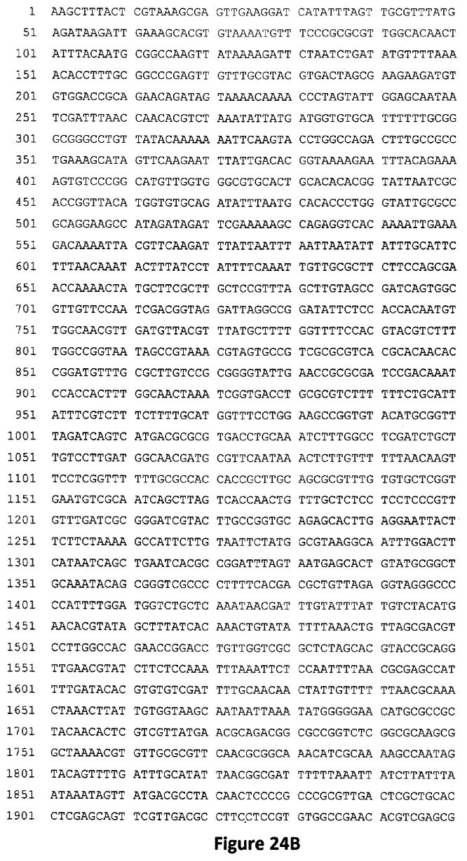

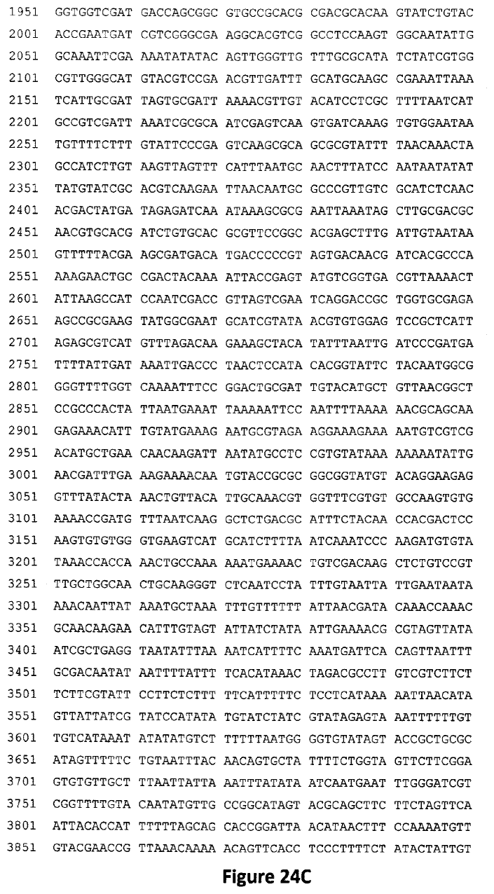

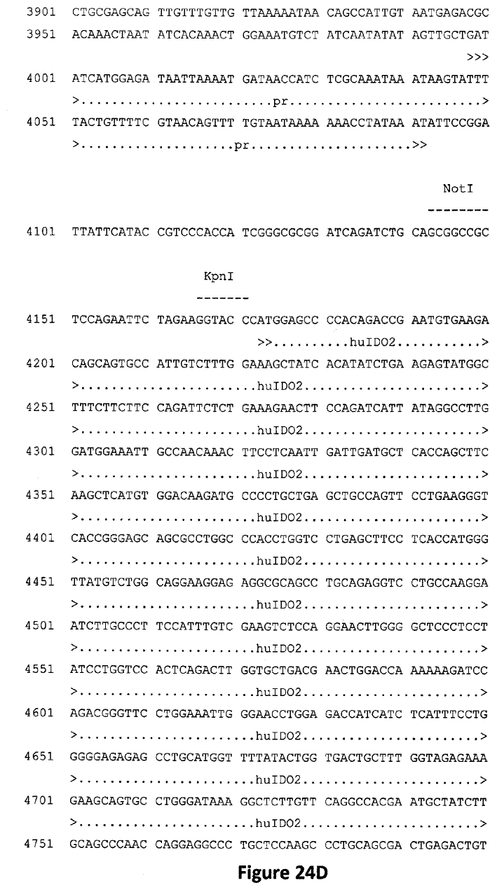

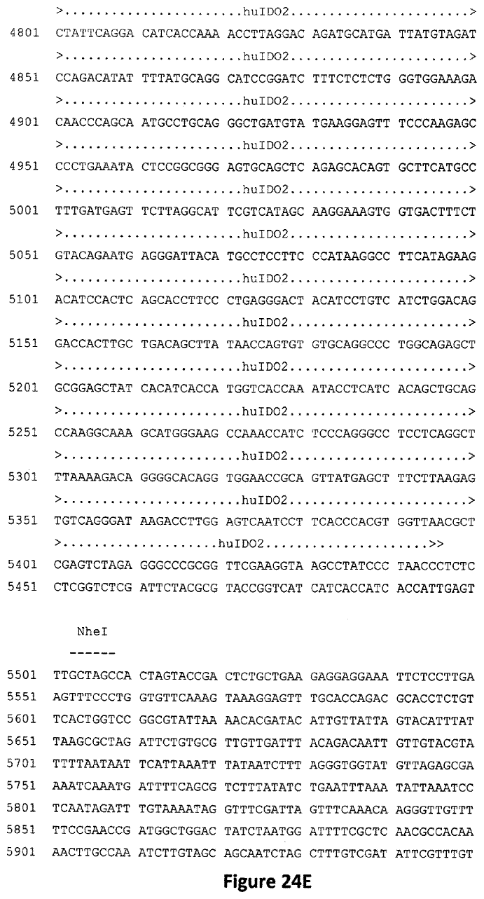

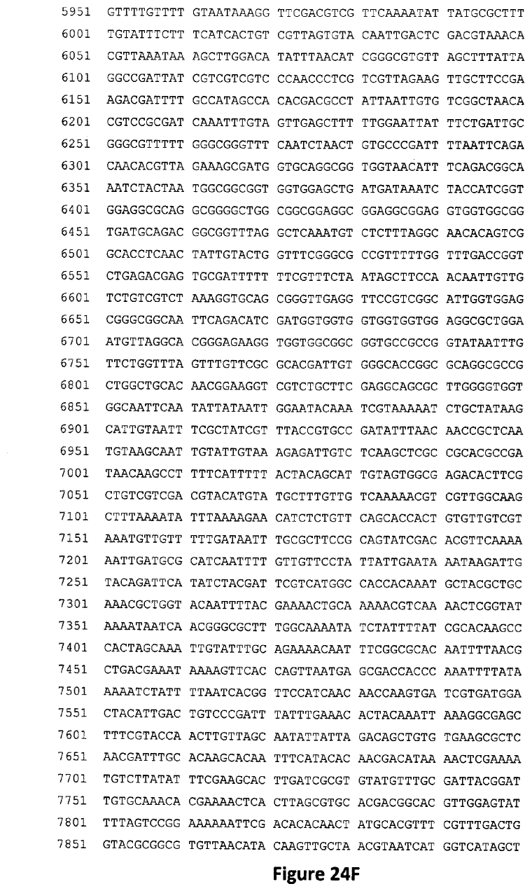

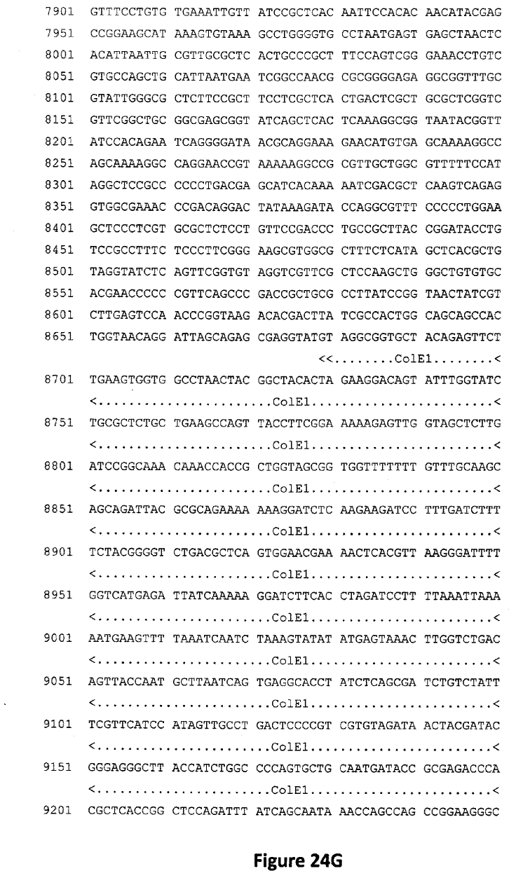

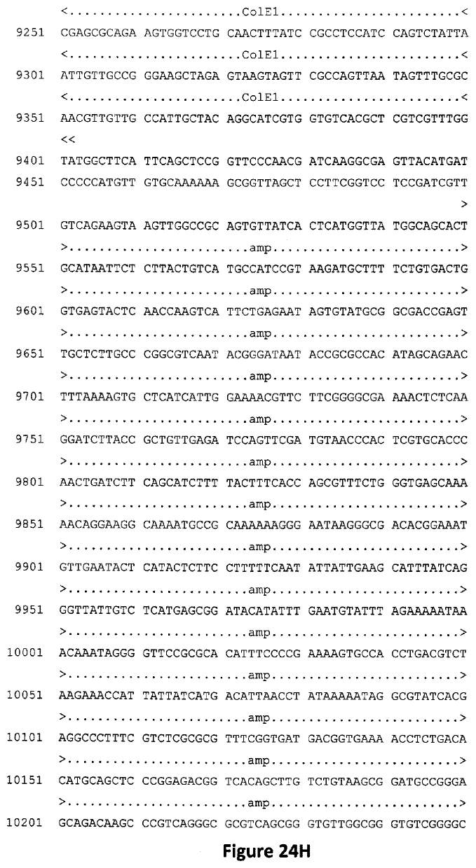

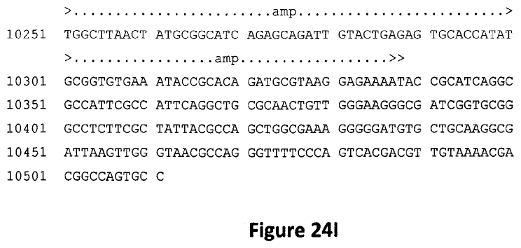

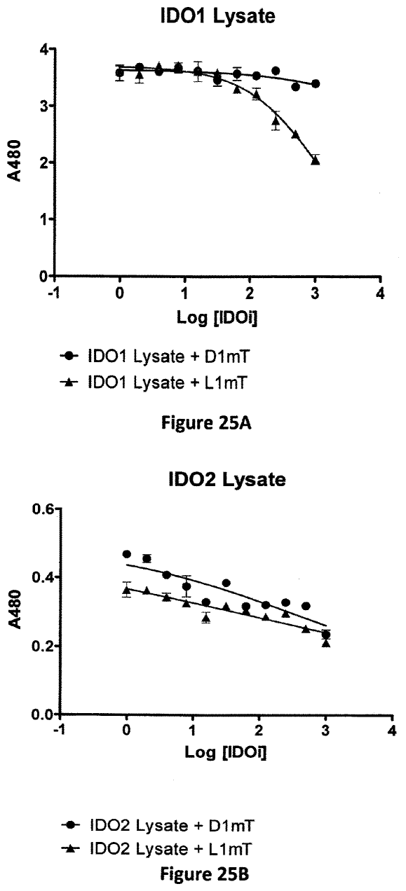

The recombinant transfer vectors (pVLmuIDO2, pVLhuIDO2; FIGS. 23 and 24, respectively) containing the complete coding regions of murine and human IDO2, respectively, were derived from eukaryotic expression vectors, pcDNAmuIDO2 and pcDNAhuIDO2. The KpnI/NheI insert fragments containing the complete ORF of pcDNAmuIDO2 and huIDO2 were cloned into KpnI/NheI digested pVL1392 recombination vector that was modified to contain KpnI, XbaI, NheI, SpeI multiple cloning sites at the original KpnI site (See FIGS. 23 and 24). The resulting vectors were analyzed by gel electrophoresis to verify the size and integrity of the inserted genes and inserted coding regions were also analyzed by standard diodeoxynucleotide automated sequencing (Macrogen, Inc., Rockville, MD).

Approximately 2 μg of each purified recombination transfer vector, pVLmuIDO2 and pVLhuIDO2, was mixed with 0.5 μg of Baculogold linearized baculovirus DNA (Pharmingen, BD Biosciences, Franklin Lakes, NJ) and added to 6 μL of Cell fectin (Invitrogen, Carlsbad, CA) in a total volume of 200 μL sf900II serum free medium (sf900II-SFM). The solution was incubated at room temperature for 30 minutes, supplemented with 800 μL of sf900II SFM (Gibco-BRL, Carlsbad, CA), mixed and added to 1×10⁶ cells of the Spodoptera frugiperda cell line, Sf9, in a 6 well plate. Cells were incubated at 28° C. for 4 hours after which the media was replaced with 2 mL sf900II-SFM 5% FCS. Supernatants containing the recombinant baculovirus (recBV) were harvested after six days and stored at 4° C. indefinitely.

“High Five” cells (Invitrogen) were plated at 1×10⁵ per well (24 well dish) in 0.5 mL “Express Five” medium (Invitrogen) and incubated at 28° C. for 24 hours. Media was replaced with 0.5 mL of media containing varying dilutions of recBVs containing the muIDO2 and huIDO2 ORFs. Typically 3, 10, 30 and 100 μL of a 1/100 dilution of the recBV viral stock were used. After 24 hours the cells were microscopically examined for signs of infection such as cell rounding, detachment and failure to divide. Optimal titers are established when 100% of the cells show signs of infection.

After 48 hours the media was removed and the cells collected and lysed in 400 μL of RIPA buffer, clarified by centrifugations and supplemented with Laemmli SDS PAGE running buffer. Briefly the lysates were heated to 80° C. for 10 minutes and 4 μL of material was subjected to SDS polyacrylamide gel electrophoresis (10% SDS-PAGE, Invitrogen) and either Coomassie blue stained or transferred to nitrocellulose membrane for Western analysis. For Western analysis, SDS-PAGE gel blots were typically blocked 1 hour at room temperature in PBS/0.1% Tween 20 and 3% nonfat dry milk, incubated with primary and secondary antibodies. Typically membranes are incubated for 1 hour at room temperature in blocking buffer, and then incubated with affinity purified rabbit anti-IDO2 antibody diluted at 1:5000 in blocking buffer. Membranes were washed at room temperature 3-5 times for 10 minutes each in washing buffer (PBS, 0.1% tween), after which the membrane was incubated with HRP-conjugated goat-anti-rabbit antibody (Santa Cruz) in blocking buffer for 1 hour at room temperature, followed by five 10 minutes washes in washing buffer. The membrane was prepared for exposure to X-ray film using a chemiluminescence method with SuperSignal® West Pico chemiluminescent substrate (Pierce cat. no. 34078, Rockford, IL). The highest dilution that gave the maximum amount of protein productions was used.

High five cells grown in Express Five-SFM media were plated at 2×10⁷ cells per T175 flask in 0.30 mL of media. After four hours the media was removed and recBV was added at 5-10 infectious units per cell in 6 mL of media. The flask was rocked every 10-15 minutes to ensure infection of every cell over the period of 1 hour, after which an additional 15 mL of media was added. After 24 hours the media in each flask was supplemented with tryptophan (final 100 μM) and hemin (5 μM final). After 64 hours, the infected cells were dislodged by the addition of glass beads. The cell suspension was separated from the glass beads by pipetting and pelleted by centrifugation (3,000 rpm 5 minutes). The cell pellet was either frozen or processed immediately.

The cell pellet, containing approximately 1.8×10⁸ cells, was resuspended in 40 mL 50 mM tris pH 8.8 and sonicated, 4×10 second pulses, on ice. The suspension was adjusted to 50 mM NaCl, 1% octylglucoside, vortexed, and clarified by centrifugation, 20 minutes at 13,000 rpm, at 4° C. The supernatant was passed over a DEAE-Sephacel column equilibrated in Tris pH 8.8, 50 mM NaCl, 1 mM EDTA (EQ buffer). The column was washed with 10 column volumes of equilibration buffer. The IDO2 proteins were eluted with 50 mM Tris pH 8.8, 0.5 M NaCl, 1 mM EDTA.

The eluted fractions were subjected to SDS-PAGE analysis followed by staining with Coomassie brilliant blue R-250. Peak IDO2 containing fractions were combined and dialyzed against EQ buffer. The dialysate was applied to a Mono Q column using the AKTAPurifier 100 fast protein liquid chromatography (FPLC) system. The column was equilibrated in EQ buffer and eluted using a linear gradient of EQ buffer with increasing NaCl 50 mM to 1 M. Elution profile was established by dual spectroscopic measurements at 280 nm and 403 nm and SDS-PAGE followed by staining with Coomassie brilliant blue R-250 and/or Western analysis. The 404/280 absorbance ratio was approximately 1.8-2.0. Peak. IDO2 eluates were contained in the 400-500 mM NaCl fractions. An additional peak containing IDO2 was also observed eluting at 250 nM NaCl which may contain mostly dimeric forms of the muIDO2 protein. Indeed, the early eluting peak contained IDO2 dimers as determined by a non-heat denatured sample analyzed by SDS-PAGE, whereby an 120 kDa band can be seen in samples not reduced or heated, while upon heating the larger bands resolves into a single major protein band at ˜55 kDa. Both mIDO2 containing peaks were pooled and concentrated approximated 5 fold by centrifugal filtration (Amicon Ultra-4, Billerica, HA).

The concentrated material was applied to a gel filtration column, Superdex™ 200 (GE Healthcare, Piscataway, NJ), equilibrated in EQ buffer. Fractions were collected and analyzed by SDS-PAGE/Coomassie staining and confirmed by Western analysis. IDO2 containing fractions were pooled and concentrated to approximately 2 μg/pL by centrifugal filtration (Amicon Ultra-4). The purified protein material exhibited 404-280 nM ratios of 1.5 to 1.8.

IDO2 activity was measured as the amount of kynurenine formed from L-tryptophan. The standard assay conditions consisted of a mixture containing 50 mM Pipes pH 6.5, between 50 and 600 nM of rmuIDO2 or rhuIDO2 (where 100 nM of rIDO2 is approximately 5.5 μg protein per mL reaction), 200 U catalase (0.2 mg/mL, sp act. ˜1000 U/mg), 200 UM L-tryptophan, 10 μM methylene blue and 20 mM ascorbic acid. The mixture was incubated at various times ranging from 15 minutes to 4 hours at 37° C. before the reaction was stopped by adding trichloroacetic acid (TCA) to 3% final. The samples were then heated to 50-60° C. for 30 minutes to convert N-formyl-kynureneine to kynurenine, clarified by centrifugation, and supernatants (100 μL) were removed to a new dish, mixed with 100 μL Ehrlich's reagent (2% p-dimethylaminobenzaldehyde w/v in glacial acetic acid), and incubated 10-30 minutes at room temperature. Absorbance at 490 nm was determined on a plate reader and the data collected and analyzed using Excel software (Microsoft). Typically, samples were analyzed in triplicate and control values were typically averaged and subtracted from sample values. Enzyme activity was estimated based on the conversion of tryptophan to kynurenine using linear regression analysis based on an absorbance standard curve derived from assay measurements on known amounts of kynurenine. Inhibitions studies were performed whereby varying amounts (0-100 μM) of D or L stereoisomers of 1-methyl tryptophan (D-1MT or L-1MT) or MTH-trp and demonstrate that the purified recIDO2 proteins are inhibited by D-1MT (FIG. 25).

### Example 7

Polyclonal rabbit anti-murine and anti-human IDO2 was prepared. Antisera were raised against a mixture of murine and human GST-IDO2 fusion protein. Antisera were screened for reactivity against the immunizing antigen by ELISA and western, and samples with high titer were purified by affinity chromatography. Specifically, antiserum was pre-absorbed to protein column containing GST. Since IDO2 and IDO1 possess conserved amino acid domains, the GST-unbound serum was passed over an IDO1-His-tagged peptide column. The unbound material was then affinity purified on an antigen specific peptide column containing human and mouse His-tagged IDO2. The resulting antibody was analyzed and determined to be IDO2 specific with no cross-reactivity with IDO1.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

Several publications and patent documents are cited in the foregoing specification in order to more fully describe the state of the art to which this invention pertains. The disclosure of each of these citations is incorporated by reference herein.

### REFERENCE TO AN ELECTRONIC SEQUENCE LISTING

The contents of the electronic sequence listing (MLH-4089USC1.xml; Size: 431,993 bytes; and Date of Creation: Oct. 20, 2023) is herein incorporated by reference in its entirety.

What is claimed:

1. A cDNA nucleic acid molecule encoding an indoleamine 2, 3-dioxygenase-2 (IDO2) protein comprising an amino acid sequence having at least 90% identity with SEQ ID NO: 2.

2. The nucleic acid molecule of claim 1, wherein said amino acid sequence has at least 95% identity with SEQ ID NO: 2.

3. The nucleic acid molecule of claim 1, wherein said amino acid sequence has at least 97% identity with SEQ ID NO: 2.

4. The nucleic acid molecule of claim 1, wherein said amino acid sequence has at least 99% identity with SEQ ID NO: 2.

5. The nucleic acid molecule of claim 1, wherein said amino acid sequence is SEQ ID NO: 2.

6. An isolated nucleic acid molecule encoding an indoleamine 2,3-dioxygenase-2 (IDO2) protein comprising an amino acid sequence having at least 90% identity with SEQ ID NO: 2, wherein said nucleic acid molecule is present in a vector.

7. The isolated nucleic acid molecule of claim 6, wherein said vector is a virus vector or a plasmid.

8. The isolated nucleic acid molecule of claim 6, wherein the encoding sequence is cDNA.

9. A cDNA nucleic acid molecule encoding an indoleamine 2, 3-dioxygenase-2 (IDO2) protein said nucleotide sequence having at least 90% identity with SEQ ID NO: 8 or 10.

10. The nucleic acid molecule of claim 9, wherein said nucleotide sequence has at least 95% identity with SEQ ID NO: 8 or 10.

11. The nucleic acid molecule of claim 9, wherein said nucleotide sequence has at least 97% identity with SEQ ID NO: 8 or 10.

12. The nucleic acid molecule of claim 9, wherein said nucleotide sequence has at least 99% identity with SEQ ID NO: 8 or 10.

13. The nucleic acid molecule of claim 9, wherein said nucleotide sequence is SEQ ID NO: 8 or 10.

14. The cDNA molecule of claim 9 present in a vector.

15. The isolated nucleic acid molecule of claim 14, wherein said vector is a virus vector or a plasmid.

16. The isolated nucleic acid molecule of claim 14, wherein the encoding sequence is cDNA.

17. A method for identifying a compound which modulates IDO2 activity comprising:
a. contacting the IDO2 protein of claim 1 with a test compound; and
b. determining tryptophan catabolism activity of the IDO2 protein;
wherein a change in the tryptophan catabolism activity in the presence of the test compound compared to the tryptophan catabolism activity in the absence of the test compound indicates that the test compound can modulate IDO2 activity.

18. The method of claim 17, wherein said IDO2 protein of step a) is expressed in a cell.

19. The method of claim 17, wherein IDO2 activity is assessed by measuring liver inhibitory protein (LIP).


SEQUENCE LISTING (163 sequences)

SEQ ID NO: 1 - protein, 420 aa, Homo sapiens
MLHFHYYDTSNKIMEPHRPNVKTAVPLSLESYHISEEYGFLLPDSLKELPDHYRPWMEIANKLPQLIDAHQLQAHVDKMPLLSCQFLKGHREQRLAHLVLSFLTMGYVWQEGEAQPAEVLPRNLALPFVEVSRNLGLPPILVHSDLVLTNWTKKDPDGNLEIGNLETIISFPGGESLHGFILVTALVEKEAVPGIKALVQATNAILQPNQEALLQALQRLRLSIQDITKTLGQMHDYVDPDIFYAGIRIFLSGWKDNPAMPAGLMYEGVSQEPLKYSGGSAAQSTVLHAFDEFLGIRHSKESGDFLYRMRDYMPPSHKAFIEDIHSAPSLRDYILSSGQDHLLTAYNQCVQALAELRSYHITMVTKYLITAAAKAKHGKPNHLPGPPQALKDRGTGGTAVMSFLKSVRDKTLESILHPRG

SEQ ID NO: 2 - protein, 405 aa, Homo sapiens
MEPHRPNVKTAVPLSLESYHISEEYGFLLPDSLKELPDHYRPWMEIANKLPQLIDAHQLQAHVDKMPLLSCQFLKGHREQRLAHLVLSFLTMGYVWQEGEAQPAEVLPRNLALPFVEVSRNLGLPPILVHSDLVLTNWTKKDPDGFLEIGNLETIISFPGGESLHGFILVTALVEKEAVPGIKALVQATNAILQPNQEALLQALQRLRLSIQDITKTLGQMHDYVDPDIFYAGIRIFLSGWKDNPAMPAGLMYEGVSQEPLKYSGGSAAQSTVLHAFDEFLGIRHSKESGDFLYRMRDYMPPSHKAFIEDIHSAPSLRDYILSSGQDHLLTAYNQCVQALAELRSYHITMVTKYLITAAAKAKHGKPNHLPGPPQALKDRGTGGTAVMSFLKSVRDKTLESILHP

SEQ ID NO: 3 - protein, 398 aa, Mus musculus
MTLEVPLSLGRYHISEEYGFLLPNPLEALPDHYKPWMEIALRLPHLIENRQLRAHVYRMPLLDCRFLKSYREQRLAHMALAAITMGFVWQEGEGQPQKVLPRSLAIPFVEVSRNLGLPPILVHSDLVLTNWTKRNPEGPLEISNLETIISFPGGESLRGFILVTVLVEKAAVPGLKALVQGMEAIRQHSQDTLLEALQQLRLSIQDITRALAQMHDYVDPDIFYSVIRIFLSGWKDNPAMPVGLVYEGAATEPLKYSGGSAAQSSVLHAFDEFLGIEHCKESVGFLHRMRDYMPPSHKAFLEDLHVAPSLRDYILASGPGDCLMAYNQCVEALGELRSYHINVVARYIISAATRARSRGLTNPSPHALEDRGTGGTAMLSFLKSVREKTMEALLCPGA

SEQ ID NO: 4 - protein, 403 aa, Mus musculus
MEPQSQSMTLEVPLSLGRYHISEEYGFLLPNPLEALPDHYKPWMEIALRLPHLIENRQLRAHVYRMPLLDCRFLKSYREQRLAHMALAAITMGFVWQEGEGQPQKVLPRSLAIPFVEVSRNLGLPPILVHSDLVLTNWTKRNPEGPLEISNLETIISFPGGESLRGFILVTVLVEKAAVPGLKALVQGMEAIRQHSQDTLLEALQQLRLSIQDITRALAQMHDYVDPDIFYSVIRIFLSGWKDNPAMPVGLVYEGAATEPLKYSGGSAAQSSVLHAFDEFLGIEHCKESVGFLHRMRDYMPPSHKAFLEDLHVAPSLRDYILASGPGDCLMAYNQCVEALGELRSYHINVVARYIISAATRARSRGLTNPSPHALEDRGTGGTAMLSFLKSVREKTMEALLCP

SEQ ID NO: 5 - protein, 403 aa, Homo sapiens [sequence omitted]

SEQ ID NO: 6 - protein, 407 aa, Mus musculus
MALSKISPTEGSRRILEDHHIDEDVGFALPHPLVELPDAYSPWVLVARNLPVLIENGQLREEVEKLPTLSTDGLRGHRLQRLAHLALGYITMAYVWNRGDDDVRKVLPRNIAVPYCELSEKLGLPPILSYADCVLANWKKKDPNGPMTYENMDILFSFPGGDCDKGFFLVSLLVEIAASPAIKAIPTVSSAVERQDLKALEKALHDIATSLEKAKEIFKRMRDFVDPDTFFHVLRIYLSGWKCSSKLPEGLLYEGVWDTPKMFSGGSAGQSSIFQSLDVLLGIKHEAGKESPAEFLQEMREYMPPAHRNFLFFLESAPPVREFVISRHNEDLTKAYNECVNGLVSVRKFHLAIVDTYIMKPSKKKPTDGDKSEEPSNVESRGTGGTNPMTFLRSVKDTTEKALLSWP

SEQ ID NO: 7 - DNA, 1224 bp, Mus musculus
  source 1..1224: mol_type = genomic DNA
atggcactcagtaaaatatctcctacagaaggttctagaaggatccttgaagaccaccacatagatgaagatgtgggctttgctctaccacatccactggtggagctgcccgacgcatacagcccctgggtccttgtggctagaaatctgcctgtgctgattgagaacgggcagcttcgagaagaagttgaaaagctgcccacactgagcacggacggactgagaggacacaggttacagcgcctggcacacctggccctggggtacatcaccatggcgtatgtgtggaaccgaggggatgacgatgttcgaaaggtgctgccccgcaatattgctgttccctactgcgagctctcagagaagttgggcctgcctcctattctgtcttatgcagactgtgtcctggcaaactggaagaaaaaggaccccaatgggcccatgacatacgagaacatggacattctgttctcatttcctggtggggactgcgacaagggcttcttcctcgtctctctattggtggaaatcgcagcttctcctgcaatcaaagcaatccccactgtatccagtgcagtagagcgtcaagacctgaaagcattggaaaaggcactgcacgacatagctaccagtctggagaaagccaaggaaatttttaagaggatgcgtgactttgtggacccagacacgtttttccacgttctccgcatatatctgtctggctggaaatgcagctccaagctgccagaaggtctgctgtatgagggggtctgggacaccccaaaaatgttttcagggggcagtgcaggccagagcagcatcttccagagtcttgatgtccttctgggaataaaacacgaggctggcaaagaatctcctgcagaattcctccaggaaatgagagagtacatgcctccagcccaccggaacttccttttcttcttagagtcagctcccccagtccgtgagtttgtcatttcaagacacaatgaagacttgacgaaagcttataacgagtgtgtgaatggtctggtctctgtgagaaagttccacctcgcaatagtagatacttacattatgaaaccttcgaagaagaagcccactgatggcgacaagtcggaagagccctcaaatgtggaaagcagagggactgggggtacgaatcccatgactttcctaaggagtgtgaaagatacaaccgagaaagctcttctgagttggccttag

SEQ ID NO: 8 - DNA, 2279 bp, Homo sapiens
  source 1..2279: mol_type = genomic DNA
agtccagatgatagttaagaaagcagtaagaatacagagagtccacaatgagatgaaaatgcactgccagttgaaacatcctcctacactggagctttataaatattttaaagacaaggattggattagatttgacattagaaatgtaccataatacagaaggcaatggacacctaaagaacagaatgaaaaccttcttaggaaatgaagcttgacacttcacccaccaggccaccacaagaatgttgcattttcattattatgatacttcaaacaaaataatggagccccacagaccgaatgtgaagacagcagtgccattgtctttggaaagctatcacatatctgaagagtatggctttcttcttccagattctctgaaagaacttccagatcattataggccttggatggaaattgccaacaaacttcctcaattgattgatgctcaccagcttcaagctcatgtggacaagatgcccctgctgagctgccagttcctgaagggtcaccgggagcagcgcctggcccacctggtcctgagcttcctcaccatgggttatgtctggcaggaaggagaggcgcagcctgcagaggtcctgccaaggaatcttgcccttccatttgtcgaagtctccaggaacttggggctccctcctatcctggtccactcagacttggtgctgacgaactggaccaaaaaagatccagacgggaacctggagaccatcatctcatttcctgggggagagagcctgcatggttttatactggtgactgctttggtagagaaagaagcagtgcctgggataaaggctcttgttcaggccacgaatgctatcttgcagcccaaccaggaggccctgctccaagccctgcagcgactgagactgtctattcaggacatcaccaaaaccttaggacagatgcatgattatgtagatccagacatattttatgcaggcatccggatctttctctctggatggaaagacaacccagcaatgcctgcagggctgatgtatgaaggagtttcccaagagcccctgaaatactccggcgggagtgcagctcagagcacagtgcttcatgcctttgatgagttcttaggcattcgtcatagcaaggaaagtggtgactttctgtacagaatgagggattacatgcctccttcccataaggccttcatagaagacatccactcagcaccttccctgagggactacatcctgtcatctggacaggaccacttgctgacagcttataaccagtgtgtgcaggccctggcagagctgcggagctatcacatcaccatggtcaccaaatacctcatcacagctgcagccaaggcaaagcatgggaagccaaaccatctcccagggcctcctcaggctttaaaagacaggggcacaggtggaaccgcagttatgagctttcttaagagtgtcagggataagaccttggagtcaatccttcacccacgtggttgagaggctgccctctccccagcaatgcagagcccccatggagggcaggtgggcctggagaatgagggtcagggttctgcctgggatcatccaggaaggatctcagccctattcatgtttctgctctacagagcactatattctccttgttgagagctgttggcttcacaaaggagagttgatgtggccaagcctttccctccctacctgatcactgcttaacggcatgtataatggatacttcctcatgcagaacccccagaggagtgactgtatgccattctctttgccaagtaatagaaaaccaatctaaatgtcaaaaatcagataaaattgcctggggatacattacttgttgattttcttaaaaaacaaattcacttaacaattcattaagttcatactgagcactgcctccaagattaaaaccaggatttctgtggtcccagaccagccctcttctccctgaatgtgttgagttggtggcaggaggttggaaatgctccagtggagatgggaagatagaggatgctgacaataaggacttggaagtcactagtgtgaaaatgagcagttaatgatatgggaacggatgagactttccacgtggtacctagatttgcaaattctattgtaatgcctttatttttagaagaattattctctcttcttactctgaaaatctgtatttgtaaaatgaatgaatggatcctatataagtaaataagaaaactgggaataagtagtaaatcaatgtgtttagtgtgcaaataaatgtaaatgcttttattg

SEQ ID NO: 9 - DNA, 1245 bp, Homo sapiens
  source 1..1245: mol_type = genomic DNA
atgttgcattttcattattatgatacttcaaacaaaataatggagccccacagaccgaatgtgaagacagcagtgccattgtctttggaaagctatcacatatctgaagagtatggctttcttcttccagattctctgaaagaacttccagatcattataggccttggatggaaattgccaacaaacttcctcaattgattgatgctcaccagcttcaagctcatgtggacaagatgcccctgctgagctgccagttcctgaagggtcaccgggagcagcgcctggcccacctggtcctgagcttcctcaccatgggttatgtctggcaggaaggagaggcgcagcctgcagaggtcctgccaaggaatcttgcccttccatttgtcgaagtctccaggaacttggggctccctcctatcctggtccactcagacttggtgctgacgaactggaccaaaaaagatccagacgggaacctggagaccatcatctcatttcctgggggagagagcctgcatggttttatactggtgactgctttggtagagaaagaagcagtgcctgggataaaggctcttgttcaggccacgaatgctatcttgcagcccaaccaggaggccctgctccaagccctgcagcgactgagactgtctattcaggacatcaccaaaaccttaggacagatgcatgattatgtagatccagacatattttatgcaggcatccggatctttctctctggatggaaagacaacccagcaatgcctgcagggctgatgtatgaaggagtttcccaagagcccctgaaatactccggcgggagtgcagctcagagcacagtgcttcatgcctttgatgagttcttaggcattcgtcatagcaaggaaagtggtgactttctgtacagaatgagggattacatgcctccttcccataaggccttcatagaagacatccactcagcaccttccctgagggactacatcctgtcatctggacaggaccacttgctgacagcttataaccagtgtgtgcaggccctggcagagctgcggagctatcacatcaccatggtcaccaaatacctcatcacagctgcagccaaggcaaagcatgggaagccaaaccatctcccagggcctcctcaggctttaaaagacaggggcacaggtggaaccgcagttatgagctttcttaagagtgtcagggataagaccttggagtcaatccttcacccacgtggt

SEQ ID NO: 10 - DNA, 2464 bp, Mus musculus
  source 1..2464: mol_type = genomic DNA
cgcacaagtacaaccacacagaagacacagctggaaagctccctggcctgggcattcctctggggcagagacctcacgcgaaaatatggagcctcaaagtcagagcatgacgctggaggtgccgttgtccttggggagataccacatttctgaggaatatggctttctccttccaaatcctctggaagcacttccagatcattacaagccttggatggaaattgccctcagacttcctcacttaatcgagaaccgccagctccgagctcacgtgtacaggatgcctctcctggactgcagattcctaaagagttaccgtgagcagcgcctggcacacatggcgctggccgctatcaccatgggattcgtctggcaggagggggaaggccaaccccaaaaggtgctgccaagatctcttgccattccttttgttgaggtatccaggaacttgggactcccgcctatcctggtccactctgacctggtgctgacaaactggaccaaaaggaacccagaaggaccgttggaaatcagtaacctggaaaccatcatctcatttccggggggagagagcctgcggggcttcatcctagtgacagtcttggtggagaaggcagcagtgcccggccttaaggccctggttcagggaatggaggccattcggcaacacagtcaggacaccctgctagaagccctgcagcagctgagactctccatccaggatatcaccagagccttggcccaaatgcatgattatgtggacccagacatattttactcggtcatccggatcttcctctctgggtggaaggacaatccagccatgcctgtggggctggtctatgaaggtgctgccacagagcctctgaagtactctggaggaagtgcagcccagagctccgtgcttcatgccttcgatgagttcctgggcattgagcattgcaaggaaagtgttggctttctacacagaatgagggactacatgccgccttcccataaggctttcctggaagatctccacgtagctccttctctgagagactacatactggcctctggtcctggggactgcctgatggcctataaccagtgtgtggaggccctgggagagctgcgcagttaccacatcaatgtcgtggccagatacattatctccgctgccaccagggccaggagcagggggctaactaatccctcaccccatgccttggaagacaggggcactgggggtactgccatgctgagcttcttgaagagtgtcagggagaagaccatggaggccctcctgtgtcctggtgcttagcagtcatgtcctgcaccctaacacttagatgttctcatcctgcatcccagcgttagaggttcacatcctgcatcctagtgcttagctgttcttgtgctatatcccagcgcttagcagtcatgtcctgcatcctagtgcttagcattttatatccagcattttagtgcttagagattcacatcctgcatcctagagcttagcattttatatccagcatccttgtgcgtatcagctatgttttgtatcctgcttagcagttaacatcctgcatcctagtacttatctgttctcatcctgcatcctagagcttagcagtcaggtcccgtgggagcaagaaccagggtctgagctctgtctgagcccaagcatggctttactgctttgttaattgtggctcccacctccaccccaccccagccagtttgcttgctagaagcctttctgcactgcctaatccccctgcctcacagcagagagctgcagccatgacctcctcattcagtattaggtggacaagtcggagatacccaaactcaattttaaaagaatcaagttgcttttggggcatgttacttcatcttttcttaccctgggcctcttcccttcttccctacctccctcgtcccttagtctttcacccctctctctttctccttttgtcaccctccccctcccctgcttactctcttttcccttcccccctctcctcatccctccttcctttcttccttccctttttgtctgtgaagcaccaggtctgatgggcctcaaactgtgatcttcctgtctcacccttcaaaggttatgtgtatgtgacgtgtgtgtgtgtgtgtgtgtgtgtgtgtgtgtgtgtgttcgtttcttttgtttttccctagtggagatgacacccaaagatttgcacataccaggcaattgctccaccacctgactacagtcccagctctctgtattcctgaaggaaagtcttgatgagttgcctaggctggtattgagctctttagcccaggcaggccttagtctgagtagctgggatgtacagggatgagccactgagccatgctgctgctgctaacgatgatgacgatgatgatgatgaagattatgataactacagtcactgcaataatgacggcaaagataatgaaaaaaaaaaaaaaa

SEQ ID NO: 11 - DNA, 1194 bp, Mus musculus
  source 1..1194: mol_type = genomic DNA
atgacgctggaggtgccgttgtccttggggagataccacatttctgaggaatatggctttctccttccaaatcctctggaagcacttccagatcattacaagccttggatggaaattgccctcagacttcctcacttaatcgagaaccgccagctccgagctcacgtgtacaggatgcctctcctggactgcagattcctaaagagttaccgtgagcagcgcctggcacacatggcgctggccgctatcaccatgggattcgtctggcaggagggggaaggccaaccccaaaaggtgctgccaagatctcttgccattccttttgttgaggtatccaggaacttgggactcccgcctatcctggtccactctgacctggtgctgacaaactggaccaaaaggaacccagaaggaccgttggaaatcagtaacctggaaaccatcatctcatttccggggggagagagcctgcggggcttcatcctagtgacagtcttggtggagaaggcagcagtgcccggccttaaggccctggttcagggaatggaggccattcggcaacacagtcaggacaccctgctagaagccctgcagcagctgagactctccatccaggatatcaccagagccttggcccaaatgcatgattatgtggacccagacatattttactcggtcatccggatcttcctctctgggtggaaggacaatccagccatgcctgtggggctggtctatgaaggtgctgccacagagcctctgaagtactctggaggaagtgcagcccagagctccgtgcttcatgccttcgatgagttcctgggcattgagcattgcaaggaaagtgttggctttctacacagaatgagggactacatgccgccttcccataaggctttcctggaagatctccacgtagctccttctctgagagactacatactggcctctggtcctggggactgcctgatggcctataaccagtgtgtggaggccctgggagagctgcgcagttaccacatcaatgtcgtggccagatacattatctccgctgccaccagggccaggagcagggggctaactaatccctcaccccatgccttggaagacaggggcactgggggtactgccatgctgagcttcttgaagagtgtcagggagaagaccatggaggccctcctgtgtcctggtgct

SEQ ID NO: 12 - DNA, 1227 bp, Homo sapiens
  source 1..1227: mol_type = genomic DNA
atggagccccacagaccgaatgtgaagacagcagtgccattgtctttggaaagctatcacatatctgaagagtatggctttcttcttccagattctctgaaagaacttccagatcattataggccttggatggaaattgccaacaaacttcctcaattgattgatgctcaccagcttcaagctcatgtggacaagatgcccctgctgagctgccagttcctgaagggtcaccgggagcagcgcctggcccacctggtcctgagcttcctcaccatgggttatgtctggcaggaaggagaggcgcagcctgcagaggtcctgccaaggaatcttgcccttccatttgtcgaagtctccaggaacttggggctccctcctatcctggtccactcagacttggtgctgacgaactggaccaaaaaagatccagacgggttcctggaaattgggaacctggagaccatcatctcatttcctgggggagagagcctgcatggttttatactggtgactgctttggtagagaaagaagcagtgcctgggataaaggctcttgttcaggccacgaatgctatcttgcagcccaaccaggaggccctgctccaagccctgcagcgactgagactgtctattcaggacatcaccaaaaccttaggacagatgcatgattatgtagatccagacatattttatgcaggcatccggatctttctctctgggtggaaagacaacccagcaatgcctgcagggctgatgtatgaaggagtttcccaagagcccctgaaatactccggcgggagtgcagctcagagcacagtgcttcatgcctttgatgagttcttaggcattcgtcatagcaaggaaagtggtgactttctgtacagaatgagggattacatgcctccttcccataaggccttcatagaagacatccactcagcaccttccctgagggactacatcctgtcatctggacaggaccacttgctgacagcttataaccagtgtgtgcaggccctggcagagctgcggagctatcacatcaccatggtcaccaaatacctcatcacagctgcagccaaggcaaagcatgggaagccaaaccatctcccagggcctcctcaggctttaaaagacaggggcacaggtggaaccgcagttatgagctttcttaagagtgtcagggataagaccttggagtcaatccttcacccacgtggttaggat

SEQ ID NO: 13 - DNA, 1218 bp, Mus musculus
  source 1..1218: mol_type = genomic DNA
atggagcctcaaagtcagagcatgacgctggaggtgccgttgtccttggggagataccacatttctgaggaatatggctttctccttccaaatcctctggaagcacttccagatcattacaagccttggatggaaattgccctcagacttcctcacttaatcgagaaccgccagctccgagctcacgtgtacaggatgcctctcctggactgcagattcctaaagagttaccgtgagcagcgcctggcacacatggcgctggccgctatcaccatgggattcgtctggcaggagggggaaggccaaccccaaaaggtgctgccaagatctcttgccattccttttgttgaggtatccaggaacttgggactcccgcctatcctggtccactctgacctggtgctgacaaactggaccaaaaggaacccagaaggaccgttggaaatcagtaacctggaaaccatcatctcatttccggggggagagagcctgcggggcttcatcctagtgacagtcttggtggagaaggcagcagtgcccggccttaaggccctggttcagggaatggaggccattcggcaacacagtcaggacaccctgctagaagccctgcagcagctgagactctccatccaggatatcaccagagccttggcccaaatgcatgattatgtggacccagacatattttactcggtcatccggatcttcctctctgggtggaaggacaatccagccatgcctgtggggctggtctatgaaggtgttgccacagagcctctgaagtactctggaggaagtgcagcccagagctccgtgcttcatgccttcgatgagttcctgggcattgagcattgcaaggaaagtgttggctttctacacagaatgagggactacatgccgccttcccataaggctttcctggaagatctccacgtagctccttctctgagagactacatactggcctctggtcctggggactgcctgatggcctataaccagtgtgtggaggccctgggagagctgcgcagttaccacatcaatgtcgtggccagatacattatctccgctgccaccagggccaggagcagggggctaactaatccctcaccccatgccttggaagacaggggcactgggggtactgccatgctgagcttcttgaagagtgtcagggagaagaccatggaggccctcctgtgtcctggtgcttag

SEQ ID NO: 14 - DNA, 52386 bp, Mus musculus
  source 1..52386: mol_type = genomic DNA
tagttagaaggcagctcgtttccagacagatcatgccgggccagcacagttccacgaggagcgatttttttggatgggggaagaaagtgaagaagaggagaaagaggaaaagggggtgaagggtcgcggtctcagtcttttatttgtgccatgacatagatgccaggtaatgaatgatgaatgatgcgcacaggtgaagcataggcctgcctggagtgtatggtggttgccatggcaacagacccaagagggtccttgtcgcttagggatgatgtcatagctggattccgaggctggttgtaaagcagtttctgatgctaacaatacatatatatgtaatgataacaaagaaaaagaggccatgaatttgaaaggaaggaaagaagttaggaaggaaggaaggaaggaaggaaggaaggaaggaaggaaggaaggaaggaaggaaggaaggaaggaaagaagatgggaatttggagagatgaaagggaagaatgatataaatacattttaatttcagcaaataaatacattttcaaagataaaaagccaataaagagaaaccctgtctcgaaaaaacaacaacaaaccaaaaacaaacaaacaaacaagccaataaaactgactcctagtttagtcattttgatacactgtcagaatccctcaggaaccaaactcatgttgagtatggatctgcacagccatagctgagagttaagttcgtgtctcccactttgtccctggatttaacagcccctttctcatactttatgaatgagaagggaaagggggtgggggcttctcttggagtcagcattttaattcctaatcttcattgttgacttactacttggctttctttcagaataaatttgcggccatcctggagttcaacctcagggcaaggttctcccctaccgctgaagcaccgcacaagtacaaccacacagaagacacagctggaaagctccctggcctgggcattcctctggggcagagacctcacgcgaaaatatggagcctcaaagtcagagcatgacgctggaggtgccgttgtccttggggagataccacatttctgaggaatatggctttctccttccaaatcctctggtaaggattggcttgtcctggtcagcacatgtgtctggattgttagtctttaccttatgtggttattggaaggttcggtataagtcaccagctggaggaagagtgggaatacagtcctgtgagtcccggaagagcagaggcaactgctactggggacagcacaaagccagtggtcccctccccccagcccccaaagtttccttaaatctatggtagttagcactgactaatcagaagcaaaggtgctatcagagaggagagactggatggagacatgccctaaggacaattagaaccctataactacagcaaagagtggacatgcacacaaccacatggtgtgacacagaataatggagtggccccactgtatatccactctgctttggagacaggttcgccatgcatcccagacctaagcagccaaagggcttagccttctgtgttgatgcaaaccagtttatcttacttgctggtgacagcagagataatgggagacctgcaatgtttttagagccagtaagaagattagatgttatcgggaatttccgatactcgtgaggaatgttggaaaacaagcaggcctgactttcagtccaactgcacagcagtctctagtatacctgctcttgttaagtgtagtctgggcttgggagacatatgccggcaaagtgctagtggcaaagcccatgcttttggcatcttttaaaaactgaccgtacctaagggagggtgaaggccactgagttaaaagtccagggacccgatgttgcagtcgcggtaactgctgatcagcaccaactcacctagttcctctcttacttgtgattaaaaatatcagggtctcaaacacctatcacagtgagactcttgttgttttgaaacagagaatattgatttatgtagcccaggctggccacttgcctccacttccttactgctgggaccatagctgaactcccacagcagatccttttgtgttgaacggtactggagggatctaattgaagagaactaagttctccacagaaagatgtcagctacgacatcggtcatagacatctgcaagctgtccaggttaggggttagtcattagaaccagggccagatctcacagcttgaatcaagccaggccttcctgctgctctctcacggacagttaggaagcagactgtactctgacatgatagcttgcagccgacggctcccggctcctctgtgctccttgtcacgcaggccctgctataaactaaagtgtctgacatgacttcctggtgcctcgtaaagttattaaaagtcagatttctcctttgttctcggaattggttcgagagccctcaggggatttgctgagggtgtgtgtgtgtgtgtgtgtgtgtgtgtgtgtgtgtgtattctaagagtgcttccctaaaagggtcctctgtactgtcaacggatctttcatagtctctctctccctgtctccctgtctctctgtctttctgtctctctgtctctctctgtttctgtctttctgtctctgtctgtctgtctgtctgtctatctctgtgtgtgtgtgtgtattttgaaggtaggaggatctgagcctgaactacacacagctagatcctgtgactctgtctcaaaacagagacacaatcaaactctcaactaatcgaaacaccacaaggcacactccattctgggtgtgtggtctcagtccagcgtattcccatccttccaagtttacaggagaggaagggctgatggcagatacacccttgtcattactgggcatgtgtcctgagtttggaaagagaacactgccaagcaggggaacgaatatcagggcaggccatagctctggggctttctgggctgctttcctctttgtaaatactgggaccaggcagacatacagataagacacaatagcaacatcaagtttcccccctatttgttagaaaggttctatggtatggtgtgatggatgggtaccccttaaaagaggtccctagtgctgttcacacagaaaggaaatattccctgtcccacccagctactcatttatagcccagtaatttcaatatgattctttctgtttcaattttctttcattcaagtgatggttttcaatgtcttttttgttaaaggaagaggaagaaaccttgactaggatcaggtcctggctgctccaaaggtcaagacagactcagttcgacttcactggacaaacagtcctgcaactttacactggttgggaaagaacagtgggtttggcccagtccagtgccctagggcatagccaagacctgttcttcttgggccaagggcaagtggtaggtggcagaccttccccatctctttcagaaagtctcacttaagaaaacctagctggctagcaagttcaagggtataaattaccctatccagctgtttctatcaattcaaagacttccaatttttagtgtctctatggaactttgctcctcgaatctgtaaaaataccacaagaattctttgctaagacatctgtcctctgtgtatatttgggaggacaagacatctttttcagatgaagaatgtgaggaaatcagtttgcaggagaggaggcagttaagaggaggcagcaagaggctggcctgcagcagagctatgaccccaagtgccaggcccactctgaaaattgatttaacacccttcctaaacacaatgtgactgctacttgagtcatttcctctcacagcactaagggcagggcaggttagagcagtgggtcctgggctgtggggtgagacctttaggggagtttcaacaaacaatccagatgccagagccccaccctgtttattcagactctagacccaagagctgtacgctgggaaggccagatagggagaacaagccagagaatgccagacagggagcacaagccagcaaagtcagccgtgaaggtgccacggccaatagagactcctaagtgcgaaaataaataaactgatgacatgcccactccatgcttaaggggaaagtagatcagagagggagagagagagagagagagagaggcacccagaggcagaggcctctggacgattccgtagtagatagagaaagcagactgaagatggccaccagactggacatggccaaggctacctgggaggaaaggaagaacaacagaagtcagagaatagagaaaacctagtgagagaagcaggaaggggttgggggggtggatggagagaagttagcaatgacagcagggttctaagaagaaagagttggggaggggagatcctgtgagctggagggagtggagtgagtggaggggaagagctgagaagggacagatgctagcaggggctttgagatgtacagcaggtacttgtgataggtgactggaggccactggtgaccacatgtcccttctgcaagaggtaagggaaaacattcctttaggtagagggaaaccagtgtcacaagtccctgaggaaagctggctttttatctaacccccagaaatcctcctgtagtccaggtgaagctcatttctggacatacagactgccttttggagtttggggaactggagtttcctttggacctgacagtcaggagacgttgcacatgtaacgtaggggtagacacaagactgacaggaatggcaatgagagaggacaacctccagggaaaaaccaacatgtcccttctcattcagaaattctaagcacttggttgaagcattgggccacagctggagagaagaccggataggaaattaattgccacggacatctgtatttttcagataagacaaaaaggaagaggtctaacattgtcctgaaatatgaggttgccactgatgatctctggacacatcagactagtcagataggggacttgaaacacaatgaatccagagtctgtagtagagacaaagaggccagattgcaaaatggttaatcttgatcaaggctggaaaatgtgtatggaaataagagagactgtaatatcttaagaatggggtttgtccttgtgccagtattaaagaacaacaacaaaaaaaaaaaaaaaagaaaaagaaaaagaaagaaagaaagaaagagagaaagaaagaaagagaaaaagaaaaatggaggaaagtcatggggaaacaggtaatcaggcttgaattaaaaaaataaaaaaataaaataaaaagttaaaggacttgactagtcccaggtttctgcacagatgaattaaaaaatgaaaagaaaagaaaagaaaagaaaagaaaagaaagggaaaaaaagaaaaaaagaaaggaaaaaaaaaggaaagagaaaaacaaaagacaggaaaagaagggaggaaggaagaagagaaagaaagaaagacagacagaaagagagagagaaaggaaggaagaaaggaagagagaaagaaagaaagaaagaaagaaagaaagaaagaaagaaaggaaggaaggaggaaggaaggaaggaaagaaagaaaagaaaagaaaagaaaagaaaagaaaagaaaagaaatgaaaagaaaagaaaagaaaagaaaagaaaagaaaagaaaagaaaagaaaagaaaagagaggaaggaagacagaggaaagggcctggctgcttgtaggtactgtagcccgggagatcatagccagaggcaaggacagctagggaaatccagagtggtcatgaccgtgaaccacttgaggagagggaagagaaaagggcagcccagcccccaggctagagaagtttagggttggggcagggtttgtcagccagtgggtctttgtaactcccgagaacttgtaggcggctttgatatgctaagtaggcacctcagcctttcctcttgggtttgaaacccagaaggcctcagcaagtgggacaagccaagcccaacttggaaagcagagttttaaagggtgaaacaaccaaccaaaaaggttcactctcttctagcaagacgcttatgctgcaaagagacttaagacaatccgggagcaaggacaggacacacgctgaggatgggatgctaaatcagccctggaaaatacgtgtctaatacaggaggctctcgcccaagatctattggtttcctagcacagccgtaaaatggctagctattttagaacagtaaccagttctctgaactccatgccagcctgtccccaagtttccagccctctgcttcaaacacttcttccaacccttccaccctcaatttcccaacagtcttcttaaacaattccttctccccattctcccaagactagccactccaaacggtaacaaccatatcttccataccaactgccttctcttctccctctgctagcccaatcccccctccttccctgactcagctggctcttttatactctatccagttcctagtcatccaagaagcaaccaatgcttagggtcatagggtcaagcctttgtaacagctaaaagttcttaaagaggccagttgactcgcaactggagaccctttattacttatttatttttatgtctatgagtacactgtagctgtcttcagacacacaccagaaaagggcatcagatcccattacagggttgtgagccaccatgtggttgctgggaattgaactcaggacctatggaagagcaatcagggctcttaaccccgccgagctcatctggagaccctttaaagggccaggagcccaagataaatcacattacagttacatatgacccaaactcaggtgagctagaccagtgaagacttagactgatttctggaattaaggcactgggcctgcagcacttccctccaaagcctgcagtgcagaatctgcccaggcctttcccaggctccggtagcttcaacagcttcctggctactagctcctggcatctttccaccccttccttatatggccttctgctgctcagtgtcttctcccctgatgccttttcttcttcttttcttacaaggatgctgctcattggtttttaagacccaagctaatcccagataatctcatcagtaaagagccctctccaaatagtatcacatacttagttcctaaggggacaacttatttgtgttctaagacttggggcaaagtatgagcagatggtgcaatcttaggaatgctgactgaaagaactgtcttgtccctagattcccaaacctgatctgtcaggaatgaatgggggaaaaaagcccaggacaatgacaggcaagagtgtaaaatatcagtggtcgccaaggggcttcgggacataaattcagtgagtgagtgcattcttaccacgtatgaagaccatgccaaacacagacataaataacgaaaattaaagattaaaagtaaataaattggccagatgtcatggcaaatgcctgcattcctagcaactagcagaagcaggaaggtatcaagttctaggcctgcctgtaacactgaacacatatgagaccagcctgagatatgtagacaaacaaaacaaaacaaaacgaaagaccattgatcaatcaataacataaacgttcaggaaacacaggccacacaatcagggaggctttgcctttgtttaggggctgggtttttttttatattttcctttccttgttcttcttcttgtttgtttgctttttgtttgtttgtttgcttgcttgcttgcttttgttttttcaagacagggtttctccgtgtaggcatctctggctatcctggaactcattctgtagataaggctgtctttgaactcagagatctgcttgcctctgccttcttaaggccgggactaaaggtgtgcacagtcactgccacctacctgctgtttctttatcccctgcattagtcaccctcccacacccgccagagctctgggatagagactctgtgttagggaatggagcctacagcttccatcaggtgctatgttttaattcacattttctattcctcttttctcatttgtctggggtgattcttttggacaaaaaaaaatgttatcttttataattactaattaggatacaaacgttaaaatatatctataatgtactcttgtatatcattcataatacctatttcgtattatacttccttatagtatatagcatattttgataacaaggtcacgaggtcccaggcctggaaccaactttttcctatgtaattggttgaagttactgacaccgtagtagacttagccctctggaaacagcctttggcaatgaatttgactctgtttcagacaaccatcactttcctggaaacggcctctggcccattcattacatatttcttgcccatctgtgacacgctctgggtacaatcagcatgactcactaagagggtgttcccgtttgcttctggtttctgtgagaaaacaaacaaaagcaactctgggaggcaagggtttattcagattataagtccctgtcacagtcaatcgctgagggatgccaggtcaagagctcaagcagggccctggatgcaggaagcagaaatcagagaaatgatgctctgtggcttcctctcaattcacacctcccgggaccacctgcctagggatggccccacccatagtgggctggacctccctccatcaatcactaatcaagaaaacaccccacagatttgtctacaggccgatctggtagagacaatttctcagtgaaggctccttctgacttaatgtcaagttggcaaaaactaacaggcacagatgggaaccagtcataacagtgctctgtacagttctcccattgctctttagaatggtctcacatggcctctcacaatgctcacacagcttctcctgggaatttaatgccccttgcttcaacttctctttgctgtgccatgatgctagcctttcctatgtccaatcccccacccacatccactgaggttcttgtagactagaaccttccacatgagacagactaccttaacccttgtcttaggggtaggctggcagggttactatttctataaaacatactttcaattccattttaacgtttcatatgtattatatatatttatattatcaatgtagtgatgctaaatagacaaacagatgacttaagtcttaatgatgatcctataagaattcctaaaatatatctgtggttattaagctcttttatagtgggacagccattaagtccttttctaattgtcaaaactgcaatgagaactctgccagtctcccaagtgtctctagttaattgctattagatcgtaaccagactttctcctactcagagcacattccaagcggttgtaagacaatcagtcaaagtttataaaaagggacaatttatatatatcctgctaggacagaagataaaatattggctggttttgtcgatactaagcttcactaataacttagttatggtttgaacccttcagtgaacagggtgatggatgttgagcaggatttttactcctcatggatatgcatgtaaactttctctgttgtaaacttatatatcaatttatgatttgatttttgtgtgtgaacttgtgatgaactttgtaacatgtgatcattctgaaagatgtataagtactaaggacaaagagatgagaggcagatagagttgaatcccccccccccccccccaagaatcaatcatttcccttcctagaatttttgctcctgtagagaatttttcactttccccacttaggatctttctgcctttccccttagttagctttcgtaggaaactttttacaacttagtaataaatgctataatcatttttcgaagtgtccccttttcttcctgtgacttccgactagcaggccgaaagttagagtggaacagcttctgctgagatagtgcgaaggctatctactttatcctttgctgtttgaggaagatgtgctaagtgccagagactgcagtttctggcctccgaggaacacagaaggcaggtcagctacagtagcatcgagaagaacccaacaagaaagaagagctccatgccagggctggcagcattcctatgttttatgtaatatttactgctgagcaggggataatatctgacatcatatgttacttatacatttttgttgattaaaaactataataatcatcccttctcagcacccacagaaaaccaaggcatgtggatctctgtgagcttggggccagcctggttcacatggtgagttccaggtcagctggtgttacataacatgaccttgtctcaaaaatataataataataacaataataatacaatattatatattttataataaataataacaatcactagtattaaaataaaaataaaagaaaatataaaatcatgcttgttaaatattgctttataggacattatttgactgtcttttaacctggcaaatacccatcagcattctttctcaaaactcagtaccactcgaggcctccttgacccactgtctcggttattcagcactaatgatctaagtgtatttttggcacacagttcatattgtagacagcactttgtaatttttttcttttatgtttactttttttttttaacttggataaataagtataggccaggacatttatctgtaaaaagaaaggatgtgtattggaaagtttctatgagctttgtgtcagaaggggtttggcctggccactgaccacatgtgctagggagggagagttaacctcccctctccccatggggagccttagctgtttattctgttgctgtggtaagatgtcctggtaagcaacagaggcagggagggaggggtttgtttgggttcacagttcagcagaagccacaggtggcctgaccttaaggcatctggccttgtcgcacccacacccagagccacggggatcttagctgccgttctcatcgttccatagtctagggtctaggcccatgtaatgatgccacccacagccatcctaactagacaaacaaccactcagagctgtgcccagaggctcacctctcagtcattccagaccccgttaaacagacggccagtactaactgttacactttctgtatttcagtgtgggaagaacaaaagagaagcctcatggcacaaacacccaccgattaagacaagcatcagcatgaagactagaaacaatattaaacaatattaaaagtgactttgggaagaggggaaatttttaagtatgatttaagaccagaaaacgagtgagatgtggtttgtttcattattccattaggaacagctatttaaaatacacataaaggacaccatacctgggatatatactgggttttagtctggactaaaggatgacacttggtttttaaacaaaggaaaaattgagtgactaaagcttatgaaaattgaacttgcagaaatctaaaaatttaaatctagaatagtaacaaattgatatcatcaattcgggagttacatagatcacctaacaaaccacattgggacctcctaagtgttatcattttagacaagagggtaagaatcggggtactctttcgtctactttagagagttcatgcttttttgaatgccctgttagcaatgttgatccagaagactacatcatcttcttctttatgaattgggaagccagcaacaaagcaactgagtagcaaaggcttattcttggctaagaataggagacatgagaaacaaactcacaagttcactccgcgatctgttgtggttataggcataggatcaaggagttgagtgggaaggagaggctagtaatcaggggacatattgttactgggagacatgttcaagtatttcctaagactaggtggagatttcccaggaaatccatcaatatatctttttcaactattttaaaaacttcactcatttattttagtatgtgtgtgaaaaagagacagagatatatagagagagaaagggggagggagggagaaggagacggggaaagaaagacagggagatagagagacagagaaaaaagacagagggaggaatgggtagagggaaagggagagagagaagagggtcagagagagggggggagagagagaaagaaaagggggggaggggaaaagggagagagaaatagagacagagagggagggagggaaagaagaaggaaagggggagggggagggggagagagagaaagacacacacacacacacacacacacacacacacacacacacacacacgggggtggggggagagagaatgctgatgtcatagtgtgccactggagatcagtggacaactgtctcaggaactagttctctcctgccaccttgtgggatgcaggatgaccttagatcctacctgctggctagcctacctttcccatcttttatggatctttcttgtcagaaacgaatgactgtgatgttactgaggttggttggcatggagcatgcaatgtaggtgttatattgtgaaggtagaggtgtttagcagtcactccggcacttatctgagacccaaataattctggccaacctacctgaagagcaacctctggcctatactatcttgttataaaaggaaatcagattaaagccaggaagacattctctaggctaactaggaagacttctctctcctgccctctacactgaaacattcctctttaaaaaaattatgtgtctttggaattattattattagtattagtagtagtattttggttgctattgttgcacatgttaagcaatgagctaacatttcccatcatgcctaccatcatataaatgttaggtttgagttcggggctaagcagaccacatttccatgtcatcttacaggtacttgcaggcccaccatagaacagttgtagaactctgacaaacttccagagctccgctgaacttcagaaaatactgaatcccagaggtcccacaagtctctatcctcctctctgcagaaccctcagagctgattctgggcttcctggtgctacacggaaatataaaacaacccgtctgaatgtatcagacaaattccctagataaccagacggtagagagcaatggctaagtcacatcccccagggatgggggaacctcactaattaattaaatggtaaatgtggtaggtacatgtacgaactgacatcaaagaagatgcatgctgtctaaccagaaacttgcctggtgctactgtctctagtatgataaatttaaatctgacagttaggagtaaccaatataattctgtactttcttcagtctctagagcagagactctcaacctgtggtttgagaccccctttggagcttgcatatcagagattttgcatagccaatatttacactattatttgtaacttgtaacaactttacagttatgaagtagcaacaaaataactttatggttgcgggggggggggggtcaccacaacatgaggaactgtattaaagagttgcacacggtattaggaagggtgagaccccgtgctatagaggaaaggctggaaagtcaaatggtttttacatgagttgtagtgctgcctaagaggagtgtttagcaatcttctgggtaagactcaaagagacccagaagcctagcaatacccgtggtagaggtgtcacctcctactcaaatgggttgtaaaacacctttcaaactgctctcctctcttctggattactggcaagccttggtccctgtctgtggttgtcagactcaggctgtgtcaatcacttagcggatggataactggctacagagctccaccagaaggtcagcttccgtatgtctggggtgaggaccaggagcctgtgggttgaatcgttcccaagggataaaagtactgtggacgctgagtgcactcggatgctttgtggttgcgtttgtctgttgagcttacagcagtccatgcatgagacattggaaacaaagagctgcagccttcctctgggatctaaggggaaagaatagactgcagcctagagtcttggttctcagaaatgaactgtttaaggtttctcacgtaagctaggaaggcagagcccagctaggtttcatttaattatgatgggattccttatttaggtaccaaagaaggaaaaggctcctctggtgatggttagtgacagcttcttttctgggtcccagccaatcaagggggcatctgagttctacagactttaagtgcgtgatttttctatttcaaagtaaacacgagatattctagcccagcatttgagtcactatgttcaaatatattctaaaatgcccagatcacaccagcgagattaagtaagttggcagttcacagatacatcaacatgaatctatctttctttctttctttctttctttctttctttctttctttctttctttctttctttcattcattatctatttatctgcctacctatcatctatctacctacctagctatctagctatgtctatcttccagtccgtaaatctatctctgtgcatctatctatctatctatctatctatctatctatctatctatctatctatctatctatctatctttcattatctatttatctgtctacctatcatctatctacctacctagctatctagctatgtctatcttccagtctgtaaatctatctctgtgcatctatctatctatctatctatctatctctatcatttatcaaccatctttctatccatcatccatctctctctctctcatatctctctcatatatatacatatgcatatgtatacgagatcctttctcctgaggaaggagtcaagattcacagtcctaggcaagtgctgtactcctgggcaatagggcagcctttgcttttattcttagtctttctcttgatgccaaccaaatgcgtatcagcttcaattctgctctcttcagctgtgtacctgcagctgtttcctggagaaaatgacaccatcaaccatttctctccaggacaaaggctgggtgggctgggaggggtgcgggtggagtgagtgcaggacaggagaccaaaggctgaaccagatgacggtcttgcccagaacgcctccacccgtccagaactacagccgctgctccccatgcagtttgactctgaaagtataacaacgtaaaaacagaattctaggcctttaggcccaggcttgagaatgtggcctttgtaaaggtatgctaatcataaaagagatagcgacattcctctacccacccgcttcctgggttcaaagagtgctcattcaaagaaagtcaccctgaggattaccagaacctgcaatgcaaatgtgctattgttagaggctcttagaagctgtcttgagagttaacacttaccaggtattccttgtgactcttgtaactttacacttccttgtgactcttaactggtatctttggtatcttccaacaatgccctccccacttccttgagtttcggtttcttcctttaaatacccccttacccagctactcggggtgccacggtcctctacccctgcgtggtgtatgaccatgggcccgagagcgcttttgaataaaaatcctcttgcaatttgcagcaagacccgtttcttgtgggtgattttggggtgtcgcctctcctgagtcagaacgtgggggagccctcacattgtgggtctttcaactcgaggcttccagttcgtcctgagttactttagagtcaaagaaagaaaagggaagattaaatgaagttcaaaacctccacagcgtatggcaagatgttggaaatactagggatgcaagtcaaccaggagttttagcccagaatcacaggagatctagatgttcatgaccccgacccccaccccctaaacccaacttccacccccacccccgccaccccaacagcaacaagaagtgacacgtgggtctcctgttgatctctgcctctccagagctggactttcaagtgtcctggcttattactgtccctctgtgttgtcacatttgctcagggtcagctctcctctcctcttcccggttcttagcataacgcttattcctctctgtagtctcacagcctgatctttacccccagtgagctgagaaactggatccatgggaagctaagcccctcacttctgcatctgccaggatgcatctccttctccgctcctcttcttcctcatgccttgctgcctctgtgtcccccgcccccctcttcttcccagtgttcctcttccagcagcagaccatgaactccatactgctcttatccctagaagacctctgtaaaagcaggttccctttcattgatgtctctcctaaaaagtccatcacatcgccccacacttccccagcacaggaccatttggacttagcttccaagatcctgttctcagtttcaatagcaaaggtgtagagaaactggcatgaacctctttgtgttgctttgtctctaagggatggccttcttttaactcttgccttggactgggctcagtgggccctcctcaatccgtgcgaatcagaggacagatgggcgccgtagagtcggcgagtgacaaacagaaacggcggcgagtgtgtagaatctgagtgtatttttacaaagtgaacaccagtcttatatagtacagaaaataaagggataggatgtcacagcaggcaaagtacattgaagttacctgacacaaaacaaagtaatgacttcaaaaggactttcaggaaccaggtaatagttacagtaaagataaaacagctctgcttagggtcagctaaggacaggtaaggatttcacaccctactcacaatttgtgctactcctttgaaccttgtgaaagctagcaccagggagttctgctctagcagaccttctcatgaataatgcaataccacaaaccccctatttcctaggacttgataaattctttcatgagtataacttggctgttcttttaagtatctgtggggaagctccatttgtcagaagaattcaccaacttgcttctaatatgcaatgtagcctgctatacctggctgtacaagattcctgtctcagtgggattctctaactctttcatggtaaacccacctattagctaggccatggtgtattcccttgtttgggttagacttggctactgtcctaagtcctaagtaatcaccctgcagaccagccctgagctattctagctctgttctttgtaatgcctaattagtttcaccatttctactagaagtaaatttgaatgttactgaataggtaacattctcactgaatttctactgaattccaagctcgtcggcttcaagaattttctaggacgttggaacactggtggaggcttacctatgttaaaattcaacctttaaaggcacttataataaaacaatactaaaagagagcatgtgcatccatataccagactaacacggggatagggtatgagtatacaggttatgagaatgccaaggttctaggaggttgagtttccttgaaactctttgcctccatgagtgcttccaggcctctcggcctgtcaagcagacttcactggagtgggtatagcaaactctgtatcgatgcccttgatcttctgattattgcctttcataaaattatcttacattttgactgccatatggtctttctctatggctcattgaaccccgagagtgttctttttatctgtgccagtggagtcagttcctgtgcctaccttgtaaatatccccagtgtgctaatctaggctttccttggtgtttcggtttgcataaccttaagaagcactggccacactctgtgctttctcttttagtgacttctgagtcacacttcttggcacatcatttgaatttctacagaagtggaagctgaattgattgagaactagctttttgcctactctgatctctacttgcatttcctgtatgtgctctgacccattgcatggctgctctgttcataaacacggtcatggttttcaccccatttcttcataactcagaggccctatgggctccatccatgttttgtgtacaattttttatttttttattttattttattttggtttttcgagacagggtttctctgtgtagtcctggctgtcctggaactcactctgtagaccaggctggcctccaactcagaaatctgcctgcctctgcctcccaagtgctgggattaaaggcgcgcttgaccaccgccctgcttcctgtacattttctgaaaggccaaatcaccttcttttgcaacgcatacagctttccgctgagatcgcccaggtctgttgcttgttttcagaagctgacacagacaatgggagtggaggagtttcagtacaagtcgattcatagagatagatgcagcctaactgggcagcccaagccttcaattgcagctatgctggaggctgaagcagggggacctcaaggtcaacggcagcctgagaaactcacagagactgtctcaaaataaaaaatatatatataaaataaaactatacacttgactcttatctacaatgtagtacgtgggaggaggctgaggtcgaagactgtgagtttgatcagcctgggctacatagcaaacatctgcctcaaaagactggaaatacaaaactaaaagtgaagaaaagagaagccacgttcagacttcaaaaccaggctccattgtcatgtttctgggactattccctatccattcccagagtagactgacagaattagcccccaccccttctcgagcgtctcaagcccacacctgagcatagagcatgtgatttacatggggatccatttttcctccacttctcccctcccgtgttatatgatcggagcctaaggtctgtaaccttgtcagctgcgtgtccgtgtggctagtctagcctaaggccaggcttgccggaagttctcttggcacacctgggttgacttgacttctcctgaagagcattccaaagctgctctctgtaaaattctcagctgtttctatttgaaatgcaggaagcacttccagatcattacaagccttggatggaaattgccctcagacttcctcacttaatcgagaaccgccagctccgagctcacgtgtacagggtacgtcgatttgttctttgcttcatgctcctcctctccccacccgttccgtctccatatccctttccttctcagctctctgggagtgaaacggttgacctggcagtgggaaccgactgtcaccactgaggctttcagcaaacctgtcttccttagtgcgcacaaacagcacctgagatgtgtcaagtcctcatcgtgtttgtgcaaaagacgatgatagatgagacttgaaaataaattctggggaaacgaggaagacacacattggagattggtgggagccgcttggcctgcttctgtaaaagcctgcctgtcaggcagagtcaagccaggggctgaatgttctgttaatctcggaggaaggaacacggaggaatagaggcctctggatgagtacagatatcagatatacatcagccgctgtctctgctggcttgtgactttgcttcccgtgatactacagagtatataaagccttagagaattaaactcaaggctgctgcagcactgactggatagccttcccggtttttaagctttgtttctgagtcttcttttccatctctcctacaatcctcactcacccctcagaagacctgttcaactttgtactggcaggaccggcaggggagatgctcagtggacttgtcccaagtcacgtggtcactgctctgcttcctctgggcaggtaccttgaccttagtgtgttacatactagtcagatcaaatgtctttctgatttttaaactcagagtcagtcttggctatgtagaagaagtgaaatccatagacaccacattagaatctgcatgcacctaagtatatgacgcacgtctgtgacagagcgagcaatttccctccaaaaggaataagaaaaatatgacccccacagaagacattcttccattcaggaattttatagtaagtgtgaaacggagagttggcctcagaatcttgcttccttctttgagaaatggatgaagttctcatttaatggttgtgagcatcaatggcttttgtaaggaccctccccctcctcatcttcatctgtcacaagcacaccacaagatgtcagatgttcaagcaaggcgctccaccctccaggaccttctttgctgatgcttaatgtccaattatgagttgatttttaaaaatattcattgaatgatccttatagcatattcagaaatcaggacaggcagctggccttggtaatactctagataaccccccctctgaaccccccactgaggctgaagaccatgtaagaaagtgcaactaaaggagttaattgttgcacaaaactcacacaaagcctcacaaaggtagtttgtacacgctgttgacacattacactaacaaaaacatcgaggggaaaaaagatgggaaacagtagaagataataattaatggtgacctatggcctacacacacacacacacacacacacacacacacacacacacacacacacacatgcacacatcaccacacacgtgtgtatgcatatgctcacacatgtacaaacatatatcaccacatatgtgacacacacatacacactcatatcaccacacacatgtgtacacacatgcacacacacacatcaccacacacatatgcatacataggaacatatagtacacacatatcaccacatagatggcacacacacactcacataccatcacatatatgcatgcatatagacatgcacacacatgcccccccacacaaacacacatatatgtacaaaacctatagtgaacacactaccctgagtcaagctatgagttttaagaacatacatactgtcccccaaaactcgtcacaaggtgttttgtcctcaaagggaaagctcctctctatctaactgatgtctagaacccacaaatatgttagttgtgtaccagaggggacggcgggtggttagttagtagagggtctcccacaaacccatgtgtctgtcttcatccaccatcttttacactgatgaatgagttactagccagtaactcatgcagtgaggcaaagcagttatgatggttctgggtgacaagcaagtgaagagtattggtgaggatactaagcacgcgcatgcactttgctacaactagtctgtcatgtgaactgccatctcacagaaacattcatacaaaaaaaagtgcagatgaatatgcaaagctgtttgctgtagccactttcatagcaacggtaaatacaatgtaacagaaatacaatgtaacagaagacaggagagcagaaggagaagcccatgaatctccattcacaaatatatctgttacataaatgaaaagggacagatattaagtggtcacgtaggcacctgcctgtatccttccgccacgctacacatctttgctctgtagtttactgagcattaggaagttcgcgacagtatctatatgcagaattattgaacctctacttcaacaacatgtgcatgtgagcttaatatttatagatgatagaggtagaaatagacatatttactatctgacagagtcctaacatgtagcccaggatggctttgaattagacatcttcctatctccgcctccagagtgctggtatcagtcttcataaagacttctatatgaaaacaaatgtcctgttttctatgtattcgccttcgtgtcaaactatacttttcaagaagttcaagaagtgatgtccacaagaacctgatgtgtgtcagaaacactcagctccccagtgaagttggcaggagggctcgcctggttcagagaagccttgcgtgctccctgagttctgagaggagagtatcgaggttaattctcccaggttaaatacgcaatcaaataatgtcagggagatgacgggtgtgtgtcgtcctcccagaccatctgtgactgtttgagtaagcaaaaggattgactaaaaagctatcatttccttttcagtccctacagtcacttttgaatcacaggtatgtttaggaggtgaggccaacaatcctgaaatgtgcagggactctccagaaagcagtcatcatcccctggacctcgttcctttacaccgaggcgtcctggcttgtaggtggccttcttcaacaactgaggggtgacaaggacaaagcctgagaagacacagcctagattgtgaagactttgaaagatttttatactgactgcatgctaaaatatcttttatacgttgactcaacaaagtacattatcagaactaattttacttcttgtttgccttaaaaaaaaaaaagtggctgctgaaagtatacgagatcctttctcctgaggaaggagtcaagattcacagtcctaggcaagtgctgtactcctgggcaatagggcagcctttgcttttattcttagtctttctcttgatgccaaccaaatgcgtatcagcttcaattctgctctcttcagctgtgtacctgcagctgtttcctggagaaaatgacaccatcaaccatttctctccaggacaaaggctgggtgggctgggaggggtgcgggtggagtgagtgcaggacaggagaccaaaggctgaaccagatgacggtcttgcccagaacgcctccacccgtccagaactacagccgctgctccccatgcagtttgactctgaaagtataacaacgtaaaaacagaattctaggcctttaggcccaggcttgagaatgtggcctttgtaaaggtatgctaatcataaaagagatagcgacattcctctacccacccgcttcctgggttcaaagagtgctcattcaaagaaagtcaccctgaggattaccagaacctgcaatgcaaatgtgctattgttagaggctcttagaagctgtcttgagagttaacacttaccaggtattccttgtgactcttgtaactttacacttccttgtgactcttaactggtatctttggtatcttccaacaatgccctccccacttccttgagtttcggtttcttcctttaaatacccccttacccagctactcggggtgccacggtcctctacccctgcgtggtgtatgaccatgggcccgagagcgcttttgaataaaaatcctcttgcaatttgcagcaagacccgtttcttgtgggtgattttggggtgtcgcctctcctgagtcagaacgtgggggagccctcacattgtgggtctttcaactcgaggcttccagttcgtcctgagttactttagagtcaaagaaagaaaagggaagattaaatgaagttcaaaacctccacagcgtatggcaagatgttggaaatactagggatgcaagtcaaccaggagttttagcccagaatcacaggagatctagatgttcatgaccccgacccccaccccctaaacccaacttccacccccacccccgccaccccaacagcaacaagaagtgacacgtgggtctcctgttgatctctgcctctccagagctggactttcaagtgtcctggcttattactgtccctctgtgttgtcacatttgctcagggtcagctctcctctcctcttcccggttcttagcataacgcttattcctctctgtagtctcacagcctgatctttacccccagtgagctgagaaactggatccatgggaagctaagcccctcacttctgcatctgccaggatgcatctccttctccgctcctcttcttcctcatgccttgctgcctctgtgtcccccgcccccctcttcttcccagtgttcctcttccagcagcagaccatgaactccatactgctcttatccctagaagacctctgtaaaagcaggttccctttcattgatgtctctcctaaaaagtccatcacatcgccccacacttccccagcacaggaccatttggacttagcttccaagatcctgttctcagtttcaatagcaaaggtgtagagaaactggcatgaacctctttgtgttgctttgtctctaagggatggccttcttttaactcttgccttggactgggctcagtgggccctcctcaatccgtgcgaatcagaggacagatgggcgccgtagagtcggcgagtgacaaacagaaacggcggcgagtgtgtagaatctgagtgtatttttacaaagtgaacaccagtcttatatagtacagaaaataaagggataggatgtcacagcaggcaaagtacattgaagttacctgacacaaaacaaagtaatgacttcaaaaggactttcaggaaccaggtaatagttacagtaaagataaaacagctctgcttagggtcagctaaggacaggtaaggatttcacaccctactcacaatttgtgctactcctttgaaccttgtgaaagctagcaccagggagttctgctctagcagaccttctcatgaataatgcaataccacaaaccccctatttcctaggacttgataaattctttcatgagtataacttggctgttcttttaagtatctgtggggaagctccatttgtcagaagaattcaccaacttgcttctaatatgcaatgtagcctgctatacctggctgtacaagattcctgtctcagtgggattctctaactctttcatggtaaacccacctattagctaggccatggtgtattcccttgtttgggttagacttggctactgtcctaagtcctaagtaatcaccctgcagaccagccctgagctattctagctctgttctttgtaatgcctaattagtttcaccatttctactagaagtaaatttgaatgttactgaataggtaacattctcactgaatttctactgaattccaagctcgtcggcttcaagaattttctaggacgttggaacactggtggaggcttacctatgttaaaattcaacctttaaaggcacttataataaaacaatactaaaagagagcatgtgcatccatataccagactaacacggggatagggtatgagtatacaggttatgagaatgccaaggttctaggaggttgagtttccttgaaactctttgcctccatgagtgcttccaggcctctcggcctgtcaagcagacttcactggagtgggtatagcaaactctgtatcgatgcccttgatcttctgattattgcctttcataaaattatcttacattttgactgccatatggtctttctctatggctcattgaaccccgagagtgttctttttatctgtgccagtggagtcagttcctgtgcctaccttgtaaatatccccagtgtgctaatctaggctttccttggtgtttcggtttgcataaccttaagaagcactggccacactctgtgctttctcttttagtgacttctgagtcacacttcttggcacatcatttgaatttctacagaagtggaagctgaattgattgagaactagctttttgcctactctgatctctacttgcatttcctgtatgtgctctgacccattgcatggctgctctgttcataaacacggtcatggttttcaccccatttcttcataactcagaggccctatgggctccatccatgttttgtgtacaattttttatttttttattttattttattttggtttttcgagacagggtttctctgtgtagtcctggctgtcctggaactcactctgtagaccaggctggcctccaactcagaaatctgcctgcctctgcctcccaagtgctgggattaaaggcgcgcttgaccaccgccctgcttcctgtacattttctgaaaggccaaatcaccttcttttgcaacgcatacagctttccgctgagatcgcccaggtctgttgcttgttttcagaagctgacacagacaatgggagtggaggagtttcagtacaagtcgattcatagagatagatgcagcctaactgggcagcccaagccttcaattgcagctatgctggaggctgaagcagggggacctcaaggtcaacggcagcctgagaaactcacagagactgtctcaaaataaaaaatatatatataaaataaaactatacacttgactcttatctacaatgtagtacgtgggaggaggctgaggtcgaagactgtgagtttgatcagcctgggctacatagcaaacatctgcctcaaaagactggaaatacaaaactaaaagtgaagaaaagagaagccacgttcagacttcaaaaccaggctccattgtcatgtttctgggactattccctatccattcccagagtagactgacagaattagcccccaccccttctcgagcgtctcaagcccacacctgagcatagagcatgtgatttacatggggatccatttttcctccacttctcccctcccgtgttatatgatcggagcctaaggtctgtaaccttgtcagctgcgtgtccgtgtggctagtctagcctaaggccaggcttgccggaagttctcttggcacacctgggttgacttgacttctcctgaagagcattccaaagctgctctctgtaaaattctcagctgtttctatttgaaatgcaggaagcacttccagatcattacaagccttggatggaaattgccctcagacttcctcacttaatcgagaaccgccagctccgagctcacgtgtacagggtacgtcgatttgttctttgcttcatgctcctcctctccccacccgttccgtctccatatccctttccttctcagctctctgggagtgaaacggttgacctggcagtgggaaccgactgtcaccactgaggctttcagcaaacctgtcttccttagtgcgcacaaacagcacctgagatgtgtcaagtcctcatcgtgtttgtgcaaaagacgatgatagatgagacttgaaaataaattctggggaaacgaggaagacacacattggagattggtgggagccgcttggcctgcttctgtaaaagcctgcctgtcaggcagagtcaagccaggggctgaatgttctgttaatctcggaggaaggaacacggaggaatagaggcctctggatgagtacagatatcagatatacatcagccgctgtctctgctggcttgtgactttgcttcccgtgatactacagagtatataaagccttagagaattaaactcaaggctgctgcagcactgactggatagccttcccggtttttaagctttgtttctgagtcttcttttccatctctcctacaatcctcactcacccctcagaagacctgttcaactttgtactggcaggaccggcaggggagatgctcagtggacttgtcccaagtcacgtggtcactgctctgcttcctctgggcaggtaccttgaccttagtgtgttacatactagtcagatcaaatgtctttctgatttttaaactcagagtcagtcttggctatgtagaagaagtgaaatccatagacaccacattagaatctgcatgcacctaagtatatgacgcacgtctgtgacagagcgagcaatttccctccaaaaggaataagaaaaatatgacccccacagaagacattcttccattcaggaattttatagtaagtgtgaaacggagagttggcctcagaatcttgcttccttctttgagaaatggatgaagttctcatttaatggttgtgagcatcaatggcttttgtaaggaccctccccctcctcatcttcatctgtcacaagcacaccacaagatgtcagatgttcaagcaaggcgctccaccctccaggaccttctttgctgatgcttaatgtccaattatgagttgatttttaaaaatattcattgaatgatccttatagcatattcagaaatcaggacaggcagctggccttggtaatactctagataaccccccctctgaaccccccactgaggctgaagaccatgtaagaaagtgcaactaaaggagttaattgttgcacaaaactcacacaaagcctcacaaaggtagtttgtacacgctgttgacacattacactaacaaaaacatcgaggggaaaaaagatgggaaacagtagaagataataattaatggtgacctatggcctacacacacacacacacacacacacacacacacacacacacacacacacacatgcacacatcaccacacacgtgtgtatgcatatgctcacacatgtacaaacatatatcaccacatatgtgacacacacatacacactcatatcaccacacacatgtgtacacacatgcacacacacacatcaccacacacatatgcatacataggaacatatagtacacacatatcaccacatagatggcacacacacactcacataccatcacatatatgcatgcatatagacatgcacacacatgcccccccacacaaacacacatatatgtacaaaacctatagtgaacacactaccctgagtcaagctatgagttttaagaacatacatactgtcccccaaaactcgtcacaaggtgttttgtcctcaaagggaaagctcctctctatctaactgatgtctagaacccacaaatatgttagttgtgtaccagaggggacggcgggtggttagttagtagagggtctcccacaaacccatgtgtctgtcttcatccaccatcttttacactgatgaatgagttactagccagtaactcatgcagtgaggcaaagcagttatgatggttctgggtgacaagcaagtgaagagtattggtgaggatactaagcacgcgcatgcactttgctacaactagtctgtcatgtgaactgccatctcacagaaacattcatacaaaaaaaagtgcagatgaatatgcaaagctgtttgctgtagccactttcatagcaacggtaaatacaatgtaacagaaatacaatgtaacagaagacaggagagcagaaggagaagcccatgaatctccattcacaaatatatctgttacataaatgaaaagggacagatattaagtggtcacgtaggcacctgcctgtatccttccgccacgctacacatctttgctctgtagtttactgagcattaggaagttcgcgacagtatctatatgcagaattattgaacctctacttcaacaacatgtgcatgtgagcttaatatttatagatgatagaggtagaaatagacatatttactatctgacagagtcctaacatgtagcccaggatggctttgaattagacatcttcctatctccgcctccagagtgctggtatcagtcttcataaagacttctatatgaaaacaaatgtcctgttttctatgtattcgccttcgtgtcaaactatacttttcaagaagttcaagaagtgatgtccacaagaacctgatgtgtgtcagaaacactcagctccccagtgaagttggcaggagggctcgcctggttcagagaagccttgcgtgctccctgagttctgagaggagagtatcgaggttaattctcccaggttaaatacgcaatcaaataatgtcagggagatgacgggtgtgtgtcgtcctcccagaccatctgtgactgtttgagtaagcaaaaggattgactaaaaagctatcatttccttttcagtccctacagtcacttttgaatcacaggtatgtttaggaggtgaggccaacaatcctgaaatgtgcagggactctccagaaagcagtcatcatcccctggacctcgttcctttacaccgaggcgtcctggcttgtaggtggccttcttcaacaactgaggggtgacaaggacaaagcctgagaagacacagcctagattgtgaagactttgaaagatttttatactgactgcatgctaaaatatcttttatacgttgactcaacaaagtacattatcagaactaattttacttcttgtttgccttaaaaaaaaaaaagtggctgctgaaaattttcaaagaaaacccgtgactcattgcatgattctcgtgatcggggctgtggtcaatccgcacgcagaactgcttcaatccttcttgattctgtgacctccgagacgaaaattcctctttggttcccgcaggggtgtttagcctcaaatttcaagtcatcctcttccccctagcggccggaggaggaagggctctgcataccccagcccgcccctaggaagccaatgtccccagcgttttacaagtggcgcatgccctctgaggcaagcctgcggagcaaagggagtgaagtggtggcgagagctgaagagatgagcaatgagccggtggcagccaggcaggacagggaagctggcgtggtcggattgtgtactgatgctgtttttcagggtggccctttccgtgtcaccaggagctgtggtttccaatggggatgaagtggggacagagctgggtggaaagccccgccacctcatctggctgggctgtggttgtttgagcatcagagcaagctttatatccagccgatccatggtttttgtgggggactttacatccctaacctcactgggtctcgctgcgttcaaaattttaatacagttacagggttcgagaagctgagggcttccaggtattacatatctattaatgctgctgtgcgggcttggagggaagaacttatctcgccctcaatgcttactctgtgatcctcaaagcaatagatggagaccagtgtatggcacaaactgggcacatcctaggccatggaccccaggacctggcacctcctctactgcattcctctaagagctggtttaagtgggtgtgcatgaagctaatagctgcaatagctgtgggaccctcaagtcacgggtcctggtcctggtcctccaccgttgttttttgttttttggtttttgtttttgttttttgttttttgttttttttttttggctttgttttgttttgttttgttttgttttgttttgttttgttttttcagctgctaatccttttaatctcttgactagatgcctctcctggactgcagattcctaaagagttaccgtgagcagcgcctggcacacatggcgctggccgctatcaccatgggattcgtctggcaggagggggaaggccaaccccaaaaggtgaggagcaaggagaaatcaggctctgctgaggtccctgcacctggaattacgacaccagctccaggttccgttgcctagtttccaaaaatcagcggaaggcaagaaaagagggttagattttttttttccatttttcttttcttttctttttctttttttttttcttttgctacggagctgattttcttttagactaatgtccactttcaaagactccagccattccaataatatgtgaggagcttccttaggccccaaggactcagaaaacaccctgcagaattttatacttctaaatacattggttattatgactttcataattaccccattgtacagacatctggggatctgtcccaagctcaaggcctttgtcccagagcagcctggggacagttgggaaagccagtttctgagtccactgaagtagccatccttctccagtacaacttggacacttccttatttccttattctgcccagctgattgctgctttagtttattaaccctttgtctaactccctccaataactttctagtttcagtctgtccctcagagcacaaaccctttcttctctacccctggaggaaactaatatttcttttgagtcatggtcttacaaagcagccccaaaccagtctagaacctgctctcttgtccaagttgttctccaacctgtgatcttcttgccccggccttccaaatgctggcattgcagtcactccaggcacgtgacaccctgtgcagttatgctcccagtcagtgttgggatggaatccaggacctgtgtgtgccgagtgaatactttatcactcaacaggacctccgttttctcttcctttcccctaagtttctctttcccagaaatcaaggtgttgacatcattccagaatgcgtgagaagcccgaggtgtgcttcaagggctcatcttacccacagagaaacacttgagaccaagaagcaaggacagcacagagctcctgttccttcagactctaacacgataccatattgtatctcatctaaaggactcgagtgtctagggtctgctgttggaaagtttaaaacccaggccagtgtgacagaacctcctgatgagttctgctgcataggccccctaaaccaatctcctgaattatgagttagagaggaggaagcaatgtccagcccccaaggccatcccaggctcatcacccctcattttggaagtcttccctgaccttcccagtggaagtgtctgactcacatcttcccattctccccagccctgacttctgtggatgcttggcagagctctacgccctgtaagctgtaactgttctctacctaaagcacacctcttccctaactcggaggaagaaagaatggcagacacagatctttagactcttcagtcctgggctggggagacgactcagtgggtaaattgggagcaagcaggccgagacactgaagctaagattcccacaaccagtgagaaaactggcacaggcagcagggagcaagagagaccctgtctcaaacatggtggaaggtgtcaactggagtccacacattccccttggcatgaatgtgcctacatttacgtctgtctgtctgtctgtctgtctgtctctctgtgtctctctcacacacacacacaaacagacagagagagacagagacagggagaaatttctattttagaaggaaaatgattggggggaggggaacataaaaatgttcttttgttgttgttgtttgtttgtttgtttgtttgtttttacacctctaccagtttggcctttccatggccgggacctcatggacctgagcatctgctctggactgtggacagatagagatctgtactcaagggacaccccaaacgttagtgggtgaaagggtagggctatggacaggggctttgcaacctgaagggtccttggggcgtgagcgagaagcttgtgtttcagtggagtcctgaacgggtgttaggaggttgatgtcataccccaggtcggaaccctagcaaatcatgttaagagtcagaagagacaaaggacggtcaagggggggggggtctgtcattgagtgactacctgacgttatggggcttgactcatgaggtgacacaaacatcagttctgcaagctgaaattctctgtcctctttatcctttctggggtgcgctctctatctctctgtctctgtctctgtctgtctgtctgtctgtctgtctgtctgtctttctcctatccccccctctctcacacacacatatacacacctctgtttttttttatctatgtacatctttacttctattttgcatcatgtgcatctagccttgtatctatatatagatcatatatattatttataatctatcatccatctagaaagcactttaccttgatagttcttagtctttgatttacattaaacattttggtgaaataattctctttctccttttctcctccttctcctcctccttctttctcctcctcttccttctcctcctttttctcctgtctcctcttcttcctcctcttcctcctcctcctctttctcctgtctcctcttcttcctcctcttcctggtacagtttccagaggttctgtttatcatgtgcacagtgtattccatctctgggttttgcatctcttgaaatgtgttctcataaccagcattctcaccgggtctctcccatgaagcatgtgtagggtataggtgtggaacattcagagaattcgtgcttggtctccaggtcgcttatcacaggcaaccgtgatgattttctctgccagtagaatatactaattgtctggattcctgtcctaaaggtgctgccaagatctcttgccattccttttgttgaggtatccaggaacttgggactcccgcctatcctggtccactctgacctggtgctgacaaactggaccaaaaggaacccagaagggtaaggagggaaaggattccttcagttagcagcagattgagaaaagactcaggacttgaactgtcttcgcagaaaagactcagggtcttagggctgttttatagtgagctgtgtgctcccattcatttaatttagggttccaataaaagggaatgcacacatttaaacctgagacatttcttcttcctgggctctcaggcacactggcttgctgcttccagtgcccatctggatctcttaatcagactgtcagcctccagctgtaataatgacactgggctgttttttctcagcatgttctgtgttccattcaagtcaggttaggatggggtgtctggaggtgatctctatggttctgaataaatgatttcacgtgggtggaattctgattcacaccatctattctgtctagtttacaccattccacacttgtcccgggcacaaaagcatctgtttattgacttttcaacaaacaaccaatccatggaaacttcattttcacagttgaatctgtctcaggcggtcgacgcaaggcagcatttacttttgttataaattaaaacagcaaacaagaagtccaggctggacataaagggacctttcttggcatgctggttgtaggtagccatgcagtgctgaaagagatggttgagatctgctgtggaccctttcaagagacccttccttttaagaactggggagggctcctcttatctacagtctgctgagccttagaacttcatgctttctgggatgatgatgtcatatgacgatgatgtcatatgataatgttacatgatgtcatacgatgttgatgttgtatggtgatgctatatgatgaatggtatcatgatggcatcatatgttgatgataagcttgtttgtgactggggactgaatgaaccttcacctgtgaggcaggagctccacccctgttttctatccccagccctctcttcagcgtttatttagatacagtttctctcacaatgccaaagctgacctgaaattcacactgtagcataagcagtccctgacttggtgatccttagacctcctcagccccttgactatctgccactaacagcatgcacaaccaggcctgacttggtctttgaccaattatacagcaacgaaaaactgatctgcattgaaagctactaaatctaacttagaattggagtaggtgagatgataaatatgtgcgcctggactattgtgaagctgggtttgtgattcaagggaaattgggagatgaagtcttaagggaggtgacagggttgaaaacaaccccaggaaatgaatgtcccatcaagtgaggtacagcacccagagatgcaaatgcaagcctggagctcccccaccgtatttcttctgagccatcatcttcctgagtcttttaagatgtctacccagggcttattaagtgctttgaagaaatgaaagaaatagaagcaacacccccccccacctccacccccaccacccaaacagttcagtattgagacaaagtagctcttaacacaagtcaaagaacaacataaacagtatcttcagaactccagggggaacagcagggaaagcatggtgcctgaccagacgctgatgtaaggccactcctaacaggtgggtaggattagagtaactgaagtggggtctgcaccgagaggcagctagagagaggcaagcaagaatgtgtacatacacagacacacgacacgggctgtcaagtgagatctggtttctgctgtcacccagtggctcactgtccatcctaggtgggtcctgatccgtacatgtgccagggcattgcagatgagtgtgggagcctgggtacccgggacctcaagaattatatctggttactaactaagttgttttctttttttttttctttttctctttgttatcaccaataactattatgcgaccagaccgttggaaatcaggtaagatgtgcctttagaaccacccactgttacctttaaaggcttgtgataaagcaagagggtggcatggtcagctgtatggatggaatagcatcagctgtctatgaactcctgaaaattcgaagtgcattaaaaagtcaatagtccaagcagtagctctgatggaggttagcttctaggggtgcagggattaatttttcttaaccacattttcctgtatgtccatgatttaaaaaaaaagtgtagtgtaagaaaagaaaccgtggctctgtgaaaactggcagagatttcttatttctgaagaacatcagcaaaaggccagaaacaggcaggtcacagtgacagaagggtccctttgtaatcccttcttacctgatgggctggagcccttgtgagtaggtgtggtctatagtcataggctccaccttccccggggagcagagctagcagaggagccagagtcacatcctgcgctctctctctctctctctctcacacacacacacacacacacacacacacacacacacacacacaccctcctcagctctgtggagcatacaggtggatggcaaatattccccaggctaacacctctgctggaccgaggagatgtgtgactcacctccgctttgctgttttgaggtttgaggctgggctttgcttatgtttgagatgctggggacaacgcagatgtgtgcagatactcatctgggttctgtatcaggtcagatttggctctgatgggaagacaggacagcagcaggttgattagaaatgacctccctcctcctcctcctcctcttcttcttcttcttcttcttcttcttcttcttcttcttcttcttcttcttcttcttcttcttcttcttcttcttcttcttcttcttcttcttcttcttcttcttcttcttcttccacacattatattctatccacagtttctcctcccctactcctcccagtcctcctctcacttctcctttccctagatctactcctacttttccaaaaaaaaaaaaaaaaaaaagcaagcctcctggagctatcctccaaacctgccctgacatagttacaaacagcagcccctcatttcaaggcttgatgaggcaacccactaggaggacaaggatccaaagagcaggcaagagtcagagacactcttccactcccactattcggagttccacaggaacacgaaactacacacaaccacaacatatgagcagacggcgtaggtcacgccgatgcaggttctgtgattgtcacttcagtctctgtgagcccccatgaggtcggcttgtttttgattccgtgggctgtgttctcctggcgttctcaacccctctgactactgtagtctgtcctgcccctcttctgtagcgttcctggagctccacctactgtttggatgtggtaatgacaggcttccggccgcggttctgtggcagcaattcatccttgggttagaactctagtcaggcacgcaggcacacacccataatggtcatagttctcaggtcataggaatttggctctaactgatctgattttcttcttttcttggtctcttcctccagtaacctggaaaccatcatctcatttccggggggagagagcctgcggggcttcatcctagtgacagtcttggtggagaaggcagcagtgcccggccttaaggtatcttcttactcacccggccctctccctatggtggttttccccacgtggacagacagaggcctcctcctcctagggttgacgatgaagagacatatctgagttctagcctgtggtgtcagtgcccttttagcctggagtgaaccgacttctccgtctacacccaggtaccacacgctgagtgcaacagacacccactgcgctagctgttccacttcatggacctgcagaagctagagactcagaggtcaaaaagaaaaagactgttttatggaaaggcagataaaggcatatccatgcatatctgtagggatgccagagtagaaacgactgtgatatgggaaacccagaaggaatgagaaagcacttgtgttgaggctcttgatgtcacaatttacgctccagagatattccagtagtgtgggctactccgtggcgatctgcatcccgcgggggttttaataaaaaaagttacttcgtagcacctcaactgagaacgtggatcttattcctaggccctggttcagggaatggaggccattcggcaacacagtcaggacaccctgctagaagccctgcagcagctgagactctccatccaggatatcaccagagccttggcccaaatgcatggtaagatactggagcagcgtctccagtggcctggacttgggcaggtcctacgtggatgatggtggtccatctctccacaagatggcgctagtgttctatatttagcgtaaagcgtcccagtcagagaattgtttttttttttttttttccagtcggcagaaatattttttttagaatcgttctttatagtttcgtgtggttggggtagcttcccttctccagggtccagatacgactttggcatgctgatctcttaacgaagcctgggagattggatgggccacaagggggaatgttggttatacaggttaggcttgctggcgggcagggctgtcccgtgggggcgtctgtaaggccctcaagagtcatttgcttttcctctctgaccatgttacaaatccagaaagaccctcatgatccgctgaaggaggtccaaggacccggagagctccccaattagactccttccaagccatctgatcctgagacaaatttctctatgtgatgactttttttcaaggttttagaactagctgtattcacacacgtggcgaaggagaaggacacgtgtccacacttttcaaagatggcaacagtatcctccacaccatcccatgaagcccttagtgtgggcacgccctcctcgcgtctccggatggaggatatgtcctagcctctgaaatacttgcagtcaaaatagggcactagtgaggacagtttagtccagtgttcagtgagttttcatccccagtgctgagacgccatgggaagggcagtgttcctggcacacaagcaggctgacaacagcctatcagacaaggacacacagctaaggggctaagggctgtccgatgatccttactcagtgtggcactgaggagcggaaacaggtcgccaccaccttgcctctatgcaggcaccttgccagcagtacagagtcagcaaggggaggcgcttgccatactataacagttaccataaaagagagatcatgtttaaaaacaaaatcaaacgctggaaggcataggcagggggtcgtgcgtgtccttggcacggagtctggtctgcatgggctgcttttgtgtgtgattcggagagcgtggctgggtggattccctccactcccaaacccccaactagattttaccaataaccaatcactgtgtttagagcccagcttcagcaagcatcctgggtttccctcaggacatccttccttccttcccaggcatgtagggtccataaaaatgccttttgtggacatcggaggcccactgcgctgcaagccaagtccccaggcctttgcccagcctaacatagttgtacaaataaggtttgtttaatgccatttgcaagcaaaaattctccactgaaatatggtcccaggactgttcgttagctcttacgtaagaaatgattcaggaagaacaggttggaaatataatcccccatactttcttctgaagttttcatcttttatacttaagttcttaactcatctattatcatctgcatgtgctatgagatagcaaatgaattcaatctccctcctgcaggtaacgtccacatggtctctccttagatttggttaaaggagcaggttttctgcagtggcctctgctctttccctttgggtgtgtctctctgctctgggtgcccttagtctcttcttccttttctctggaaagttccctccctggttttcttcttcaggggcctggctgtctctcgggagccttagcaggaaccacaaaaagaaacacagaaaccaaaccctcaaatggggacttagttcaagctgcattaagtatggattctgatattatacgtgcaatttctatcacggaaggaaaacaggccaagggactagaagcagcataaaatattagtctgcatttcctgatgacttcttctaactcatttgtcacggttgctgggtggtctttatctgtcacatgctgtcttgacacaaggacccctctctggtatacaccttcattggctacccagtttccctgtgtgagaattcagcatctgcattttcatgcgcagggttccttgagtttcttccaaaggaaggcagatgcctaaattctgttttatcaacagttatttgcattcttgtgtgtggcaaacatatctttttatttagttacttctgcgctgtttgtttctaccttatagaatgaagcaggtatttaaacgttaacttggaatccagccctttggtaaactacattcttattgattacagcagtaatttacagggttagctgggtttctatgcagagatcttgttgctgagacgtatgcttaggttcatcattaattatctttttcttaaaaataattttccctcacataatatatccttccccttttctcccctcagtttctgcccaccttccctcccacccagatccaccaccttctgtgttacactggaaaaccaacaggcttctaaggggtaataataaaataaagtaagagaaaacaaaaactatcatgttaagttggacaagacaaacaaacagatggaaaggagcctgggaaaaggcacatgcacacacacacacacacacacacacacacacacacacaccaagagacaaaaagtcagctgtaagtgcacatgcatccacacactcaggataccacaaaacactaaaccagaaatcatagcatatatacaaaggacctgtagggtaaaaaaaaaaaaaaaaaaaaaaaaaaaaagtgtgtgtgtgtgtgtgtgtgtgtgtgtgtgtgtgtgatagagagagagagagagagatggggggagggagagggagagggagagggggagggaggagaaggagagggagaggaagagagggaggagagggagagagagggagagagggagagggggagggaggagaggaagagagggagagagggagagggaggagagagagggagagagggagagggggagggaggagaaggagagagagagggaggagaggagggagggggaggggagggggaggggagggggaggggagggggagggggaggggaggagaaacagacagagagagacagacacagagatatgagacagagagacagatcctgtctagagcgctggcgagttttatgtgaactcagcacatatagatcatctaagaggagagctcctcagtgcagaagaggccctcatagggcagggctgtgctcaagcctgtggggcattttctcaattagtgattgatggggaggacccagcccatcgtgagtggagccacccctgggctggtggtccaggtttctataagagagcaggctgagcaacctatgaggatcaagccagtaagcagcgtccctccacggtctctgcatcagctcctgcctccaggtgccatccctgttggcgttcctgtcctgagttcccttgattaggaagcacggtgtggaagcatacgatggataaatgctttgttcctcaagttgcttgggtcaggttggtttttcacagtgatagcaaccgtaaccaagacaacatgtgaagtaaaataaaagataaaaataagatacactcttgaaaatggagggggtaggagtccctgttgtgacattatgagagactgaacctccaaagacgccattttgtttgacttctgctgcagtctagtactggacatgcagcctgccctaagaggtggggaccaggaggggaagcagtgatcgggatgtaaaatgaatgaatgaatgagtgaatgaatgaatgaatgaatgaggaaaagagtattttgttttcccagtgagacttccttagagaaaactaaatttttgttttcaagtagctgtcacttggaggcagcatctgggtgaggggtgagtgctgtgtccacttctcctctcagctgtgaccctgtgtggtgcagaccagtgaaggccctgtgcatgctgtgacttcgttgaccttggtgattgaaaaggccttgttttcttggtgtccttcacccccccccctccgcccaactcttaatttctgttttctctaccatatggttctctgagcactgaggggtgggggttgatggaaacatcaaagaccatttataactgagtgttccgaggtctctcactctctgcacactgtctggctgtgggtctctgcgtttgtttcctatctgctgcaggaggaagcctgtccgatggtgacggagcaaggcgctgatctctgagaacagcagaacatcatcaagagtcattgtgttgcgttttgttttttgtttttgtttttgtttttgttttaaagtcaatattatttagtttcccccctaggtccctgggctatctagtttcagcatgttggtcacccaatcaatttcgggcatgaattccattgtgtggagtgggacttaagtcaaatcagacgtagcttagatgcttctataaactttgtacaactgcactagcatatcttgcaaacaggataccattgtagaacaaagggtctgtaactggctatacacacacacacacacacacacacacacacacacacacacacatatatattatattatatattatatattaattatatatttatgaatcacttttgaaatatctaccgaatatattttatattttcatgtaaattctattgtatctatcaacatgatcacttggctttacctttaattcattaagttgtgtaaaatagtaatgcatacacatcccacttcctatgcaaagggctggatactgtacttaacaatctggagaagaagcaaacatcaaagtctaggggaggtcccagaaaacaggctagctcaggggacacagtcgcactggtgaagaccaccctggggagtcacaccctagaagggagagtgtaaccatactaaggaatcacacttgaggagaggtgagactaccctggggagtcacacctgagaagagagggtgtgaccaccctagggagtcaggaagtcacactggtgaagagtgggggtgtgggatgaccacactgaggagtcacacttgaggaaggggggtggccacactgaggagtgtctgaaagcattgagactgtacatctcaggttatcagggcttcagagaaaacagacagggagaagagacaagaactggattctgtgcgcaaagggggaaaagcaagcagatgtgaagggtgtgctgttagagtttatctaaagatgtttctttcagaaataaagagatataagctttaatttggatgaaataaatgtggtctaatttcccagaatgtagaggaactcactaatgtagcaagattggcctttcaaagcagaccaaagacattgagaattaagatagctatgatggcgtgcttccttaggtggaagtcctatatggaatcccatactccccaaatgtgactggtcggtgcagcttggggctaacagctctgttgacctgtggtgacccctggaaagccctgtgggatagaggaactcctggatgtcagtcatttgctctgcctctcttgtgaaagcagtggtctctgtttttcttccttctctcggcttttcatattctcttgcatatcctgtttatttgaaatgtttcttaaaggatgctcatagtctctcactggtggtggtgaagtacaaaaccctgctgcacagcaaacagagggggccagcatacggaacggattaacagccctatgagagcccacacgctcttcaagagaacattccatctgtatttgctgctgccattctgggatctgcatacatcagataggagtgggaagccctgggaaaacagcagggtccaaggcacatgtggccaattgctttttgcccatggtaaagttcacatttgattgctttccagattatgtggacccagacatattttactcggtcatccggatcttcctctctgggtaagtacagcttaattggtttcctgcttgagacctgtagagttccccagatttgtgcgaggatagacagacagaagcttcctaaagtaagaagtatagcaagtgactcaagctggcaaagtcaccacctgggtccactctaggaggctggaccccaaaatgatcagaaggaaaaaggttccaacaaagctgggaacttaaatccaaaaggggcaaaaggggctgtcttcagccaccagagggagaccatggaccctctgagaaccccaacgtgatgacaggggcaaaaattaaagatgggagagagagtgggcagggagaatgtggggaggagatggaagggagggtagaggagagaaaggctggaaaatgatagctacttctttgtcccttgacctttgaggacatagcattcaggggacccatcaggagacaagagtccaggctgtcacctgacagaaccatcaatgccttgatcttagactccctctccttcggagttgtgagaaatacagtatgattgatcatattcagcttgaagtattttgctatggtggtagcaggtacccttgatttgtctcacggctatacacttctctgtctgtctgcctacctcgcatgccccctgctccatcaagcacatgtcttcacccagttatttctaccttgacaagctttaaccaggttgccatccaattcaccaaccaacagagccgattcacttttgaacgtttgtgtcttgttgacttggaggtgccggtgatgttatcagaagcatccaagctgtgcctggagacagcaagagacagaccagatgccgaatctattaccattactgccaagtcccattgggagaaaaactaaagtgtgcattcgtgcatgtgtgcgtgtacgtgtgtgtctagaactgaaaattttatttccttgcaagtatcagactagagttttcctggcctgctaggtcctctgttgcctctccccaccatgtccccaatttgactcctgacacagcactggcacttggcatttttctagaattacacattttcctgacttttctcttgactaccgccagaggaaatttctcttaaaggggctcctgtggctgtgctcaatctagtcggatgagtcagggaaattcccacattaaagctaaactgatcagtggccttaatcccatctgaaaagtcctcctgccctgtcctgtaataaatatatcatggtgaccggaacagctcatagtattaagagtcctaggaattcggttgggaaccttggggagctatttttagaatcctaccctccagtgtctttcaaaggaatcatttctacttcctcttggcagagacttgataagaaccagaaggggactttgtttgcatcgacataagttccgggcaatgacactttttatctggtatttggcataaaggccatccctccacttcaaatgctgagactgtttactgtgccgctaagtggctgtacaagatcctaaatgtagctgtagtttcaacaaacatctggattgttgggagtttccagtagacttctctttaaaatgtcagcctcgctattctgcatctatcccgagtttctcatttgcttctttaaaacacagttttatttaagagtggatgtcctgtggaaatgagatgtattccctccagttcccagcctgccacaggtctcagagaggccaggaacggggggcccaggttgctctgcagggaaacatcgcctcattttgttgtctgccttaacttcctctttccttgggattgctagaaatacaccaggagcaggtctaaaaataaccttaccgtagaggccagaatttaaaatggccccatccctttacaaatggcagctcagccctacgtggagaaagaggcagttgtgtctcccagtgctgctggggatggggtgtaaaaacagacccaagttcatcatctgacctcagagctggtgttttctgcatcaccttcagcatggctggctttggcgaaggcactccagcgagtgctgaaaggacattgcctgatgacgtccttggcatttgtcatatctgatagatcccttaagcactggttgtgccgtactgactgaaaagctcttttctccttccttccttccttccttccttccttccttccttccttccttccttccaataaatccatagtgagggcctaccacatcctagacaccactgtatgtgctggaaacagcagtaaagaaaagcagtcacgtggtccttgtggagcctgtgacttcaggatggggagaaggtattgacaaagcaaaccgaatcaaagcaaacatccaacagtatgtgttacaactagaggctgcatgaagcaatgccgggtaggatgcgggctgctagggagggaccactgggtgtggtggctcaggtatcttgtttactgatatctagaaataccacatccccagtgatatgggaacagatgtcacaggcaatggagtgctctcagaagaagattcctgttgtaagaggagggaacagttcctgtgacaggaggagggagcagacatgaagaatagacttagatgtatgtgagaaggaggatgcctgccattgtggaggactctcagtgtgggcagtgctgccttcctttctgagtggcacagtcctgtttggggctgcaggttgtcagtacccctcctgagtggggtagggtggcagagaacaagattacaaagaaagcatcagaggtggacccaaggggaagatgccgagatggagatgaatgtcaaccatcttgaagatgaggggagaggtggagagtatacagcctgtgggtcctgacgcctgtcacagagaaacagaaacatgtgcgtgcccttggccgctctggacacagattgcagcccatgtgctggtgtggaccaggtccattatccacaccgtcgtcatgtctgcaacatcacttgctcataaaataccattctacagtttctctagccccgaactcttatatttgtttttgggttgttcacaggcctctttggattccattcaaatcctcgaacaggtgcaagaagcagattcccctacagactaggggaactgagggtcagaaaggctgtgcactgtagctgagagatgagagtggcagcaggcattcttagcctccagggttgctagaacaataaatagctggtgcttaccgaatgtctaatgtgctccagacacctggcaaatagtatttgagtcggatcttacaatgacccataagctaagtatgaatgtcaatgccaaacatagctcttatctcaaaggaataagagttagtttattctttggccaaatattaatgcccagaaacacagattccagttgtcccaaattaacatgttccactggggaaatgaaggagttttcatagctcaagggcaaagcaagttaccaatcaaggggttttttgagaacactggtgggggccccaggcaggcaagttacagcaaacaagaactctgtacatagtcacatgcagacgggtagtggaagcctgttgtctactaggtcaatacattccaaaccatcaaagatactgggtcaggcaggacggagttaaagatagcccattgtcgtttgactctggatcttcaaactttccaactctccacgatcaatcagttttgtagaatctttaacataggcatggcttccccctccctcctcccacacaccccagcgccacctggaacttcagtttatactaacactactcagacaagggagccaaggctctgagaggtcccatgtgctaacccagggctagcaaccaatgcacagtagagcttagattctgtatctgcccagaatagagaccttgtcccacagtttgtgtgacgctcccttctacctgtgtatcctttcttcttctttcttatgctgtatctcttttctgaacatgacttcctcagttggttaccattgaaattcactaatcggttgactttaaacagccaccaaatctggcaacctggccacttgctgtgttggcagcctcagagcagagtctggggaaaggtctaggatgcaggtggggggagctagcctgaacacaaggcttggattgacagggagcagaaacagccatgtaaacctgctacttgccctgtcaaccaggggctggtttgcttgctggcaaaagaggtcaatgtgcttcttggcgaaggtagacatctatgggataaagaaaagtagactgcaacttagagatgcatgtgtgtgtgtgtgtgtgtgtgtgtgtgtgtgtgtgtgttcctggaatgctgggggtctgggaacttagctcaagtctttgattctttaggaaagaaggtaaaaattgcctggtcctatctactgtcaagtggtcggggtgagagagtgagtgtatgctcgtgtatgtatgtgagcactgcaggtgtctgcatggtgtgccatggtgctccactgggcttcatgctgtgctcccctaggcttcagtcaagtcaagactaggtcaagtcatggagggtaaacagaagagagagagagcagaaaatgagggacacaggaagggtagagggggaaagagagggttgtgaagctcacgcgtgatcaggagccccaggctttcttcttccagccgcccataggctctggtgcccacaacattggttacaaccccgctctccatcatcatgcttgtcatcactgctgtgacaaaccgcttaataatggttctcacggaacattaaaagccaagccaagtttaacacctcgaacatttccaagtgttatggggaataacagttaagtgtctgggtgtgcttgtgtgtaattgggaatctgtagtggtggggttaccagtgtcaggccacagtgtttgtgatgagcagaggggtcggggtctttctcagatcccttatcttgtcctgtcaatggtggtgatgtaataggtgcacgcctgtgacagagctgtttaaagcattgtaagaccaatgagtaaagttcctacccttgcttctcctttaagtgaggcagaaaaaggctccaccatgacgtggtgtaaagatgaagtcaatctaatacttccttggatactctagcaagcttcattcacactttttatttcttcctcttcctcttcctcttcctcttcctcttcctcttcctcttcccctcctcctcttcctcctcctcttcctcctcctcttcctcctcctcctcttcctccttctcctcttcctcctccccctccccttcccctgtgctcctcattattattattattgctcctgtctaggtggaaggacaatccagccatgcctgtggggctggtctatgaaggtgttgccacagagcctctgaagtactctggaggaagtgcagcccagagctccgtgcttcatgccttcgatgagttcctgggcattgagcattgcaaggaaagtggtgagcagcagtctgatctcacctatgctttgatgggacagcgaggtagactagggagacatctctagcaactgataaagacgggtgtaaatgaaaatgtcctgaagtttatccttgcctaagccagcaggcagctgtgtgcatgtgccctctcttacactgagttagtcagtattggggcatcggatcttattagggtcttccaacagtcctgtgacctgggttgttcactgtcctgttggctggggtcttttatccgcagattcccctttctacaatgaggtgataatgtcacattgaaaggccagtctggagcagcaagtgatagtgctgaacttctctgctaaagcctttcccatgaaatggcccagcctcccactgaatctatgtggaccaggcgagggagcccatcgctttgaagcctttaaaatagttgttattgatttttctggcagcatagagctatcctttaatgactaccaaaaaaaaaaatattgctaccaaactttaatttggatcagaaccaaaaagaattaaaactctctggcacgaacctgttctctggaacagtcagggttgtttctgggtttcagcatttgattttagtcactgactctcaacaaccaaccaaccaaacaaacaaacaaacaaccaaacaaccaaacaacaacaacaaaacctaacccaaacaacaacagttaaaggtgtaatggttttcaagaaaacactgagttttcagtgtttcggcttctctgggtttttactaaaatacctgcttaccaatgtcgtctctacgtgcacagtaacttccatttcctgcccccctgcccgccccccatttataagtatctagcaaacagaattcaccagagtgttctgtgtttgtgtgtgtgcgtgtagactcgtctcttagtcctcagcagggaataccacatcagtcatacacacggagcagtagatttctgtccaactttattatctgagagatgtttgcttctttttctcccctgtgtctcctgtcaacaaccagaaatgaacatttgagcatttggcagctataacaaaagcccgacaaggctgagggagagccctatcaagcatttctggtacctgagtgtttggaacagtgggcaaaccctcccaaatgtctgcctcgagctaacgtatttctcccggctgtttctttcagttggctttctacacagaatgagggactacatgccgccttcccataaggctttcctggaagatctccacgtagctccttctctgagagactacatactggcctctggtcctggggactgcctgatggcctataaccagtgtgtggaggccctgggagagctgcgcagttaccacatcaatgtcgtggccagatacattatctccgctgccaccagggccaggagcagggggctaactaatccctcaccccatgccttggaagacaggggcactgggggtactgccatgctgagcttcttgaagagtgtcagggagaagaccatggaggccctcctgtgtcctggtgcttagcagtcatgtcctgcaccctaacacttagatgttctcatcctgcatcccagcgttagaggttcacatcctgcatcctagtgcttagctgttcttgtgctatatcccagcgcttagcagtcatgtcctgcatcctagtgcttagcattttatatccagcatcttagtgcttagagattcacatcctgcatcctagagcttagcattttatatccagcatccttgtgcgtatcagctatgttttgtatcctgcttagcagttaacatcctgcatcctagtacttatctgttctcatcctgcatcctagagcttagcagtcaggtcccgtgggagcaagaaccagggtctgagctctgtctgagcccaagcatggctttactgctttgttaattgtggctcccacctccaccccaccccagccagtttgcttgctagaagcctttctgcactgcctaatccccctgcctcacagcagagagctgcagccatgacctcctcattcagtattaggtggacaagtcggagatacccaaactcaattttaaaagaatcaagttgcttttggggcatgttacttcatcttttcttaccctgggcctcttcccttcttccctacctccctcgtcccttagtctttcacccctctctctttctccttttgtcaccctccccctcccctgcttactctcttttcccttcccccctctcctcatccctccttcctttcttccttccctttttgtctgtgaagcaccaggtctgatgggcctcaaactgtgatcttcctgtctcacccttcaaaggttatgtgtatgtgacgtgtgtgtgtgtgtgtgtgtgtgtgtgtgtgtgtgtgtgttcgtttcttttgtttttccctagtggagatgacacccaaagatttgcacataccaggcaattgctccaccacctgactacagtcccagctctctgtattcctgaaggaaagtcttgatgagttgcctaggctggtattgagctctttagcccaggcaggccttagtctgagtagctgggatgtacagggatgagccactgagccatgctgctgctgctaacgatgatgacgatgatgatgatgaagattatgataactacagtcactgcaataatga

SEQ ID NO: 15 - DNA, 61540 bp, Homo sapiens
  source 1..61540: mol_type = genomic DNA
ccaaaagttgtttggtaggagagggttgaggctgggagaggtggctcatgcctgtaatcccagcactttgggaggccaaggtgggtggatctcctgaggtcaggagttccagaccagcctggtcaacatggtgaaaccccatctctactaaaaatacaaaaaattggcgtggtggtgggtgcctacaatcccaattacttgggagactgaggcaggataatcgctcgaacctgggaggcagaggttgcagtgagcagagatcgcgccactgcactcaagcctaaccaacaggggcaaaactctaggactagagctaaggtatcaaaaaaaaaaaaaaaaaaaaaaagaagtagagtgtttaattaaataatttgttcttgctgtaaaatgtaaagtagatattcctcttcaaagactttcctccccgtctaattaggaataaatagtaacttctcttagaagcaaaatttattcaaagacctgtgctaacattcttaaatatctgctagccacaataaggaaatcaatgtactttatgttcttagctcccacaatttagcctaaatattttccctggcatgtttatactggtctaagcaagcattaggtcatagcctgttcctcttccttatttaaaagtgtttttacctttctcagcgttccacaagttacttcctccttcctttgttctcctctacctgtgcctcttttaaaaagttctaagttgctagccaattgggacaaatacagaatgtaaggtcccattccagccaacggaaactggacacagcagtagggtggatgtgtcaggttataaatgaccctgtctcctttgtttggtgtactctagtggcaaaactgctggcaagtgtaccttttctgcaggaagtaaaaatggccttactaaataaattaaatttatgttcaagtgctatttctttttttttttttttcgagatggaatttcacttttgttgcccagcctggagtgcaatggcgcgatctcggctcactgcaacctccacctcccaggttcaagcaattctcctgcctcagcctcccgagtagctgggattacaggcatgcgccaccacgctcggctaattttgtatttttagtagagatggggtttctccatgatgaggctggtctcaaactcctgacctcaggtgatccgcctgccttggccttccaaagtgccttggccttccaaagtggcgtgagccactgcgcccagcctcgagtgctatttctttacggcacggaagaacaaacatttcaaacaatgctattaccaagtttgttagtatttattatctcatttgctaaacctaaaaaatatatatccttctttaacgtgatcgaatatttcaaaaagttattgtgttgtttcttaaaataaatcaatcataatcctagactatgttactcaaactacatacaacaccttctgagcttctggcaggcccttcctcccctccctgctcaccacagatcactggaataattgtctgcatgtaacttctaattttgaagtggttgtggtttatcaaacctggaacatggcacttccaagtacatgagctaaggtcacagtaagactcaagccccttcaacagaatacctggaatttctctgttaaagattttctcctttacctgactacatgtttgtaatgcagatccctccaggagcgcttacttataaactgtcctggatcactaacgcgacattttgatgtaaattagtttatcttgacgtgctaatggtagaaaaaaagagaacatgaggaaacttgggtgctttcagggctggtaggaaggattaaatctttgcggcaatttctgagaaggggaaggaaaccttgctaacaattttgatagtttactccatttggctggagtaactctgatccatttgtcaaattcacgatggagcaggtacctgttagggtacaggtttgataaaccacaaccacaggtctatttcatttctccttttccaaagtggaacaaatttgtctctggggttaaaactgcttttctcatattggtgtgtaagagaaaatgagggaatttctttgagtttgtttggtttgtctgtttgtttaagcagcattttttaaataatttactcagccctgtctcagagaaagtccatgatgatctggaattcaacctcagggaaaagttctctcctgtgcctgagacactgcgcaactaactggaaccgaaggatggaacctgggtgtttaatttattaggaacaattgattcttcagtgacactttccatgcagatacttcaaacaaaataatggagccccacagaccgaatgtgaagacagcagtgccattgtctttggaaagctatcacatatctgaagagtatggctttcttcttccagattctctggtaaggatagagccttggtaaggataggtcagaatatgtttcttgagatgttggttggtttgttttttaaaaatgtatgtgattattaagagaccaatataaatatcaagttgtttacctgagaaagatgctacaaagagcatagattatcattactatcaaaagagaagtgacagataccacagagaacaggtcaaatggaacattttttgtttcagtttcttttgactagattgtcaggccagagaaattataagcaaacctgtagttatcaagaaaaagcatgaacttaaatataaataaagaacaaatacagagcctcagcacctggaacatggcacttccaagtacatgagctaaggtcacagtaagactcaagccccttcaacagagtacctggaatttctctgttaaagattttctcctttgcatgactacatgtttgtaatgcagatccccccaggagcgcttacttataaactgtcctggatcactatcgcgacattttgatgtaaattagtttatcttgacttgctaatggtagaaaaaaagagaacatgaggaaacttgggtgctttcagggctggtaggaaggattaaatctttgtggcaatttctgagaaggggaaggaaaccttgctaacaaacaatacctctttcttaattctacttagggctcaaattgtaatgcaaatctttttcatcatttagcccttataaacactgtttttctcatctggtgtggtccaaggcctagaacattaaaactatcaaagcttttacagaccatcaggtgtcatccccctctttctacatctgagctagctgaaatccagaggaaatgacttgctgaaagtcatgagtggcaaaagcagaactagttctgcttataactcttgacttttagttattattattattaattattattattacatcctaaatgagggccaaggccactcagttaaaaatcgtggggtccaggccaggtgcagtggctcacgcctataatcccagcacttttgggaggccaaggcaggtggatcacttgaggttcaggagttcaagaccaggttgatcaacatggtgaaaccccgtctctactaaaaatacaaaaattggccaggcgtggtggcacatgcctgtagttccagctattggggaggctgaggcaggagaatccttgaacccaggagggggaggttgcaatgagtggagatcatgctgttgggaatgaagtttttggtgtcacagaaaaagaatgaacatgggaacaaatgatctctcagcaaaaggacctttactttctgcagaaagggtgctactcaatagctgtccagccacgagagcacaccaaacaaaggagacagagttatttataacctgacgcatctaccctactgctgtgtccagcttccattggctggaataggacctcacattttacactttacccaatcggctattagtttaaaacttttttaattggataagggaacagaacaaagaaagaaaagcaagttgcccagggatagttaaggaaacatctccatataaggaatggcatgcactatgggctggggcttttctagttctgtacagacatgccggagcaagctacgacagctgatttggacagccactaatagtggctagcaatcttatagtaagaaattgtgactttttataatctttgaagaactttcccatttctgacagtgccactgcactccagcctgggcaacaagagcgaaactcgtctcaaaacaaaacaaaacaaaacaaaacaaaacaaaacagctctctactcttggaagcagcagagtttttatcttcatttatatcactccggtaacactcagaagtagacaagcctcagggtaggtattcagtaaaagcccactgaattccacactattctttaatcatagttaaatggcaaattaggctggagggtgggggtggaacctctccaaaattactgcaatgactgcaacatcggaccccaagatttttttttttttttctgagatagaatctctttctatcgcccaggctggagtgcagtggcactgtgagaaatgggaatggaccggactgtttcctctgacactgccactaggttgaccaagtgtccctatttgttaggtactggatggacgcctgacatgcaagactctcagtgctaaatcaggaaagtgctgggacaattcggatgagtcggtcacgctaagttgcacttaatagctcttgtgactttgactgaattacaaacatcccctgaccctcaattttcacatttactggatggagatctggtgccacctccactagattgctatggagaatgaatgtgaaagcattttcataaatccagtgtaaggaccagaagccagtcttctgaccttgagccagtgcttgttaaaaactccactctatacatctaacccaattcaggaatatcctgcctagttccaaaggaagaaaagaccaaattgctcttattgggattaaatgcgtacactgagctgaggaaaaacagtattacaaatgagctaaacatgacgtagatccacagttgtagaattcccctctttgttctttcctctttcataactacggaaacagatgagaaacatttacggcatcaggttcttgtgatgctccctgcctgatatgctatggttttgttaatggaatgtccattcctgagcttatgcagaaaaaagtcccttgggaaagtggttttactgtgttatgttcattttccccatagttctcaaaatgtacttccttgtttcagttttaattttctttcattggtgtgaccattttcaactgctccctttctgggaagaggtagcagacggacattttcatcaaaatctgccccaggttgcttcacagataaggagggacccagccactaaaatcaccaggcagagtgttgcaagagtagatagagaatcacaattggctgccctgctcaaggggacaccagatcttactttcgtttagttgaaaggcaagcgtcagagtcgggaggctgtaccttcatgtccagtggcctcacagaagttccttcagtatctcttttagatgaaactcttttagaagttccttcagtatctcttttggtttctcactatagatagttacttgaacatgtctgaagaaaacgtggtcaagacagtgaataaaaaaaattctggttttgggaagcagtctgacttagtttcaaatattctatcccactgtttctgtcaatgttcaaacctttccaagctccaacatttattgtggaaaatgtgtgcctcaccaactcatgcaaataaatgtttcatgtgccctacgtgtgtagagggggcatggatgtgtgtttttggagggagggctaatttttctttagacatggagaatacgaggaaattagcttggcatcaagaaggttacagcaggagacaagagtgaagagaactgagagagcccggaaatgaggctctggagttcagatttttttttttttgagatggcgtcttgtaccccaggttggagtgcaatggcaaaatctcagctcactgcaacctccgcctcccgggttcaagcgattctcctgcctcagcctcctgagtagctgggattacaggcatgagccaccatgcctggctaattctgtagttttagtacagatggggtttctccatgttggtcaggctggtctcaaactcccaacctcaggtgatccaccctccttggcctcccaaagttcaaggattacagccatgaaccactgcgcctggcctaatttttgtatttttagtagagacagggtttcaccatgttggtcaggctggtcttgaactcctgacctcgtgatctgcccacctcagcctcatgaagtgctgggattacaggcatgagccacagggccaggcctggagttcagatttaacacatcctgtaaatgacatgatgcatctgatatttgaagagttttcctcaaagaatgttacatgcaaggtggtttagagttgttgtttccggctatatagcaaaagtacttggggagttttaaaaaatactgatgccgaagctccacctagaatagttcattcagaatctctagcataattgacctcagtacttgaaatatgattattataaatgttagtcaactgcttttttaggctctatgactgatagaaatctttcacttttatatcatctccagttaatgagtcccataaattgaaatctagtgtttaaatttttacttcatatttatttttactgattgttttattattattatttttgagacagagtccgctttgtcgcccaggctagagtgcagtgacgccatctcggctccctgcaacctccgcctcctgggttcaaacgattctcctgcctcagcctcctgagtagctgggattacaggagcccaccaccaaccacacccagctaatttttgtatttttagtagacggggtttcgccatgttggccaggctggtctcgaacccctgacctcaagtgatccacccgcctcggccctctgtctcaaaacaaaaacaaaaacaataacaaaacttttctcttctacccgagatgtttaagtttaaatcacaccatttgtacaaaaattccctgtcttgtccttaaaaataatttgtaatcactagctagttttgagatcgattgccatctaaccgaatgccatttgttctctctctctagtttcaacttaataaccctttctgcattttctattctttcaaaatttttccggccattttattgtttctatttagtgaaaatttattcactggtttctatgcctaagggcatttaggaagttgcttaggatacagacgtgataaaaagaccagtgtaaaaactctccactcctagacattatattctagtcctcatctcctgtcatttaagtcctcagtgattctatgcacttttgcttttggtttgggcagatgctctgagtttaatgtttctctgagatgaggaccccctattcaactcacaaatcccataaggaggcctctgtgcctttgctggtgccccagacagggtgctgatgcttacttatcttcaagattgtgaagtcagatttaatagtatagtcgtttgccagagctgctgtaacagtagccacaaacagttgggcttaaaataccacaaacaggagggcttaaatcacagaagttgattttctcacagttcttgaggcaggaagtccaagatcaaggtgtcgtggagttggtttcttctgatgtcttgctccttggcttgtagatggcctccttattattgtgtcctcacatggtcttttctccactatgcacaaattccctatgtctctctctcttttttttttttttttttttttttgagacagagttgcactctgtcacccaggctggagtgcagtggtgcaatctcggctcactgcaacctttgcctcccgggttcaagcaattctcctgcctcagtctcctgaatagctggtattacaggtgcgcaccacaaagcccagctaattttttgtatttttagtagagatagggtttcgctattttggtcaggttggttttgaactcctggcctcaagtgatccgcgcacctcggcctctcaaagtgctaggattacaggcataagctactgtgcccagtctccccctgtctctttgtgtccaaatttcctcttctttagggacaccaatcagattaaattggacccaccctaaaggcctcatttcaatgtacctccttcaagggcctatctccaaatacagttatatttttaggtactagggctagggcttcagcataggaatttgggggagacacaatttagcacatagcagaaaatataaggccaggaaaaaatattctggcatgctagatggactcattaacaaatattaaccaatataaaccaattaacaaatatttctttaatatttgcctttttttttttttttttttgaaacagagtttcactcttgttgcctaggctggagtgcagtggcacgttcttggctcactgaaacctctgcctcctgggttcaagtgattctcctgcctcatcctcccaagtagctgggattacaggtgcgggccatcacacccggcttattttttgtatttttagtagagatggggtttcactatgttggccaggctggtctcgaacttctgacctcaggtgatccacctgccttggcatctgaaaatgctggggttacaggttgcctggtgatttttaagaggaatgactgagctctcatgccaggtggggggaggggacagagaaagttgaatactctgacgatagccatgatccatagctctgaagcttagactcgaatctacccatcccgcaaggaagaaaacaaagaaataaaaaagaagaaaagaaatctcccaatgtcaggtcccaccctctttagaagtaatttcagcaaaactttgttgctattttggcatgtcctctactgtagtagcttgtcaaaatactgtccccaaacgttttctatatttctagattttactgtttaatgtataataataatgttctaacattaaaacgtaaccatagcaatgctccgactcacttgatctttaaatatattgttgaactcaattttgtgacatcttcagaattgtctttttgtattcataaatagcaacagtggatagttgccttggttagtgttattatttcaaggattaatctttaggttacgttttcttcataagatgcattagatgacttttttttttttttttttgagacagagtcttgctctgttgctcagactggagtgcagtggtgcagtctcagctcactgcaatctccacctcctggctcaagagagtctctcgactcagcctcctgagtagctgggattacaggcacgcaccaccatgcctggctaatttttgtatcctttttagtaaagacgggatttccccatgttggccaggctgatctccaactcctgacctcaagtgatccatctggcttggcctcccaaagtgctgggattacaggcatgagccaccacacgcagccaattagatgccttctcatgcttttctgtgttgtgaaacagatcatctatccgttgatatagcatagctcttcagactacagacattttaggctataagttttgaattacattttctattccctttatgcttcttgttctagttaggttttttatttctaaataaaaatatgaatttttgcaatttcccaaatgcgaactcaactgaaattttcaagtgtattagcaaaatttattcatagcagcctcttacatttcattaagaattgattttttcttattccctgttgaagtttgtttataaaatatagtaatagtaataactatatagaaagtgttcactttgtagtaggccctatgttaactttaactacactttttaaatctaagcctcatagtagtcttggatggatgcggtggctcacgcctgtaaccccagcactttgggaggctgaggcgggtggatcacgatgtcaggagttcaagaccagcctggccaacatggtgacactgtctctactaaaaagacaaaaatcagccggacgtggtggtatacacctgtagtcccagctatttgggatgctgagtcaggagaattgcttgaacccaggtggtggaggttgcagtgaaccgagatcacacaagtgcactctagcctggatgacagagtgagactccatctcaaaaaaaaaaaaaaaaaaaaaaagagatcaataaataaaataaataaataatcctcacaatagtcttcaccatttgcaaatgatccatttgacaataaatgaattcagcactatcatggaaatatatttccaagctgaccagttttctccatttccaccccacttcaatccaccttcatgtagccctaggactattgtctcctccccttgtttccttatttccattcctgttcagctgtaacacattctgttcatagcagtcaagtgatgcttacaaatggaaatcaggctagaggtggtagttcacacctataattccagcattttgggaggctgaggcaggaggatcacatgaggccaggagtttgagaccagcctgggcaacatagcgagaccccatctctacaaaaataaaaaagaattagctgtgcatgatcctatgtgcctgtgttccagctacttgggaggctgaggtgggaagattgcttgacccagggagtttgaggctgcaataagctatatttgtaccactacactccagtgtgggtgacagagtgagatcctgtctctaaaaaacgtaaaatgaaaataaaaccttgatagtttgctctttaaaactcttcctacagggcccctgtgatgctcacctgtctctagaagggcatgtaatagctctttctccttcactttactttgatgcaatgtcagaacagcttctttccatcaaaacttaaacctttgatttcatttaaaatcatctgcttcaaattctaatctttctgatagtttaggttctaatttttctgatgttaatattgtcacccaagtttcctgttcatatttacctggtttattttatttttatttttatttatgtatttgagatggagtctagctctgtcacccaggctggagtgcagtggtgcgatctcagctcactgcaaccttcgcctcctgggttcacgccattctcctgcctcagcctcccgagtagctgggattacagggacccgccaccatgcccggctcattttttgtatttctactagagacgtggtttcaccgtgttagccaggatggtcttgatctcctgacctcgtgatctgcccgcatcggcctcctagagtgctgggattacaggcgtgagccaccgcgcccagactttattttattttttgagacgaagtcttgctctcttccccaggctggagtgcagtggcttgatctcagctcactgcaacctctgcctcccaggttcaggcgattctcccgcctctgcctcccaggttcaggcgattctcccgcctcagcctcccgaacagctggggttacagatgcctgctaccacacccagctaatttttttctttttttggagacagtctcactctgtcgcccaggctggagtgcactggcgtgatctcagctcactgcaacctccgcctcctgggttcaagcgattctcctgcatcaacctcctaagtagctgggattacagacgtctgccaccacatcaaactaatttttgtatttttagtagctgagattataggctcgtgtcaccacgcctggctaatttttgtatttttagtagagacggggtttcaccatgatggccaggctcgtcttgaacctctgacctcaagtgatctgcccatctcagcctcccaaagtgctgggattacaggtgtgagccactgggcctggcacctggtttatttttgtgcatgcttttatttttaattttcctatgctactttcttagccaaaatttatacttaatctaatcaagcattaatctaacaaagagtttagtgttcatataaaatacagttttacaaatctgtttttctttaaattataaatttgttaagaaaattatccaaagaatgatccagaaacaaaagaatggctgtgtgtcttttcaatatcatcctggagcattgtctcaaccatctcactttacggtgactaaaacatctagaggttttccctttgttttctgtacttcttagtattgattaatactgttgtgctacttcagtctgaagttccatgttaatctgtagatttttttttttttttttgagacagtgtctcgctctgtcgcccaggctggagtgcagtggtgcgattggctcactgcaagctctgcctcccaggttcaggccattctcctgccttagcctcccgagtagctgggactacaggtgcccgccaccacgctgggctaattttttctatttttttttttaggagagacggggtttcaccgtgttagccaggatggtcttgatctcctgacttcgtgatctgcctaccttggcctcccaaagtgctgggattacaggcgtgagccactgtgcccggctgttaatttgtagatttttatacagaaaagcagcaaaatatttctgttgagtagaaaatataactccaatgcttatgactgtattccttataggacactaactcattatgtgtctaacctagcaattttatgtcaacactattttctcaaacctctataaactttggctgggcacagtgggtcacacctgtaatcttagcactttgagaggctgaggcaggtggatcaccttaggtcaggagttcaagacaaggctggccaacatggcaaaaccccatctctactaaagatacaaaaaattagccaggcatggtgacatgcccctgtaatcccagctactcaggaggctgaggcaggagaatctcttgaactcaggaggtggagccaagatcatgccactgcattccagcctgagcaatagggtgaaactgtgcctcaaaatgaataaataaaataaataaataagtcagagattgtgaataggatgttggatatacccaagttatgaattaattaggagcttgaacccaggaggcagaggttgcagtgagctgagatcgcaccactgcactttagcctgagcgatagagtgaaactgtgtctcaatcaatcaatcaatcagagattgtgagtaggatgttggatgtacccaagttatgaattaattaggagcttgaacccaggtttgtctcagatcctcagggactgaagacttccaagtgaattatgggtaatgtataggtctatactactccaaatttacagttttcagacttccctgggttctcatggaccttccatgtcatttctatgtttaggttgaggtccctggtcttttctcctctgtaattaatttcacacccacccctatctcaactcacacaacttgatcttcactcccatctgctaagaaattgagtccataaaaagtgaactcctttaaactctagatcttctactgctgcaaggacagacattccattctggctctctctctcctcctttgcttcttccagtcctttgctccttctgcattctgttactctcctctctcctttgtcttcaatctctccctcttgcaatagccaactataaactgctcaagcttctcattcttaaaagatctctctgaaatgcaaattccctactgccttatggctctccttcaaaagtaatctacattttctctattttctaattcctcaacacactaacgtttgaaccctgcttctatgaccctgacctaaatttctattaaatgtacatagataaactaatatatatttgtgacttcctaatattgttttgttttttaaagaggtcaatcttactttaactctgtaatgatgcggttgaccttctgatgattctctacttctgtgaaatcctctactgtcttgactttttaattaatttatttttttttgagacggagtctcgctctgttgcccaggatggagtgcagtggcacaatctcggctcactgcaagctccacctcccgggttcatgccattctcctgcctcagcctcccgagtagctgggactacaggtgcccgccaacacgcccagctaatttttttgtatttttagtagagacggggtttcaccgtgttagccaggatggtcttgatctcctgacctcgtgatctgcctgcattggcctcccaaagagttgggattataggcgtgagccaccgcacctggccctactgtcttgacttttatagggtcattctattcaagctcattgagcgtctgtcactatgtttttgatctgtattgctggaatcagttcctctatctgccttgtgaatattctccattgtgctaatctaggcttctcctttcatttttcatattccctcacatggctttatacactcttgtggttttaaccacaatatagggtttttactgtagttatgctttcaaattgtatacttcttttttttttttttttttgaaatggagtgttactctgttactccagtctggagtgcagtggtacgatcttggctcactgcaactttcgcctcccaggttcaagcgattctcctacatcagcctcccgagtagctgggattacaggcatgtgccaacacgcctgcctaattttttatttttagtagagacaggatttcaccatgttggtcaagcccgtcacaaactcctgaccttaggtgatccgccctcctcggcctccccaagtgctgaaattacaggtatgagccatcgtgcccagccccaattgtatgtttctaatatgacttttctcctgaactttaaactgtgtatccaacttctcagcacagtcatatcttttgattccacaggtaatttaatgtcaatatatttaaaaatgaatttaccacctttatccccactctttgactttcacctgcatttctgtttcaattcttgtttccatccattcatttgctcacctaattcataaacatggaaatcatcctcaattcctcttcttcttagcccaaaaattcaattgtgcaggttatgcactgagaaaaagagcctcaggttagggggataaatcagagattggtgaactttttctgaagggccaaatactaactgctttaagcttgcttaccatatggtttatgttgcaactaccaactctcctgctgtaatgtaaaagcaaccatagatagcatgtaaacaaatgagacaggctgggtgccaatgaaaattcacgaaaattaatgtagtttactgtcccttgggtgagagttgggggtcactgaaattcggactatgtcttacttggctaaaccacaggcctagagtgggccataaatggagctattgggctagtgattttcttgccttaagcccccagccccaaatttaataatcacatcattttaatttcattttccaagtgtatctttaatatatggcttctcctttccaaattcactgtcattacctaagtttagtccttgaacaatattttaaaggcttccttatctgactttatatcttaaagtcctacaaatttatcttcctaaaattcaaatcaaaccatgtcaccaacttacagaaagggaaaattcatatattctacacacagcacatttcatgtaactttctaggctcatctttcatcatccttttgatgcaggattttctgctcctcagctcagcgaaatccaggatcttgtctcatgaccaggaagaattaggcaggtggacatagtgaagggtgaggatgacggaatttattaagcaaaaggggagttctctgcaaagagaggggtttcaccagcagtctcccacctcacaatggagcaccaggactttcacacacaaactgaaaaggctaggctcctccccagcataaggcatgaattcctggtggttccaccagttttcctactatgcatgtgggtgtgcccaagcaaaccataggtagtatcagaaaaggcaacatttgattggttaaaaggcattattcacccaagcaaaccataggtagtatcagaaaaggcaacatttgattggttaaaaggcattattcagaaagaatcaatcgggaaagggtgagccaataggggaagttctccctctgggtcacgggtttcatctgggaccaggagtctggcctttcagcctttagactgttttaggcttgaaggtgggtttcacagggacccttccctatctgcctaggcatctgtctgcctcctgcctctatcactttctcttgagttttatattttagcaacactgagtcatctttgtcccaggaagcacctacatctgtttatctgctgtcccctctacctttactaccttcccttcttcacatttatacccagaaaagtcacttcccctccaaaaattgggatcaactgtcatatttttatgaatatttcactttaattcctcacaacagctgacagaattaactacttcctcttctttgcaagttatttggctcacacagatatcagtaattaaacatattttactgcattggcatatatctgactaatgtgtttttctcccgtactaggcaatatgctccttagtcatctgtgtatctgaggtgagcacagggcctaactagcatatggtgcattctcaatgttcgttcaactgcattgacttgaattcccctgaagactgaaatgtgaaaatagctactctcggaagcccctttccagagaggtctaaaatatttacatgtttctattttaaatgcagaaagaacttccagatcattataggccttggatggaaattgccaacaaacttcctcaattgattgatgctcaccagcttcaagctcatgtggacaaggtattcttctcttcaccccctcatcacattctgttttcatcatcataccacttttctttcttagccttgtggaagtgtgtcaattgtcctgggaaactgttcattaccattgaacttatcagcaaagctatatcttccttcctgaaaaacagaatgaccccttcgtaatctgatacatgtgttttcctaaggttttcagagccagcacaaaacaatgcctgacacatgccaataactcaccaaatgtttgtttaaagaagaatctgggtgggaatgataaactaactaatggacaaggtatcgcctaagaaggtcagcttggaaattctcaggttcctcattccatgtacgtactcaaggctctgttgttactgagggggtctaacttgattttgtcctaggtgttatagaatagttaaatggagggaatttctgaattataaaattggccatgggttctacaaaacatccaataagcctgtaaattccacaaaagtgttgattaggctgatacaaaggtaattgcagtttttgccattacttttaatgacaaaaaccacaaacacttttgtaccaacctaatagctatgtaaccctgaaaaagttactcaactctgtaatcccatttccttatttataaaatgagagaaactctggtctcacagtattgttatgggaagtaaatcactttcaaagtggcccttttgtagttcttgtcctataatagcattcagtatacattcattacttctctgtagtctcttctccatctgtcctaatctatcagtttggagtaccacataattgcggaagtccatgaaaagttttccgctctccaaaatttccctttgctgatggataatatttaatgtctagaattacaaattctttttaaaatactcattgaatgtttgctttgtgcaaagcactagaaccttgtaaaagatgagtaagggactggcttcaatgtctgtgaagatagcaaactaaacagagtaatttctttgcctgatagataaaatgttgtgttgacatgaccaaagaaatccaaaaataagaaaaaaactatctgtaaacacagaaaaatagagaaaagttccaatgatggaataaaaatttaaaggatttttttgaacgtattaagcaaatcatgtataaaatccagaaataagtttacaggacccatgtcaaggatttaaccaaagcagagggagatccccatgagtccccttttcccatctcagaatagcagagaagagaagcaagggaagcctggaacagttggcaagagggcaggttagaattcagtttgtgaattatgaggtcgtctgccgtaggcatttaccaggctttatttgatttaactgccataaaggaagagaaggacttgttaaattggggctcctcttagcacagcattgaaaccagtccctattccttcttggccttttggctaaaattgagtgtgaaatctatcacctaacatttgtactgggtttaggctgggtgtggtggctcacgcctgtaatcctagcactttgggaggccaaggctggcggattgcctgagctcaggagttcgagaccagcctgagaaacatggtgaaaccatgtctctactaaaaatagaaaaaattagcagggtatggtggcacatgcctgtagtcccagctatttgggaggctggggcagaagaatcacttgaacccaggagacagaggttgcagttagctgagatcacaccactgaactctagcctgggccacagagtgagactctgtctcaaaaaacaaaacaaaacaaacaaacaaatatatatatattaaaatacaaatttttactgggtttaatagtgtcttcctagaagtcatgttcatgcataatctgtgaaagtggtcttatttggaaatagggtgtgtacagttgtattcgagttaagctgaggtgatactggattaaattgtatatgatgagtgtccttataagaagaggaaaaattaaacacaggaacatagactcaagggaaaacatcacgtgaagatggaggtagaattggaatgatgcattgacaagccaagatgtgccaaggattgctggcagtcaccaggagttaggagacaggcatggaacagaatggaacaaattctccctcagaggctccagaagaaatcaaccctattgataccttaatttgggacttctatcttccataactgtggcagagtacatttctgctattttaagtcatgatgtttgtggtcattagttatggcagcgcataaaactaacacaacactcttggtctctatcgcttcttttttttttttttttttttgagacagagtctcactctgtctcccaggctggaatgcagtggtgcaatcttggctcactgcaacctccagctcccaggttcaagcaattctcctgcctcagcctcctgagtagctgggactacaggcacccgccaccatgcccatgtaatttttgtatttgtagtagagacggggtttcaccatattggccaggctggtctcgaactcctaaccttgtgatccaccagcctcagcctcccaaagccctgggattacaggcctgagccaccatgcacggcctctatccctactcttaattgcctggaaaatacctaacaaatgaaggccagtttttagactttaccaccaaaggctgaaattgaaacaggaattgtttgtgagaagcaaacacaatagtttcgagagactgaatcagttagcaatttcctgtgagaggcaaatgtaatagtttctagaaccacagatggagctataacaaaaacatgtgttctctggatcctttacttgctacagacaacacaataagtgaatttacagctttgatcttacagtgcacctaagccaaccaccttgtcttagaatgtctcaacatacctatctgtatcttgaaacaaaatatattaattgccttagacccattcactcacatttcctaggaagacatgatcagagggagctatgcaagaagaaatccagcagaactctggaaatacaataagaaaatccatattagacactaatcttaataaaactaaccttcgttcatgaatttgaatagacaaaattaccaaataatatggaaaaaatgggcaactcaaaaagaagagggtagcccacttggcattcaggaccaatggcctccaataaataagatgatattagtgctttaaatattttatttagtgtatccagtatattgccttcctaaattaagtgaaagctgatatataaaaagaactattagaaataaaaaaccacacacatcccagaactcctcaatataaacctaacaaattcagtaaaacattgattgcaaaaaatatatatagtgacttagggacttccttgataaatttcataaagcataaagaaaaatcaaagagtgcaaaccatcagaaaaatacaaatatataaaaaagagaaatacaggtgatccaaattccttttaatggaatcccataagcagatgggtggaggaaaagtaaaacttccaacatataagcacaaaatttttagaccttaagaaatatttgagttttttatatcagaaagacagtgttggggtgagggttggggggcacatgtaaatactcagataaaattgtgaattttctagggtaaagaaatctgcatattcatagaaaacaaaaatgaaaaatagtttatttacagagtaaatacatacgactgtttacctgtaatgctaaatattaaaagacagtttcttttcttttttttgagacagagtttcactcttgtcgcccaggctggagtgcagtggtgctatatcgactcactgcaacctctgcctctgggttcaagcaattgtcctgcctcagcttcccgagtagctgggattacaggcacccgccaccacacccagctcatttttgtatttttattagagacggggtttcaccatgttggccaggctggtctcaaactcctgacctcaggtgatccacccgccttggcctcccaaagtgctgggattacaggcgtgagccaccgtgctggcctaaaagacaatttcatacctatttgtatggctaattttttaaaaatctggccatatcaaaatactgaagaggacgtatagcaatagagactttcattcattgctggttgaaatgcaaaatggtacagccagtttgaaaactagcttggcagattcttataaaatgaaacatagatttaccatgcaactcagcaatggcattcctaagcatttatccaagtaaatggaaaatgtatgttcccagaaaaaaaatccatatatgaatgtttataacagctttattcataatcaccaaaaaaaaaaaaaaatctggaagaaaacaggatatccttcaaccggggaatgaataaaccaaattataataattgtaaattgtgggatggatagtggaatagtattcagcgatacaaatgattgagcaattaatttgtgcaatgacagggatgaaccttaaatacatttacctaaatgaaagatgtcaggcctatattgtatgattcttttcaaatgactttttagaaagggcaaaactagaaggattaaatatagctttatggttactagagacaggtaaggagtgggtagttgactgcaaaggtaatatataggggaatgtttagcatgatgaaactgttttatatggcactcaggtgatggatatatggctctaggcactgaaaaacccatggaattgtatgtcacaaagaatggactttcatgtatgcaaattttaaaaaataaaccagaaaattgggagaatactaggatggaatgcagactgtgataaataaaactaactggactctcagcaaactaacacaggaacagaacaccaaacaccgcatgttcttacttataagtgggagttgaacaatgagaacacaaggacacagagaggggaacatcacacaccggggcctgttgtggggtggggggctaggggagggagagcattaggacaaatacctaaagcatgaggggcttaaaacctagatgatgggttgacaggcgcagcaaaccaccatggcacatgtatacatatgtaacaaaccagcacattctgcacatgtatcccagaacttaaagcaaatttttaaaaaagtaaaaaaaaaaaaaacaaacaacaacacctaactggactacaaatgcactatataacttcgatgaagagagtggggagtagggaaaggaacggacttaaattactccagaaaatagtgttgtgttgtgactagaatctataaggcttacggtaaatgaaactttacaggatcactatactctaattggtaaatcagtttttcatggggtgcgggtgaacagttgtgaaactgctttacatgtagtcatacctttgcattttgcagatatttcaatttttacaaattgaagattcgtagcaaccttgcatcaagcaagtctgtcaaccccatttttccaatagtgtgtacgcatttggtgtctgtgtgtcatattttgataattataacaatagttaaaactttttctttactattacatctgttacagtgatctgtgatcagtgatctttaatgttactatcataatcgttttgaaggtgccataaactgtgcccctataagtcctgaaacttaattgataaatgtatgtgttctgactgctccactgaccagccattgccccatctctctccccctcctcaggcctcctgatttcctgagacataataatattgaaattaggccaattaataatcctacaatggcctctaagtgttcaagtgaaaggagttgcatgtctctcactttaaaaatctaaaactagaggctggtcatggtggctcaggcctctaatcccagcactttgggaagccaaggcggggagatcacctgagttcaggacttcgagaccagcctggccaacatggcgaaactctgtcttgactaaaaatgcaaaaattagccaggcatggtggtgcacacctgtaatcctagctactcaggagactgaggcagaacaatcgtttgaaccctggaaatggaggttgcagtgagcctagattgtgcgattgcactccagccagggcaacaagagtaaaactccttctcaaaaaaaaaaaaaaaaatatctaaagctagaaatgattaagcttggtgagaaagtcatgtcaaaaccagataggcttaaagctgggcctcttttgccaaacagccaagctgggagtgcaaaggaaaagcttttgaagaaaattaaaagtgctactccagtgaacatacgaatgataagaaagcaaaagagccttattgctggtatagaggaagtttgagtattttggatagaagatcaaaccagccacaacttcccttaaaccaaagcctaattcaaagaaaggccctaattctcttcaattctacaaagtctgagagggctgaagaagctgtagtaaaaaagttttaaaccagcagaatctggttcatgaagtatgaggctagaagccatctccacaacataaaagtgcaaggtgaagcagcaagtgctgatggagaagctgcagctaattatccagaagatccagctaaaatcatcaatgaaggtggctacatttattttttatttttgttatttattaatttatttattttgagacaaagtcttgctctgtcccccaggctggagtgtggtggcatgatgttggctcactgcaacctccacctcctaggttcaagcaattctcctgcctcagccttcccagtacctgggattacaggcatctgccacaacgcctgactaatttttgtatctttggtagagacggggtttcaccacattggccaggctggtcttgaactcctgacctcaggtgatccacccgccttggcctcccaaagagctggattacaggcatgagccaccacgcctggccagtggctacatttaaaaataggtgttcaatgcagacaaaaccatcttttattggaagaagattccaaccaggactttttttttttttttttttttgagacagagtctcactctgtcgccaggctggagtgcagtgatgcgatctcagctcactgcaatctctgcctcccgggttcaagtgtttcccctgcctcagcctcctgagtagctgggactacaggcacgtgccaccatgcccagctaatttttgcagttttagtagagacggggtttcaccatgttggccaagatggtctctatctctgacctcgtgatccaccccccttggcctcccaaagtgctgggattacaggtgtgagccactgcgcccggcccaggactttcatagttagaaaaaagtcaatgcctagcttgaaaggacaggctgactctcttgttaggggctagtgcagctggtgactttaagttgaagccagtgctcatttaccattcctgaaatcttagggcccttaccagctatgctaaatctactctgcctgtgctctgtaaacggacaataaggcctggatgatggcatatctatttacagcatgctttactgaatactttaagctcactgttgcgacctactgctcagaaaaaaatattcctttcaaaatattactgcttattaacaatgcctctggtcacccaagagctctactggagatatacaggaaataaatgttgttttcatgtctgctaacacatttgttctgcaacctatggatcaaggggtcattttggctttcaagtcttattatttaagaactatactttgtaaggctattcctgacacagataatgatccctctgaagaatctgggcaaagtcaaatggaaacctggaaaggattcactattctagatactattaaaagcattcatgattcatggaggacgtcaaaataaaaacattaataggagtttgcaagaagtcaaccctcatggatgactttgaggggttcaagacttgagcagaggaagtcactggagatgtcgcagaaatagtatgagaactaaaattagaagtggaatctgattttataactgagttgctgcaatcttacgattgaacttttcttttcttttttttttgagatgcccctgctgagctgccagttcctgaagggtcaccgggagcagcgcctggcccacctggtcctgagcttcctcaccatgggttatgtctggcaggaaggagaggcgcagcctgcagaggtgagggccagagagcagcttctcctgttacccggcaggttacctgcgcctggagtaacgtgctccctgcttggtgctaccctgttttcctggaaaatgggtactttcttcttctcgatgggcatcagtttaagcaacgatgaagggctcatttattatttattattattatttttttattttattttgagccagtctcactctgtcactcaggctggagggcagtggggtgatcttggctcactgcaacctccccttccaggttcaagcaattctcctgcctcagcctttcttgtagctgagactacaggcacccaccaccacacctggctaatttttgtatttttagtagagatgggtttcaccatgttgcccaggctggtctcgaactcttgacctcaggtaatctgcctgcctgggcttcccacagtgctgggattataggcgtgagccactgcgttcagcctgaagggccatttaaatgaaggatttttttattttaatttttctgactaagagctaatttgttttttaaactggtagctatttcttccttttataagcttttgaatgtttgtttgtttgtttttggcactctcttccaagaatgtttgaagacctgcatttgaaggcagattgcctttttgctttaaaacagggttgcaccatgttgcccaggctggagtgcagtggtgcaatcatagctcactgcagcctcaactcctcccaggctcaagcaaccctcccacctcagcctcctgagaagctggggctaccagcatgtaccgccacacccagctaatgttaaaaattttttgtagagatgagggtcttgctgttttgcccaggctgatcttaaactcctggcctcaagtgatcctcctgcctttgcctcctgtgctgggattacaggcgtgagccaccatgccgggcctgaagacagactctgagaattcataaaaacctcacagcattttgtactcttatgtatataaattatctaggttgctcttcataatcctgtaaagtaacaagagccataccggcccattttacaactgaaaagcacagacacttatttccttaatcaaggtcagacagcaaattagtggaaaagccaaggccagaacccaggtcttctgattttactagtgcagccttctttccccaggggacacattgacatttacaacactcatctttattttttttttaatactgctttctatccagccaattattagtctgtcttttaataattcatccaaatctcttctgaatcattgcataactttgtacagtttccacccacagtgtcttttacttttatttttggaagtaactgtttttaaaagttactgttatttttaaaagtgtgccttccccagaaatcagggagttacccatgtcctagaactccacggtgaagagaacagcctgtgcccatcgtgtttgcctgattgatcctacctcttgtctctcggggaaacacaggagactcagggaagaggaaaagtgtagagtcattgcagccttgttatttgtcaatgcatctcttttctttttctttttctttttttgatacagagtttcactcttgtcgcccaggctagagtgcagtggcgtgatctcggctcactgcaacctcggcttcctgggtccaagggattctcctgactcagtctcctgagtagctgggattacaggcacctgccgccacggccagctaattttttttgtatttttagtagagacgggtttcaccacgttggccaggctgatctcgaactcctgacctcaggtgatccacccacctgagcctcccaaagtgctgtgattacaggcatgggccccagcacccggccagtgcattgcattttttttttttttttcgagacggagtctcactctgtcacccaggctggagtgcagtggcacgatcttggctcactgcaagctccgcctcccaggttcacgccagtctcctatctcagcctcccaagtaactgggactacaggcgcccaccacaacgcctggctaatttttatatttttagtaaagacggcgtttcaccatgttagccaggatggtctcgatctcttgacctcgtgatctgcccgccttcgcctcccaaagtgctgggattacaggcgtgagccaccgtgcccggcgtgcatttttaaaagtgtgtctgatgctgaaaagtttgaagtctaggcacgtcccagtgggtcctctttataccatcccctctgcaaaccattatcctaaattggggtttgggggagagaagagtgacagtggaaagaagtctccacctcccagctgtgccctggtagttccaggggacccggaggctccccacacccaccaccccgcctcagatcacctttcactttctttgtttctcctcccttgacttttcagctcagaaagtacctggctctccaatgccttctgaggaaagtttacccgaggttcacattgcaagactcattaaagctctttagtgttttccacccgagaaaaaattcaagggaaaaatgaagacaaaagcagggcattcttaatggatattttatcttaaggagaaatgaaaatggagatggaagagggggcacaaggatggggtttgaatctagactcgttcagcctttacctccgatagagaacctcatacagcttttctggacttctggctgataaagagccgtggagggttccttggataaaaaaggttgaagggggtctgtcctgtggtggcttacttgaaggtattactgggtttgacttatggagtaagagacggagtcagtttccccacaggctgaggcagtctgtcctcatgcttttctagggcactgtggtctcccaggctcatacctaggtgcacacacaggtttctgcatctagctttgtatctctatgagtcggtcaatcaataaatctatctatcatctgtctactgatctatcatctatctatctagctagctatcatctctctatcatctatctatgtatctatcatctctctctatatatgtgtgtatatatatatatatatgtatatatatatttctatccttccatctacttacctatctatcaaaatttttttccgttgataatattctcgggccccagtttatgtttaattgttttggtaatgcctttctttgcacagtcagtttacagaggttattttatattctatatgtatgtgtggtccagcgttgtaattttcacatatattgcaccgtgtactcataagcagtatttccactgggtcattaacagaaagatatgtgtgcggcatatgaatgtgcatcactcaggtaattcaagcttggttcccagatcatttctgtaccacaggattgccgaaataaaagacaaccatggttatttcctctgctgcaagctttctagaatatgctatttgtctggatttatatctgaaaggtcctgccaaggaatcttgcccttccatttgtcgaagtctccaggaacttggggctccctcctatcctggtccactcagacttggtgctgacgaactggaccaaaaaagatccagacgggtaaggaaggaagagaatgctttgaatttccataactttcccccaggaaacacccaggcttttttttataattagggaagttcatatttatggtctgccgtatggttccaaagaaggggtgagcttgaccaaaaattcaaatatcacaggccccagaagtttcctcttaatccattctgaacacattggctcagaccattttgtcttgtttgtttccacatgacgtgtgaatttctcaacctgaccttcaagctcctgcaaaatcagcttttatttgttctttctcttcaaactgtttattccctaagatgccctccattcatatcaggttaaaaccagttggctttgataagtaatcattatataatgatcagaagagaatgattatggatgaattcagagcagatgctccaggtgggttggattgagaatttgattaataattccatctattccaccaaagtcacatcattcctttgacagttgggctgggaataggggcatttgtctacagaaggaatagcatgagattttaacaaacaagaaattcaacaaacagaattagacagatgatctgagatgttaaattttcctttcaccttaatttttgcagccaaatttatttcagctctagatgaaagagacagcactttcttttgtggctgactacaacagctgaagattcactgaggtttgatatgaggaagaacttcctcagccatgggattgccagaggcgatgatgggaatctacttaaaagggttacatatttagtagacagcctagattttaagactaatttatgtgccccggccgggcccggtggctcacccctgtaatcccggcactttgggaggctgaggcaggtggatcatctgaggtcaggagttgaagaccagcctggccaacatggtgaaaccccgtctctactaaaaatacaaaaaaattagccgggcatggtggcacatacctgtaatgccagctgctcgggaggctgaggcaggagaattgcttgaacgaaggaagtagaggttgcagtgagcgaaatcatgccattgcactccagcctaggtgacaaactctgtctcaaaagaaaaaaaaattatgtgccccattggaagaagtgagattcggccatctcatctctctcgggagctctgagccctggagttttatgttttctgcaattatgaattgtgatccttgattaattatgctttaataataaaatgggtgactactgaaagctgctgaatctgggtaagaatttggatgaaaaaaataatatatgtgcgtaatttattctgttcaaggtactgaatattgaataagctggatttattactcaaagagaaagacagaataagagaaggttgaagggaaaaatgactgtactagaatggtagtcaaaaatgcaacaacaggcaggtgcagcggctcatgcctataatcccagcacattgggaggcccaggtgggcaggtcacctgagatcaggagtttgagaccagcgtggccaacatggccaaaccccctctctactaaaaatacaaaaattagccaggtgtggcagtgggtgcctgcaatcccagctactcaggaggctgaggcaggagaatcacttgaacctgggaggcagaggttgcagtaagctgagactgcaccactgcactccagcctgggtgacagagtgagatcctatctcaaaaacaaacaaaacaaaacaaaacaataacaaaaaagctattaatagcttcctagggagtaagagtgaagggctagtttaattccagagatgcggacacagtcctgggtctcaccaattattctgcttggtaattaccttttgaagccttttaatatgcctaacacagagctaagtgctatgaagaaatgaaagaaatagaagcaaagtactccccatgtggttaaataacaagacactacatgacaaatgtcaaagagtgactcaaacaatatgtcctttagaatttcagagaaatgacatcaatgcaggctttacaagtcagtaaaaatcttggtgatgaggcagaacttgatgtaaggcaaatcctaaaagttgagtaggaatcaactagctagaataaaatgtggggttgtggtaaatacaaaaatgtaagatgagtgaaataacttatttatttatttatttattttcagagacggagtctccctctgtctcccaggctggagtgcagtggcatgatctcggctcactgcaacctctgcctcctgggtttgagcaattctcctgcctcagcctcctgagtagctgggattgcaggcacctaccaccacacccgactaatttttgtatttttagagagatggggttttaccacgttggccagtctggtatcgaatcctcgacctcataatccacctgcctcagccttccaaagtgctaggattacaggcatgagccactgtgcccagcctaataatatattaagatggccacaggccaaatattctggggctggaatgtgagtggaaatgtcgctcaccctttatcacatagcaccccatagtccagccacaacttgcagaattcaaagtaagtgtggatgtgtgtgtgcctgcagtgccttgcacacaagtgtgcatgcctgtggacatgtgaccctagaagttattaatatatctggtttacaaactgaattgttcttttattttttttctctcttggtggtcatcaataactgaaattgggctagattcctggaaattgggtaagttctcagaaatcatttacgcactttagaatccaggccaaatttaaaatcttacaataaaacaaagaacaaagcatgctaaattatatgtataatataatatcaaccatataaaatgcataaaaaatatactagaaggaaatgtgcctaaaattcacagtctaaaatttaacagtgattgcctcttccttgtatagataagggatttttttttactgtattttccagtctgtacataataaaacaagtaatgtgcaatgcaaacaaaacaaaatgaaactttatcaaatttcagtaactccttgaagtttaattttttttttgagactgagtcttagtctgttgcccaggttggagtgcagtggtgtgatctcggctcactgcaacctctgcctctgggttcaagggattctcctgcctcagcctcccgagtacctgagattacaggcacccaccaccacacctggctaatttttgtatttttagttgagacagcgtttcactatattggccaggcaggtcttgaactcctgacctcaggtgatccacccgccttggcctcccaaagtgctgggattgcaggcgtgagccactgcacccagttgaagtttaatagtgtgaaaaaaatatttctcatctcactatatcttctatgggaggccagattgcagattgtctacagaaaaatcccttcaaaagaccttgttattacatagactggagctcatggggcaggtctggtccacacatccttaggctccgcttctcctggaaaacaaaaatagcctctgatccagtgttgcctctcccatcaccaaacctcagcttctatcgccaaactcatcaaataagagtgtccagtagaaaaactgggcagatgggggcacagaaggtgaagacatcatttcccaagctaatgttgctgctggaacaatgtaagtcttgactttgtcttggtttggtttggtttgacattggtttgtttttcatctttgtctcatgcttaaaatgtgaagggcaaatatgatccttagagttaaggttttaggttttgtagatgttttactccatttaaatgacagcagatcatttagaaatgattcctctgtaacagccttccagatcccattcgattgtacagcattgagatagatagatagatagatagatagatagatagatagatagatagacggaatttggccctgtgttcccacccatatctcatgtcaaattgtaacccccacatgtcaggagagggatccagtgggaggtgacaggatcatggggttggatttccccaatgctattctcatgatagtgagttctcacaatatctcattattattattattattattattattgcaacagagtctcactctatctcccaggctggagtgcagtggtgtcatctcggctctctgcaacctcttgcctcagtctcttgcgtagctgggattacaggcatgccccgccatgcccagctaatttttgtatttttagtagagacggagtttcaccatgttggccaggctgatctcgaactcctgacctcaggtaatctgcctacctcgctctcccaaagtgctggaattacaggcgtgagccactgtgcctggccagttctcacaagatctgatggtttaaaagtgtggcacttcccccacctcctgccgccatgtaagatgcttgcttacccttccaccatgattgaaaagtttcatgaggcctcctagccatgcttcctggtaagcctaaggatctctgagtcaattacacctcgtttctttataaattacccagtctcaggtatttctttatagcagtgtaagaatgaactaatacacacataaacagattagaggcagcactggcctgagttgtgaaactcttcccagcctggtcctgcgattagctggctatatgaccttggacaagctgctttgcttctctgggccatggtttcatacctgcaaaaaaaagagcatggacttggctgttgcctgggtctctctagccctgtggagaatcagctacatctcttactaggaacttctcattcagccagttattccactgcggagatggtccaggaccattagggccatgctagacattgggaggctgcctgtcaggtgaacatgaaattgaacttatctgttctctttcctccctgaatgttgctgaaggtagatgcccatcctcagggctgtcttacggagaggagaaagttgtgcagtgattccaccctgcagttatctaactcggcagggaactctgggcagtgagtactcacggtacagtctccacacctctaatcatgtgctcctctccttcccaaggaacctggagaccatcatctcatttcctgggggagagagcctgcatggttttatactggtgactgctttggtagagaaagaagcagtgcctgggataaaggtatcttctcacttgatagcaccttttctttttaaatgagcttgagctttacttcccactcagtgcctttcctgcagtggatttctcaacacaaatgaacatagaccttgtcctgcttagttcaagtctgagagaagagatctaagctctaggccaccatatttgctcccttttctcaattcctataaaactcggaatggaccttttgtccattcaacaaacaggcattggtttgggcaatgggaaattggatcgaacaagacagacattttcccagccctgacagaagcttatgatggatacagtggatgaagatggattaacgtggattacaggtgtgagccactgcaccgggcctcaaactggaaattcttcaggagtcagacaggtatcaggaaggctggatagaagacaaaagacagtgatgcagcttgtgatcaactacagcgttaatgccttgcctaaaaatatttcagttagatttctgccttcgctctgtcgctcaggccagagtgcaatggcgtggttttagctcactgcaatctccacctcccaggttcaagcaattctcctccctcagcctcctaagtagtgcacgccaccacgcctggctaatttttgtatttttagtagagacagggtttcaccatgttggtcaggctggcctcgaactcctgacctcgtgatctgcttgcctcagcctcccaaagtgctgggattacaggtgtgagccaccctgcccagccaacactacctcccttgataagcatatgttgagcacctactggtcctcaatagggtgacccatttctgctatattatagcgctttctttctctctcagtagttaaactccatggttactttagttctcatccatgtgtttagtccattagaagatacagagtcaaatatcggccttccaagtgtagttcagatgaagtagagactcaaggaagacaaggaagtcttcccagcagaggggattctagagctgggggctctgtagaatctgtctgtgtattagtccattttcacactgttataaacatactacctgagactgggtaatttataaaggaaagaagtttaattgactcatagttctgcatggatggggaggcctcaggaaacttacagccatggaggaaagtgaaggggaagcaagaacctcttcacgaggcagcaggagagagagcaaaggggggagctgccaaacacttttatacaatcagattttgtgaaaactctccctcgtatcatgagaacagtatgggagagcccacccccataattcaatcacctcccaccaggtccctccatcagcctgtggggattaccatccaagatgagatttgggtggggacacagatttcaacacagatttaaatctgactttatatgagagcttcggagcaaggatgccccagttggagatgcagtagaactgatcataacgtgacaaatccgagagaagaagagtaaaataatagtactcaggcccttgggaggtgcaagaagtaacagccagatgaaattccagaaacacttacctaggggtctgtctgggaggtccccagggagcttctggctgtcaggccaaccccacagtggatctagcttaggacgttcccaggaagctctgacaaactgtccggtcctcccctgggttccaacagtatgaggcttactctgcctgcatggactttaagggagtgctaataagttgtgtacatgcatctcatccctaggctcttgttcaggccacgaatgctatcttgcagcccaaccaggaggccctgctccaagccctgcagcgactgagactgtctattcaggacatcaccaaaaccttaggacagatgcatggtaagatgcttccgaagctcctgaaggatcccccaggggtcctgggctctgcttaggggaagagggcctggggaccaggcatgtcctgaagggggtgataatacattcatccaccagatgacgctggtggactatctttgttttaggttaaacacatattatcttggagagctattgtcacagctttgtattctccctctcctttatattctcccgtgattaagatggtttcccttctgcagtggccagatatttcttaggcatgttgaggtcttgcctgaagcttgagaggaggggatgggatgcacagtaatgttggtcgcgcgtgccccatcctgcagtgttaggtactgcagagcaggttgtctacactctgtaatgccccttttattctaaccccctgtgttggttcctgagatgtctgaccttggttttaagccttgtctaatggatggcctgtattccctttctgtagctagggcaggctgatttgtcaaaggtaggaaagttgtcagaatcaaaatggagtcacttgtgttgaataaaaatttttaaaccttgacaaatagagctggggaaggctacaaagagagagctcccgtgtataaatgcctgataacaaaatcttttccaaaggactgaaaaaatcaccaccttgcacaaaggccatcacaaccttacatacacaaaaaaatacttacacaacgacatctgcccagcaactgcctttccaacattggccttgtgccaccctttttattgatgctcatagccaaggttaatgatctcaaaacagttacataattgtcctcatttttcctttaaaaacctttgtcttcctttatctttctgaatacccacatggtttattatggcacatgtattcccattgcaatgccctattccagaataaatatcagtttccattagggagcctctccctgttaatctgcttaacacaggcatggtcagttacggggcccaaccttcctggaccggttacatcttattctggttactgcattcagtttccaacatctgggaggctcttcaaattcttctctcagaggaatctgaagaatgtatgtgtttaggaggatgtgagagaggggtgtggtttcttaacaagagaatatcagagtctaagtatcattttccctgaatcttgcttccctgcaggaaagaaagattctgggaaaagagagtgtttacaagaagcaggactggagggagggagaaagacgctaggtactgccaagctttattatcttgtattaaaaaagtaaatataatttgtcatcccagcctctcagcactagtagaaatctatctgaagtcacaggattaggtattatccacttcctggttttatagtttatatttgtattttctctatttccttgaattttaattttaaagccctcatgatcacatcagtaggtcttctgccaaacagctcccttaagttgtatggtggctttgccaagctgaaatgagatgagatgtgttttagctttgccaagaaagcctgagtccatcacttaggatagcaaggctattagggagatagtgcaggtgtcttcagatcacatggatgagcaaaaggaagcaattttggaagattatgagaaaccttccaacaggtcccagtgtacatagcagtaagatggtgcatgcagtgtctaactgtcacaggctttcctagggctcactttcagactcacttctttttttttttttttttttttgtgagatggagtctcactctgtcactcaggctggagtgcagtggcacgatcttggctcactgcaagctctgcctcccgggttcaagcgattctcttgcctcagtctccctagtagctgggattataggcatgcaccaccatgcccagctaatttttgtatttttagtagagatggggtttcgccatgttggccaggctggtctcgaactcctgacctcaggtgatctgcctaccttggcctcccaaagtgctgggattacagccgtgagccactgcgcccagctcagactcactttttaggcccaggccaacctgctgtgttctcctgctcagcttctgcaggaggtctcatcgtctaaggaggtcccagggctaccgcccttgttttctcaaaaggcacattttcccacacagacataatttcgtttcagtgttttactcctagtcacattcatctatatggacaaatagctgaatggattggactgtgttttctaaagatggccccatgccatcctgcgcgctcctccacagcgtggcctgggcattcctttcgaccaggggtggactctgtgcccctacttgtgatgtacgtgttgccaatagaatgtagtataggggatagcacagacttctgaggcaagactagaagaggtgatgcaggtttaaccttgtgtcctgagccactaggtaaaaagtccacctaccctgagatcactctgctgtgcaaacgacacaggcaaaccacatcaaagagccatgtgggttctccagttggcttctgcccaggagtgaaggtgccctcagatggttctaggctcccatcccaacttatgtcctgtcttgaagtcttcccaactgaggcaccagccactgtggagcagagtcaagccattgccatcttgttctgcccagattccccatcaacagaatatagtggttttttcacacctctatgtttggagtggtttctatgcagcaatagtaaccacaagaaataaagttataaaaatagtaacaaacactagaaactgcaagatttaaggaatcacctgaatgctccagtcattgtctctggtttgtaatgataaactttcttctgcgtgataaatagagcttggctggactttttccctctgcttccattccccaaaatggagcgtaccaaacaattgcttctttcagagcccagctttagcaagagtcatgagctctaatcccttcatccataaatacttcctttccaggcacgtagagcccttcacactcagggctgagtagaaaatgcctgttgcagactgcagtggcgttttggagagccaccctcctggtacctgagtcactagtcctttgcccagcttttctcagttttgtaaagcgctcacttctgagtggaagacagaaccagcccggtctattttcataatctgcccccataaggcagaagtccacacggtactggaaacataaccatatctatgtcagtctgccactgcctgccgcctgtcaaccgtgcgtgtctgcatccagtccttctttggagctgctttccagcgactcagccagatctgagctttctgactcattggaaggttaaactattttcagactttcaagtgttgagattattaagacatgtgtttttttttttcttctttttttggcatgcttactgatcgcccctgttctctaggaagtaatgttcttcttggaaaaggtgaaggatatttttctccccaaaaagccatggaaatgtttgcctttatttacttccaaattaacagaatttccagcttttgcttgacccatcggcgcattggcagcgttaagaattttttcttttagcagtaatgaggagtcgaagggtttcttcctaaccatttagtgtatgcatttaaatcaggtttctttttgagtaaattggggctaagctgtagtgtgaacttctgctactgttcctttctcattagttcacttgatttcatggaaggaattttccatctcagcctgtgaacttattttgtctaaactcaattggaaagtaattaacactgagattcttctttaataaattctatatgataataaaatcatacaaacatctctttatttttctttttgacctagaaaagatgttcacaggccagacctggtggctgaagcctgtaatcccagcacactgagaggaagaggcgggtggatcacatgaggtcagtccgagggcagggtagccaacgtggtgaaaccccatctctagtaaaaacacaaaaattagctgggcatggtggcacatgcctgcaatcccagctactcaggaggctgaggcaggagaatcacttgaacctgggagatggaagttgcagtgagcctagatcctgccactgcactccagcctgggcgacagagtgagactctgtccccaaaaaaataaataaataaaagatgttcacaatatattgttaagtgaaaaaagcaggctacataacttgcataatatgagtgcattttaataaaaatatacatatttagcaaaataaaaggacaaatggtatttattaaaatatttacgttgattatttcaacacagaggatgatagttgattttgcttaatttcttccctccttcttttcaatttgaattttctataatgaagattgtttctttttctttctttcttttttttttttgagatggagtttcgctcttgttgcccaggctggggtgcgatggcgccatctcggttcaccacaacctctgcgtcccaggtttaagtgattcttctgcctcagcctccctagtagctgggattataggcgtgtgccaccacacccggctgattttgtatttttagtagagacgggctttctccatgttggtcaggctggtctcgaactcctgaccttaggtgatctgcccacctcagcctcccaaagtgctgggattacaggcatgagccaccgcgcctggactgattattttttaaatagggttagaaagtgaggaagttactaaactccgattagcccaaatatgccccagtgggtccttctggcagagtaaatgtctcggttcagcctgaatctgggaaattgttcctacggttcagcctgaatctgggaaattgttcctaccttataaactggagtatccttcagaaatgacatttactcaaacttccttttaggcagactgcataatagcaatttttaatattaaccatttaaaaaaaaccttcagattaattaacaccaaaagaataattgggaaaatacaactcctcactttaaaaaagaaacaaccaaagtaaatactaaaaaactatatgatgttatattatctaagcttagtttactgcaaagatcaagagcacactactagttgacggcctctattcacactgttcatagccctcgctcgcttctccagccattcactcactcatgcaaaaggtctgtacacacaatgatgcctgatggtataataggaaccttaacatttcaattaaaaggcaaaatgaggacacttaccatcagcctataaaattattcttattattcttcttcttcttctcctcctcctcctcctcttcttccttcttcttcttcttcttcttctccttctccttctccttctccttctccttctccttctccttctccttctccttcttcttcttcctcttcttcttcttcttcttcttcttttttttttgagatggagtcttgctctgttgcccaggctggagtgcagtggtgtgatctcggctcactgcaacctctgcctcccaggttcaagctattctcctgcctcagcctcccaagtaactgggattacaggtgcatgccaccacgcccggataattttttgtatttttactagggatggggtttcaccatgttggccaggctagtctctaactcctgacctcaagtgatccacctgcctcggcctcccaaagtgctgggtgtgggcggcaagccacccaggtgccaaggcaagagacagagggcacgagctgttccagtataatgaggaaaatatatagaataagaatagttatactagaaatagattatagatatgattacatatgaatatcattcttcattagtttgtagcactactctttattccagtattataataatctttgttctacaattataacctaggaaaaaccaggccatacagagataggagctaaagggacagggtgagaagtgaccagaagagtgtgagccttctgttatgcccggacagggccactagagggctccttggtctagcggtaacgcccgcgtctgggaagatgcctgtcacctaacggaccgtggtctagcggtagcgtcagtgcctagaaaaggcactctttttaaatatactttttatttttgtttaatcttccctgatttcctatagatctgagatatgtcatgcttattttcattgctatctaaaaatctcaataaactttatacctaagagtaaaaaaaaaaaaaaagaaaagaaaaggcgctcgttacttagccgaccgggaaagggagtctccctttccccgggggagttagagaagactctgctccaccacctcttgtggagggcctgacatgagtcaggcctgcctgcagtcatctggaggcctaaccgtctccctgtgatgctgtgcttcagcggtcacgctcctagtcctgaacacctggctccgccttttagatagcagtagcagaattagtgaaagtactaaaagtctttgaaatgcagaagtaatggcgtaagctgtcacgtctctctctccgcctcagctgccaaacagagaagggtcccctgtccagtggacacgtgacttgggtgaccttacctgtcattggagacgactcatactccttaccctgccccttgccttgtatctaataaataacagctcaatctggcatttggggccactactggtctccgcatcttggtggtagtggtcccccgggcccagccgtcttttattctatctctttgtcttgtgtctttatttctaccatctcttgtctccgcacacgaggagaaaaacccacagaccctgtagggctggcccctacagctgggaattacaggcatgagccaccgcatccagccagcctaaaattcttctgaaggataataatatagtacttgaagacacggtttgaaaaaaatcatactaaatgaaagggcaccattttacaagcactagaactacattaaacttaaatgaattccaacactcttaataatgtaactcaaaaacaagtctagtgttaacaaaagctccaataactaaaactacattaacaggcacaatgaacattgtaaacgccgctaattggcaccaagtttaatagggcagacaatattttcttctgcattcacacttactcagttacactgttgaaaaatgctgctgctcaagctatgaatgctttacaaaagaaatcattttaataaatacagtaaatgctaaaactctagctaaactattatgcaagatatacaaccaagacaaatacaaattcataatacaagcaacttgcattcaaaatgaactctaccactatattttattaaaagggcagactttatgaattaacccagctgcttcctgaattacaaaagtggcatgactcaatatgaaaataagaaactgtctacaaatttctgacagtaataaattgtaatatacaatacatgcaggagtcttacggaagaataaactctcctaggaaacaaaaatattttatacttttaaaatccaaagtaaaaaaaaaagaaatcattgccagatgcggtggctcatgcctgtaatccaagcactttgggaggccaaggcaggatcgcttgagcccaggagtttgagaccagcctgggcaacatagcaaaaccccatctctacaaaaaaatacaaaaattagatggtaatggtggtgagcgcctgtggttccagctacccaggaggctgaggtgggaggatgcacctcaaggctgcaatgagccaaggtcacaccattgtactgaagcctggggacagagtgagaccctgtctcaataagtaaataaataaatatcttttatgaaaaagattctctagtcagaattaacacctcaactagccaaacatcaggaagttacattacagctacttaatacacaaagggacacattttcaccagtcgttgtcttctgatatttctattccagaaacacacactctcacttccctacactccccatcccatcatttcttcagagcatggaaacagaatttgttgaacaccagaaatctcttgctatggtggtacataagtcataacatttgttgctgcccagcagcaggtatgaagccggctggtgactggctagcaaatgcctattctgtaagctcctcacttagcccatctgtagctctgacttctccaccaattcccttctctcctttcacagcctttctgagtttctgagggataatttcagaggttccatataactgtcaaagcctatggtagacatggcaaagtgaaaatcctctccactggccatttctgtttctcttgggggcatctttcacttgcctcaggtgttataaagctgatgaacacacgtacacgttgtttaacactttcttgggcatttcccatttgagatatggcatgtttcattatcctagtgacatgtgcaatcagaaaatgtatattttgttctctgcaactttcttttgaaaaatgtatatttgaacaaaatatacattttttgtatcttcatgaccattcatgctgtcctcactgtcatcatgaggctctatataacataatgactcctccagggcagtcttcggaaattcccagtgcagaagcacgtgtcatacagcagtccccattcatctccagtgcagctctggctggctcccatgtctgatcagctgtttggttggacagaaaatgactgcaagggaatcagttccagtgtgagctctgtttgcagaactcagactcccctccctcccatgttaatgctttttttcttcttcttttttttttttttttttttttgacagagtctcattctgtcacccaggctggagtgcaatgctatgatctcggctcactacaacctgtgcctccccggttcaagcaattctcgtgcctcaacttcccgagtagctgagattacaggtgcacaccaccacaccccactaatttttttgtatttttagtagagacggggttttgccatgttgccaaggctggtgtcaaactcctgagctcaggaaatccaccttcctcagcctcccaaagtgctaggattacaggcgtgagccaccatgcccagccccatgttaatgcttctaaagtttgccctcacttctttagaaattccttcagtacatcctttaagacttcctctagtgagtgtctgctggtggtaaactctcccctctaaaagttgctttatttctcctcaattcctgaaggatatttttgctagcagctatattcttttagatgttgaacatatcggtacaaaagcttctggtttccatggttgctattgagatgttagctgtcggtttatctttctcccctgactatgtgtacctgtttctctgtatctgtctactttttgggttgctcaatttattggccttgggcttcattctgctgctgctttttttttttttttttttttgagatggagtcttcctctgttgcccaggctggagtgcagtggtgcaatcttggctcaatgcaacctctgcctctcggttcaagcgattctcctgcctcagcctaccgagtagctgggattacaggcacctgccaacacgccaggctaatttttgtatttttagtagaaataggatttcactatgttggccaggctggtctcaaactcttgacttcaggtgatccacccacctcagtctcccaaagtgctaagattacaggcctgagccaccacgcctggccacaatttttaaactttttatttttacaggcacctgccaacatgcacaactaatttttgtatttttagtagaaacaggatttcactgtgttggccaggctggtctcaaactcttgacctcaggtcatccaccaccttggtctcccaaagtgctaggattacaggcgggagccaccatgcctggccaaaattgttaaactttttattttcttcctcaagaggatgagaagaaaggtcaattgtaagctttagaagtcttgcccaatagccaatctgagaatattctccgtaaacattcaccagaggcagccagtgaccatgggatacttttggtgagaggaattgattgctggggtcaggaatgggaggaaagcatacttctcattagataccttgttgaacttcgtaaattgtgtgccaaatgcatgtcttacctagacttcataaattaatttctttaaaaataatcaaagacaattttttaaagacttatttaatttaaggtgattataaaacatccagtatactttcactattaaaaaagtaagtattcctgtctgggcttggtggatcacacctgtaatcccagcactctgggaggctgaggtggtcggatcatgaggtcaagagattgagaccatcctggccaatatgatgaaaccatgtctctcctaaaaatacaaaaactagctgggcgtagtggcgtgcctgtagtcctagttactcaggaagctgaggcaggagaatcgcttgaacccaggaggcggaggttgcagtgaactgagatcgtgccactgcactccagtgtggcaacagagtgagactccatcttcaaaaaaaaaaaaaagtattcctaaacagcatattatcatgatatattattttgttttgtagggttttgaaccttgtctaaaaagaattaaaatgtataaatttcttcctgcaatttccctatttcactaagggtcattcacattggtcatatagacatagcacattttcaccactatatagcagcattttgtacaaatagactacaatttacttattctgcacttatttctgtttgtttgttttgctatgaaaagcaatgtcattacatatattcatgcccatagctacaagtttacatatttcaggttttctgtagggtggacaccagggagttgaattgttcaacaggactttacattcatctttagttttattggccaacaccaaattgttcttcacaatgtttgaactaagttgaaattccacctccccatcacatttagttttgtcaacttcatttcttcattcattaattcattcattcagtcttttgtttatttgtttattgccagtctgataggcgtatagtggtgcttcatcatggttttactttgcatttctctgattttctttttaaatttttaaaaaattatttttatgtagaaacaaggtctcgctacatggcccaggctggtcttgaactcctggcttcaaatgatcctcccacattggcctttcaaagtaccgagattgattataggcgtgtgccactgtggccagctgatttccctgatttctgatgagttaacaatctcttctttctctctctctctctctgtgtatacaggtactcaccattcgtgcctattttctgtaaaatatgtggctttcctcattttttttttttttttttttttttgaggcagagtctcgctctgttgcaggctagagtgcagtggtgcgatcttggctcaccacaacctccacttcctgggttcgagcaattctcctgcctcagccttcagagtagctgggactacaggcgtgcaccaccatgcccagctaatttttgtatttttagtagagattgggtttcactatgttggccagactggtctcaaactcctgactttgtgatctgcccacctcagcctcccaaagtgctgggattacaggagtgagccactgcgcccagccatctttcctcatttttatactaattaggcttttatcttacttgttttttttaatgttttttgtacactctgaaggctgatttttgttaattgtatgtgttgcattttttatggtttgtcttatgccttttgaaagtaaaagttcttaatttaaatatagccaacctgtaaatcatttgtgaaagtctgtggtttaagaggtcttgaataagaaattatcccatcatcataagtcataaatacttttttgttgttgttgagacagaatctcattttgttgtccaggctggagtgcagtgggttgatctcagctcactgcaacctctgcctcctgggttcagcaattctcctgcctcagcctcccaagtagctgtgataataagcatgtgccaccacaccagtctaatttttgtatttttagtggagacaggatttcatcatgttggccaggctggtctcaaactcctgacttcaagtgatccacctgtctcagcctcccaaagtgctgggattataggtgtgaaccaccatgcctggcccataaatacatttttatgtattttcttctaaagttgttttgtctttcactttttagtttttaattcacatataattactacttgctacttataattatctgtaagtagtatgagatgagaaataaattctatttccctcctatggataagcacaaacctgcagtattagcacagtcttatgtcagattttctaaaatgaatgggtgtgttttctaggctctctgttctgtttcattatctgtcttttcctgcaacgatatcatctgccttaaaaactctagccttgtggtattcctcattttcaagcagagcaaaccccgtcaccttgcttttctcctccagcatcgcttgtgctatcctggactaagaccttcatatagactgttagaatcatctagccaagttccattttaaaaatctatgttggagctgggcgcggtggctcacgcctgtaatcccagcactttgggaggctgaggagggcagatcacttgaggtcaggagttggagaccagcctgatgaaaccccgtctctactaaaaatacaaaaattagctggacgttgggcacttgaattctagctactcaggaggctgaggcaggagaatcgcttgaacctggcaggcggggggtgcagtgagccgcgatcatgccactgtactccagcctgggtgacagagtgaggctccatctccaaaaataaataaataaataaaataaaatatctatgttggaaattttgtacaaattttattaaatgtctacattaatttggagaaaaatgacttgattttgattatctattcaatatttctgtattatgaataaggcaaaaagagaggcagagaatagcataaaataataactaaaattcctgggtaaaccacctcaaatcatttcttcatatggctcaatattcttttgtgacatggcctgaaatatatccagacagagaactcttctcttcaatacatttcttcttttaggtattcatattgagttttcctgtccatgaacatggtataagagagtatatcccttcgggaggccaaggtgggtggatcacctgaggtcaggagtttgagacgagcctggccaacatggtaaagtcccatctctactaaaaacccaagaattatccaggtgtggtgacacatgcctgtagtcccagctactcaggatgctgaagcaggagaattgcttgaaccaaggaggcggaggttgcaatgagccaaggtcatgccattgcactccagcctgggtgaagagcgagactccatctcaaaaaaaaaaaaaaaaaaaaaaagaaaacgagaatatatcctttcatttactagtttttcttcaatttctttcaagataaagggcttacctatcttctgctttattcatagttacttgatatttttgtttctaataaatatggtgtctgtctatttacctgcctcttacctgttcatttccagtttcaaaaatgatgttgatatttgaatattaaccttaaatctagcaccttggtaaacactattattcattctaataattatcagtagattatatgtgttttttatttataaatcatattgtttgagtagcatgctttgcttcttcatttataaaatttacaacttttatttctttttaataatttttttcttattctcctggctaggacttctaacacagtattgagtggaagtgctgatccttgtttagtttcacatttgaaaaaagattgcttttactatttcactgttaagtataatatgcaccataggctttctgtggattccttttatccatttaagaacatctcttattcctaattagctgaagttttctgcatgtttgttttcatcatgagtggatttttttacatctattgaaatcattttacatagaagatatttcacacctattgaaatggtcatttcacttttccttctttaatatgttaagttgggcaaaatattaaagtatcacctgtcattctgcttcagcaaaaagtagtagtgtcttagcagtattggtgaaaagacagcatcaaataaaaaagatgtagaagtaggacccagtaaaaatctagcgcatggggcattgtcacatgtaagcagacagaatgtgacaccaccaaggagcatctgaagggctggaggctgaaggaagacatgagtcacccaggctcatggacacttcagagaaattagggagcaggaagaagaaataggatcaaagactacgtatgttggttggaaaaggaagctgatggtatggagatgttattatttaggtctcacataaaagatgtagataaataggtagataggtagatagatgatagatagagagatagatagataaatacatagatagatagatgataaatagatgttgttatttaggcctcacataaagatgtagacagattagacagacagatgatagatagatagatagatagatagatagatagatagatagacgatagatagatagatagataatctcagaaacagagacacagtgatctcagtaagataggcatatgccaggtgacagaattcagaggggtcccactacgtgaaaacaatagaacaaccttcgaaaagaaatttagtacaaataagagggcaggcttccttacatacaagttagtaaactggaagaatcagttatcctcaaacattggaatagatcaaaaatagttgtttatattaatgaaggtagctaaacatgaagctaagtgaacctgtctctgacctagtgtggcaatccctgggcaagggacacttgctccgctcttgtatccttcactgaatattcagactttcagttaagcatcggtgaatttagttttcatctcttgtgaaaaccttgagagaggtaattctctctgcttttcttcttttcccttccttcattttctcaaacattgcctgtttaaaatacgaaattttaaaagatggccttgttctcttttttgttgttattattaagtacagagaaaggaagaaccacaaatagcaaagggcaacatatggaatagtttagaagttccgggagcacccatgagggcaactgcagaagagaacattctatcccccgttgctgcagctttcattccaggtctccatgcatatcagatagggaaggaactccgggacagcagcagggcccatgcacatgtaaccaattgctttctttgcctgtagtaaagttcacattttgattgcttctccagattatgtagatccagacatattttatgcaggcatccggatctttctctctgggtaagtatagttcagttgttttcctgtgtgaagtctctgtagcattgactgaatgtataaggggacgaagagacagaagcttcctagcgtaagaaacataccaagtgactcttgctagggatccactctcaggtaaaagaagtgggataccatctgcacaacaaataacactgagggctaagtatttcagttaagagtgtttgttcctaggcagttcagatccatttatattcacttttcttagaatcctagctcaatgacagaagaagaaaaacacagtatgtcactcacacagttctatcacttacatctactttttcttcttgttattaaggcatgtagaaggctggggagtgtagtatagagttggatagcatcgaagctttcttctaaagttcctggaagagctacactgtggtttgaacgaatgtgtccctccaaaattcatatgttaaaacctaattgtgatggtgaggtattcgaaggtgggttctctgggaggtgattatgtctcctctgagaggagaagacaatcccctgagtggaactaatgcccttataaaggggctggagggagttcacttggccctttttgctccttttttcttccattccttgtcccttccactatgtgaggacacgggagttgaggcattaccttggacgtggagaccaggccctcaccagacactgaacttgccagcaccttgatcttggacttcccagcctccagaactgtgagaaatacatttctgttatgggtagtccccaactcaatacagtttgacttaccattttttgactttatgttggtgcaaaagcatacatattcagtagaaactactttgagtacccatacaaccattctgtttttcacatttggtacagtatttaataaattccataacatattcaacatgttgacataaataagctttgcgttaggtgattttgcccaactgtgggctaatgtaagtgttctgagcacatttaagggcagctaggctaagctaaggtgtttggtaggtcagatgtattaaatgcattttttgtcttatgatattttcaacttacattgggtttatcaggatgtaacccactgtgaattaaggaatatctgtatttacaaattacccagtctaatgtattttgttgtagcaacaggaacaaactaaaacactaccccaaaccatttttttcatattttctgagtactcttcttttgtcacatggcttgaaatttcttcacatagagaactctactattattttattttattttattttattttattttttgagacagagtcttgctcttttcacccaggctggagtgcagtggtgccatcttggctcacggcaacctctgcctcctggattcaaactactctcctgcctcagcctcccgagtagctgcgattacaggtggctgccaccaagcctggctaattcttgtatttttagtagagacggagtattgccatgttagtcaggctggtctcgaactcctgacctcaggtgatctgcccaccttggcctcccaaaatgttgggattacaggcttgagccaccacgtctggctgataactctacttttagatactctctttgcttaaacaaattagccattccttccttgacatgttttaatcagattgccttcctattaacttcggaaattaagagtttctgctatgtttttgtttaatttttaaacagtgaaaaatgaaaggtggaggcaatggctggatgaggtgaatatgtcaaatagatatcatctagtgggccatcttattaactaggagacacctgaagtgctatcaatagaaataatctgaagctgtgtctggatacagcaagagacatgcaaatgctaaaaatctactatattacattggtgcaaggacagagtagcaacacatactaagtattttctccagattgggtgtgtttgacgtgtgaagcacttcaaacagagtccagcctgggagggagtggggatggaatcctccttgtagagggtacagagtggaagcaagaaggtttccaagattgagagtaatgggtgtatggtttatgggggaaagggaaaacaaaaagaagggtgcagaaatggagctgggagtgtgttttagtatttaggctttctctgtatacctttgataggattagaaaaagaaaaatggaccatttttaaaaatttcatgctaccacatagcaggcttatactatagatgcagaaacagactgggatttaggaagcaccccaattctggaaaatccctttttgcttcacattgctctctaaatctgtatgttttcccttgttacgtaacaatttaccacaaatttagcagcttaaaacaatatgcattcattgtttcacaattctgtaagtcggaatcataggcaagctcaactgacttttccatttagggtcgcaaaaggccgaagtcaatttatctattgggctgggctcttaactgaagatctggggaagaattcccttcaaaactcatttaggttgttgtcagaattcagtgttttgtggttctagaactgagatctgtttccttgttggctgtcagacaggagctgctctcagcttcttgaggcatccagtattccttatcatgtgtttttttttttccatctcagcactggcactttttttttttttttttttgagacagagtcttgctttgtcacccaggctggggtgcagtggcacgatctcggctcaccacaacctccatctcccgggttcaagtgattctcctgcctcagcctcccgagtagctgggattacaggcacccgccaccagcccggctgatttttgtatttttcatagagatgtggtctcaccatgttggccaggctggtcttgaactcctgacctcaagtgatcctcccacttcagcctcccaaagtgctgagattacaggcatgagccatcatgcccagccagcactgacaattctaatccttctagcactttgattccttctcacctctccttctgcctctagccagagaaaactctctgattttaaaggtcttatgtgattagattcagcttacctaggtaattcaggataactccctatgtcaaggtcaactgattaataaccttaattacacctgcaaagtcccctttgccataatatatcatactgacagacatgctatagcataatactaatagttctaagaattatgataagaatcttggaaaaccatttttagaattatacctaccacagtatccttcaagggataaattgattctacttcttctctatgtcagaagcatctgatgaggatgaactatatattctgaaatccccatgattagatgtgtactagaaggtgattttactttcattaaaataaattcggagtcattgacacattttatctttgatttacataaatgcctccgctctgtttcttaccctcaaaatatttcccatgtagctaagtggccagtaccgaatcctacatgcattaataagtgtagatggacaaaaatatctggattactgagaattcccattagcattgtctagaaaaatgtaaatttgcttttttgttcttgatttatcctatttttgatttattatttatatttatttatttatttatttatttttagatggaggtctcgctttgtcgcccaggctggagcgcaatggcgcaatcttggctcactgcaacctctgcctcccaggttcaagctttcctcctgcctcagcctcccaagtagctgggactacaggcacctgccacagtgcccggctaatttttgtattttcagtagagacagggttttgccctgtgggccaggctgttcttgaactcctgacctcaggtgatctgcccaccttggtcccccaaagtgctgggattacaggcatgagccaccacacctggcctttttgcttactttttaaaaacatttttatttaggagaatggagatatttcatatgtagatgacacatattcattccctttagttcccacacacattcaatttcttgaggaagttagcctttgcaaaaaaaaaaaaatgatctcatttttttttccccactaaaacttctcattttcttggggttgctagaaagttgctacaagaaaggctaaaaataattgtgcctacagatatttgaaaggaaaatagttcctcttttttcacagtagcagcttggacctgagaatgtatgggagcaataattgggctgctcaaagaaacacaatttcccttcctcagactagaattaccaacctagagaacatgagtttttaaagtagatgtgcttcttttatctttttggacttgtatgctggtgttttctctgtcaccttcactgtggaaatcctcttgagggtgaggcactgaaagcagattgattaatgtctcttggccatttgagacattggatggctcttttaagttggccacgttctttcaagaactatgcttgggctacatattctggatatataatacatacttgtaggatgttatttttaaatcattcatttatcacatatttagtgagtgcctaccccatgtcagttctaggtgctggaaatagagcagtaaaaccaaccctcaacttggcccctctggagcttacatttcaatgcggtgggtggggggatggacaattaatacacaagtaaatctaataaaagcgtcatacaatatacattacaatggtaagcacaatgaagaaatggaaagctggatagagagtattagagactgtcgaatgtagtggccaaattttcctgtttattgtggtccaagagtgccacagccctctatgacatttgagcagacacctggaggaagtgagggagtgagccgccaagaaggaatggcaagtgcaataaccctgaggtgggagcgtgttggtcgtggtggaagagctgcaggaagccagcagggccgcaacactcaggggagaataagaaagagtgaggtgacagtagagaccggatcatgtagagctttgtcagcttcttttctgagtgagatggacaacacggacacgttttgaaaagaataacaatgtgatctgccttcagttgcaaatcatctctgtattgactgagtaggaaataaactccaagaataaagacggaaacagggaaaatatttaagaagcgatcattacaatccagggtggtggcttgtactagggtacaagggctaaaggtgttgagaaatggtcagattctggatatatactgaaatcaaagttgatggaaagatatgagtcaaagataatttgcaggttttggggtcctggttcactgaaagaacagagacatcatttactccaatgaggaagactataggaggaacaggtttagcaaagaagaaaggaaatcaggagatcagtttggggacacggtcatatcaagtaggcagttggatgtatgggtctggaatataggggagtggtctagctatcagtgtaaatagctttttacatttgtaaatagtcaggatataggcttttctttttctttttgtgagacacagtcttgctctttcgcccaaactggagtgcaatggcacaatctcagctcactgcaacctctgcctctgggttcaagcgatactcctgcctcagcctcccgagtagctgggactacaggtgtgcaccaccataccaggctaatttttgtgtttttaatggaaatggggtttcaccatgttgaccaggctggtgtcaaactcctgacctcaatcgatctccccgcctgggcctcccaaagtgctgggattacaggcatgagccaccccatccagctgggatatagttgttttctaaatctatgaggctagagaagaccatttgggaattgagagcagtgagcagtccacggactgaccctttgagagtgcaacatttagcagtactcaagatgggaaggagctagtgaaaaagccccaaatgactacataagaccatcaaccagattacaaagaaagacccgaacatgtgtgcccatatatcacccaacagcagttatgtctttcatgtttcttccccataaaatgttgttcatcaactttattagactagggtcttaacattggacaaatcacaaaacctctctggagcctattttatttttcaacagctgtaggaagcaaatacaaattggaaatctaaggctcagaaagatttgtacaaagttacacagtaatgaaaggggagccgggattcccactcactctaaagaatatgataaaatggctagtattcactgaatgcttaacatgttccaggccctgggcaggtattattttaattagttctcacaataatccaataagggagatactaatttactcagatgagaaagctgaggctcagagaggttaatgaactaagccaaggctcactgttaataaatagcaaaggtaaaattaaattccatatctgcttgagatagaggccttgctcctaatagctgcagcctgtcagggcctggcagcagtaacctctcctttcctcttcccaccattcccctgcactgctttctgtaccgcatctcttttcagagtgatgttgccccaattgcggaggccactgtgctgtttatccagtgaaagctgtagcacagccaacccaaagcgtccccagtgaaaacaacctggctccttacagcacttccagcctcagagcagtatttgaaaaatatcatgaacagcaaacacagcagtctgtctgtggcttttatatgtgtatatggtgtgtgtgtatgtcccttctcttgagcaaaataacttttagaattatagaaaaaaaatgtgcaacatcaatgtggatctgctgtttaaactcataacagagaaagtagcttgtttctggctataggaggaaaagacgatattccttagtaaaaatggaaatccacatatggggttcttgtaaaaatgaagatagaaaattgcaagtttggggatcaagttctggttctatcatcctttaacagtatgaccctggaaccttaattgctttgagtctttgttactttatctatgaaatgaagtatttaaaaaaactccaaaaatctgtcctgatgtacacacaagaggtcaaatgagaaaatgaatgtgaagatgctttataaactatacagcatggtaggtgcaaatgtgacatgaacttgttttggacacattataaagtcacccccacaaactgtgattgttcaagactatgcaaagtcagacacaggaaaataagtaaaacagatggaggcataaagagggggaactcagagaaaacagtgaagaacaggaatcaggaagacaaaggagaggaaaggtggggaggagaggagaaggaaagggggaagggaatggaggagaggagaacagctgcttcacagagcatggccggcagcccagtcccagcctttctgcatgtccctgacttcagcctctggcgaggcacaggcttactctgtgcttcctgctgttactcttcttatccatccttattatcaatacctgtggtcaacaaagtatttgataaaggcatcctcaaagtcaggtaacatctgtacgttatagattacaaagttgagtaatatccagaattggtagtttaacgtgatgacttcttaacaattatcactgtttcagggaagggcaaaggtgtgtgtgtgtgtgtgttcatctgtgtgtatctgtgtatgtaattgtgggtgtttgtgtatatttgtgaggctctttacttggcggagttaaaaagtatctgctcatcaaggttgagattagcaaaggaagtgaagatttttccagagcccctaaaatgtgccttttgaccaacactgaggacatctttataactgagtatgtgcaataaatatgtcttgggacctgtgccacaaattcctctctaaatagcctttacctctctggaataaccctttagatgaggaagaaaagggctgtgattttatagcttgttatgaagctggagtgaagatgatgcttcagtacttaccctacaaagatacccccaatccctcaccctaaaattaccattgaaatcatgttccctttctcattcactctcagtttccatgtcagaaaatataccattacctccctgcacccctttcatctctctcacttttctcttgcttagatggaaagacaacccagcaatgcctgcagggctgatgtatgaaggagtttcccaagagcccctgaaatactccggcgggagtgcagctcagagcacagtgcttcatgcctttgatgagttcttaggcattcgtcatagcaaggaaagtggtaagtcagacattttgttttcccttgagagtagagggaggaagaggagaggtgttttttttttttccaattgataaaaccaaatataaattaaaatgtcatgaagtttatacttctctaagtcagccaagaaactgcatgactgccaatgtttttgtgtcaagccaattaatattggaatatcagatgtcagcttgatcttgggttttacttccaaatcttaaaatgttgctctgtttccaactgttcactatcactttggtttggatctttagacactagcttcctttttctgaaatgggggagagatgtggagtttgaaggctatgagtctgggccagctggaaacaggtctgggatcttccaagaaagtccttccccacaaaatggtgcaacttctagccaaatctatttataccagcagagggatctatcaccctggaagcttgaaattgttcattttcttacctgccaggatcaagttaagtttttaacagttgcaaaaagacacttcatactatggagttttcaagttggattagaagaaaaagaatcaccagaacttagtgtcgtagattcaagtcacttctctaaaactgtcataatttttcacggattttggcatttggtgacattaatggttgatttacttaccatgcataatattaaacccataacgaatttcctataaatatctattgatttgatttttaaatcacttggcttcaagaggctattactaaaacagtgactcattctttatcttttttgccttcacgggctttatataactttctccttttcttgtgctccctccaaaacaaagcactgagaaaacaaaattcaccagagtattcagctagtcagttcaagggtttgtgttctacatttgaagatattccttatagcagctaccaacgggatactttgtttacatttgttgtgtagtaaatatttatatattggcaaacaaatctagttccaactctgtcatctgagatgttcttactttgtttcctcttctccatctcctgtcaactgttagaaatatacatttgagtacgtgaagtctgcaaacaaaagggccaaggtagatttgagttagaacaccagcaacagtttctggtgcattcttgtctgaaaaagcagagaagattgggcaccgtgggtcatgcctgtaatcccaaaattttggaagtctgaggcaggtggatcgcttgagcccaggagttcaagacaagcctgggcaacatagcaagaacccgtctctactaaaaggaatacaaaaaaatattagctgggtgtggtggcgcacacctgtagtttcagctactcagaagactgaggtgggaggatcacttgaactcaggggcagaggtggcagtaagctgagatcacaccactgcactccagcctgggcaacagagcaagatctcatctagagaaaaaaaaaataaaaaagaagaagaagcagacgagctctgctatatttccatgtggagctatgacgttatgctgtattgtttctttcaggtgactttctgtacagaatgagggattacatgcctccttcccataaggccttcatagaagacatccactcagcaccttccctgagggactacatcctgtcatctggacaggaccacttgctgacagcttataaccagtgtgtgcaggccctggcagagctgcggagctatcacatcaccatggtcaccaaatacctcatcacagctgcagccaaggcaaagcatgggaagccaaaccatctcccagggcctcctcaggctttaaaagacaggggcacaggtggaaccgcagttatgagctttcttaagagtgtcagggataagaccttggagtcaatccttcacccacgtggttag

SEQ ID NO: 16 - DNA, 27 bp, synthetic construct
  misc_feature 1..27: note = Primer
  source 1..27: mol_type = other DNA
tccggtaccatggagcctcaaagtcag

SEQ ID NO: 17 - DNA, 28 bp, synthetic construct
  misc_feature 1..28: note = Primer
  source 1..28: mol_type = other DNA
atccggtaccatggcactcagtaaaata

SEQ ID NO: 18 - DNA, 30 bp, synthetic construct
  misc_feature 1..30: note = Primer
  source 1..30: mol_type = other DNA
tgtcctggtgctaagggtcaagacaattct

SEQ ID NO: 19 - DNA, 29 bp, synthetic construct
  misc_feature 1..29: note = Primer
  source 1..29: mol_type = other DNA
tgagttggcctaagggtcaagacaattct

SEQ ID NO: 20 - DNA, 1266 bp, Homo sapiens
  source 1..1266: mol_type = genomic DNA
atgttgcattttcattattatgatacttcaaacaaaataatggagccccacagaccgaatgtgaagacagcagtgccattgtctttggaaagctatcacatatctgaagagtatggctttcttcttccagattctctgaaagaacttccagatcattataggccttggatggaaattgccaacaaacttcctcaattgattgatgctcaccagcttcaagctcatgtggacaagatgcccctgctgagctgccagttcctgaagggtcaccgggagcagcgcctggcccacctggtcctgagcttcctcaccatgggttatgtctggcaggaaggagaggcgcagcctgcagaggtcctgccaaggaatcttgcccttccatttgtcgaagtctccaggaacttggggctccctcctatcctggtccactcagacttggtgctgacgaactggaccaaaaaagatccagacgggttcctggaaattgggaacctggagaccatcatctcatttcctgggggagagagcctgcatggttttatactggtgactgctttggtagagaaagaagcagtgcctgggataaaggctcttgttcaggccacgaatgctatcttgcagcccaaccaggaggccctgctccaagccctgcagcgactgagactgtctattcaggacatcaccaaaaccttaggacagatgcatgattatgtagatccagacatattttatgcaggcatccggatctttctctctgggtggaaagacaacccagcaatgcctgcagggctgatgtatgaaggagtttcccaagagcccctgaaatactccggcgggagtgcagctcagagcacagtgcttcatgcctttgatgagttcttaggcattcgtcatagcaaggaaagtggtgactttctgtacagaatgagggattacatgcctccttcccataaggccttcatagaagacatccactcagcaccttccctgagggactacatcctgtcatctggacaggaccacttgctgacagcttataaccagtgtgtgcaggccctggcagagctgcggagctatcacatcaccatggtcaccaaatacctcatcacagctgcagccaaggcaaagcatgggaagccaaaccatctcccagggcctcctcaggctttaaaagacaggggcacaggtggaaccgcagttatgagctttcttaagagtgtcagggataagaccttggagtcaatccttcacccacgtggttaggat

SEQ ID NO: 21 - DNA, 73660 bp, Homo sapiens
  source 1..73660: mol_type = genomic DNA
ccctggccttggaagatgccctagagacgctgaggtggttgtacttttgccataggagtggcagccagagaactgagcccaatgaatgcaaaggctggtgcctggaaatattgtgactttgccacagaaagaagatggagaattttaaagttggaaatctgcctggtaagggatcatttgctggtgtctgcaaagttgagtccatacacactggtttggaaatttcagtccagatgatagttaagaaagcagtaagaatacagagagtccacaatgagatgaaaatgcactgccagttgaaacatcctcctacactggagctttataaatattttaaagacaaggattggattagatttgacattagaaatgtaccataatacagaaggcaatggacacctaaagaacagaatgaaaaccttcttaggaaatgaagcttgacacttcacccaccaggccaccacaagaatgttgcattttcattattatggtaagtacactggtaacttcttttctaacctcgtatgcataatgtaaacatcaagactgacaatttggggctagcaactcaattggaaaatcactgttctctattgtaaaattcctaattatgtttccctagaaattgcaaagtgcacatgtacttgcatttaatgtttggtccatgggctgaatgctttgtgcattacttttctggttgttgttgttgttgttgttgttgttgttgttgttgttgttttgagacagagtctcactctgttgccaggctggagtgcagtggcatgatctcggctcactgcaacctctgcctcccaggttcaagcaattcttctgccacagccttccaagtaactgggattacaggcgctcaccaccacaccctgctaatttttgtacttttagtagagacggggtttcaccatgttggccaggctggcctcaaactcctgacctcaagtgatccacctgctttgacctcccaaaatgctgggattacaggcatgagccaccttgcccagcctgtgcattacttttgaggacacatttgacaatggcacagtcaagaacaccacataaagtagtattggcagattatgaaatagcaattgttttggaagacagcaaaacctgaagtattttcagacatgtacattatgaagtattataccttctttaactatgtgcaagttaaaatcagtcataactgaaaatcaaaagggcaataatgaacatacttcatccagaagaggtatccacaaaacttctgaaagaggtcttagtatatccagggatggagatattattacaatgtattatccaaatgatggaagttttctcttgctgatagaatgcatccacctactaacatcagtaagtacaggtttaacaatttaccagaaatgtactggaggaaatatgaatatgcttccagatttattctacctaaaggatctggctctattttacaaggtcatctattttacaaggtactgtagatgcattttgatgaaaaaaattaccctgctattgattttgaggtctggtattatgatagagcaaaaactatccaaaagagatttaattcaagtcattgcaaaatcttggaaaatgtgtgagaaatcttacacatggaaaggaggaagtaaagatggtagcttaaacatgtgttccactgtgaacaattctctgcaaacccacccccaaaagtcaaggaggctgacaggctgaagaaagaggctgtcaaatctagtttctcagaaaaagaaaaattaatagggacttaagaacagaagccatatatctgccctgagatgagctggtggatcctcacactgttacccccagacccagggcttatataccacagggaaagacaattgcagtaatttgcttaacggcaggatttacggcaagtacatgctcttaagagtagattaagtagtaaccttagaggcattcctagaactggagttaatcagaagtcaacacagtggattagcatccaagatggagttgctttagcctccacaaagagaaaatgaagataaatttggtccatgctaattagggggtgttccatttgtttgaaactgaaatctataatttccaaagaagaaaataaacgtggaagtcgtccttcctttttggtatgcatagacaatctggcagtgttaggcccctaaggccttatcacctcctccttctgtgggtcaaactttacagtgagaggaaagacatcttttaaaagaagaataatcatgactagtttaatcatgactagttcatcttccctgcaatagatacccatatttaacatacctattgtacaaagaatatgattgcacagctacaactttgggacaaactccttttctgccgctttcaatgatcgtcttccaaaatcagaacagcttttgaattttattttgtgaaaaatgtcgtttgagtgacacagtaaactctgtggagctctgtggatacagttgaatgatggctcccagtttatcatgcaggtgggattatattccaacagttatgtttcacagaaaggcaaaataaccaggtacggaaaaaatgaaaaatttgcagcttacatcaaacagaaattgcagtgtctttcatctctttaaagttttctaatcaaaattctagttttcatcaattaaaactttatttaggtagatacatttaataagtaactttttgttggcttaaagggaatgaactttatgtaatgtaactgtctatttcaggaagcttttctatgagaatttaaaactacaccaaaatatcttcttttttttttttttgagacaaggtgtcacattgtctctcaggctggagtgcagtggtgtgatcctagctcactgtagccttgaattcctgggctcaactgatcctgctacctcaaccccccacgtagctaggactacaggcctgcaccaccacatctggctaattttttatatttttgtagagaaatggtctggctacgttgcccaggctggtcccaaactcctggcctcaggtgatcctcctgcgtcaacctcccaaagtgttgggattacaggtgtgagcaaacctgccatctcaaaacatgtgtattttaatgagacaacaaagatcaagggaactttagtccttacaatgtaatgatcaaaaaagtgaaatagataatcaacataacattgcttgtagaccatgcaaacaatcttattgcatttgtatatttgtaatgaactgttttataataacaaaatgtaaaatgtgtattttatttgtccttttattgttttcctacttcttagacatgttaagaactatggaaaaagatatgggagtttgtgaaaatttaaataattcatatttatattacaagaaacagatattagaatatgtttgttttatatttcttttgtagcagaattttctagccaggcattgtggcttacacctgtaattccagcactttgggaggctgaggtgggacaatcacttaagcccaagagtttgaaatcagtgagggcaacatagtgagactctatctctacaaaaaattctctatctatatctatataaaggaattttgtgaggaaaactatatattttgtatataaatgaattttatcaggaaatatatctgtaaatgaattttataaaatatagcatcactttatccttgatttatttcatttaataattattttaaagtaatatactaataaactcattttaaaaactgctatgaccataaaactattagagataacataagagaaaatctaggtgaccttgggtttggagataactttttatatataacaccaaaggcgcaatcaatgaaaaaaaatgacaagctctatatcacatatcaatattaaaatgtgatctgtgaaaggcatcgtcaagagaacaaaaagacaagccacagactgggagaaaatatttgcaaaagacatatgtgataaaagacaattatcaaaatatacaaataattcttaaaactcaaaaataagaaaatgaacaactcagttaaataatgggcaaaagatctcaacagaaaccccatcaaagaagaaacacagtggcaagcatatgaaaatatgatctacatcatatgccattaggaaattgcaaattaaaacaatgagataccactacatttctacattggtgaaaattccaagtactgacagcactctcagtgctggtgaggatatggtgcagtagaaatccacactcatgccgggcacagtggctcatgcctgtaatcccagcactttgggaggctgaggcgggtggatcaccaggtcaggagtttgagatcagcctgaccaacatggtgaaaccccggctctactaaaaatacaaaaattagcccggtgtggtggcgggcgcctgctactcgggagtctgaggcaggagaatcgcttgaacccaggaggtggaggttgcagtgagccgagatggcgccactgcactctagcctgggcgacagagcaagactccatctcaaaaaaaaaaaaaaaaaaaagaggccaggagtggtggctcacacctgtaatcccagcactttgggagactgaggtaggcggatcacttgaggtcaggagttggagaccagccttgccaacatgacaaaaccctgtctctactaaaaccacaaaaattagccaggagtattggcacatgcctgtagtcccagttactcgggaggctgagacaggagaatttcttgaacccgggaggcggaggctgcagtgagccgatatcgcaccactgtactccagcctgaacgacagggcgagactctgtctcaaaaaaaaaaaaaaaaaaaaaaaaaaggaaagaaagaaagaaagaaaaaagaaaattccgtgattgcagtctttacgtatttatttgttattaagtacagtaaaataaagaaggatagatgtcatggaaaatgtcacgaaaataaaagagttaaaaaaaaaaaaaagtaggctgcaatgccagatgcctgaaaagttaatcaacgaaaggacttaaatgtccccattgaatttaggaacaaagaagtaattaatgaactgggcaaaaacactcaatgtaccagcgttatcgatttagaaactgaaactaagtatatctgatgttgcttttaggaaacaagtaaatgaggtcctaaaaagttaaactgtgaccatattttctttcctttttctaatttctccttgggccatttccaaaaagccctaataccccgactgatagaaatggataccttgctgtgcactggtactactgtgattcatggaaagctgatcatgcaacccaagacgccaaaattcccagccttactgttacgaaagaaagtttctaagcacaattgtctctagccaacttcctcttagtaagaaagaggccaggcagggcttcatgcagggtacagccctgagtttcttactgcgtggtaagtttctggggctgggagtaaagcagcgtgaccgaaagcagtacaaagttctaccggacacgcagatcccggtcctacaaatatgaggtccataatgagactgagatatcattcatccaacaaatatttattgaataccaaacattgggcttacggctaaaggaaaatacaaagttgtgttggatatgggttctgttttcaaggagcttataatctaataggaaagatgaggttactacattagtagcaatcagaccagatagaactggaagtgtgatgtaaatgaggtacagattgattgtagaattttgcagaaagaaaacaattctgaagaaatgttcatggaagtattagcagttgagatgtatcttaaaagataagtggggttcctgttggaaacattcaaaactctctcttctagctattttaaaatacgcaatatattattaactatggtcaccttactgtgctatagaacactagaacttattcctactatctaactataattttgtacccactaaccaacctctccctgtccctcctccccctattcatcccaccctctggtaaccactgttctactctctgcttcccaaaacaaagaaatgatcactgtttcggtgatggagatgtcaattaccctgatttgatcattacacattgcatacatgtatagaaatatcacatgcaactcttaagtatataccattcttatgtaccaaatacaaataaaattgaaaaaaaaaaaaaactttggctgggcgcggtaggtcatgccagcactttgggaggctgaggcaggcggatcacctgaggtcaggagttccagaccagcctggccaacatggtgaaacccaatctcttctaaaaaatacaaaaattagcttggtgtggtggcaggggcccgtaatcccagctactcaggaggctgaggcaggagaaatgcttgaacctggaaggcagaggctacagtgagccgagatcatgccactgcacttcagcctgggtgacaaagcaagactccatctcaaaaaaaaaaaaaaaaaagcctcctgagtaactgggactacaggcatgcgacaatactcccggctctctttttaaaaatgagaagtagaagtagtggtgactgtcaagaaagtattttggaaaaatactatgaataaccagaccaaagaaaccaaataccagttgtttcaggagcagtaaatcatttttgagggcttgagcaaaggtcttgctagaaaggtgcttaggggtcagattttggacataatgccaggccaagaaaatttgcactttatttcattaacaatgaggagctcttgaaagttttattggcaggacctaagatagttacagacccacaagtaacatgggtctagaccttgggaacaagaaaagtcaagatcgagtatgcagacttgggaaccgagtataactaatggttaagattaaataaaaatacataagaacactgagatagagaaatggctcattaatgctgcaaatgtctgcatgtttagggatgaaatagaaaaagaagtcagaaaaatacagttcaaacaataggaaagaaaaccagaaatgcatgattctaagaaatgaatgggtggacatggtggctcacatctgtaatcccagcactttgggaggccgaggcagaaggatagctacagcccaggagttcaagaccacccagagcaacacagcaaaaccccatctctacaaaaaacaaaaataaaaattagccaagcatggtggtacacacctgtagtcccagctactcactcaacaggctgaggcagaaggatcacttgagcccaggaggtgaaggatacagtaagctatgattgcaccattgcactccagcctggctgagagaatgagagcttgtttccaaaaataaaaaataagaaagaatttttagaatgagaggaaaacaacatgaaacatgagaaaaataacattccctgttatatggtaggtgcttcgcttatagtttctcattaattcatcatgtaatgtctctgggataaacattatggatttcctgcagaaacagaagctcagaattttatgccaggtgttgaaaggccatgtagctaatagatgatagaacacagactcacactctggtagtcctgagttaacagtagaaaagtcctgggctgggcgtggtggttcacgcctgtaatcccagcattttgggaggttgaggcaggcagatcaccagaggtcaggagtttgagaccagcctggccaacatgactaaagcctgtctctaccaaaaccacaattacccagcgtggtggcgggcacttatagtaccaactactcaggaggctgaggcagaagaattgcttgaacctgggaggtagaggtttcagtgaggcaagatcaagccacggcactccagcctgggtgacagaatgagactctgtataacaaacaaacaaacaaaatcagtaagaaagtcccagactaagaggcatcaaatcaggattctactccaactctgatgccagcttccaggaatacacttgataagttgtttcattcccataaatcttgggtgattcgtgtttaatgagagcattgaactgaatcatttattctatgactcagttctaatatttcacaattctatggttgtataatattacaggaaattcttgagaaggtgcagagggaatggatgaaaaaaaccacatgactaacataaaaaataatggggccatcttttcatttgagattgaaggaaagaacgagaggacaattaaacatgcagagtctgagaacttgcatttaggaggcataagatgctgaactgcaaaattggttagatattgggctgaagagaattgagaattttttaataataaaaactcttggcagggcgcagtggctcacgcctgtaatcccagcagtttgggaggctgaggtgggtggatcacttgaggtcaggagtttgagaccagcgtgactaacgtggggaaaccccgtctttaagaaaaatgcaaaaaatattagctgggtgtggtggtacaagcctgtaatcccagctacttgagaggcttaggcaggaggatcacgtgagcccaggaggtggaggttgcagtaagccgagattgtgccactgtactccagtctggatgacagagggacactatctcaaaaaacaaacaaacactcttaagaaattaggtgtagaaagaatgttcctcaataccataaaggccatatgtaagaaacctatagctggccgggcgcggtggctcaagcctgtaatcccagcactctaggaggccgaggcgggcagatcgcgaggtcaggagatcgagaccatcctggctaacacagtgaaaccccgtctctactaaaaatacaaaaaattagccgggcgaggtggctggtgcctgtagtcccagctactcgggaggctgaggcaggagaatggcgtgaaccccggggggcggagcctgcagtgagctgagatcgcgccactgcactccagcctgggcgacagcgagactccatctcaaaaaaaaaaaaaaaaaaaaaaaggaaacctatagctaacatcatactgaatggtgaacagttgaatgctttcgtctaagaactggaacaagacaagcacgccaactctcaccactcttactcaacatagtactttaagtcctagccagagcaatcaaacaggggaagtggataattgagaatggctcgagaggtgctgtggctcagtcctgttgtcctggcacagaaggagctgaggccaggcgttcaagaatggctcctagatgtctcataaggaaccataaatcaaacaactttcaaaactgaaacctgcgtgagaacggttgcattagttaatctgggagcttcctgcttttttttagctttcatatttatcttagagaaagggaggaaggagagatgtgtatggatacataagcattcaaatacatttgtgtataatcttatgaccagaattcaggtccaatgaacaaaaaggtagggtcttcggaatttccccagtgagatctatgacctgaatattattacgcaaggatccactttgggattacaggcgtgaaccactacacccagcccaggactttcctgctgttaactcaggactaccacaatgtgagtctctgttctgtaatgcccaaccttgtttttactaaccccgcttttagactccccgttttcctttaatcacctagccttgtttccacctgaattgactctcccttagctaagagagccagacagactccatcttggctctttcactggcagccccttcctcaaggacttagcttgtgcaagctgactcccagcacatccaggaatgcaattaactggtaagatactgtggcaagctatatccgcaattcacaggaattcgtctgattgataacgcccaaagccccgagtctatcaccttgtaatagtcttaaagcccctgcacctggaactgtttactttcctgtaaccatttatccttttaactttttgcctaatttatttctgtaaaattgttttaactggacccccctcccctttctaaaccaaagtataaaagaaaatctagtcccttctttggggctgagagaattttgagcgatagccgtctctcggtcgctggctaataaaggattcttaatttgtctgaaagtgtggcgtttttccaactcgttcaggtacaacagttctagcatctattagctatgtggcctttcaacacctggcataaaattctgaacaatggcttggaaattaagataccttagctctagtccttgctctgctaataattaatagagtgaaactggacagggttattcacatgtgtgtgcctcagtttactgttaactgaagaatgacaaagttcataaatttgaaaaggagaggtttcttatatggggttgcaacctgcagggtggccatgctacagtctgggaagcattgcctctggctggaagccagaaacaggcactttcagggtcagaagaataagacagagatttatgctgaatggggtgaccaaatatacatattcaataggctataggaggagttatgaatatttatgaaaggagaaatgtgtacatgtgcaatttggcttcatgccccttcatgggacctacattcaaaaaatggcagccttagcatgatctgagggaggatctttcagccctctgaggtcaaaagtgaaggagaggacaggaaaccctcactgtgtgttctccgtaggggccagaaccactgcatgtttggtgaacctggctggttgttatgttgaaactgcaaaagggagggataacagtcaggtgcttgtttgataccaggggtagaggagactttcaaaagggctggtttctgtttagcccttagggaagaatgtctaaagagaattacctacaagggtataacgaggagtgtctgacattccatctagtaatgaactagaactcagttttcaagttaactctgggaccctcttagccaagaagaggtccattcagttggttgaagagcttaggatttcatttatatctctcatcactaatctgcaaaagctggtagtgaaaccgcctttgcaaaattatgactgagacagcgaaagagatctaacttaatcgattccgtcttgcttctaacctccaagctgtccttattcattcccgggcataggctgaactaactccgggagaagcttagtttatagtttttagtttaaaacaaagatagtaacagccctttcccaaagcagacctccttcttgccttggaactagactgcctttagtgggactaacattagccacaagattagaaactgtggcttaggagtcatacagctggaggctacaagattctgactctccctaaactgctcctaagatcagtccttgagatattttgcagaccctgtacttgatggatcaggtggcaccacccagattgataaagtggctcatctgatcttgtggcccccacccaggaactgactcagcacaagaagagagctttgactctctatgatttcatctctgacccgtcagcactcctggctcgctggcctccctcaggccaccaagttgtccttaaaaactctgctcccactgggcggtggctcatgcctgtaatcccagcactttgggaggccaaggcaggcggatcacaaggtcaggagattgagaccatctggctaacatggtaaaccctgtctctactaaaagtacaaaaaattagctgggcatggtggcacgcacctgtagtcccagctactcgggaggctgaggcaggagaatcacttgaacctgggaggcagaggttacagtgagccgagatcatgccactgcactccagcctgggcaacagagtgagagacttcatctcaaaaaaataaatagataaataacctccgctccctgaatgaatgctcccggagactgatttgaatcataataaaactccagtctcccgcacagcctgctcttcatgaattactctttctctattgcaaatcccctgtcttgataaattggctctgtctagacagtgggcaaggtgacctcactgggcagttacagtagtacccacttcataagtgaaatcacttatcttagtggtagggtcccaaaagttgtttggtaggagagggttgaggctgggagaggtggctcatgcctgtaatcccagcactttgggaggccaaggtgggtggatctcctgaggtcaggagttccagaccagcctggtcaacatggtgaaaccccatctctactaaaaatacaaaaaattggcgtggtggtgggtgcctacaatcccaattacttgggagactgaggcaggataatcgctcgaacctgggaggcagaggttgcagtgagcagagatcgcgccactgcactcaagcctaaccaacaggggcaaaactctaggactagagctaaggtatcaaaaaaaaaaaaaaaaaaaaaaagaagtagagtgtttaattaaataatttgttcttgctgtaaaatgtaaagtagatattcctcttcaaagactttcctccccgtctaattaggaataaatagtaacttctcttagaagcaaaatttattcaaagacctgtgctaacattcttaaatatctgctagccacaataaggaaatcaatgtactttatgttcttagctcccacaatttagcctaaatattttccctggcatgtttatactggtctaagcaagcattaggtcatagcctgttcctcttccttatttaaaagtgtttttacctttctcagcgttccacaagttacttcctccttcctttgttctcctctacctgtgcctcttttaaaaagttctaagttgctagccaattgggacaaatacagaatgtaaggtcccattccagccaacggaaactggacacagcagtagggtggatgtgtcaggttataaatgaccctgtctcctttgtttggtgtactctagtggcaaaactgctggcaagtgtaccttttctgcaggaagtaaaaatggccttactaaataaattaaatttatgttcaagtgctatttctttttttttttttttcgagatggaatttcacttttgttgcccagcctggagtgcaatggcgcgatctcggctcactgcaacctccacctcccaggttcaagcaattctcctgcctcagcctcccgagtagctgggattacaggcatgcgccaccacgctcggctaattttgtatttttagtagagatggggtttctccatgatgaggctggtctcaaactcctgacctcaggtgatccgcctgccttggccttccaaagtgccttggccttccaaagtggcgtgagccactgcgcccagcctcgagtgctatttctttacggcacggaagaacaaacatttcaaacaatgctattaccaagtttgttagtatttattatctcatttgctaaacctaaaaaatatatatccttctttaacgtgatcgaatatttcaaaaagttattgtgttgtttcttaaaataaatcaatcataatcctagactatgttactcaaactacatacaacaccttctgagcttctggcaggcccttcctcccctccctgctcaccacagatcactggaataattgtctgcatgtaacttctaattttgaagtggttgtggtttatcaaacctggaacatggcacttccaagtacatgagctaaggtcacagtaagactcaagccccttcaacagaatacctggaatttctctgttaaagattttctcctttacctgactacatgtttgtaatgcagatccctccaggagcgcttacttataaactgtcctggatcactaacgcgacattttgatgtaaattagtttatcttgacgtgctaatggtagaaaaaaagagaacatgaggaaacttgggtgctttcagggctggtaggaaggattaaatctttgcggcaatttctgagaaggggaaggaaaccttgctaacaattttgatagtttactccatttggctggagtaactctgatccatttgtcaaattcacgatggagcaggtacctgttagggtacaggtttgataaaccacaaccacaggtctatttcatttctccttttccaaagtggaacaaatttgtctctggggttaaaactgcttttctcatattggtgtgtaagagaaaatgagggaatttctttgagtttgtttggtttgtctgtttgtttaagcagcattttttaaataatttactcagccctgtctcagagaaagtccatgatgatctggaattcaacctcagggaaaagttctctcctgtgcctgagacactgcgcaactaactggaaccgaaggatggaacctgggtgtttaatttattaggaacaattgattcttcagtgacactttccatgcagatacttcaaacaaaataatggagccccacagaccgaatgtgaagacagcagtgccattgtctttggaaagctatcacatatctgaagagtatggctttcttcttccagattctctggtaaggatagagccttggtaaggataggtcagaatatgtttcttgagatgttggttggtttgttttttaaaaatgtatgtgattattaagagaccaatataaatatcaagttgtttacctgagaaagatgctacaaagagcatagattatcattactatcaaaagagaagtgacagataccacagagaacaggtcaaatggaacattttttgtttcagtttcttttgactagattgtcaggccagagaaattataagcaaacctgtagttatcaagaaaaagcatgaacttaaatataaataaagaacaaatacagagcctcagcacctggaacatggcacttccaagtacatgagctaaggtcacagtaagactcaagccccttcaacagagtacctggaatttctctgttaaagattttctcctttgcatgactacatgtttgtaatgcagatccccccaggagcgcttacttataaactgtcctggatcactatcgcgacattttgatgtaaattagtttatcttgacttgctaatggtagaaaaaaagagaacatgaggaaacttgggtgctttcagggctggtaggaaggattaaatctttgtggcaatttctgagaaggggaaggaaaccttgctaacaaacaatacctctttcttaattctacttagggctcaaattgtaatgcaaatctttttcatcatttagcccttataaacactgtttttctcatctggtgtggtccaaggcctagaacattaaaactatcaaagcttttacagaccatcaggtgtcatccccctctttctacatctgagctagctgaaatccagaggaaatgacttgctgaaagtcatgagtggcaaaagcagaactagttctgcttataactcttgacttttagttattattattattaattattattattacatcctaaatgagggccaaggccactcagttaaaaatcgtggggtccaggccaggtgcagtggctcacgcctataatcccagcacttttgggaggccaaggcaggtggatcacttgaggttcaggagttcaagaccaggttgatcaacatggtgaaaccccgtctctactaaaaatacaaaaattggccaggcgtggtggcacatgcctgtagttccagctattggggaggctgaggcaggagaatccttgaacccaggagggggaggttgcaatgagtggagatcatgctgttgggaatgaagtttttggtgtcacagaaaaagaatgaacatgggaacaaatgatctctcagcaaaaggacctttactttctgcagaaagggtgctactcaatagctgtccagccacgagagcacaccaaacaaaggagacagagttatttataacctgacgcatctaccctactgctgtgtccagcttccattggctggaataggacctcacattttacactttacccaatcggctattagtttaaaacttttttaattggataagggaacagaacaaagaaagaaaagcaagttgcccagggatagttaaggaaacatctccatataaggaatggcatgcactatgggctggggcttttctagttctgtacagacatgccggagcaagctacgacagctgatttggacagccactaatagtggctagcaatcttatagtaagaaattgtgactttttataatctttgaagaactttcccatttctgacagtgccactgcactccagcctgggcaacaagagcgaaactcgtctcaaaacaaaacaaaacaaaacaaaacaaaacaaaacagctctctactcttggaagcagcagagtttttatcttcatttatatcactccggtaacactcagaagtagacaagcctcagggtaggtattcagtaaaagcccactgaattccacactattctttaatcatagttaaatggcaaattaggctggagggtgggggtggaacctctccaaaattactgcaatgactgcaacatcggaccccaagatttttttttttttttctgagatagaatctctttctatcgcccaggctggagtgcagtggcactgtgagaaatgggaatggaccggactgtttcctctgacactgccactaggttgaccaagtgtccctatttgttaggtactggatggacgcctgacatgcaagactctcagtgctaaatcaggaaagtgctgggacaattcggatgagtcggtcacgctaagttgcacttaatagctcttgtgactttgactgaattacaaacatcccctgaccctcaattttcacatttactggatggagatctggtgccacctccactagattgctatggagaatgaatgtgaaagcattttcataaatccagtgtaaggaccagaagccagtcttctgaccttgagccagtgcttgttaaaaactccactctatacatctaacccaattcaggaatatcctgcctagttccaaaggaagaaaagaccaaattgctcttattgggattaaatgcgtacactgagctgaggaaaaacagtattacaaatgagctaaacatgacgtagatccacagttgtagaattcccctctttgttctttcctctttcataactacggaaacagatgagaaacatttacggcatcaggttcttgtgatgctccctgcctgatatgctatggttttgttaatggaatgtccattcctgagcttatgcagaaaaaagtcccttgggaaagtggttttactgtgttatgttcattttccccatagttctcaaaatgtacttccttgtttcagttttaattttctttcattggtgtgaccattttcaactgctccctttctgggaagaggtagcagacggacattttcatcaaaatctgccccaggttgcttcacagataaggagggacccagccactaaaatcaccaggcagagtgttgcaagagtagatagagaatcacaattggctgccctgctcaaggggacaccagatcttactttcgtttagttgaaaggcaagcgtcagagtcgggaggctgtaccttcatgtccagtggcctcacagaagttccttcagtatctcttttagatgaaactcttttagaagttccttcagtatctcttttggtttctcactatagatagttacttgaacatgtctgaagaaaacgtggtcaagacagtgaataaaaaaaattctggttttgggaagcagtctgacttagtttcaaatattctatcccactgtttctgtcaatgttcaaacctttccaagctccaacatttattgtggaaaatgtgtgcctcaccaactcatgcaaataaatgtttcatgtgccctacgtgtgtagagggggcatggatgtgtgtttttggagggagggctaatttttctttagacatggagaatacgaggaaattagcttggcatcaagaaggttacagcaggagacaagagtgaagagaactgagagagcccggaaatgaggctctggagttcagatttttttttttttgagatggcgtcttgtaccccaggttggagtgcaatggcaaaatctcagctcactgcaacctccgcctcccgggttcaagcgattctcctgcctcagcctcctgagtagctgggattacaggcatgagccaccatgcctggctaattctgtagttttagtacagatggggtttctccatgttggtcaggctggtctcaaactcccaacctcaggtgatccaccctccttggcctcccaaagttcaaggattacagccatgaaccactgcgcctggcctaatttttgtatttttagtagagacagggtttcaccatgttggtcaggctggtcttgaactcctgacctcgtgatctgcccacctcagcctcatgaagtgctgggattacaggcatgagccacagggccaggcctggagttcagatttaacacatcctgtaaatgacatgatgcatctgatatttgaagagttttcctcaaagaatgttacatgcaaggtggtttagagttgttgtttccggctatatagcaaaagtacttggggagttttaaaaaatactgatgccgaagctccacctagaatagttcattcagaatctctagcataattgacctcagtacttgaaatatgattattataaatgttagtcaactgcttttttaggctctatgactgatagaaatctttcacttttatatcatctccagttaatgagtcccataaattgaaatctagtgtttaaatttttacttcatatttatttttactgattgttttattattattatttttgagacagagtccgctttgtcgcccaggctagagtgcagtgacgccatctcggctccctgcaacctccgcctcctgggttcaaacgattctcctgcctcagcctcctgagtagctgggattacaggagcccaccaccaaccacacccagctaatttttgtatttttagtagacggggtttcgccatgttggccaggctggtctcgaacccctgacctcaagtgatccacccgcctcggccctctgtctcaaaacaaaaacaaaaacaataacaaaacttttctcttctacccgagatgtttaagtttaaatcacaccatttgtacaaaaattccctgtcttgtccttaaaaataatttgtaatcactagctagttttgagatcgattgccatctaaccgaatgccatttgttctctctctctagtttcaacttaataaccctttctgcattttctattctttcaaaatttttccggccattttattgtttctatttagtgaaaatttattcactggtttctatgcctaagggcatttaggaagttgcttaggatacagacgtgataaaaagaccagtgtaaaaactctccactcctagacattatattctagtcctcatctcctgtcatttaagtcctcagtgattctatgcacttttgcttttggtttgggcagatgctctgagtttaatgtttctctgagatgaggaccccctattcaactcacaaatcccataaggaggcctctgtgcctttgctggtgccccagacagggtgctgatgcttacttatcttcaagattgtgaagtcagatttaatagtatagtcgtttgccagagctgctgtaacagtagccacaaacagttgggcttaaaataccacaaacaggagggcttaaatcacagaagttgattttctcacagttcttgaggcaggaagtccaagatcaaggtgtcgtggagttggtttcttctgatgtcttgctccttggcttgtagatggcctccttattattgtgtcctcacatggtcttttctccactatgcacaaattccctatgtctctctctcttttttttttttttttttttttttgagacagagttgcactctgtcacccaggctggagtgcagtggtgcaatctcggctcactgcaacctttgcctcccgggttcaagcaattctcctgcctcagtctcctgaatagctggtattacaggtgcgcaccacaaagcccagctaattttttgtatttttagtagagatagggtttcgctattttggtcaggttggttttgaactcctggcctcaagtgatccgcgcacctcggcctctcaaagtgctaggattacaggcataagctactgtgcccagtctccccctgtctctttgtgtccaaatttcctcttctttagggacaccaatcagattaaattggacccaccctaaaggcctcatttcaatgtacctccttcaagggcctatctccaaatacagttatatttttaggtactagggctagggcttcagcataggaatttgggggagacacaatttagcacatagcagaaaatataaggccaggaaaaaatattctggcatgctagatggactcattaacaaatattaaccaatataaaccaattaacaaatatttctttaatatttgcctttttttttttttttttttgaaacagagtttcactcttgttgcctaggctggagtgcagtggcacgttcttggctcactgaaacctctgcctcctgggttcaagtgattctcctgcctcatcctcccaagtagctgggattacaggtgcgggccatcacacccggcttattttttgtatttttagtagagatggggtttcactatgttggccaggctggtctcgaacttctgacctcaggtgatccacctgccttggcatctgaaaatgctggggttacaggttgcctggtgatttttaagaggaatgactgagctctcatgccaggtggggggaggggacagagaaagttgaatactctgacgatagccatgatccatagctctgaagcttagactcgaatctacccatcccgcaaggaagaaaacaaagaaataaaaaagaagaaaagaaatctcccaatgtcaggtcccaccctctttagaagtaatttcagcaaaactttgttgctattttggcatgtcctctactgtagtagcttgtcaaaatactgtccccaaacgttttctatatttctagattttactgtttaatgtataataataatgttctaacattaaaacgtaaccatagcaatgctccgactcacttgatctttaaatatattgttgaactcaattttgtgacatcttcagaattgtctttttgtattcataaatagcaacagtggatagttgccttggttagtgttattatttcaaggattaatctttaggttacgttttcttcataagatgcattagatgacttttttttttttttttttgagacagagtcttgctctgttgctcagactggagtgcagtggtgcagtctcagctcactgcaatctccacctcctggctcaagagagtctctcgactcagcctcctgagtagctgggattacaggcacgcaccaccatgcctggctaatttttgtatcctttttagtaaagacgggatttccccatgttggccaggctgatctccaactcctgacctcaagtgatccatctggcttggcctcccaaagtgctgggattacaggcatgagccaccacacgcagccaattagatgccttctcatgcttttctgtgttgtgaaacagatcatctatccgttgatatagcatagctcttcagactacagacattttaggctataagttttgaattacattttctattccctttatgcttcttgttctagttaggttttttatttctaaataaaaatatgaatttttgcaatttcccaaatgcgaactcaactgaaattttcaagtgtattagcaaaatttattcatagcagcctcttacatttcattaagaattgattttttcttattccctgttgaagtttgtttataaaatatagtaatagtaataactatatagaaagtgttcactttgtagtaggccctatgttaactttaactacactttttaaatctaagcctcatagtagtcttggatggatgcggtggctcacgcctgtaaccccagcactttgggaggctgaggcgggtggatcacgatgtcaggagttcaagaccagcctggccaacatggtgacactgtctctactaaaaagacaaaaatcagccggacgtggtggtatacacctgtagtcccagctatttgggatgctgagtcaggagaattgcttgaacccaggtggtggaggttgcagtgaaccgagatcacacaagtgcactctagcctggatgacagagtgagactccatctcaaaaaaaaaaaaaaaaaaaaaaagagatcaataaataaaataaataaataatcctcacaatagtcttcaccatttgcaaatgatccatttgacaataaatgaattcagcactatcatggaaatatatttccaagctgaccagttttctccatttccaccccacttcaatccaccttcatgtagccctaggactattgtctcctccccttgtttccttatttccattcctgttcagctgtaacacattctgttcatagcagtcaagtgatgcttacaaatggaaatcaggctagaggtggtagttcacacctataattccagcattttgggaggctgaggcaggaggatcacatgaggccaggagtttgagaccagcctgggcaacatagcgagaccccatctctacaaaaataaaaaagaattagctgtgcatgatcctatgtgcctgtgttccagctacttgggaggctgaggtgggaagattgcttgacccagggagtttgaggctgcaataagctatatttgtaccactacactccagtgtgggtgacagagtgagatcctgtctctaaaaaacgtaaaatgaaaataaaaccttgatagtttgctctttaaaactcttcctacagggcccctgtgatgctcacctgtctctagaagggcatgtaatagctctttctccttcactttactttgatgcaatgtcagaacagcttctttccatcaaaacttaaacctttgatttcatttaaaatcatctgcttcaaattctaatctttctgatagtttaggttctaatttttctgatgttaatattgtcacccaagtttcctgttcatatttacctggtttattttatttttatttttatttatgtatttgagatggagtctagctctgtcacccaggctggagtgcagtggtgcgatctcagctcactgcaaccttcgcctcctgggttcacgccattctcctgcctcagcctcccgagtagctgggattacagggacccgccaccatgcccggctcattttttgtatttctactagagacgtggtttcaccgtgttagccaggatggtcttgatctcctgacctcgtgatctgcccgcatcggcctcctagagtgctgggattacaggcgtgagccaccgcgcccagactttattttattttttgagacgaagtcttgctctcttccccaggctggagtgcagtggcttgatctcagctcactgcaacctctgcctcccaggttcaggcgattctcccgcctctgcctcccaggttcaggcgattctcccgcctcagcctcccgaacagctggggttacagatgcctgctaccacacccagctaatttttttctttttttggagacagtctcactctgtcgcccaggctggagtgcactggcgtgatctcagctcactgcaacctccgcctcctgggttcaagcgattctcctgcatcaacctcctaagtagctgggattacagacgtctgccaccacatcaaactaatttttgtatttttagtagctgagattataggctcgtgtcaccacgcctggctaatttttgtatttttagtagagacggggtttcaccatgatggccaggctcgtcttgaacctctgacctcaagtgatctgcccatctcagcctcccaaagtgctgggattacaggtgtgagccactgggcctggcacctggtttatttttgtgcatgcttttatttttaattttcctatgctactttcttagccaaaatttatacttaatctaatcaagcattaatctaacaaagagtttagtgttcatataaaatacagttttacaaatctgtttttctttaaattataaatttgttaagaaaattatccaaagaatgatccagaaacaaaagaatggctgtgtgtcttttcaatatcatcctggagcattgtctcaaccatctcactttacggtgactaaaacatctagaggttttccctttgttttctgtacttcttagtattgattaatactgttgtgctacttcagtctgaagttccatgttaatctgtagatttttttttttttttttgagacagtgtctcgctctgtcgcccaggctggagtgcagtggtgcgattggctcactgcaagctctgcctcccaggttcaggccattctcctgccttagcctcccgagtagctgggactacaggtgcccgccaccacgctgggctaattttttctatttttttttttaggagagacggggtttcaccgtgttagccaggatggtcttgatctcctgacttcgtgatctgcctaccttggcctcccaaagtgctgggattacaggcgtgagccactgtgcccggctgttaatttgtagatttttatacagaaaagcagcaaaatatttctgttgagtagaaaatataactccaatgcttatgactgtattccttataggacactaactcattatgtgtctaacctagcaattttatgtcaacactattttctcaaacctctataaactttggctgggcacagtgggtcacacctgtaatcttagcactttgagaggctgaggcaggtggatcaccttaggtcaggagttcaagacaaggctggccaacatggcaaaaccccatctctactaaagatacaaaaaattagccaggcatggtgacatgcccctgtaatcccagctactcaggaggctgaggcaggagaatctcttgaactcaggaggtggagccaagatcatgccactgcattccagcctgagcaatagggtgaaactgtgcctcaaaatgaataaataaaataaataaataagtcagagattgtgaataggatgttggatatacccaagttatgaattaattaggagcttgaacccaggaggcagaggttgcagtgagctgagatcgcaccactgcactttagcctgagcgatagagtgaaactgtgtctcaatcaatcaatcaatcagagattgtgagtaggatgttggatgtacccaagttatgaattaattaggagcttgaacccaggtttgtctcagatcctcagggactgaagacttccaagtgaattatgggtaatgtataggtctatactactccaaatttacagttttcagacttccctgggttctcatggaccttccatgtcatttctatgtttaggttgaggtccctggtcttttctcctctgtaattaatttcacacccacccctatctcaactcacacaacttgatcttcactcccatctgctaagaaattgagtccataaaaagtgaactcctttaaactctagatcttctactgctgcaaggacagacattccattctggctctctctctcctcctttgcttcttccagtcctttgctccttctgcattctgttactctcctctctcctttgtcttcaatctctccctcttgcaatagccaactataaactgctcaagcttctcattcttaaaagatctctctgaaatgcaaattccctactgccttatggctctccttcaaaagtaatctacattttctctattttctaattcctcaacacactaacgtttgaaccctgcttctatgaccctgacctaaatttctattaaatgtacatagataaactaatatatatttgtgacttcctaatattgttttgttttttaaagaggtcaatcttactttaactctgtaatgatgcggttgaccttctgatgattctctacttctgtgaaatcctctactgtcttgactttttaattaatttatttttttttgagacggagtctcgctctgttgcccaggatggagtgcagtggcacaatctcggctcactgcaagctccacctcccgggttcatgccattctcctgcctcagcctcccgagtagctgggactacaggtgcccgccaacacgcccagctaatttttttgtatttttagtagagacggggtttcaccgtgttagccaggatggtcttgatctcctgacctcgtgatctgcctgcattggcctcccaaagagttgggattataggcgtgagccaccgcacctggccctactgtcttgacttttatagggtcattctattcaagctcattgagcgtctgtcactatgtttttgatctgtattgctggaatcagttcctctatctgccttgtgaatattctccattgtgctaatctaggcttctcctttcatttttcatattccctcacatggctttatacactcttgtggttttaaccacaatatagggtttttactgtagttatgctttcaaattgtatacttcttttttttttttttttttgaaatggagtgttactctgttactccagtctggagtgcagtggtacgatcttggctcactgcaactttcgcctcccaggttcaagcgattctcctacatcagcctcccgagtagctgggattacaggcatgtgccaacacgcctgcctaattttttatttttagtagagacaggatttcaccatgttggtcaagcccgtcacaaactcctgaccttaggtgatccgccctcctcggcctccccaagtgctgaaattacaggtatgagccatcgtgcccagccccaattgtatgtttctaatatgacttttctcctgaactttaaactgtgtatccaacttctcagcacagtcatatcttttgattccacaggtaatttaatgtcaatatatttaaaaatgaatttaccacctttatccccactctttgactttcacctgcatttctgtttcaattcttgtttccatccattcatttgctcacctaattcataaacatggaaatcatcctcaattcctcttcttcttagcccaaaaattcaattgtgcaggttatgcactgagaaaaagagcctcaggttagggggataaatcagagattggtgaactttttctgaagggccaaatactaactgctttaagcttgcttaccatatggtttatgttgcaactaccaactctcctgctgtaatgtaaaagcaaccatagatagcatgtaaacaaatgagacaggctgggtgccaatgaaaattcacgaaaattaatgtagtttactgtcccttgggtgagagttgggggtcactgaaattcggactatgtcttacttggctaaaccacaggcctagagtgggccataaatggagctattgggctagtgattttcttgccttaagcccccagccccaaatttaataatcacatcattttaatttcattttccaagtgtatctttaatatatggcttctcctttccaaattcactgtcattacctaagtttagtccttgaacaatattttaaaggcttccttatctgactttatatcttaaagtcctacaaatttatcttcctaaaattcaaatcaaaccatgtcaccaacttacagaaagggaaaattcatatattctacacacagcacatttcatgtaactttctaggctcatctttcatcatccttttgatgcaggattttctgctcctcagctcagcgaaatccaggatcttgtctcatgaccaggaagaattaggcaggtggacatagtgaagggtgaggatgacggaatttattaagcaaaaggggagttctctgcaaagagaggggtttcaccagcagtctcccacctcacaatggagcaccaggactttcacacacaaactgaaaaggctaggctcctccccagcataaggcatgaattcctggtggttccaccagttttcctactatgcatgtgggtgtgcccaagcaaaccataggtagtatcagaaaaggcaacatttgattggttaaaaggcattattcacccaagcaaaccataggtagtatcagaaaaggcaacatttgattggttaaaaggcattattcagaaagaatcaatcgggaaagggtgagccaataggggaagttctccctctgggtcacgggtttcatctgggaccaggagtctggcctttcagcctttagactgttttaggcttgaaggtgggtttcacagggacccttccctatctgcctaggcatctgtctgcctcctgcctctatcactttctcttgagttttatattttagcaacactgagtcatctttgtcccaggaagcacctacatctgtttatctgctgtcccctctacctttactaccttcccttcttcacatttatacccagaaaagtcacttcccctccaaaaattgggatcaactgtcatatttttatgaatatttcactttaattcctcacaacagctgacagaattaactacttcctcttctttgcaagttatttggctcacacagatatcagtaattaaacatattttactgcattggcatatatctgactaatgtgtttttctcccgtactaggcaatatgctccttagtcatctgtgtatctgaggtgagcacagggcctaactagcatatggtgcattctcaatgttcgttcaactgcattgacttgaattcccctgaagactgaaatgtgaaaatagctactctcggaagcccctttccagagaggtctaaaatatttacatgtttctattttaaatgcagaaagaacttccagatcattataggccttggatggaaattgccaacaaacttcctcaattgattgatgctcaccagcttcaagctcatgtggacaaggtattcttctcttcaccccctcatcacattctgttttcatcatcataccacttttctttcttagccttgtggaagtgtgtcaattgtcctgggaaactgttcattaccattgaacttatcagcaaagctatatcttccttcctgaaaaacagaatgaccccttcgtaatctgatacatgtgttttcctaaggttttcagagccagcacaaaacaatgcctgacacatgccaataactcaccaaatgtttgtttaaagaagaatctgggtgggaatgataaactaactaatggacaaggtatcgcctaagaaggtcagcttggaaattctcaggttcctcattccatgtacgtactcaaggctctgttgttactgagggggtctaacttgattttgtcctaggtgttatagaatagttaaatggagggaatttctgaattataaaattggccatgggttctacaaaacatccaataagcctgtaaattccacaaaagtgttgattaggctgatacaaaggtaattgcagtttttgccattacttttaatgacaaaaaccacaaacacttttgtaccaacctaatagctatgtaaccctgaaaaagttactcaactctgtaatcccatttccttatttataaaatgagagaaactctggtctcacagtattgttatgggaagtaaatcactttcaaagtggcccttttgtagttcttgtcctataatagcattcagtatacattcattacttctctgtagtctcttctccatctgtcctaatctatcagtttggagtaccacataattgcggaagtccatgaaaagttttccgctctccaaaatttccctttgctgatggataatatttaatgtctagaattacaaattctttttaaaatactcattgaatgtttgctttgtgcaaagcactagaaccttgtaaaagatgagtaagggactggcttcaatgtctgtgaagatagcaaactaaacagagtaatttctttgcctgatagataaaatgttgtgttgacatgaccaaagaaatccaaaaataagaaaaaaactatctgtaaacacagaaaaatagagaaaagttccaatgatggaataaaaatttaaaggatttttttgaacgtattaagcaaatcatgtataaaatccagaaataagtttacaggacccatgtcaaggatttaaccaaagcagagggagatccccatgagtccccttttcccatctcagaatagcagagaagagaagcaagggaagcctggaacagttggcaagagggcaggttagaattcagtttgtgaattatgaggtcgtctgccgtaggcatttaccaggctttatttgatttaactgccataaaggaagagaaggacttgttaaattggggctcctcttagcacagcattgaaaccagtccctattccttcttggccttttggctaaaattgagtgtgaaatctatcacctaacatttgtactgggtttaggctgggtgtggtggctcacgcctgtaatcctagcactttgggaggccaaggctggcggattgcctgagctcaggagttcgagaccagcctgagaaacatggtgaaaccatgtctctactaaaaatagaaaaaattagcagggtatggtggcacatgcctgtagtcccagctatttgggaggctggggcagaagaatcacttgaacccaggagacagaggttgcagttagctgagatcacaccactgaactctagcctgggccacagagtgagactctgtctcaaaaaacaaaacaaaacaaacaaacaaatatatatatattaaaatacaaatttttactgggtttaatagtgtcttcctagaagtcatgttcatgcataatctgtgaaagtggtcttatttggaaatagggtgtgtacagttgtattcgagttaagctgaggtgatactggattaaattgtatatgatgagtgtccttataagaagaggaaaaattaaacacaggaacatagactcaagggaaaacatcacgtgaagatggaggtagaattggaatgatgcattgacaagccaagatgtgccaaggattgctggcagtcaccaggagttaggagacaggcatggaacagaatggaacaaattctccctcagaggctccagaagaaatcaaccctattgataccttaatttgggacttctatcttccataactgtggcagagtacatttctgctattttaagtcatgatgtttgtggtcattagttatggcagcgcataaaactaacacaacactcttggtctctatcgcttcttttttttttttttttttttgagacagagtctcactctgtctcccaggctggaatgcagtggtgcaatcttggctcactgcaacctccagctcccaggttcaagcaattctcctgcctcagcctcctgagtagctgggactacaggcacccgccaccatgcccatgtaatttttgtatttgtagtagagacggggtttcaccatattggccaggctggtctcgaactcctaaccttgtgatccaccagcctcagcctcccaaagccctgggattacaggcctgagccaccatgcacggcctctatccctactcttaattgcctggaaaatacctaacaaatgaaggccagtttttagactttaccaccaaaggctgaaattgaaacaggaattgtttgtgagaagcaaacacaatagtttcgagagactgaatcagttagcaatttcctgtgagaggcaaatgtaatagtttctagaaccacagatggagctataacaaaaacatgtgttctctggatcctttacttgctacagacaacacaataagtgaatttacagctttgatcttacagtgcacctaagccaaccaccttgtcttagaatgtctcaacatacctatctgtatcttgaaacaaaatatattaattgccttagacccattcactcacatttcctaggaagacatgatcagagggagctatgcaagaagaaatccagcagaactctggaaatacaataagaaaatccatattagacactaatcttaataaaactaaccttcgttcatgaatttgaatagacaaaattaccaaataatatggaaaaaatgggcaactcaaaaagaagagggtagcccacttggcattcaggaccaatggcctccaataaataagatgatattagtgctttaaatattttatttagtgtatccagtatattgccttcctaaattaagtgaaagctgatatataaaaagaactattagaaataaaaaaccacacacatcccagaactcctcaatataaacctaacaaattcagtaaaacattgattgcaaaaaatatatatagtgacttagggacttccttgataaatttcataaagcataaagaaaaatcaaagagtgcaaaccatcagaaaaatacaaatatataaaaaagagaaatacaggtgatccaaattccttttaatggaatcccataagcagatgggtggaggaaaagtaaaacttccaacatataagcacaaaatttttagaccttaagaaatatttgagttttttatatcagaaagacagtgttggggtgagggttggggggcacatgtaaatactcagataaaattgtgaattttctagggtaaagaaatctgcatattcatagaaaacaaaaatgaaaaatagtttatttacagagtaaatacatacgactgtttacctgtaatgctaaatattaaaagacagtttcttttcttttttttgagacagagtttcactcttgtcgcccaggctggagtgcagtggtgctatatcgactcactgcaacctctgcctctgggttcaagcaattgtcctgcctcagcttcccgagtagctgggattacaggcacccgccaccacacccagctcatttttgtatttttattagagacggggtttcaccatgttggccaggctggtctcaaactcctgacctcaggtgatccacccgccttggcctcccaaagtgctgggattacaggcgtgagccaccgtgctggcctaaaagacaatttcatacctatttgtatggctaattttttaaaaatctggccatatcaaaatactgaagaggacgtatagcaatagagactttcattcattgctggttgaaatgcaaaatggtacagccagtttgaaaactagcttggcagattcttataaaatgaaacatagatttaccatgcaactcagcaatggcattcctaagcatttatccaagtaaatggaaaatgtatgttcccagaaaaaaaatccatatatgaatgtttataacagctttattcataatcaccaaaaaaaaaaaaaaatctggaagaaaacaggatatccttcaaccggggaatgaataaaccaaattataataattgtaaattgtgggatggatagtggaatagtattcagcgatacaaatgattgagcaattaatttgtgcaatgacagggatgaaccttaaatacatttacctaaatgaaagatgtcaggcctatattgtatgattcttttcaaatgactttttagaaagggcaaaactagaaggattaaatatagctttatggttactagagacaggtaaggagtgggtagttgactgcaaaggtaatatataggggaatgtttagcatgatgaaactgttttatatggcactcaggtgatggatatatggctctaggcactgaaaaacccatggaattgtatgtcacaaagaatggactttcatgtatgcaaattttaaaaaataaaccagaaaattgggagaatactaggatggaatgcagactgtgataaataaaactaactggactctcagcaaactaacacaggaacagaacaccaaacaccgcatgttcttacttataagtgggagttgaacaatgagaacacaaggacacagagaggggaacatcacacaccggggcctgttgtggggtggggggctaggggagggagagcattaggacaaatacctaaagcatgaggggcttaaaacctagatgatgggttgacaggcgcagcaaaccaccatggcacatgtatacatatgtaacaaaccagcacattctgcacatgtatcccagaacttaaagcaaatttttaaaaaagtaaaaaaaaaaaaaacaaacaacaacacctaactggactacaaatgcactatataacttcgatgaagagagtggggagtagggaaaggaacggacttaaattactccagaaaatagtgttgtgttgtgactagaatctataaggcttacggtaaatgaaactttacaggatcactatactctaattggtaaatcagtttttcatggggtgcgggtgaacagttgtgaaactgctttacatgtagtcatacctttgcattttgcagatatttcaatttttacaaattgaagattcgtagcaaccttgcatcaagcaagtctgtcaaccccatttttccaatagtgtgtacgcatttggtgtctgtgtgtcatattttgataattataacaatagttaaaactttttctttactattacatctgttacagtgatctgtgatcagtgatctttaatgttactatcataatcgttttgaaggtgccataaactgtgcccctataagtcctgaaacttaattgataaatgtatgtgttctgactgctccactgaccagccattgccccatctctctccccctcctcaggcctcctgatttcctgagacataataatattgaaattaggccaattaataatcctacaatggcctctaagtgttcaagtgaaaggagttgcatgtctctcactttaaaaatctaaaactagaggctggtcatggtggctcaggcctctaatcccagcactttgggaagccaaggcggggagatcacctgagttcaggacttcgagaccagcctggccaacatggcgaaactctgtcttgactaaaaatgcaaaaattagccaggcatggtggtgcacacctgtaatcctagctactcaggagactgaggcagaacaatcgtttgaaccctggaaatggaggttgcagtgagcctagattgtgcgattgcactccagccagggcaacaagagtaaaactccttctcaaaaaaaaaaaaaaaaatatctaaagctagaaatgattaagcttggtgagaaagtcatgtcaaaaccagataggcttaaagctgggcctcttttgccaaacagccaagctgggagtgcaaaggaaaagcttttgaagaaaattaaaagtgctactccagtgaacatacgaatgataagaaagcaaaagagccttattgctggtatagaggaagtttgagtattttggatagaagatcaaaccagccacaacttcccttaaaccaaagcctaattcaaagaaaggccctaattctcttcaattctacaaagtctgagagggctgaagaagctgtagtaaaaaagttttaaaccagcagaatctggttcatgaagtatgaggctagaagccatctccacaacataaaagtgcaaggtgaagcagcaagtgctgatggagaagctgcagctaattatccagaagatccagctaaaatcatcaatgaaggtggctacatttattttttatttttgttatttattaatttatttattttgagacaaagtcttgctctgtcccccaggctggagtgtggtggcatgatgttggctcactgcaacctccacctcctaggttcaagcaattctcctgcctcagccttcccagtacctgggattacaggcatctgccacaacgcctgactaatttttgtatctttggtagagacggggtttcaccacattggccaggctggtcttgaactcctgacctcaggtgatccacccgccttggcctcccaaagagctggattacaggcatgagccaccacgcctggccagtggctacatttaaaaataggtgttcaatgcagacaaaaccatcttttattggaagaagattccaaccaggactttttttttttttttttttttgagacagagtctcactctgtcgccaggctggagtgcagtgatgcgatctcagctcactgcaatctctgcctcccgggttcaagtgtttcccctgcctcagcctcctgagtagctgggactacaggcacgtgccaccatgcccagctaatttttgcagttttagtagagacggggtttcaccatgttggccaagatggtctctatctctgacctcgtgatccaccccccttggcctcccaaagtgctgggattacaggtgtgagccactgcgcccggcccaggactttcatagttagaaaaaagtcaatgcctagcttgaaaggacaggctgactctcttgttaggggctagtgcagctggtgactttaagttgaagccagtgctcatttaccattcctgaaatcttagggcccttaccagctatgctaaatctactctgcctgtgctctgtaaacggacaataaggcctggatgatggcatatctatttacagcatgctttactgaatactttaagctcactgttgcgacctactgctcagaaaaaaatattcctttcaaaatattactgcttattaacaatgcctctggtcacccaagagctctactggagatatacaggaaataaatgttgttttcatgtctgctaacacatttgttctgcaacctatggatcaaggggtcattttggctttcaagtcttattatttaagaactatactttgtaaggctattcctgacacagataatgatccctctgaagaatctgggcaaagtcaaatggaaacctggaaaggattcactattctagatactattaaaagcattcatgattcatggaggacgtcaaaataaaaacattaataggagtttgcaagaagtcaaccctcatggatgactttgaggggttcaagacttgagcagaggaagtcactggagatgtcgcagaaatagtatgagaactaaaattagaagtggaatctgattttataactgagttgctgcaatcttacgattgaacttttcttttcttttttttttgagatgcccctgctgagctgccagttcctgaagggtcaccgggagcagcgcctggcccacctggtcctgagcttcctcaccatgggttatgtctggcaggaaggagaggcgcagcctgcagaggtgagggccagagagcagcttctcctgttacccggcaggttacctgcgcctggagtaacgtgctccctgcttggtgctaccctgttttcctggaaaatgggtactttcttcttctcgatgggcatcagtttaagcaacgatgaagggctcatttattatttattattattatttttttattttattttgagccagtctcactctgtcactcaggctggagggcagtggggtgatcttggctcactgcaacctccccttccaggttcaagcaattctcctgcctcagcctttcttgtagctgagactacaggcacccaccaccacacctggctaatttttgtatttttagtagagatgggtttcaccatgttgcccaggctggtctcgaactcttgacctcaggtaatctgcctgcctgggcttcccacagtgctgggattataggcgtgagccactgcgttcagcctgaagggccatttaaatgaaggatttttttattttaatttttctgactaagagctaatttgttttttaaactggtagctatttcttccttttataagcttttgaatgtttgtttgtttgtttttggcactctcttccaagaatgtttgaagacctgcatttgaaggcagattgcctttttgctttaaaacagggttgcaccatgttgcccaggctggagtgcagtggtgcaatcatagctcactgcagcctcaactcctcccaggctcaagcaaccctcccacctcagcctcctgagaagctggggctaccagcatgtaccgccacacccagctaatgttaaaaattttttgtagagatgagggtcttgctgttttgcccaggctgatcttaaactcctggcctcaagtgatcctcctgcctttgcctcctgtgctgggattacaggcgtgagccaccatgccgggcctgaagacagactctgagaattcataaaaacctcacagcattttgtactcttatgtatataaattatctaggttgctcttcataatcctgtaaagtaacaagagccataccggcccattttacaactgaaaagcacagacacttatttccttaatcaaggtcagacagcaaattagtggaaaagccaaggccagaacccaggtcttctgattttactagtgcagccttctttccccaggggacacattgacatttacaacactcatctttattttttttttaatactgctttctatccagccaattattagtctgtcttttaataattcatccaaatctcttctgaatcattgcataactttgtacagtttccacccacagtgtcttttacttttatttttggaagtaactgtttttaaaagttactgttatttttaaaagtgtgccttccccagaaatcagggagttacccatgtcctagaactccacggtgaagagaacagcctgtgcccatcgtgtttgcctgattgatcctacctcttgtctctcggggaaacacaggagactcagggaagaggaaaagtgtagagtcattgcagccttgttatttgtcaatgcatctcttttctttttctttttctttttttgatacagagtttcactcttgtcgcccaggctagagtgcagtggcgtgatctcggctcactgcaacctcggcttcctgggtccaagggattctcctgactcagtctcctgagtagctgggattacaggcacctgccgccacggccagctaattttttttgtatttttagtagagacgggtttcaccacgttggccaggctgatctcgaactcctgacctcaggtgatccacccacctgagcctcccaaagtgctgtgattacaggcatgggccccagcacccggccagtgcattgcattttttttttttttttcgagacggagtctcactctgtcacccaggctggagtgcagtggcacgatcttggctcactgcaagctccgcctcccaggttcacgccagtctcctatctcagcctcccaagtaactgggactacaggcgcccaccacaacgcctggctaatttttatatttttagtaaagacggcgtttcaccatgttagccaggatggtctcgatctcttgacctcgtgatctgcccgccttcgcctcccaaagtgctgggattacaggcgtgagccaccgtgcccggcgtgcatttttaaaagtgtgtctgatgctgaaaagtttgaagtctaggcacgtcccagtgggtcctctttataccatcccctctgcaaaccattatcctaaattggggtttgggggagagaagagtgacagtggaaagaagtctccacctcccagctgtgccctggtagttccaggggacccggaggctccccacacccaccaccccgcctcagatcacctttcactttctttgtttctcctcccttgacttttcagctcagaaagtacctggctctccaatgccttctgaggaaagtttacccgaggttcacattgcaagactcattaaagctctttagtgttttccacccgagaaaaaattcaagggaaaaatgaagacaaaagcagggcattcttaatggatattttatcttaaggagaaatgaaaatggagatggaagagggggcacaaggatggggtttgaatctagactcgttcagcctttacctccgatagagaacctcatacagcttttctggacttctggctgataaagagccgtggagggttccttggataaaaaaggttgaagggggtctgtcctgtggtggcttacttgaaggtattactgggtttgacttatggagtaagagacggagtcagtttccccacaggctgaggcagtctgtcctcatgcttttctagggcactgtggtctcccaggctcatacctaggtgcacacacaggtttctgcatctagctttgtatctctatgagtcggtcaatcaataaatctatctatcatctgtctactgatctatcatctatctatctagctagctatcatctctctatcatctatctatgtatctatcatctctctctatatatgtgtgtatatatatatatatatgtatatatatatttctatccttccatctacttacctatctatcaaaatttttttccgttgataatattctcgggccccagtttatgtttaattgttttggtaatgcctttctttgcacagtcagtttacagaggttattttatattctatatgtatgtgtggtccagcgttgtaattttcacatatattgcaccgtgtactcataagcagtatttccactgggtcattaacagaaagatatgtgtgcggcatatgaatgtgcatcactcaggtaattcaagcttggttcccagatcatttctgtaccacaggattgccgaaataaaagacaaccatggttatttcctctgctgcaagctttctagaatatgctatttgtctggatttatatctgaaaggtcctgccaaggaatcttgcccttccatttgtcgaagtctccaggaacttggggctccctcctatcctggtccactcagacttggtgctgacgaactggaccaaaaaagatccagacgggtaaggaaggaagagaatgctttgaatttccataactttcccccaggaaacacccaggcttttttttataattagggaagttcatatttatggtctgccgtatggttccaaagaaggggtgagcttgaccaaaaattcaaatatcacaggccccagaagtttcctcttaatccattctgaacacattggctcagaccattttgtcttgtttgtttccacatgacgtgtgaatttctcaacctgaccttcaagctcctgcaaaatcagcttttatttgttctttctcttcaaactgtttattccctaagatgccctccattcatatcaggttaaaaccagttggctttgataagtaatcattatataatgatcagaagagaatgattatggatgaattcagagcagatgctccaggtgggttggattgagaatttgattaataattccatctattccaccaaagtcacatcattcctttgacagttgggctgggaataggggcatttgtctacagaaggaatagcatgagattttaacaaacaagaaattcaacaaacagaattagacagatgatctgagatgttaaattttcctttcaccttaatttttgcagccaaatttatttcagctctagatgaaagagacagcactttcttttgtggctgactacaacagctgaagattcactgaggtttgatatgaggaagaacttcctcagccatgggattgccagaggcgatgatgggaatctacttaaaagggttacatatttagtagacagcctagattttaagactaatttatgtgccccggccgggcccggtggctcacccctgtaatcccggcactttgggaggctgaggcaggtggatcatctgaggtcaggagttgaagaccagcctggccaacatggtgaaaccccgtctctactaaaaatacaaaaaaattagccgggcatggtggcacatacctgtaatgccagctgctcgggaggctgaggcaggagaattgcttgaacgaaggaagtagaggttgcagtgagcgaaatcatgccattgcactccagcctaggtgacaaactctgtctcaaaagaaaaaaaaattatgtgccccattggaagaagtgagattcggccatctcatctctctcgggagctctgagccctggagttttatgttttctgcaattatgaattgtgatccttgattaattatgctttaataataaaatgggtgactactgaaagctgctgaatctgggtaagaatttggatgaaaaaaataatatatgtgcgtaatttattctgttcaaggtactgaatattgaataagctggatttattactcaaagagaaagacagaataagagaaggttgaagggaaaaatgactgtactagaatggtagtcaaaaatgcaacaacaggcaggtgcagcggctcatgcctataatcccagcacattgggaggcccaggtgggcaggtcacctgagatcaggagtttgagaccagcgtggccaacatggccaaaccccctctctactaaaaatacaaaaattagccaggtgtggcagtgggtgcctgcaatcccagctactcaggaggctgaggcaggagaatcacttgaacctgggaggcagaggttgcagtaagctgagactgcaccactgcactccagcctgggtgacagagtgagatcctatctcaaaaacaaacaaaacaaaacaaaacaataacaaaaaagctattaatagcttcctagggagtaagagtgaagggctagtttaattccagagatgcggacacagtcctgggtctcaccaattattctgcttggtaattaccttttgaagccttttaatatgcctaacacagagctaagtgctatgaagaaatgaaagaaatagaagcaaagtactccccatgtggttaaataacaagacactacatgacaaatgtcaaagagtgactcaaacaatatgtcctttagaatttcagagaaatgacatcaatgcaggctttacaagtcagtaaaaatcttggtgatgaggcagaacttgatgtaaggcaaatcctaaaagttgagtaggaatcaactagctagaataaaatgtggggttgtggtaaatacaaaaatgtaagatgagtgaaataacttatttatttatttatttattttcagagacggagtctccctctgtctcccaggctggagtgcagtggcatgatctcggctcactgcaacctctgcctcctgggtttgagcaattctcctgcctcagcctcctgagtagctgggattgcaggcacctaccaccacacccgactaatttttgtatttttagagagatggggttttaccacgttggccagtctggtatcgaatcctcgacctcataatccacctgcctcagccttccaaagtgctaggattacaggcatgagccactgtgcccagcctaataatatattaagatggccacaggccaaatattctggggctggaatgtgagtggaaatgtcgctcaccctttatcacatagcaccccatagtccagccacaacttgcagaattcaaagtaagtgtggatgtgtgtgtgcctgcagtgccttgcacacaagtgtgcatgcctgtggacatgtgaccctagaagttattaatatatctggtttacaaactgaattgttcttttattttttttctctcttggtggtcatcaataactgaaattgggctagattcctggaaattgggtaagttctcagaaatcatttacgcactttagaatccaggccaaatttaaaatcttacaataaaacaaagaacaaagcatgctaaattatatgtataatataatatcaaccatataaaatgcataaaaaatatactagaaggaaatgtgcctaaaattcacagtctaaaatttaacagtgattgcctcttccttgtatagataagggatttttttttactgtattttccagtctgtacataataaaacaagtaatgtgcaatgcaaacaaaacaaaatgaaactttatcaaatttcagtaactccttgaagtttaattttttttttgagactgagtcttagtctgttgcccaggttggagtgcagtggtgtgatctcggctcactgcaacctctgcctctgggttcaagggattctcctgcctcagcctcccgagtacctgagattacaggcacccaccaccacacctggctaatttttgtatttttagttgagacagcgtttcactatattggccaggcaggtcttgaactcctgacctcaggtgatccacccgccttggcctcccaaagtgctgggattgcaggcgtgagccactgcacccagttgaagtttaatagtgtgaaaaaaatatttctcatctcactatatcttctatgggaggccagattgcagattgtctacagaaaaatcccttcaaaagaccttgttattacatagactggagctcatggggcaggtctggtccacacatccttaggctccgcttctcctggaaaacaaaaatagcctctgatccagtgttgcctctcccatcaccaaacctcagcttctatcgccaaactcatcaaataagagtgtccagtagaaaaactgggcagatgggggcacagaaggtgaagacatcatttcccaagctaatgttgctgctggaacaatgtaagtcttgactttgtcttggtttggtttggtttgacattggtttgtttttcatctttgtctcatgcttaaaatgtgaagggcaaatatgatccttagagttaaggttttaggttttgtagatgttttactccatttaaatgacagcagatcatttagaaatgattcctctgtaacagccttccagatcccattcgattgtacagcattgagatagatagatagatagatagatagatagatagatagatagatagacggaatttggccctgtgttcccacccatatctcatgtcaaattgtaacccccacatgtcaggagagggatccagtgggaggtgacaggatcatggggttggatttccccaatgctattctcatgatagtgagttctcacaatatctcattattattattattattattattattgcaacagagtctcactctatctcccaggctggagtgcagtggtgtcatctcggctctctgcaacctcttgcctcagtctcttgcgtagctgggattacaggcatgccccgccatgcccagctaatttttgtatttttagtagagacggagtttcaccatgttggccaggctgatctcgaactcctgacctcaggtaatctgcctacctcgctctcccaaagtgctggaattacaggcgtgagccactgtgcctggccagttctcacaagatctgatggtttaaaagtgtggcacttcccccacctcctgccgccatgtaagatgcttgcttacccttccaccatgattgaaaagtttcatgaggcctcctagccatgcttcctggtaagcctaaggatctctgagtcaattacacctcgtttctttataaattacccagtctcaggtatttctttatagcagtgtaagaatgaactaatacacacataaacagattagaggcagcactggcctgagttgtgaaactcttcccagcctggtcctgcgattagctggctatatgaccttggacaagctgctttgcttctctgggccatggtttcatacctgcaaaaaaaagagcatggacttggctgttgcctgggtctctctagccctgtggagaatcagctacatctcttactaggaacttctcattcagccagttattccactgcggagatggtccaggaccattagggccatgctagacattgggaggctgcctgtcaggtgaacatgaaattgaacttatctgttctctttcctccctgaatgttgctgaaggtagatgcccatcctcagggctgtcttacggagaggagaaagttgtgcagtgattccaccctgcagttatctaactcggcagggaactctgggcagtgagtactcacggtacagtctccacacctctaatcatgtgctcctctccttcccaaggaacctggagaccatcatctcatttcctgggggagagagcctgcatggttttatactggtgactgctttggtagagaaagaagcagtgcctgggataaaggtatcttctcacttgatagcaccttttctttttaaatgagcttgagctttacttcccactcagtgcctttcctgcagtggatttctcaacacaaatgaacatagaccttgtcctgcttagttcaagtctgagagaagagatctaagctctaggccaccatatttgctcccttttctcaattcctataaaactcggaatggaccttttgtccattcaacaaacaggcattggtttgggcaatgggaaattggatcgaacaagacagacattttcccagccctgacagaagcttatgatggatacagtggatgaagatggattaacgtggattacaggtgtgagccactgcaccgggcctcaaactggaaattcttcaggagtcagacaggtatcaggaaggctggatagaagacaaaagacagtgatgcagcttgtgatcaactacagcgttaatgccttgcctaaaaatatttcagttagatttctgccttcgctctgtcgctcaggccagagtgcaatggcgtggttttagctcactgcaatctccacctcccaggttcaagcaattctcctccctcagcctcctaagtagtgcacgccaccacgcctggctaatttttgtatttttagtagagacagggtttcaccatgttggtcaggctggcctcgaactcctgacctcgtgatctgcttgcctcagcctcccaaagtgctgggattacaggtgtgagccaccctgcccagccaacactacctcccttgataagcatatgttgagcacctactggtcctcaatagggtgacccatttctgctatattatagcgctttctttctctctcagtagttaaactccatggttactttagttctcatccatgtgtttagtccattagaagatacagagtcaaatatcggccttccaagtgtagttcagatgaagtagagactcaaggaagacaaggaagtcttcccagcagaggggattctagagctgggggctctgtagaatctgtctgtgtattagtccattttcacactgttataaacatactacctgagactgggtaatttataaaggaaagaagtttaattgactcatagttctgcatggatggggaggcctcaggaaacttacagccatggaggaaagtgaaggggaagcaagaacctcttcacgaggcagcaggagagagagcaaaggggggagctgccaaacacttttatacaatcagattttgtgaaaactctccctcgtatcatgagaacagtatgggagagcccacccccataattcaatcacctcccaccaggtccctccatcagcctgtggggattaccatccaagatgagatttgggtggggacacagatttcaacacagatttaaatctgactttatatgagagcttcggagcaaggatgccccagttggagatgcagtagaactgatcataacgtgacaaatccgagagaagaagagtaaaataatagtactcaggcccttgggaggtgcaagaagtaacagccagatgaaattccagaaacacttacctaggggtctgtctgggaggtccccagggagcttctggctgtcaggccaaccccacagtggatctagcttaggacgttcccaggaagctctgacaaactgtccggtcctcccctgggttccaacagtatgaggcttactctgcctgcatggactttaagggagtgctaataagttgtgtacatgcatctcatccctaggctcttgttcaggccacgaatgctatcttgcagcccaaccaggaggccctgctccaagccctgcagcgactgagactgtctattcaggacatcaccaaaaccttaggacagatgcatggtaagatgcttccgaagctcctgaaggatcccccaggggtcctgggctctgcttaggggaagagggcctggggaccaggcatgtcctgaagggggtgataatacattcatccaccagatgacgctggtggactatctttgttttaggttaaacacatattatcttggagagctattgtcacagctttgtattctccctctcctttatattctcccgtgattaagatggtttcccttctgcagtggccagatatttcttaggcatgttgaggtcttgcctgaagcttgagaggaggggatgggatgcacagtaatgttggtcgcgcgtgccccatcctgcagtgttaggtactgcagagcaggttgtctacactctgtaatgccccttttattctaaccccctgtgttggttcctgagatgtctgaccttggttttaagccttgtctaatggatggcctgtattccctttctgtagctagggcaggctgatttgtcaaaggtaggaaagttgtcagaatcaaaatggagtcacttgtgttgaataaaaatttttaaaccttgacaaatagagctggggaaggctacaaagagagagctcccgtgtataaatgcctgataacaaaatcttttccaaaggactgaaaaaatcaccaccttgcacaaaggccatcacaaccttacatacacaaaaaaatacttacacaacgacatctgcccagcaactgcctttccaacattggccttgtgccaccctttttattgatgctcatagccaaggttaatgatctcaaaacagttacataattgtcctcatttttcctttaaaaacctttgtcttcctttatctttctgaatacccacatggtttattatggcacatgtattcccattgcaatgccctattccagaataaatatcagtttccattagggagcctctccctgttaatctgcttaacacaggcatggtcagttacggggcccaaccttcctggaccggttacatcttattctggttactgcattcagtttccaacatctgggaggctcttcaaattcttctctcagaggaatctgaagaatgtatgtgtttaggaggatgtgagagaggggtgtggtttcttaacaagagaatatcagagtctaagtatcattttccctgaatcttgcttccctgcaggaaagaaagattctgggaaaagagagtgtttacaagaagcaggactggagggagggagaaagacgctaggtactgccaagctttattatcttgtattaaaaaagtaaatataatttgtcatcccagcctctcagcactagtagaaatctatctgaagtcacaggattaggtattatccacttcctggttttatagtttatatttgtattttctctatttccttgaattttaattttaaagccctcatgatcacatcagtaggtcttctgccaaacagctcccttaagttgtatggtggctttgccaagctgaaatgagatgagatgtgttttagctttgccaagaaagcctgagtccatcacttaggatagcaaggctattagggagatagtgcaggtgtcttcagatcacatggatgagcaaaaggaagcaattttggaagattatgagaaaccttccaacaggtcccagtgtacatagcagtaagatggtgcatgcagtgtctaactgtcacaggctttcctagggctcactttcagactcacttctttttttttttttttttttttgtgagatggagtctcactctgtcactcaggctggagtgcagtggcacgatcttggctcactgcaagctctgcctcccgggttcaagcgattctcttgcctcagtctccctagtagctgggattataggcatgcaccaccatgcccagctaatttttgtatttttagtagagatggggtttcgccatgttggccaggctggtctcgaactcctgacctcaggtgatctgcctaccttggcctcccaaagtgctgggattacagccgtgagccactgcgcccagctcagactcactttttaggcccaggccaacctgctgtgttctcctgctcagcttctgcaggaggtctcatcgtctaaggaggtcccagggctaccgcccttgttttctcaaaaggcacattttcccacacagacataatttcgtttcagtgttttactcctagtcacattcatctatatggacaaatagctgaatggattggactgtgttttctaaagatggccccatgccatcctgcgcgctcctccacagcgtggcctgggcattcctttcgaccaggggtggactctgtgcccctacttgtgatgtacgtgttgccaatagaatgtagtataggggatagcacagacttctgaggcaagactagaagaggtgatgcaggtttaaccttgtgtcctgagccactaggtaaaaagtccacctaccctgagatcactctgctgtgcaaacgacacaggcaaaccacatcaaagagccatgtgggttctccagttggcttctgcccaggagtgaaggtgccctcagatggttctaggctcccatcccaacttatgtcctgtcttgaagtcttcccaactgaggcaccagccactgtggagcagagtcaagccattgccatcttgttctgcccagattccccatcaacagaatatagtggttttttcacacctctatgtttggagtggtttctatgcagcaatagtaaccacaagaaataaagttataaaaatagtaacaaacactagaaactgcaagatttaaggaatcacctgaatgctccagtcattgtctctggtttgtaatgataaactttcttctgcgtgataaatagagcttggctggactttttccctctgcttccattccccaaaatggagcgtaccaaacaattgcttctttcagagcccagctttagcaagagtcatgagctctaatcccttcatccataaatacttcctttccaggcacgtagagcccttcacactcagggctgagtagaaaatgcctgttgcagactgcagtggcgttttggagagccaccctcctggtacctgagtcactagtcctttgcccagcttttctcagttttgtaaagcgctcacttctgagtggaagacagaaccagcccggtctattttcataatctgcccccataaggcagaagtccacacggtactggaaacataaccatatctatgtcagtctgccactgcctgccgcctgtcaaccgtgcgtgtctgcatccagtccttctttggagctgctttccagcgactcagccagatctgagctttctgactcattggaaggttaaactattttcagactttcaagtgttgagattattaagacatgtgtttttttttttcttctttttttggcatgcttactgatcgcccctgttctctaggaagtaatgttcttcttggaaaaggtgaaggatatttttctccccaaaaagccatggaaatgtttgcctttatttacttccaaattaacagaatttccagcttttgcttgacccatcggcgcattggcagcgttaagaattttttcttttagcagtaatgaggagtcgaagggtttcttcctaaccatttagtgtatgcatttaaatcaggtttctttttgagtaaattggggctaagctgtagtgtgaacttctgctactgttcctttctcattagttcacttgatttcatggaaggaattttccatctcagcctgtgaacttattttgtctaaactcaattggaaagtaattaacactgagattcttctttaataaattctatatgataataaaatcatacaaacatctctttatttttctttttgacctagaaaagatgttcacaggccagacctggtggctgaagcctgtaatcccagcacactgagaggaagaggcgggtggatcacatgaggtcagtccgagggcagggtagccaacgtggtgaaaccccatctctagtaaaaacacaaaaattagctgggcatggtggcacatgcctgcaatcccagctactcaggaggctgaggcaggagaatcacttgaacctgggagatggaagttgcagtgagcctagatcctgccactgcactccagcctgggcgacagagtgagactctgtccccaaaaaaataaataaataaaagatgttcacaatatattgttaagtgaaaaaagcaggctacataacttgcataatatgagtgcattttaataaaaatatacatatttagcaaaataaaaggacaaatggtatttattaaaatatttacgttgattatttcaacacagaggatgatagttgattttgcttaatttcttccctccttcttttcaatttgaattttctataatgaagattgtttctttttctttctttcttttttttttttgagatggagtttcgctcttgttgcccaggctggggtgcgatggcgccatctcggttcaccacaacctctgcgtcccaggtttaagtgattcttctgcctcagcctccctagtagctgggattataggcgtgtgccaccacacccggctgattttgtatttttagtagagacgggctttctccatgttggtcaggctggtctcgaactcctgaccttaggtgatctgcccacctcagcctcccaaagtgctgggattacaggcatgagccaccgcgcctggactgattattttttaaatagggttagaaagtgaggaagttactaaactccgattagcccaaatatgccccagtgggtccttctggcagagtaaatgtctcggttcagcctgaatctgggaaattgttcctacggttcagcctgaatctgggaaattgttcctaccttataaactggagtatccttcagaaatgacatttactcaaacttccttttaggcagactgcataatagcaatttttaatattaaccatttaaaaaaaaccttcagattaattaacaccaaaagaataattgggaaaatacaactcctcactttaaaaaagaaacaaccaaagtaaatactaaaaaactatatgatgttatattatctaagcttagtttactgcaaagatcaagagcacactactagttgacggcctctattcacactgttcatagccctcgctcgcttctccagccattcactcactcatgcaaaaggtctgtacacacaatgatgcctgatggtataataggaaccttaacatttcaattaaaaggcaaaatgaggacacttaccatcagcctataaaattattcttattattcttcttcttcttctcctcctcctcctcctcttcttccttcttcttcttcttcttcttctccttctccttctccttctccttctccttctccttctccttctccttctccttcttcttcttcctcttcttcttcttcttcttcttcttttttttttgagatggagtcttgctctgttgcccaggctggagtgcagtggtgtgatctcggctcactgcaacctctgcctcccaggttcaagctattctcctgcctcagcctcccaagtaactgggattacaggtgcatgccaccacgcccggataattttttgtatttttactagggatggggtttcaccatgttggccaggctagtctctaactcctgacctcaagtgatccacctgcctcggcctcccaaagtgctgggtgtgggcggcaagccacccaggtgccaaggcaagagacagagggcacgagctgttccagtataatgaggaaaatatatagaataagaatagttatactagaaatagattatagatatgattacatatgaatatcattcttcattagtttgtagcactactctttattccagtattataataatctttgttctacaattataacctaggaaaaaccaggccatacagagataggagctaaagggacagggtgagaagtgaccagaagagtgtgagccttctgttatgcccggacagggccactagagggctccttggtctagcggtaacgcccgcgtctgggaagatgcctgtcacctaacggaccgtggtctagcggtagcgtcagtgcctagaaaaggcactctttttaaatatactttttatttttgtttaatcttccctgatttcctatagatctgagatatgtcatgcttattttcattgctatctaaaaatctcaataaactttatacctaagagtaaaaaaaaaaaaaaagaaaagaaaaggcgctcgttacttagccgaccgggaaagggagtctccctttccccgggggagttagagaagactctgctccaccacctcttgtggagggcctgacatgagtcaggcctgcctgcagtcatctggaggcctaaccgtctccctgtgatgctgtgcttcagcggtcacgctcctagtcctgaacacctggctccgccttttagatagcagtagcagaattagtgaaagtactaaaagtctttgaaatgcagaagtaatggcgtaagctgtcacgtctctctctccgcctcagctgccaaacagagaagggtcccctgtccagtggacacgtgacttgggtgaccttacctgtcattggagacgactcatactccttaccctgccccttgccttgtatctaataaataacagctcaatctggcatttggggccactactggtctccgcatcttggtggtagtggtcccccgggcccagccgtcttttattctatctctttgtcttgtgtctttatttctaccatctcttgtctccgcacacgaggagaaaaacccacagaccctgtagggctggcccctacagctgggaattacaggcatgagccaccgcatccagccagcctaaaattcttctgaaggataataatatagtacttgaagacacggtttgaaaaaaatcatactaaatgaaagggcaccattttacaagcactagaactacattaaacttaaatgaattccaacactcttaataatgtaactcaaaaacaagtctagtgttaacaaaagctccaataactaaaactacattaacaggcacaatgaacattgtaaacgccgctaattggcaccaagtttaatagggcagacaatattttcttctgcattcacacttactcagttacactgttgaaaaatgctgctgctcaagctatgaatgctttacaaaagaaatcattttaataaatacagtaaatgctaaaactctagctaaactattatgcaagatatacaaccaagacaaatacaaattcataatacaagcaacttgcattcaaaatgaactctaccactatattttattaaaagggcagactttatgaattaacccagctgcttcctgaattacaaaagtggcatgactcaatatgaaaataagaaactgtctacaaatttctgacagtaataaattgtaatatacaatacatgcaggagtcttacggaagaataaactctcctaggaaacaaaaatattttatacttttaaaatccaaagtaaaaaaaaaagaaatcattgccagatgcggtggctcatgcctgtaatccaagcactttgggaggccaaggcaggatcgcttgagcccaggagtttgagaccagcctgggcaacatagcaaaaccccatctctacaaaaaaatacaaaaattagatggtaatggtggtgagcgcctgtggttccagctacccaggaggctgaggtgggaggatgcacctcaaggctgcaatgagccaaggtcacaccattgtactgaagcctggggacagagtgagaccctgtctcaataagtaaataaataaatatcttttatgaaaaagattctctagtcagaattaacacctcaactagccaaacatcaggaagttacattacagctacttaatacacaaagggacacattttcaccagtcgttgtcttctgatatttctattccagaaacacacactctcacttccctacactccccatcccatcatttcttcagagcatggaaacagaatttgttgaacaccagaaatctcttgctatggtggtacataagtcataacatttgttgctgcccagcagcaggtatgaagccggctggtgactggctagcaaatgcctattctgtaagctcctcacttagcccatctgtagctctgacttctccaccaattcccttctctcctttcacagcctttctgagtttctgagggataatttcagaggttccatataactgtcaaagcctatggtagacatggcaaagtgaaaatcctctccactggccatttctgtttctcttgggggcatctttcacttgcctcaggtgttataaagctgatgaacacacgtacacgttgtttaacactttcttgggcatttcccatttgagatatggcatgtttcattatcctagtgacatgtgcaatcagaaaatgtatattttgttctctgcaactttcttttgaaaaatgtatatttgaacaaaatatacattttttgtatcttcatgaccattcatgctgtcctcactgtcatcatgaggctctatataacataatgactcctccagggcagtcttcggaaattcccagtgcagaagcacgtgtcatacagcagtccccattcatctccagtgcagctctggctggctcccatgtctgatcagctgtttggttggacagaaaatgactgcaagggaatcagttccagtgtgagctctgtttgcagaactcagactcccctccctcccatgttaatgctttttttcttcttcttttttttttttttttttttttgacagagtctcattctgtcacccaggctggagtgcaatgctatgatctcggctcactacaacctgtgcctccccggttcaagcaattctcgtgcctcaacttcccgagtagctgagattacaggtgcacaccaccacaccccactaatttttttgtatttttagtagagacggggttttgccatgttgccaaggctggtgtcaaactcctgagctcaggaaatccaccttcctcagcctcccaaagtgctaggattacaggcgtgagccaccatgcccagccccatgttaatgcttctaaagtttgccctcacttctttagaaattccttcagtacatcctttaagacttcctctagtgagtgtctgctggtggtaaactctcccctctaaaagttgctttatttctcctcaattcctgaaggatatttttgctagcagctatattcttttagatgttgaacatatcggtacaaaagcttctggtttccatggttgctattgagatgttagctgtcggtttatctttctcccctgactatgtgtacctgtttctctgtatctgtctactttttgggttgctcaatttattggccttgggcttcattctgctgctgctttttttttttttttttttttgagatggagtcttcctctgttgcccaggctggagtgcagtggtgcaatcttggctcaatgcaacctctgcctctcggttcaagcgattctcctgcctcagcctaccgagtagctgggattacaggcacctgccaacacgccaggctaatttttgtatttttagtagaaataggatttcactatgttggccaggctggtctcaaactcttgacttcaggtgatccacccacctcagtctcccaaagtgctaagattacaggcctgagccaccacgcctggccacaatttttaaactttttatttttacaggcacctgccaacatgcacaactaatttttgtatttttagtagaaacaggatttcactgtgttggccaggctggtctcaaactcttgacctcaggtcatccaccaccttggtctcccaaagtgctaggattacaggcgggagccaccatgcctggccaaaattgttaaactttttattttcttcctcaagaggatgagaagaaaggtcaattgtaagctttagaagtcttgcccaatagccaatctgagaatattctccgtaaacattcaccagaggcagccagtgaccatgggatacttttggtgagaggaattgattgctggggtcaggaatgggaggaaagcatacttctcattagataccttgttgaacttcgtaaattgtgtgccaaatgcatgtcttacctagacttcataaattaatttctttaaaaataatcaaagacaattttttaaagacttatttaatttaaggtgattataaaacatccagtatactttcactattaaaaaagtaagtattcctgtctgggcttggtggatcacacctgtaatcccagcactctgggaggctgaggtggtcggatcatgaggtcaagagattgagaccatcctggccaatatgatgaaaccatgtctctcctaaaaatacaaaaactagctgggcgtagtggcgtgcctgtagtcctagttactcaggaagctgaggcaggagaatcgcttgaacccaggaggcggaggttgcagtgaactgagatcgtgccactgcactccagtgtggcaacagagtgagactccatcttcaaaaaaaaaaaaaagtattcctaaacagcatattatcatgatatattattttgttttgtagggttttgaaccttgtctaaaaagaattaaaatgtataaatttcttcctgcaatttccctatttcactaagggtcattcacattggtcatatagacatagcacattttcaccactatatagcagcattttgtacaaatagactacaatttacttattctgcacttatttctgtttgtttgttttgctatgaaaagcaatgtcattacatatattcatgcccatagctacaagtttacatatttcaggttttctgtagggtggacaccagggagttgaattgttcaacaggactttacattcatctttagttttattggccaacaccaaattgttcttcacaatgtttgaactaagttgaaattccacctccccatcacatttagttttgtcaacttcatttcttcattcattaattcattcattcagtcttttgtttatttgtttattgccagtctgataggcgtatagtggtgcttcatcatggttttactttgcatttctctgattttctttttaaatttttaaaaaattatttttatgtagaaacaaggtctcgctacatggcccaggctggtcttgaactcctggcttcaaatgatcctcccacattggcctttcaaagtaccgagattgattataggcgtgtgccactgtggccagctgatttccctgatttctgatgagttaacaatctcttctttctctctctctctctctgtgtatacaggtactcaccattcgtgcctattttctgtaaaatatgtggctttcctcattttttttttttttttttttttttgaggcagagtctcgctctgttgcaggctagagtgcagtggtgcgatcttggctcaccacaacctccacttcctgggttcgagcaattctcctgcctcagccttcagagtagctgggactacaggcgtgcaccaccatgcccagctaatttttgtatttttagtagagattgggtttcactatgttggccagactggtctcaaactcctgactttgtgatctgcccacctcagcctcccaaagtgctgggattacaggagtgagccactgcgcccagccatctttcctcatttttatactaattaggcttttatcttacttgttttttttaatgttttttgtacactctgaaggctgatttttgttaattgtatgtgttgcattttttatggtttgtcttatgccttttgaaagtaaaagttcttaatttaaatatagccaacctgtaaatcatttgtgaaagtctgtggtttaagaggtcttgaataagaaattatcccatcatcataagtcataaatacttttttgttgttgttgagacagaatctcattttgttgtccaggctggagtgcagtgggttgatctcagctcactgcaacctctgcctcctgggttcagcaattctcctgcctcagcctcccaagtagctgtgataataagcatgtgccaccacaccagtctaatttttgtatttttagtggagacaggatttcatcatgttggccaggctggtctcaaactcctgacttcaagtgatccacctgtctcagcctcccaaagtgctgggattataggtgtgaaccaccatgcctggcccataaatacatttttatgtattttcttctaaagttgttttgtctttcactttttagtttttaattcacatataattactacttgctacttataattatctgtaagtagtatgagatgagaaataaattctatttccctcctatggataagcacaaacctgcagtattagcacagtcttatgtcagattttctaaaatgaatgggtgtgttttctaggctctctgttctgtttcattatctgtcttttcctgcaacgatatcatctgccttaaaaactctagccttgtggtattcctcattttcaagcagagcaaaccccgtcaccttgcttttctcctccagcatcgcttgtgctatcctggactaagaccttcatatagactgttagaatcatctagccaagttccattttaaaaatctatgttggagctgggcgcggtggctcacgcctgtaatcccagcactttgggaggctgaggagggcagatcacttgaggtcaggagttggagaccagcctgatgaaaccccgtctctactaaaaatacaaaaattagctggacgttgggcacttgaattctagctactcaggaggctgaggcaggagaatcgcttgaacctggcaggcggggggtgcagtgagccgcgatcatgccactgtactccagcctgggtgacagagtgaggctccatctccaaaaataaataaataaataaaataaaatatctatgttggaaattttgtacaaattttattaaatgtctacattaatttggagaaaaatgacttgattttgattatctattcaatatttctgtattatgaataaggcaaaaagagaggcagagaatagcataaaataataactaaaattcctgggtaaaccacctcaaatcatttcttcatatggctcaatattcttttgtgacatggcctgaaatatatccagacagagaactcttctcttcaatacatttcttcttttaggtattcatattgagttttcctgtccatgaacatggtataagagagtatatcccttcgggaggccaaggtgggtggatcacctgaggtcaggagtttgagacgagcctggccaacatggtaaagtcccatctctactaaaaacccaagaattatccaggtgtggtgacacatgcctgtagtcccagctactcaggatgctgaagcaggagaattgcttgaaccaaggaggcggaggttgcaatgagccaaggtcatgccattgcactccagcctgggtgaagagcgagactccatctcaaaaaaaaaaaaaaaaaaaaaaagaaaacgagaatatatcctttcatttactagtttttcttcaatttctttcaagataaagggcttacctatcttctgctttattcatagttacttgatatttttgtttctaataaatatggtgtctgtctatttacctgcctcttacctgttcatttccagtttcaaaaatgatgttgatatttgaatattaaccttaaatctagcaccttggtaaacactattattcattctaataattatcagtagattatatgtgttttttatttataaatcatattgtttgagtagcatgctttgcttcttcatttataaaatttacaacttttatttctttttaataatttttttcttattctcctggctaggacttctaacacagtattgagtggaagtgctgatccttgtttagtttcacatttgaaaaaagattgcttttactatttcactgttaagtataatatgcaccataggctttctgtggattccttttatccatttaagaacatctcttattcctaattagctgaagttttctgcatgtttgttttcatcatgagtggatttttttacatctattgaaatcattttacatagaagatatttcacacctattgaaatggtcatttcacttttccttctttaatatgttaagttgggcaaaatattaaagtatcacctgtcattctgcttcagcaaaaagtagtagtgtcttagcagtattggtgaaaagacagcatcaaataaaaaagatgtagaagtaggacccagtaaaaatctagcgcatggggcattgtcacatgtaagcagacagaatgtgacaccaccaaggagcatctgaagggctggaggctgaaggaagacatgagtcacccaggctcatggacacttcagagaaattagggagcaggaagaagaaataggatcaaagactacgtatgttggttggaaaaggaagctgatggtatggagatgttattatttaggtctcacataaaagatgtagataaataggtagataggtagatagatgatagatagagagatagatagataaatacatagatagatagatgataaatagatgttgttatttaggcctcacataaagatgtagacagattagacagacagatgatagatagatagatagatagatagatagatagatagatagacgatagatagatagatagataatctcagaaacagagacacagtgatctcagtaagataggcatatgccaggtgacagaattcagaggggtcccactacgtgaaaacaatagaacaaccttcgaaaagaaatttagtacaaataagagggcaggcttccttacatacaagttagtaaactggaagaatcagttatcctcaaacattggaatagatcaaaaatagttgtttatattaatgaaggtagctaaacatgaagctaagtgaacctgtctctgacctagtgtggcaatccctgggcaagggacacttgctccgctcttgtatccttcactgaatattcagactttcagttaagcatcggtgaatttagttttcatctcttgtgaaaaccttgagagaggtaattctctctgcttttcttcttttcccttccttcattttctcaaacattgcctgtttaaaatacgaaattttaaaagatggccttgttctcttttttgttgttattattaagtacagagaaaggaagaaccacaaatagcaaagggcaacatatggaatagtttagaagttccgggagcacccatgagggcaactgcagaagagaacattctatcccccgttgctgcagctttcattccaggtctccatgcatatcagatagggaaggaactccgggacagcagcagggcccatgcacatgtaaccaattgctttctttgcctgtagtaaagttcacattttgattgcttctccagattatgtagatccagacatattttatgcaggcatccggatctttctctctgggtaagtatagttcagttgttttcctgtgtgaagtctctgtagcattgactgaatgtataaggggacgaagagacagaagcttcctagcgtaagaaacataccaagtgactcttgctagggatccactctcaggtaaaagaagtgggataccatctgcacaacaaataacactgagggctaagtatttcagttaagagtgtttgttcctaggcagttcagatccatttatattcacttttcttagaatcctagctcaatgacagaagaagaaaaacacagtatgtcactcacacagttctatcacttacatctactttttcttcttgttattaaggcatgtagaaggctggggagtgtagtatagagttggatagcatcgaagctttcttctaaagttcctggaagagctacactgtggtttgaacgaatgtgtccctccaaaattcatatgttaaaacctaattgtgatggtgaggtattcgaaggtgggttctctgggaggtgattatgtctcctctgagaggagaagacaatcccctgagtggaactaatgcccttataaaggggctggagggagttcacttggccctttttgctccttttttcttccattccttgtcccttccactatgtgaggacacgggagttgaggcattaccttggacgtggagaccaggccctcaccagacactgaacttgccagcaccttgatcttggacttcccagcctccagaactgtgagaaatacatttctgttatgggtagtccccaactcaatacagtttgacttaccattttttgactttatgttggtgcaaaagcatacatattcagtagaaactactttgagtacccatacaaccattctgtttttcacatttggtacagtatttaataaattccataacatattcaacatgttgacataaataagctttgcgttaggtgattttgcccaactgtgggctaatgtaagtgttctgagcacatttaagggcagctaggctaagctaaggtgtttggtaggtcagatgtattaaatgcattttttgtcttatgatattttcaacttacattgggtttatcaggatgtaacccactgtgaattaaggaatatctgtatttacaaattacccagtctaatgtattttgttgtagcaacaggaacaaactaaaacactaccccaaaccatttttttcatattttctgagtactcttcttttgtcacatggcttgaaatttcttcacatagagaactctactattattttattttattttattttattttattttttgagacagagtcttgctcttttcacccaggctggagtgcagtggtgccatcttggctcacggcaacctctgcctcctggattcaaactactctcctgcctcagcctcccgagtagctgcgattacaggtggctgccaccaagcctggctaattcttgtatttttagtagagacggagtattgccatgttagtcaggctggtctcgaactcctgacctcaggtgatctgcccaccttggcctcccaaaatgttgggattacaggcttgagccaccacgtctggctgataactctacttttagatactctctttgcttaaacaaattagccattccttccttgacatgttttaatcagattgccttcctattaacttcggaaattaagagtttctgctatgtttttgtttaatttttaaacagtgaaaaatgaaaggtggaggcaatggctggatgaggtgaatatgtcaaatagatatcatctagtgggccatcttattaactaggagacacctgaagtgctatcaatagaaataatctgaagctgtgtctggatacagcaagagacatgcaaatgctaaaaatctactatattacattggtgcaaggacagagtagcaacacatactaagtattttctccagattgggtgtgtttgacgtgtgaagcacttcaaacagagtccagcctgggagggagtggggatggaatcctccttgtagagggtacagagtggaagcaagaaggtttccaagattgagagtaatgggtgtatggtttatgggggaaagggaaaacaaaaagaagggtgcagaaatggagctgggagtgtgttttagtatttaggctttctctgtatacctttgataggattagaaaaagaaaaatggaccatttttaaaaatttcatgctaccacatagcaggcttatactatagatgcagaaacagactgggatttaggaagcaccccaattctggaaaatccctttttgcttcacattgctctctaaatctgtatgttttcccttgttacgtaacaatttaccacaaatttagcagcttaaaacaatatgcattcattgtttcacaattctgtaagtcggaatcataggcaagctcaactgacttttccatttagggtcgcaaaaggccgaagtcaatttatctattgggctgggctcttaactgaagatctggggaagaattcccttcaaaactcatttaggttgttgtcagaattcagtgttttgtggttctagaactgagatctgtttccttgttggctgtcagacaggagctgctctcagcttcttgaggcatccagtattccttatcatgtgtttttttttttccatctcagcactggcactttttttttttttttttttgagacagagtcttgctttgtcacccaggctggggtgcagtggcacgatctcggctcaccacaacctccatctcccgggttcaagtgattctcctgcctcagcctcccgagtagctgggattacaggcacccgccaccagcccggctgatttttgtatttttcatagagatgtggtctcaccatgttggccaggctggtcttgaactcctgacctcaagtgatcctcccacttcagcctcccaaagtgctgagattacaggcatgagccatcatgcccagccagcactgacaattctaatccttctagcactttgattccttctcacctctccttctgcctctagccagagaaaactctctgattttaaaggtcttatgtgattagattcagcttacctaggtaattcaggataactccctatgtcaaggtcaactgattaataaccttaattacacctgcaaagtcccctttgccataatatatcatactgacagacatgctatagcataatactaatagttctaagaattatgataagaatcttggaaaaccatttttagaattatacctaccacagtatccttcaagggataaattgattctacttcttctctatgtcagaagcatctgatgaggatgaactatatattctgaaatccccatgattagatgtgtactagaaggtgattttactttcattaaaataaattcggagtcattgacacattttatctttgatttacataaatgcctccgctctgtttcttaccctcaaaatatttcccatgtagctaagtggccagtaccgaatcctacatgcattaataagtgtagatggacaaaaatatctggattactgagaattcccattagcattgtctagaaaaatgtaaatttgcttttttgttcttgatttatcctatttttgatttattatttatatttatttatttatttatttatttttagatggaggtctcgctttgtcgcccaggctggagcgcaatggcgcaatcttggctcactgcaacctctgcctcccaggttcaagctttcctcctgcctcagcctcccaagtagctgggactacaggcacctgccacagtgcccggctaatttttgtattttcagtagagacagggttttgccctgtgggccaggctgttcttgaactcctgacctcaggtgatctgcccaccttggtcccccaaagtgctgggattacaggcatgagccaccacacctggcctttttgcttactttttaaaaacatttttatttaggagaatggagatatttcatatgtagatgacacatattcattccctttagttcccacacacattcaatttcttgaggaagttagcctttgcaaaaaaaaaaaaatgatctcatttttttttccccactaaaacttctcattttcttggggttgctagaaagttgctacaagaaaggctaaaaataattgtgcctacagatatttgaaaggaaaatagttcctcttttttcacagtagcagcttggacctgagaatgtatgggagcaataattgggctgctcaaagaaacacaatttcccttcctcagactagaattaccaacctagagaacatgagtttttaaagtagatgtgcttcttttatctttttggacttgtatgctggtgttttctctgtcaccttcactgtggaaatcctcttgagggtgaggcactgaaagcagattgattaatgtctcttggccatttgagacattggatggctcttttaagttggccacgttctttcaagaactatgcttgggctacatattctggatatataatacatacttgtaggatgttatttttaaatcattcatttatcacatatttagtgagtgcctaccccatgtcagttctaggtgctggaaatagagcagtaaaaccaaccctcaacttggcccctctggagcttacatttcaatgcggtgggtggggggatggacaattaatacacaagtaaatctaataaaagcgtcatacaatatacattacaatggtaagcacaatgaagaaatggaaagctggatagagagtattagagactgtcgaatgtagtggccaaattttcctgtttattgtggtccaagagtgccacagccctctatgacatttgagcagacacctggaggaagtgagggagtgagccgccaagaaggaatggcaagtgcaataaccctgaggtgggagcgtgttggtcgtggtggaagagctgcaggaagccagcagggccgcaacactcaggggagaataagaaagagtgaggtgacagtagagaccggatcatgtagagctttgtcagcttcttttctgagtgagatggacaacacggacacgttttgaaaagaataacaatgtgatctgccttcagttgcaaatcatctctgtattgactgagtaggaaataaactccaagaataaagacggaaacagggaaaatatttaagaagcgatcattacaatccagggtggtggcttgtactagggtacaagggctaaaggtgttgagaaatggtcagattctggatatatactgaaatcaaagttgatggaaagatatgagtcaaagataatttgcaggttttggggtcctggttcactgaaagaacagagacatcatttactccaatgaggaagactataggaggaacaggtttagcaaagaagaaaggaaatcaggagatcagtttggggacacggtcatatcaagtaggcagttggatgtatgggtctggaatataggggagtggtctagctatcagtgtaaatagctttttacatttgtaaatagtcaggatataggcttttctttttctttttgtgagacacagtcttgctctttcgcccaaactggagtgcaatggcacaatctcagctcactgcaacctctgcctctgggttcaagcgatactcctgcctcagcctcccgagtagctgggactacaggtgtgcaccaccataccaggctaatttttgtgtttttaatggaaatggggtttcaccatgttgaccaggctggtgtcaaactcctgacctcaatcgatctccccgcctgggcctcccaaagtgctgggattacaggcatgagccaccccatccagctgggatatagttgttttctaaatctatgaggctagagaagaccatttgggaattgagagcagtgagcagtccacggactgaccctttgagagtgcaacatttagcagtactcaagatgggaaggagctagtgaaaaagccccaaatgactacataagaccatcaaccagattacaaagaaagacccgaacatgtgtgcccatatatcacccaacagcagttatgtctttcatgtttcttccccataaaatgttgttcatcaactttattagactagggtcttaacattggacaaatcacaaaacctctctggagcctattttatttttcaacagctgtaggaagcaaatacaaattggaaatctaaggctcagaaagatttgtacaaagttacacagtaatgaaaggggagccgggattcccactcactctaaagaatatgataaaatggctagtattcactgaatgcttaacatgttccaggccctgggcaggtattattttaattagttctcacaataatccaataagggagatactaatttactcagatgagaaagctgaggctcagagaggttaatgaactaagccaaggctcactgttaataaatagcaaaggtaaaattaaattccatatctgcttgagatagaggccttgctcctaatagctgcagcctgtcagggcctggcagcagtaacctctcctttcctcttcccaccattcccctgcactgctttctgtaccgcatctcttttcagagtgatgttgccccaattgcggaggccactgtgctgtttatccagtgaaagctgtagcacagccaacccaaagcgtccccagtgaaaacaacctggctccttacagcacttccagcctcagagcagtatttgaaaaatatcatgaacagcaaacacagcagtctgtctgtggcttttatatgtgtatatggtgtgtgtgtatgtcccttctcttgagcaaaataacttttagaattatagaaaaaaaatgtgcaacatcaatgtggatctgctgtttaaactcataacagagaaagtagcttgtttctggctataggaggaaaagacgatattccttagtaaaaatggaaatccacatatggggttcttgtaaaaatgaagatagaaaattgcaagtttggggatcaagttctggttctatcatcctttaacagtatgaccctggaaccttaattgctttgagtctttgttactttatctatgaaatgaagtatttaaaaaaactccaaaaatctgtcctgatgtacacacaagaggtcaaatgagaaaatgaatgtgaagatgctttataaactatacagcatggtaggtgcaaatgtgacatgaacttgttttggacacattataaagtcacccccacaaactgtgattgttcaagactatgcaaagtcagacacaggaaaataagtaaaacagatggaggcataaagagggggaactcagagaaaacagtgaagaacaggaatcaggaagacaaaggagaggaaaggtggggaggagaggagaaggaaagggggaagggaatggaggagaggagaacagctgcttcacagagcatggccggcagcccagtcccagcctttctgcatgtccctgacttcagcctctggcgaggcacaggcttactctgtgcttcctgctgttactcttcttatccatccttattatcaatacctgtggtcaacaaagtatttgataaaggcatcctcaaagtcaggtaacatctgtacgttatagattacaaagttgagtaatatccagaattggtagtttaacgtgatgacttcttaacaattatcactgtttcagggaagggcaaaggtgtgtgtgtgtgtgtgttcatctgtgtgtatctgtgtatgtaattgtgggtgtttgtgtatatttgtgaggctctttacttggcggagttaaaaagtatctgctcatcaaggttgagattagcaaaggaagtgaagatttttccagagcccctaaaatgtgccttttgaccaacactgaggacatctttataactgagtatgtgcaataaatatgtcttgggacctgtgccacaaattcctctctaaatagcctttacctctctggaataaccctttagatgaggaagaaaagggctgtgattttatagcttgttatgaagctggagtgaagatgatgcttcagtacttaccctacaaagatacccccaatccctcaccctaaaattaccattgaaatcatgttccctttctcattcactctcagtttccatgtcagaaaatataccattacctccctgcacccctttcatctctctcacttttctcttgcttagatggaaagacaacccagcaatgcctgcagggctgatgtatgaaggagtttcccaagagcccctgaaatactccggcgggagtgcagctcagagcacagtgcttcatgcctttgatgagttcttaggcattcgtcatagcaaggaaagtggtaagtcagacattttgttttcccttgagagtagagggaggaagaggagaggtgttttttttttttccaattgataaaaccaaatataaattaaaatgtcatgaagtttatacttctctaagtcagccaagaaactgcatgactgccaatgtttttgtgtcaagccaattaatattggaatatcagatgtcagcttgatcttgggttttacttccaaatcttaaaatgttgctctgtttccaactgttcactatcactttggtttggatctttagacactagcttcctttttctgaaatgggggagagatgtggagtttgaaggctatgagtctgggccagctggaaacaggtctgggatcttccaagaaagtccttccccacaaaatggtgcaacttctagccaaatctatttataccagcagagggatctatcaccctggaagcttgaaattgttcattttcttacctgccaggatcaagttaagtttttaacagttgcaaaaagacacttcatactatggagttttcaagttggattagaagaaaaagaatcaccagaacttagtgtcgtagattcaagtcacttctctaaaactgtcataatttttcacggattttggcatttggtgacattaatggttgatttacttaccatgcataatattaaacccataacgaatttcctataaatatctattgatttgatttttaaatcacttggcttcaagaggctattactaaaacagtgactcattctttatcttttttgccttcacgggctttatataactttctccttttcttgtgctccctccaaaacaaagcactgagaaaacaaaattcaccagagtattcagctagtcagttcaagggtttgtgttctacatttgaagatattccttatagcagctaccaacgggatactttgtttacatttgttgtgtagtaaatatttatatattggcaaacaaatctagttccaactctgtcatctgagatgttcttactttgtttcctcttctccatctcctgtcaactgttagaaatatacatttgagtacgtgaagtctgcaaacaaaagggccaaggtagatttgagttagaacaccagcaacagtttctggtgcattcttgtctgaaaaagcagagaagattgggcaccgtgggtcatgcctgtaatcccaaaattttggaagtctgaggcaggtggatcgcttgagcccaggagttcaagacaagcctgggcaacatagcaagaacccgtctctactaaaaggaatacaaaaaaatattagctgggtgtggtggcgcacacctgtagtttcagctactcagaagactgaggtgggaggatcacttgaactcaggggcagaggtggcagtaagctgagatcacaccactgcactccagcctgggcaacagagcaagatctcatctagagaaaaaaaaaataaaaaagaagaagaagcagacgagctctgctatatttccatgtggagctatgacgttatgctgtattgtttctttcaggtgactttctgtacagaatgagggattacatgcctccttcccataaggccttcatagaagacatccactcagcaccttccctgagggactacatcctgtcatctggacaggaccacttgctgacagcttataaccagtgtgtgcaggccctggcagagctgcggagctatcacatcaccatggtcaccaaatacctcatcacagctgcagccaaggcaaagcatgggaagccaaaccatctcccagggcctcctcaggctttaaaagacaggggcacaggtggaaccgcagttatgagctttcttaagagtgtcagggataagaccttggagtcaatccttcacccacgtggttag

SEQ ID NO: 22 - DNA, 890 bp, Homo sapiens
  source 1..890: mol_type = genomic DNA
ggcttatggagccccacagaccgaatgtgaagacagcagtgccattgtctttggaaagctatcacatatctgaagagtatggctttcttcttccagattctctgaaagaacttccagatcattataggccttggatggaaattgccaacaaacttcctcaattgattgatgctcaccagcttcaagctcatgtggacaagattcctggaaattgggctcttgttcaggccacgaatgctatcttgcagcccaaccaggaggccctgctccaagccctgcagcgactgagactgtctattcaggacatcaccaaaaccttaggacagatgcatgattatgtagatccagacatattttatgcaggcatccggatctttctctctggatggaaagacaacccagcaatgcctgcagggctgatgtatgaaggagtttcccaagagcccctgaaatactccggcgggagtgcagctcagagcacagtgcttcatgcctttgatgagttcttaggcattcgtcatagcaaggaaagtggtgactttctgtacagaatgagggattacatgcctccttcccataaggccttcatagaagacatccactcagcaccttccctgagggactacatcctgtcctctggacaggaccacttgctgacagcttataaccagtgtgtgcaggccctggcagagctgcggagctatcacatcaccatggtcaccaaatacctcatcacagctgcagccaaggcaaagcatgggaagccaaaccatctcccagggcctcctcaggctttaaaagacaggggcacaggtggaaccgcagttatgagctttcttaagagtgtcagggataagaccttggagtcaatccttcacccacgtggttag

SEQ ID NO: 23 - DNA, 1177 bp, Homo sapiens
  source 1..1177: mol_type = genomic DNA
ggcttatggagccccacagaccgaatgtgaagacagcagtgccattgtctttggaaagctatcacatatctgaagagtatggctttcttcttccagattctctgaaagaacttccagatcattataggccttggatggaaattgccaacaaacttcctcaattgattgatgctcaccagcttcaagctcatgtggacaagatgcccctgctgagctgccagttcctgaagggtcaccgggagcagcgcctggcccacctggtcctgagcttcctcaccatgggttatgtctggcaggaaggagaggcgcagcctgcagaggtcctgccaaggaatcttgcccttccatttgtcgaagtctccaggaacttggggctccctcctatcctggtccactcagacttggtgctgacgaactggaccaaaaaagatccagacggattcctggaaattgggaacctggagaccatcatctcatttcctgggggagagagcctgcatggttttatactggtgactgctttggtagagaaagaagcagtgcctgggataaaggctcttgttcaggccacgaatgctatcttgcagcccaaccaggaggccctgctccaagccctgcagcgactgagactgtctattcaggacatcaccaaaaccttaggacagatgcatgatggaaagacaacccagcaatgcctgcagggctgatgtatgaaggagtttcccaagagcccctgaaatactccggcgggagtgcagctcagagcacagtgcttcatgcctttgatgagttcttaggcattcgtcatagcaaggaaagtggtgactttctgtacagaatgagggattacatgcctccttcccataaggccttcatagaagacatccactcagcaccttccctgagggactacatcctgtcctctggacaggaccacttgctgacagcttataaccagtgtgtgcaggccctggcagagctgcggagctatcacatcaccatggtcaccaaatacctcatcacagctgcagccaaggcaaagcatgggaagccaaaccatctcccagggcctcctcaggctttaaaagacaggggcacaggtggaaccgcagttatgagctttcttaagagtgtcagggataagaccttggagtcaatccttcacccacgtggttag

SEQ ID NO: 24 - DNA, 1077 bp, Homo sapiens
  source 1..1077: mol_type = genomic DNA
ggcttatggagccccacagaccgaatgtgaagacagcagtgccattgtctttggaaagctatcacatatctgaagagtatggctttcttcttccagattctctgaaagaacttccagatcattataggccttggatggaaattgccaacaaacttcctcaattgattgatgctcaccagcttcaagctcatgtggacaagatgcccctgctgagctgccagttcctgaagggtcaccgggagcagcgcctggcccacctggtcctgagcttcctcaccatgggttatgtctggcaggaaggagaggcgcagcctgcagaggtcctgccaaggaatcttgcccttccatttgtcgaagtctccaggaacttggggctccctcctatcctggtccactcagacttggtgctgacgaactggaccaaaaaagatccagacggattcctggaaattgggctcttgttcaggccacgaatgctatcttgcagcccaaccaggaggccctgctccaagccctgcagcgactgagactgtctattcaggacatcaccaaaaccttaggacagatgcatgatggaaagacaacccagcaatgcctgcagggctgatgtatgaaggagtttcccaagagcccctgaaatactccggcgggagtgcagctcagagcacagtgcttcatgcctttgatgagttcttaggcattcgtcatagcaaggaaagtggtgactttctgtacagaatgagggattacatgcctccttcccataaggccttcatagaagacatccactcagcaccttccctgagggactacatcctgtcctctggacaggaccacttgctgacagcttataaccagtgtgtgcaggccctggcagagctgcggagctatcacatcaccatggtcaccaaatacctcatcacagctgcagccaaggcaaagcatgggaagccaaaccatctcccagggcctcctcaggctttaaaagacaggggcacaggtggaaccgcagttatgagctttcttaagagtgtcagggataagaccttggagtcaatccttcacccacgtggttag

SEQ ID NO: 25 - DNA, 1096 bp, Homo sapiens
  source 1..1096: mol_type = genomic DNA
ggcttatggagccccacagaccgaatgtgaagacagcagtgccattgtctttggaaagctatcacatatctgaagagtatggctttcttcttccagattctctgaaagaacttccagatcattataggccttggatggaaattgccaacaaacttcctcaattgattgatgctcaccagcttcaagctcatgtggacaagatgcccctgctgagctgccagttcctgaagggtcaccgggagcagcgcctggcccacctggtcctgagcttcctcaccatgggttatgtctggcaggaaggagaggcgcagcctgcagagggaacctggagaccatcatctcatttcctgggggagagagcctgcatggttttatactggtgactgctttggtagagaaagaagcagtgcctgggataaaggctcttgttcaggccacgaatgctatcttgcagcccaaccaggaggccctgctccaagccctgcagcgactgagactgtctattcaggacatcaccaaaaccttaggacagatgcatgattatgtagatccagacatattttatgcaggcatccggatctttctctctggatggaaagacaacccagcaatgcctgcagggctgatgtatgaaggagtttcccaagagcccctgaaatactccggcgggagtgcagctcagagcacagtgcttcatgcctttgatgagttcttaggcattcgtcatagcaaggaaagtggtgactttctgtacagaatgagggattacatgcctccttcccataaggccttcatagaagacatccactcagcaccttccctgagggactacatcctgtcctctggacaggaccacttgctgacagcttataaccagtgtgtgcaggccctggcagagctgcggagctatcacatcaccatggtcaccaaatacctcatcacagctgcagccaaggcaaagcatgggaagccaaaccatctcccagggcctcctcaggctttaaaagacaggggcacaggtggaaccgcagttatgagctttcttaagagtgtcagggataagaccttggagtcaatccttcacccacgtggttag

SEQ ID NO: 26 - DNA, 1229 bp, Homo sapiens
  source 1..1229: mol_type = genomic DNA
ggcttatggagccccacagaccgaatgtgaagacagcagtgccattgtctttggaaagctatcacatatctgaagagtatggctttcttcttccagattctctgaaagaacttccagatcattataggccttggatggaaattgccaacaaacttcctcaattgattgatgctcaccagcttcaagctcatgtggacaagatgcccctgctgagctgccagttcctgaagggtcaccgggagcagcgcctggcccacctggtcctgagcttcctcaccatgggttatgtctggcaggaaggagaggcgcagcctgcagaggtcctgccaaggaatcttgcccttccatttgtcgaagtctccaggaacttggggctccctcctatcctggtccactcagacttggtgctgacgaactggaccaaaaaagatccagacggattcctggaaattgggaacctggagaccatcatctcatttcctgggggagagagcctgcatggttttatactggtgactgctttggtagagaaagaagcagtgcctgggataaaggctcttgttcaggccacgaatgctatcttgcagcccaaccaggaggccctgctccaagccctgcagcgactgagactgtctattcaggacatcaccaaaaccttaggacagatgcatgattatgtagatccagacatattttatgcaggcatctggatctttctctctggatggaaagacaacccagcaatgcctgcagggctgatgtatgaaggagtttcccaagagcccctgaaatactccggcgggagtgcagctcagagcacagtgcttcatgcctttgatgagttcttaggcattcgtcatagcaaggaaagtggtgactttctgtacagaatgagggattacatgcctccttcccataaggccttcatagaagacatccactcagcaccttccctgagggactacatcctgtcctctggacaggaccacttgctgacagcttataaccagtgtgtgcaggccctggcagagctgcggagctatcacatcaccatggtcaccaaatacctcatcacagctgcagccaaggcaaagcatgggaagccaaaccatctcccagggcctcctcaggctttaaaagacaggggcacaggtggaaccgcagttatgagctttcttaagagtgtcagggataagaccttggagtcaatccttcacccacgtggttag

SEQ ID NO: 27 - DNA, 1225 bp, Homo sapiens
  source 1..1225: mol_type = genomic DNA
tatggagccccacagaccgaatgtgaagacagcagtgccattgtctttggaaagctatcacatatctgaagagtatggctttcttcttccagattctctgaaagaacttccagatcattataggccttggatggaaattgccaacaaacttcctcaattgattgatgctcaccagcttcaagctcatgtggacaagatgcccctgctgagctgccagttcctgaagggtcaccgggagcagcgcctggcccacctggtcctgagcttcctcaccatgggttatgtctggcaggaaggagaggcgcagcctgcagaggtcctgccaaggaatcttgcccttccatttgtcgaagtctccaggaacttggggctccctcctgtcctggtccactcagacttggtgctgacgaactggaccaaaaaagatccagacggattcctggaaattgggaacctggagaccatcatctcatttcctgggggagagagcctgcatggttttatactggtgactgctttggtagagaaagaagcagtgcctgggataaaggctcttgttcaggccacgaatgctatcttgcagcccaaccaggaggccctgctccaagccctgcagcgactgagactgtctattcaggacatcaccaaaaccttaggacagatgcatgattatgtagatccagacatattttatgcaggcatccggatctttctctctggatggaaagacaacccagcaatgcctgcagggctgatgtatgaaggagtttcccaagagcccctgaaatactccggcgggagtgcagctcagagcacagtgcttcatgcctttgatgagttcttaggcattcgtcatagcaaggaaagtggtgactttctgtacagaatgagggattacatgcctccttcccataaggccttcatagaagacatccactcagcaccttccctgagggactacatcctgtcctctggacaggaccacttgctgacagcttataaccagtgtgtgcaggccctggcagagctgcggagctaacacatcaccatggtcaccaaatacctcatcacagctgcagccaaggcaaagcatgggaagccaaaccatctcccagggcctcctcaggctttaaaagacaggggcacaggtggaaccgcagttatgagctttcttaagagtgtcagggataagaccttggagtcaatccttcacccacgtggttag

SEQ ID NO: 28 - protein, 407 aa, Homo sapiens
MEPHRPNVKTAVPLSLESYHISEEYGFLLPDSLKELPDHYRPWMEIANKLPQLIDAHQLQAHVDKMPLLSCQFLKGHREQRLAHLVLSFLTMGYVWQEGEAQPAEVLPRNLALPFVEVSRNLGLPPILVHSDLVLTNWTKKDPDGFLEIGNLETIISFPGGESLHGFILVTALVEKEAVPGIKALVQATNAILQPNQEALLQALQRLRLSIQDITKTLGQMHDYVDPDIFYAGIRIFLSGWKDNPAMPAGLMYEGVSQEPLKYSGGSAAQSTVLHAFDEFLGIRHSKESGDFLYRMRDYMPPSHKAFIEDIHSAPSLRDYILSSGQDHLLTAYNQCVQALAELRSYHITMVTKYLITAAAKAKHGKPNHLPGPPQALKDRGTGGTAVMSFLKSVRDKTLESILHPRG

SEQ ID NO: 29 - DNA, 101 bp, Homo sapiens
  source 1..101: mol_type = genomic DNA
gacacgggagttgaggcattaccttggacgtggagaccaggccctcaccagacactgaacttgccagcaccttgatcttggacttcccagcctccagaact

SEQ ID NO: 30 - DNA, 24 bp, synthetic construct
  misc_feature 1..24: note = Primer
  source 1..24: mol_type = other DNA
actctgacctggtgctgacaaact

SEQ ID NO: 31 - DNA, 24 bp, synthetic construct
  misc_feature 1..24: note = Primer
  source 1..24: mol_type = other DNA
tgcaggatgtgaacctctaacgct

SEQ ID NO: 32 - DNA, 24 bp, synthetic construct
  misc_feature 1..24: note = Primer
  source 1..24: mol_type = other DNA
tggagcctcaaagtcagagcatga

SEQ ID NO: 33 - DNA, 24 bp, synthetic construct
  misc_feature 1..24: note = Primer
  source 1..24: mol_type = other DNA
agtttgtcagcaccaggtcagagt

SEQ ID NO: 34 - DNA, 24 bp, synthetic construct
  misc_feature 1..24: note = Primer
  source 1..24: mol_type = other DNA
tgcctgatggcctataaccagtgt

SEQ ID NO: 35 - DNA, 24 bp, synthetic construct
  misc_feature 1..24: note = Primer
  source 1..24: mol_type = other DNA
tgcaggatgtgaacctctaacgct

SEQ ID NO: 36 - DNA, 24 bp, synthetic construct
  misc_feature 1..24: note = Primer
  source 1..24: mol_type = other DNA
aaccacacagaagacacagctgga

SEQ ID NO: 37 - DNA, 24 bp, synthetic construct
  misc_feature 1..24: note = Primer
  source 1..24: mol_type = other DNA
tgagggcaatttccatccaaggct

SEQ ID NO: 38 - DNA, 24 bp, synthetic construct
  misc_feature 1..24: note = Primer
  source 1..24: mol_type = other DNA
accgcacaagtacaaccacacaga

SEQ ID NO: 39 - DNA, 22 bp, synthetic construct
  misc_feature 1..22: note = Primer
  source 1..22: mol_type = other DNA
agccttatgggaaggcggcatg

SEQ ID NO: 40 - DNA, 21 bp, synthetic construct
  misc_feature 1..21: note = Primer
  source 1..21: mol_type = other DNA
actgatttccaacggtccttc

SEQ ID NO: 41 - DNA, 21 bp, synthetic construct
  misc_feature 1..21: note = Primer
  source 1..21: mol_type = other DNA
tcggagctggcggttctcgat

SEQ ID NO: 42 - DNA, 19 bp, synthetic construct
  misc_feature 1..19: note = Primer
  source 1..19: mol_type = other DNA
aaggtgctgccaagatctc

SEQ ID NO: 43 - DNA, 18 bp, synthetic construct
  misc_feature 1..18: note = Primer
  source 1..18: mol_type = other DNA
cttcatgccttcgatgag

SEQ ID NO: 44 - DNA, 24 bp, synthetic construct
  misc_feature 1..24: note = Primer
  source 1..24: mol_type = other DNA
agagaaagaagcagtgcctgggat

SEQ ID NO: 45 - DNA, 24 bp, synthetic construct
  misc_feature 1..24: note = Primer
  source 1..24: mol_type = other DNA
taagctgtcagcaagtggtcctgt

SEQ ID NO: 46 - DNA, 24 bp, synthetic construct
  misc_feature 1..24: note = Primer
  source 1..24: mol_type = other DNA
tgagacactgcgcaactaactgga

SEQ ID NO: 47 - DNA, 24 bp, synthetic construct
  misc_feature 1..24: note = Primer
  source 1..24: mol_type = other DNA
atcccaggcactgcttctttctct

SEQ ID NO: 48 - DNA, 24 bp, synthetic construct
  misc_feature 1..24: note = Primer
  source 1..24: mol_type = other DNA
acaggaccacttgctgacagctta

SEQ ID NO: 49 - DNA, 24 bp, synthetic construct
  misc_feature 1..24: note = Primer
  source 1..24: mol_type = other DNA
acgtgggtgaaggattgactccaa

SEQ ID NO: 50 - DNA, 23 bp, synthetic construct
  misc_feature 1..23: note = Primer
  source 1..23: mol_type = other DNA
tgttggcaatttccatccaaggc

SEQ ID NO: 51 - DNA, 24 bp, synthetic construct
  misc_feature 1..24: note = Primer
  source 1..24: mol_type = other DNA
aaccacgtgggtgaaggattgact

SEQ ID NO: 52 - DNA, 22 bp, synthetic construct
  misc_feature 1..22: note = Primer
  source 1..22: mol_type = other DNA
ttcagtgacactttccatgcag

SEQ ID NO: 53 - DNA, 22 bp, synthetic construct
  misc_feature 1..22: note = Primer
  source 1..22: mol_type = other DNA
atggagccccacagaccgaatg

SEQ ID NO: 54 - DNA, 22 bp, synthetic construct
  misc_feature 1..22: note = Primer
  source 1..22: mol_type = other DNA
gaagggcaagattccttggcag

SEQ ID NO: 55 - DNA, 22 bp, synthetic construct
  misc_feature 1..22: note = Primer
  source 1..22: mol_type = other DNA
attgatgctcaccagcttcaag

SEQ ID NO: 56 - DNA, 22 bp, synthetic construct
  misc_feature 1..22: note = Primer
  source 1..22: mol_type = other DNA
cttcatacatcagccctgcagg

SEQ ID NO: 57 - DNA, 22 bp, synthetic construct
  misc_feature 1..22: note = Primer
  source 1..22: mol_type = other DNA
gtagagaaagaagcagtgcctg

SEQ ID NO: 58 - DNA, 22 bp, synthetic construct
  misc_feature 1..22: note = Primer
  source 1..22: mol_type = other DNA
ggcatccggatctttctctctg

SEQ ID NO: 59 - DNA, 23 bp, synthetic construct
  misc_feature 1..23: note = Primer
  source 1..23: mol_type = other DNA
ctaaccacgtgggtgaaggattg

SEQ ID NO: 60 - DNA, 18 bp, synthetic construct
  misc_feature 1..18: note = Primer
  source 1..18: mol_type = other DNA
ggtactgccatgctgagc

SEQ ID NO: 61 - DNA, 21 bp, synthetic construct
  misc_feature 1..21: note = Primer
  source 1..21: mol_type = other DNA
aagctgcccgttctcaatcag

SEQ ID NO: 62 - DNA, 22 bp, synthetic construct
  misc_feature 1..22: note = Primer
  source 1..22: mol_type = other DNA
ctcgtatgtcatgggcccattg

SEQ ID NO: 63 - DNA, 22 bp, synthetic construct
  misc_feature 1..22: note = Primer
  source 1..22: mol_type = other DNA
gaagttccggtgggctggaggc

SEQ ID NO: 64 - DNA, 21 bp, synthetic construct
  misc_feature 1..21: note = Primer
  source 1..21: mol_type = other DNA
ggtgctgccccgcaataatgc

SEQ ID NO: 65 - DNA, 20 bp, synthetic construct
  misc_feature 1..20: note = Primer
  source 1..20: mol_type = other DNA
ccagagtcttgatgtccttc

SEQ ID NO: 66 - DNA, 21 bp, synthetic construct
  misc_feature 1..21: note = Primer
  source 1..21: mol_type = other DNA
cctcaaatgtggaaagcagag

SEQ ID NO: 67 - DNA, 28 bp, synthetic construct
  misc_feature 1..28: note = Primer
  source 1..28: mol_type = other DNA
atccggtaccatggagcctcaaagtcag

SEQ ID NO: 68 - DNA, 24 bp, synthetic construct
  misc_feature 1..24: note = Primer
  source 1..24: mol_type = other DNA
gttgagctctccaggaacttggga

SEQ ID NO: 69 - DNA, 27 bp, synthetic construct
  misc_feature 1..27: note = Primer
  source 1..27: mol_type = other DNA
agtgttgaattcctacaggaaatgagg

SEQ ID NO: 70 - DNA, 27 bp, synthetic construct
  misc_feature 1..27: note = Primer
  source 1..27: mol_type = other DNA
ctgaaagcttataaccagtgtgtggag

SEQ ID NO: 71 - DNA, 21 bp, synthetic construct
  misc_feature 1..21: note = Primer
  source 1..21: mol_type = other DNA
catcctgtacacgtgagctcg

SEQ ID NO: 72 - DNA, 24 bp, synthetic construct
  misc_feature 1..24: note = Primer
  source 1..24: mol_type = other DNA
tggagagctcaacaaaaggaatgg

SEQ ID NO: 73 - DNA, 30 bp, synthetic construct
  misc_feature 1..30: note = Primer
  source 1..30: mol_type = other DNA
ctgtaggaattcaacactttccttgcaatg

SEQ ID NO: 74 - DNA, 24 bp, synthetic construct
  misc_feature 1..24: note = Primer
  source 1..24: mol_type = other DNA
tataagctttcaggcagtccccag

SEQ ID NO: 75 - DNA, 21 bp, synthetic construct
  misc_feature 1..21: note = Primer
  source 1..21: mol_type = other DNA
gaagacacagctggaaagctc

SEQ ID NO: 76 - DNA, 20 bp, synthetic construct
  misc_feature 1..20: note = Primer
  source 1..20: mol_type = other DNA
ccatgctgacctccaggctc

SEQ ID NO: 77 - DNA, 28 bp, synthetic construct
  misc_feature 1..28: note = Primer
  source 1..28: mol_type = other DNA
atccggtaccatggcactcagtaaaata

SEQ ID NO: 78 - DNA, 24 bp, synthetic construct
  misc_feature 1..24: note = Primer
  source 1..24: mol_type = other DNA
gaagtgtacaagctgcccacactg

SEQ ID NO: 79 - DNA, 22 bp, synthetic construct
  misc_feature 1..22: note = Primer
  source 1..22: mol_type = other DNA
gaggatgcatgactttgtggac

SEQ ID NO: 80 - DNA, 23 bp, synthetic construct
  misc_feature 1..23: note = Primer
  source 1..23: mol_type = other DNA
agcttgtacacttcttctcgaag

SEQ ID NO: 81 - DNA, 24 bp, synthetic construct
  misc_feature 1..24: note = Primer
  source 1..24: mol_type = other DNA
gtcatgcatcctcttaaaaataac

SEQ ID NO: 82 - DNA, 33 bp, synthetic construct
  misc_feature 1..33: note = Primer
  source 1..33: mol_type = other DNA
gtcgcgctagcaaactcaatggtgatggtgatg

SEQ ID NO: 83 - DNA, 23 bp, synthetic construct
  misc_feature 1..23: note = Target sequence
  source 1..23: mol_type = other DNA
aatgtgaagacagcagtgccatt

SEQ ID NO: 84 - DNA, 23 bp, synthetic construct
  misc_feature 1..23: note = Target sequence
  source 1..23: mol_type = other DNA
aagacagcagtgccattgtcttt

SEQ ID NO: 85 - DNA, 23 bp, synthetic construct
  misc_feature 1..23: note = Target sequence
  source 1..23: mol_type = other DNA
aattgattgatgctcaccagctt

SEQ ID NO: 86 - DNA, 23 bp, synthetic construct
  misc_feature 1..23: note = Target sequence
  source 1..23: mol_type = other DNA
aacctggagaccatcatctcatt

SEQ ID NO: 87 - DNA, 23 bp, synthetic construct
  misc_feature 1..23: note = Target sequence
  source 1..23: mol_type = other DNA
aaccttaggacagatgcatgatt

SEQ ID NO: 88 - DNA, 23 bp, synthetic construct
  misc_feature 1..23: note = Target sequence
  source 1..23: mol_type = other DNA
aaggagtttcccaagagcccctt

SEQ ID NO: 89 - DNA, 23 bp, synthetic construct
  misc_feature 1..23: note = Target sequence
  source 1..23: mol_type = other DNA
aatgagggattacatgcctcctt

SEQ ID NO: 90 - DNA, 23 bp, synthetic construct
  misc_feature 1..23: note = Target sequence
  source 1..23: mol_type = other DNA
aagagtgtcagggataagacctt

SEQ ID NO: 91 - RNA, 21 nt, synthetic construct
  misc_feature 1..21: note = siRNA
  source 1..21: mol_type = other RNA
tgtgaagacagcagtgccatt

SEQ ID NO: 92 - RNA, 21 nt, synthetic construct
  misc_feature 1..21: note = siRNA
  source 1..21: mol_type = other RNA
gacagcagtgccattgtcttt

SEQ ID NO: 93 - RNA, 21 nt, synthetic construct
  misc_feature 1..21: note = siRNA
  source 1..21: mol_type = other RNA
ttgattgatgctcaccagctt

SEQ ID NO: 94 - RNA, 21 nt, synthetic construct
  misc_feature 1..21: note = siRNA
  source 1..21: mol_type = other RNA
cctggagaccatcatctcatt

SEQ ID NO: 95 - RNA, 21 nt, synthetic construct
  misc_feature 1..21: note = siRNA
  source 1..21: mol_type = other RNA
ccttaggacagatgcatgatt

SEQ ID NO: 96 - RNA, 21 nt, synthetic construct
  misc_feature 1..21: note = siRNA
  source 1..21: mol_type = other RNA
ggagtttcccaagagcccctt

SEQ ID NO: 97 - RNA, 21 nt, synthetic construct
  misc_feature 1..21: note = siRNA
  source 1..21: mol_type = other RNA
tgagggattacatgcctcctt

SEQ ID NO: 98 - RNA, 21 nt, synthetic construct
  misc_feature 1..21: note = siRNA
  source 1..21: mol_type = other RNA
gagtgtcagggataagacctt

SEQ ID NO: 99 - DNA, 25 bp, synthetic construct
  misc_feature 1..25: note = siRNA
  source 1..25: mol_type = other DNA
caaacttcctcaattgattgatgct

SEQ ID NO: 100 - DNA, 25 bp, synthetic construct
  misc_feature 1..25: note = siRNA
  source 1..25: mol_type = other DNA
gtgccattgtctttggaaagctatc

SEQ ID NO: 101 - DNA, 25 bp, synthetic construct
  misc_feature 1..25: note = siRNA
  source 1..25: mol_type = other DNA
gtgccattgtctttggaaagctatc

SEQ ID NO: 102 - RNA, 25 nt, synthetic construct
  misc_feature 1..25: note = siRNA
  source 1..25: mol_type = other RNA
caaggaaagtggtgactttctgtac

SEQ ID NO: 103 - DNA, 25 bp, synthetic construct
  misc_feature 1..25: note = siRNA
  source 1..25: mol_type = other DNA
gcagctcagagcacagtgcttcatg

SEQ ID NO: 104 - RNA, 25 nt, synthetic construct
  misc_feature 1..25: note = siRNA
  source 1..25: mol_type = other RNA
gcatgggaagccaaaccatctccca

SEQ ID NO: 105 - DNA, 25 bp, synthetic construct
  misc_feature 1..25: note = siRNA
  source 1..25: mol_type = other DNA
agagaaagaagcagtgcctgggata

SEQ ID NO: 106 - DNA, 25 bp, synthetic construct
  misc_feature 1..25: note = siRNA
  source 1..25: mol_type = other DNA
ccttaggacagatgcatgattatgt

SEQ ID NO: 107 - RNA, 21 nt, synthetic construct
  misc_feature 1..21: note = siRNA
  source 1..21: mol_type = other RNA
aggcactgctgtcttcacatt

SEQ ID NO: 108 - RNA, 21 nt, synthetic construct
  misc_feature 1..21: note = siRNA
  source 1..21: mol_type = other RNA
agacaatggcactgctgtctt

SEQ ID NO: 109 - RNA, 21 nt, synthetic construct
  misc_feature 1..21: note = siRNA
  source 1..21: mol_type = other RNA
gctggtgagcatcaatcaatt

SEQ ID NO: 110 - RNA, 21 nt, synthetic construct
  misc_feature 1..21: note = siRNA
  source 1..21: mol_type = other RNA
tgacatgatggtctccaggtt

SEQ ID NO: 111 - RNA, 21 nt, synthetic construct
  misc_feature 1..21: note = siRNA
  source 1..21: mol_type = other RNA
tcatgcatctgtccgaaggtt

SEQ ID NO: 112 - RNA, 21 nt, synthetic construct
  misc_feature 1..21: note = siRNA
  source 1..21: mol_type = other RNA
ggggctcttgggaaactcctt

SEQ ID NO: 113 - RNA, 21 nt, synthetic construct
  misc_feature 1..21: note = siRNA
  source 1..21: mol_type = other RNA
ggaggcatgtaatccctcatt

SEQ ID NO: 114 - RNA, 21 nt, synthetic construct
  misc_feature 1..21: note = siRNA
  source 1..21: mol_type = other RNA
ggtcttatccctgacactctt

SEQ ID NO: 115 - RNA, 27 nt, synthetic construct
  misc_feature 1..27: note = siRNA
  source 1..27: mol_type = other RNA
agcatcaatcaattgaggaagtttgtt

SEQ ID NO: 116 - RNA, 27 nt, synthetic construct
  misc_feature 1..27: note = siRNA
  source 1..27: mol_type = other RNA
gatagctttccaaagacaatggcactg

SEQ ID NO: 117 - RNA, 27 nt, synthetic construct
  misc_feature 1..27: note = siRNA
  source 1..27: mol_type = other RNA
ctccaaggtcttatccctgacactctt

SEQ ID NO: 118 - RNA, 27 nt, synthetic construct
  misc_feature 1..27: note = siRNA
  source 1..27: mol_type = other RNA
gtacagaaagtcaccactttccttgct

SEQ ID NO: 119 - RNA, 27 nt, synthetic construct
  misc_feature 1..27: note = siRNA
  source 1..27: mol_type = other RNA
catgaagcactgtgctctgagctgcac

SEQ ID NO: 120 - RNA, 27 nt, synthetic construct
  misc_feature 1..27: note = siRNA
  source 1..27: mol_type = other RNA
tgggagatggtttggcttcccatgctt

SEQ ID NO: 121 - RNA, 27 nt, synthetic construct
  misc_feature 1..27: note = siRNA
  source 1..27: mol_type = other RNA
tatcccaggcactgcttctttctctac

SEQ ID NO: 122 - RNA, 27 nt, synthetic construct
  misc_feature 1..27: note = siRNA
  source 1..27: mol_type = other RNA
tatcccaggcactgcttctttctctac

SEQ ID NO: 123 - RNA, 27 nt, synthetic construct
  misc_feature 1..27: note = siRNA
  source 1..27: mol_type = other RNA
acataatcatgcatctgtcctaaggtt

SEQ ID NO: 124 - RNA, 21 nt, synthetic construct
  misc_feature 1..21: note = siRNA
  source 1..21: mol_type = other RNA
aaggaacccagaaggaccgtt

SEQ ID NO: 125 - RNA, 21 nt, synthetic construct
  misc_feature 1..21: note = siRNA
  source 1..21: mol_type = other RNA
cctggaaaccatcatctcatt

SEQ ID NO: 126 - RNA, 21 nt, synthetic construct
  misc_feature 1..21: note = siRNA
  source 1..21: mol_type = other RNA
tgagggactacatgccgcctt

SEQ ID NO: 127 - RNA, 21 nt, synthetic construct
  misc_feature 1..21: note = siRNA
  source 1..21: mol_type = other RNA
aaggtgctgccaagatctctt

SEQ ID NO: 128 - DNA, 25 bp, synthetic construct
  misc_feature 1..25: note = siRNA
  source 1..25: mol_type = other DNA
ccagaaggaccgttggaaatcagta

SEQ ID NO: 129 - RNA, 25 nt, synthetic construct
  misc_feature 1..25: note = siRNA
  source 1..25: mol_type = other RNA
gaagtactctggaggaagtgcagcc

SEQ ID NO: 130 - RNA, 25 nt, synthetic construct
  misc_feature 1..25: note = siRNA
  source 1..25: mol_type = other RNA
caaggaaagtgttggctttctacac

SEQ ID NO: 131 - RNA, 25 nt, synthetic construct
  misc_feature 1..25: note = siRNA
  source 1..25: mol_type = other RNA
ccatgggattcgtctggcaggaggg

SEQ ID NO: 132 - RNA, 25 nt, synthetic construct
  misc_feature 1..25: note = siRNA
  source 1..25: mol_type = other RNA
gtactgccatgctgagcttcttgaa

SEQ ID NO: 133 - DNA, 25 bp, synthetic construct
  misc_feature 1..25: note = siRNA
  source 1..25: mol_type = other DNA
ctatgaaggtgctgccacagagcct

SEQ ID NO: 134 - RNA, 25 nt, synthetic construct
  misc_feature 1..25: note = siRNA
  source 1..25: mol_type = other RNA
gtgtcagggagaagaccatggaggc

SEQ ID NO: 135 - RNA, 25 nt, synthetic construct
  misc_feature 1..25: note = siRNA
  source 1..25: mol_type = other RNA
gtcagagcatgacgctggaggtgcc

SEQ ID NO: 136 - RNA, 21 nt, synthetic construct
  misc_feature 1..21: note = siRNA
  source 1..21: mol_type = other RNA
cggtccttctgggttcctttt

SEQ ID NO: 137 - RNA, 21 nt, synthetic construct
  misc_feature 1..21: note = siRNA
  source 1..21: mol_type = other RNA
tgtgatgatggtttccaggtt

SEQ ID NO: 138 - RNA, 21 nt, synthetic construct
  misc_feature 1..21: note = siRNA
  source 1..21: mol_type = other RNA
ggcggcatgtagtccctcatt

SEQ ID NO: 139 - RNA, 21 nt, synthetic construct
  misc_feature 1..21: note = siRNA
  source 1..21: mol_type = other RNA
gagatcttggcagcacctttt

SEQ ID NO: 140 - RNA, 27 nt, synthetic construct
  misc_feature 1..27: note = siRNA
  source 1..27: mol_type = other RNA
tactgatttccaacggtccttctgggt

SEQ ID NO: 141 - RNA, 27 nt, synthetic construct
  misc_feature 1..27: note = siRNA
  source 1..27: mol_type = other RNA
ggctgcacttcctccagagtacttcag

SEQ ID NO: 142 - RNA, 27 nt, synthetic construct
  misc_feature 1..27: note = siRNA
  source 1..27: mol_type = other RNA
gtgtagaaagccaacactttccttgca

SEQ ID NO: 143 - RNA, 27 nt, synthetic construct
  misc_feature 1..27: note = siRNA
  source 1..27: mol_type = other RNA
ccctcctgccagacgaatcccatggtg

SEQ ID NO: 144 - RNA, 27 nt, synthetic construct
  misc_feature 1..27: note = siRNA
  source 1..27: mol_type = other RNA
ttcaagaagctcagcatggcagtaccc

SEQ ID NO: 145 - RNA, 27 nt, synthetic construct
  misc_feature 1..27: note = siRNA
  source 1..27: mol_type = other RNA
aggctctgtggcagcaccttcatagac

SEQ ID NO: 146 - RNA, 27 nt, synthetic construct
  misc_feature 1..27: note = siRNA
  source 1..27: mol_type = other RNA
gcctccatggtcttctccctgacactc

SEQ ID NO: 147 - RNA, 27 nt, synthetic construct
  misc_feature 1..27: note = siRNA
  source 1..27: mol_type = other RNA
ggcacctccagcgtcatgctctgactt

SEQ ID NO: 148 - DNA, 38 bp, synthetic construct
  misc_feature 1..38: note = Primer
  source 1..38: mol_type = other DNA
actggcggccgcatgccgaatctattaccattactgcc

SEQ ID NO: 149 - DNA, 34 bp, synthetic construct
  misc_feature 1..34: note = Primer
  source 1..34: mol_type = other DNA
actgggtaccacaccttcatagaccagccccaca

SEQ ID NO: 150 - DNA, 40 bp, synthetic construct
  misc_feature 1..40: note = Primer
  source 1..40: mol_type = other DNA
actgcgtacgcatatgtgtggggctggtctatgaaggtgt

SEQ ID NO: 151 - DNA, 35 bp, synthetic construct
  misc_feature 1..35: note = Primer
  source 1..35: mol_type = other DNA
actggtcgacgggatatagcacaagaacagctaag

SEQ ID NO: 152 - DNA, 37 bp, synthetic construct
  misc_feature 1..37: note = Primer
  source 1..37: mol_type = other DNA
actgggcgcgcccttagctgttcttgtgctatatccc

SEQ ID NO: 153 - DNA, 38 bp, synthetic construct
  misc_feature 1..38: note = Primer
  source 1..38: mol_type = other DNA
actgcccgggggtgtctgtgagattttgagaatagtcc

SEQ ID NO: 154 - DNA, 865 bp, synthetic construct
  misc_feature 1..865: note = Synthetic Sequence
  source 1..865: mol_type = other DNA
atgccgaatctattaccattactgccaagtcccattgggagaaaaactaaagtgtgcattcgtgcatgtgtgcgtgtacgtgtgtgtctagaactgaaaattttatttccttgcaagtatcagactagagttttcctggcctgctaggtcctctgttgcctctccccaccatgtccccaatttgactcctgacacagcactggcacttggcatttttctagaattacacattttcctgacttttctcttgactaccgccagaggaaatttctcttaaaggggctcctgtggctgtgctcaatctagtcggatgagtcagggaaattcccacattaaagctaaactgatcagtggccttaatcccatctgaaaagtcctcctgccctgtcctgtaataaatatatcatggtgaccggaacagctcatagtattaagagtcctaggaattcggttgggaaccttggggagctatttttagaatcctaccctccagtgtctttcaaaggaatcatttctacttcctcttggcagagacttgataagaaccagaaggggactttgtttgcatcgacataagttccgggcaatgacactttttatctggtatttggcataaaggccatccctccacttcaaatgctgagactgtttactgtgccgctaagtggctgtacaagatcctaaatgtagctgtagtttcaacaaacatctggattgttgggagtttccagtagacttctctttaaaatgtcagcctcgctattctgcatctatcccgagtttctcatttgcttctttaaaacacagttttatttaagagtggatgtcctgtggaaatgagatgtattccctccagttcccagcc

SEQ ID NO: 155 - DNA, 948 bp, synthetic construct
  misc_feature 1..948: note = Synthetic Sequence
  source 1..948: mol_type = other DNA
tgagtgtatgctcgtgtatgtatgtgagcactgcaggtgtctgcatggtgtgccatggtgctccactgggcttcatgctgtgctcccctaggcttcagtcaagtcaagactaggtcaagtcatggagggtaaacagaagagagagagagcagaaaatgagggacacaggaagggtagagggggaaagagagggttgtgaagctcacgcgtgatcaggagccccaggctttcttcttccagccgcccataggctctggtgcccacaacattggttacaaccccgctctccatcatcatgcttgtcatcactgctgtgacaaaccgcttaataatggttctcacggaacattaaaagccaagccaagtttaacacctcgaacatttccaagtgttatggggaataacagttaagtgtctgggtgtgcttgtgtgtaattgggaatctgtagtggtggggttaccagtgtcaggccacagtgtttgtgatgagcagaggggtcggggtctttctcagatcccttatcttgtcctgtcaatggtggtgatgtaataggtgcacgcctgtgacagagctgtttaaagcattgtaagaccaatgagtaaagttcctacccttgcttctcctttaagtgaggcagaaaaaggctccaccatgacgtggtgtaaagatgaagtcaatctaatacttccttggatactctagcaagcttcattcacactttttatttcttcctcttcctcttcctcttcctcttcctcttcctcttcctcttcccctcctcctcttcctcctcctcttcctcctcctcttcctcctcctcctcttcctccttctcctcttcctcctccccctccccttcccctgtgctcctcattattattattattgctcctgtctaggtggaaggacaatccagccatgcctgtggggctggtctatgaaggtgt

SEQ ID NO: 156 - DNA, 700 bp, synthetic construct
  misc_feature 1..700: note = Synthetic Sequence
  source 1..700: mol_type = other DNA
ggtaccaaaggccgcaatggccaaacctgcaggaagtaccagagctccctaggttctagaaccggtgacgtcaagctcgaataacttcgtataatgtatgctatacgaagttatcctgcaggtcgatcgagaccgtacgctatgtgtggggctggtctatgaaggtgttgccacagagcctctgaagtactctggaggaagtgcagcccagagctccgtgcttcatgccttcgatgagttcctgggcattgagcattgcaaggaaagtggtgagcagcagtctgatctcacctatgctttgatgggacagcgaggtagactagggagacatctctagcaactgataaagacgggtgtaaatgaaaatgtcctgaagtttatccttgcctaagccagcaggcagctgtgtgcatgtgccctctcttacactgagttagtcagtattggggcatcggatcttattagggtcttccaacagtcctgtgacctgggttgttcactgtcctgttggctggggtcttttatccgcagattcccctttctacaatgaggtgataatgtcacattgaaaggccagtctggagcagcaagtgatagtgctgaacttctctgctaaagcctttcccatgaaatggcccagcctcccactgaatctatgtggaccaggcgagggagcccatcgctttgaagcctttaaaat

SEQ ID NO: 157 - DNA, 803 bp, synthetic construct
  misc_feature 1..803: note = Synthetic Sequence
  misc_feature 744: note = n = a, c, t, g
  source 1..803: mol_type = other DNA
cagaaatgaacatttgagcatttggcagctataacaaaagcccgacaaggctgagggagagccctatcaagcatttctggtacctgagtgtttggaacagtgggcaaaccctcccaaatgtctgcctcgagctaacgtatttctcccggctgtttctttcagttggctttctacacagaatgagggactacatgccgccttcccataaggctttcctggaagatctccacgtagctccttctctgagagactacatactggcctctggtcctggggactgcctgatggcctataaccagtgtgtggaggccctgggagagctgcgcagttaccacatcaatgtcgtggccagatacattatctccgctgccaccagggccaggagcagggggctaactaatccctcaccccatgccttggaagacaggggcactgggggtactgccatgctgagcttcttgaagagtgtcagggagaagaccatggaggccctcctgtgtcctggtgcttagcagtcatgtcctgcaccctaacacttagatgttctcatcctgcatcccagcgttagaggttcacatcctgcatcctagtgcttagctgttcttgtgctatatcccgtcgacgaagttcctatactttctagagaataggaacttcggatccacgattcgagggcccctgcaggtcaattctaccgggtaggggaggcgcttttcccaaggcagtctggagcatgcgctttancagccccgctgggcacttggcgctacacaagtggcctctggcctcgcacacattccaca

SEQ ID NO: 158 - DNA, 575 bp, synthetic construct
  misc_feature 1..575: note = Synthetic Sequence
  source 1..575: mol_type = other DNA
atcccagcgcttagcagtcatgtcctgcatcctagtgcttagcattttatatccagcatcttagtgcttagagattcacatcctgcatcctagagcttagcattttatatccagcatccttgtgcgtatcagctatgttttgtatcctgcttagcagttaacatcctgcatcctagtacttatctgttctcatcctgcatcctagagcttagcagtcaggtcccgtgggagcaagaaccagggtctgagctctgtctgagcccaagcatggctttactgctttgttaattgtggctcccacctccaccccaccccagccagtttgcttgctagaagcctttctgcactgcctaatccccctgcctcacagcagagagctgcagccatgacctcctcattcagtattaggtggacaagtcggagatacccaaactcaattttaaaagaatcaagttgcttttggggcatgttacttcatcttttcttaccctgggcctctatgacctcctcattcagtattaggtggacaagtcggagatacccaaactcaattttaaaagaatcaagttgctttt

SEQ ID NO: 159 - DNA, 753 bp, synthetic construct
  misc_feature 1..753: note = Synthetic Sequence
  source 1..753: mol_type = other DNA
agctctaagagaaggaatacagcttgggacagagtttgttttgaagtggggttcaggtgcatttcctgggtgtgtccctcatttgggggtgttaggaggcagacaatgctaagggcatggtttgatatggtagactgaccatcctggggtcccttcagcttgtgacactcgctgtactgctggactctgctgagccctttgaagccaggactcctcctctgctgcaggagtgcagtgtccttcttgctgtatgaagctgggacaatgctctttggccttcatactggacatcccattgagaagcttgtcactctgtagagaatagacatcgcccccttgtggttgtgaggctgcccaggacttactgcggggggggggggggcatgttgtccagcataaggagagaagaccccactgcatgctgctgggaaaaggaaagtaacgttcagagtagtttctactggctgcctgcgctctcacgcctgtaagaacaaacgtcctaatgtctgcatgtggaggaaggagccaggggtgcttagggtgctgttggtcccaccaatgtactactcatctggaagaccttgtcttggttttcttgccactgggacaaagtgggattgggtatcagtctccccatgtcgggcaaatgtacttgaacaagcagcatggtggaccagacatgggactattctcaaaatctcacagacacccccggggggcatgttacttcatcttttcttaccctgggcctct

SEQ ID NO: 160 - DNA, 453 bp, synthetic construct
  misc_feature 1..453: note = Synthetic Sequence
  source 1..453: mol_type = other DNA
ctagggagtcaggaagtcacactggtgaagagtgggggtgtgggatgaccacactgaggagtcacacttgaggaaggggggtggccacactgaggagtgtctgaaagcattgagactgtacatctcaggttatcagggcttcagagaaaacagacagggagaagagacaagaactggattctgtgcgcaaagggggaaaagcaagcagatgtgaagggtgtgctgttagagtttatctaaagatgtttctttcagaaataaagagatataagctttaatttggatgaaataaatgtggtctaatttcccagaatgtagaggaactcactaatgtagcaagattggcctttcaaagcagaccaaagacattgagaattaagatagctatgatggcgtgcttccttaggtggaagtcctatatggaatcccatactccccaaatgtgactggtcg

SEQ ID NO: 161 - DNA, 361 bp, synthetic construct
  misc_feature 1..361: note = Synthetic Sequence
  source 1..361: mol_type = other DNA
aggcaggagggtcaggagttcaaggccagcctagtatacatgcaacagtgtctcaaaaatcaaaacagagaggagggcaggaaaggagaagggagccagggagagaagggaagaggaagggagggagggggaaatggagggagggggagatggagggagggggagatggagggagggggagatggagggagggggagatggagggagggggagatggagggagggggatgggaaagagggtttacagccctggtttatcttgaacagaatccttactttgtccctcagagtgacaggactgaaaagattgtcccaggattttggctgcaaagcaaggtccactcagagaccacagagctcg

SEQ ID NO: 162 - DNA, 10543 bp, synthetic construct
  misc_feature 1..10543: note = Synthetic Sequence
  source 1..10543: mol_type = other DNA
aagctttactcgtaaagcgagttgaaggatcatatttagttgcgtttatgagataagattgaaagcacgtgtaaaatgtttcccgcgcgttggcacaactatttacaatgcggccaagttataaaagattctaatctgatatgttttaaaacacctttgcggcccgagttgtttgcgtacgtgactagcgaagaagatgtgtggaccgcagaacagatagtaaaacaaaaccctagtattggagcaataatcgatttaaccaacacgtctaaatattatgatggtgtgcattttttgcgggcgggcctgttatacaaaaaaattcaagtacctggccagactttgccgcctgaaagcatagttcaagaatttattgacacggtaaaagaatttacagaaaagtgtcccggcatgttggtgggcgtgcactgcacacacggtattaatcgcaccggttacatggtgtgcagatatttaatgcacaccctgggtattgcgccgcaggaagccatagatagattcgaaaaagccagaggtcacaaaattgaaagacaaaattacgttcaagatttattaatttaattaatattatttgcattctttaacaaatactttatcctattttcaaattgttgcgcttcttccagcgaaccaaaactatgcttcgcttgctccgtttagcttgtagccgatcagtggcgttgttccaatcgacggtaggattaggccggatattctccaccacaatgttggcaacgttgatgttacgtttatgcttttggttttccacgtacgtcttttggccggtaatagccgtaaacgtagtgccgtcgcgcgtcacgcacaacaccggatgtttgcgcttgtccgcggggtattgaaccgcgcgatccgacaaatccaccactttggcaactaaatcggtgacctgcgcgtcttttttctgcattatttcgtctttcttttgcatggtttcctggaagccggtgtacatgcggtttagatcagtcatgacgcgcgtgacctgcaaatctttggcctcgatctgcttgtccttgatggcaacgatgcgttcaataaactcttgttttttaacaagttcctcggttttttgcgccaccaccgcttgcagcgcgtttgtgtgctcggtgaatgtcgcaatcagcttagtcaccaactgtttgctctcctcctcccgttgtttgatcgcgggatcgtacttgccggtgcagagcacttgaggaattacttcttctaaaagccattcttgtaattctatggcgtaaggcaatttggacttcataatcagctgaatcacgccggatttagtaatgagcactgtatgcggctgcaaatacagcgggtcgccccttttcacgacgctgttagaggtagggcccccattttggatggtctgctcaaataacgatttgtatttattgtctacatgaacacgtatagctttatcacaaactgtatattttaaactgttagcgacgtccttggccacgaaccggacctgttggtcgcgctctagcacgtaccgcaggttgaacgtatcttctccaaatttaaattctccaattttaacgcgagccattttgatacacgtgtgtcgattttgcaacaactattgttttttaacgcaaactaaacttattgtggtaagcaataattaaatatgggggaacatgcgccgctacaacactcgtcgttatgaacgcagacggcgccggtctcggcgcaagcggctaaaacgtgttgcgcgttcaacgcggcaaacatcgcaaaagccaatagtacagttttgatttgcatattaacggcgattttttaaattatcttatttaataaatagttatgacgcctacaactccccgcccgcgttgactcgctgcacctcgagcagttcgttgacgccttcctccgtgtggccgaacacgtcgagcgggtggtcgatgaccagcggcgtgccgcacgcgacgcacaagtatctgtacaccgaatgatcgtcgggcgaaggcacgtcggcctccaagtggcaatattggcaaattcgaaaatatatacagttgggttgtttgcgcatatctatcgtggcgttgggcatgtacgtccgaacgttgatttgcatgcaagccgaaattaaatcattgcgattagtgcgattaaaacgttgtacatcctcgcttttaatcatgccgtcgattaaatcgcgcaatcgagtcaagtgatcaaagtgtggaataatgttttctttgtattcccgagtcaagcgcagcgcgtattttaacaaactagccatcttgtaagttagtttcatttaatgcaactttatccaataatatattatgtatcgcacgtcaagaattaacaatgcgcccgttgtcgcatctcaacacgactatgatagagatcaaataaagcgcgaattaaatagcttgcgacgcaacgtgcacgatctgtgcacgcgttccggcacgagctttgattgtaataagtttttacgaagcgatgacatgacccccgtagtgacaacgatcacgcccaaaagaactgccgactacaaaattaccgagtatgtcggtgacgttaaaactattaagccatccaatcgaccgttagtcgaatcaggaccgctggtgcgagaagccgcgaagtatggcgaatgcatcgtataacgtgtggagtccgctcattagagcgtcatgtttagacaagaaagctacatatttaattgatcccgatgattttattgataaattgaccctaactccatacacggtattctacaatggcggggttttggtcaaaatttccggactgcgattgtacatgctgttaacggctccgcccactattaatgaaattaaaaattccaattttaaaaaacgcagcaagagaaacatttgtatgaaagaatgcgtagaaggaaagaaaaatgtcgtcgacatgctgaacaacaagattaatatgcctccgtgtataaaaaaaatattgaacgatttgaaagaaaacaatgtaccgcgcggcggtatgtacaggaagaggtttatactaaactgttacattgcaaacgtggtttcgtgtgccaagtgtgaaaaccgatgtttaatcaaggctctgacgcatttctacaaccacgactccaagtgtgtgggtgaagtcatgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgctaaatttgttttttattaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcctcataaaaattaacatagttattatcgtatccatatatgtatctatcgtatagagtaaattttttgttgtcataaatatatatgtcttttttaatggggtgtatagtaccgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccattgtaatgagacgcacaaactaatatcacaaactggaaatgtctatcaatatatagttgctgatatcatggagataattaaaatgataaccatctcgcaaataaataagtattttactgttttcgtaacagttttgtaataaaaaaacctataaatattccggattattcataccgtcccaccatcgggcgcggatcagatctgcagcggccgctccagaattctagaaggtaccatggagcctcaaagtcagagcatgacgctggaggtgccgttgtccttggggagataccacatttctgaggaatatggctttctccttccaaatcctctggaagcacttccagatcattacaagccttggatggaaattgccctcagacttcctcacttaatcgagaaccgccagctccgagctcacgtgtacaggatgcctctcctggactgcagattcctaaagagttaccgtgagcagcgcctggcacacatggcgctggccgctatcaccatgggattcgtctggcaggagggggaaggccaaccccaaaaggtgctgccaagatctcttgccattccttttgttgaggtatccaggaacttgggactcccgcctatcctggtccactctgacctggtgctgacaaactggaccaaaaggaacccagaaggaccgttggaaatcagtaacctggaaaccatcatctcatttccggggggagagagcctgcggggcttcatcctagtgacagtcttggtggagaaggcagcagtgcccggccttaaggccctggttcagggaatggaggccattcggcaacacagtcaggacaccctgctagaagccctgcagcagctgagactctccatccaggatatcaccagagccttggcccaaatgcatgattatgtggacccagacatattttactcggtcatccggatcttcctctctgggtggaaggacaatccagccatgcctgtggggctggtctatgaaggtgctgccacagagcctctgaagtactctggaggaagtgcagcccagagctccgtgcttcatgccttcgatgagttcctgggcattgagcattgcaaggaaagtgttggctttctacacagaatgagggactacatgccgccttcccataaggctttcctggaagatctccacgtagctccttctctgagagactacatactggcctctggtcctggggactgcctgatggcctataaccagtgtgtggaggccctgggagagctgcgcagttaccacatcaatgtcgtggccagatacattatctccgctgccaccagggccaggagcagggggctaactaatccctcaccccatgccttggaagacaggggcactgggggtactgccatgctgagcttcttgaagagtgtcagggagaagaccatggaggccctcctgtgtcctggtgcttagggtcaagacaattctgcagatatccagcacagtggcggccgctcgagtctagagggcccgcggttcgaaggtaagcctatccctaaccctctcctcggtctcgattctacgcgtaccggtcatcatcaccatcaccattgagtttgctagccactagtaccgactctgctgaagaggaggaaattctccttgaagtttccctggtgttcaaagtaaaggagtttgcaccagacgcacctctgttcactggtccggcgtattaaaacacgatacattgttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaataaagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattatttctgattgcgggcgtttttgggcgggtttcaatctaactgtgcccgattttaattcagacaacacgttagaaagcgatggtgcaggcggtggtaacatttcagacggcaaatctactaatggcggcggtggtggagctgatgataaatctaccatcggtggaggcgcaggcggggctggcggcggaggcggaggcggaggtggtggcggtgatgcagacggcggtttaggctcaaatgtctctttaggcaacacagtcggcacctcaactattgtactggtttcgggcgccgtttttggtttgaccggtctgagacgagtgcgatttttttcgtttctaatagcttccaacaattgttgtctgtcgtctaaaggtgcagcgggttgaggttccgtcggcattggtggagcgggcggcaattcagacatcgatggtggtggtggtggtggaggcgctggaatgttaggcacgggagaaggtggtggcggcggtgccgccggtataatttgttctggtttagtttgttcgcgcacgattgtgggcaccggcgcaggcgccgctggctgcacaacggaaggtcgtctgcttcgaggcagcgcttggggtggtggcaattcaatattataattggaatacaaatcgtaaaaatctgctataagcattgtaatttcgctatcgtttaccgtgccgatatttaacaaccgctcaatgtaagcaattgtattgtaaagagattgtctcaagctcgccgcacgccgataacaagccttttcatttttactacagcattgtagtggcgagacacttcgctgtcgtcgacgtacatgtatgctttgttgtcaaaaacgtcgttggcaagctttaaaatatttaaaagaacatctctgttcagcaccactgtgttgtcgtaaatgttgtttttgataatttgcgcttccgcagtatcgacacgttcaaaaaattgatgcgcatcaattttgttgttcctattattgaataaataagattgtacagattcatatctacgattcgtcatggccaccacaaatgctacgctgcaaacgctggtacaattttacgaaaactgcaaaaacgtcaaaactcggtataaaataatcaacgggcgctttggcaaaatatctattttatcgcacaagcccactagcaaattgtatttgcagaaaacaatttcggcgcacaattttaacgctgacgaaataaaagttcaccagttaatgagcgaccacccaaattttataaaaatctattttaatcacggttccatcaacaaccaagtgatcgtgatggactacattgactgtcccgatttatttgaaacactacaaattaaaggcgagctttcgtaccaacttgttagcaatattattagacagctgtgtgaagcgctcaacgatttgcacaagcacaatttcatacacaacgacataaaactcgaaaatgtcttatatttcgaagcacttgatcgcgtgtatgtttgcgattacggattgtgcaaacacgaaaactcacttagcgtgcacgacggcacgttggagtattttagtccggaaaaaattcgacacacaactatgcacgtttcgtttgactggtacgcggcgtgttaacatacaagttgctaacgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcc

SEQ ID NO: 163 - DNA, 10511 bp, synthetic construct
  misc_feature 1..10511: note = Synthetic Sequence
  source 1..10511: mol_type = other DNA
aagctttactcgtaaagcgagttgaaggatcatatttagttgcgtttatgagataagattgaaagcacgtgtaaaatgtttcccgcgcgttggcacaactatttacaatgcggccaagttataaaagattctaatctgatatgttttaaaacacctttgcggcccgagttgtttgcgtacgtgactagcgaagaagatgtgtggaccgcagaacagatagtaaaacaaaaccctagtattggagcaataatcgatttaaccaacacgtctaaatattatgatggtgtgcattttttgcgggcgggcctgttatacaaaaaaattcaagtacctggccagactttgccgcctgaaagcatagttcaagaatttattgacacggtaaaagaatttacagaaaagtgtcccggcatgttggtgggcgtgcactgcacacacggtattaatcgcaccggttacatggtgtgcagatatttaatgcacaccctgggtattgcgccgcaggaagccatagatagattcgaaaaagccagaggtcacaaaattgaaagacaaaattacgttcaagatttattaatttaattaatattatttgcattctttaacaaatactttatcctattttcaaattgttgcgcttcttccagcgaaccaaaactatgcttcgcttgctccgtttagcttgtagccgatcagtggcgttgttccaatcgacggtaggattaggccggatattctccaccacaatgttggcaacgttgatgttacgtttatgcttttggttttccacgtacgtcttttggccggtaatagccgtaaacgtagtgccgtcgcgcgtcacgcacaacaccggatgtttgcgcttgtccgcggggtattgaaccgcgcgatccgacaaatccaccactttggcaactaaatcggtgacctgcgcgtcttttttctgcattatttcgtctttcttttgcatggtttcctggaagccggtgtacatgcggtttagatcagtcatgacgcgcgtgacctgcaaatctttggcctcgatctgcttgtccttgatggcaacgatgcgttcaataaactcttgttttttaacaagttcctcggttttttgcgccaccaccgcttgcagcgcgtttgtgtgctcggtgaatgtcgcaatcagcttagtcaccaactgtttgctctcctcctcccgttgtttgatcgcgggatcgtacttgccggtgcagagcacttgaggaattacttcttctaaaagccattcttgtaattctatggcgtaaggcaatttggacttcataatcagctgaatcacgccggatttagtaatgagcactgtatgcggctgcaaatacagcgggtcgccccttttcacgacgctgttagaggtagggcccccattttggatggtctgctcaaataacgatttgtatttattgtctacatgaacacgtatagctttatcacaaactgtatattttaaactgttagcgacgtccttggccacgaaccggacctgttggtcgcgctctagcacgtaccgcaggttgaacgtatcttctccaaatttaaattctccaattttaacgcgagccattttgatacacgtgtgtcgattttgcaacaactattgttttttaacgcaaactaaacttattgtggtaagcaataattaaatatgggggaacatgcgccgctacaacactcgtcgttatgaacgcagacggcgccggtctcggcgcaagcggctaaaacgtgttgcgcgttcaacgcggcaaacatcgcaaaagccaatagtacagttttgatttgcatattaacggcgattttttaaattatcttatttaataaatagttatgacgcctacaactccccgcccgcgttgactcgctgcacctcgagcagttcgttgacgccttcctccgtgtggccgaacacgtcgagcgggtggtcgatgaccagcggcgtgccgcacgcgacgcacaagtatctgtacaccgaatgatcgtcgggcgaaggcacgtcggcctccaagtggcaatattggcaaattcgaaaatatatacagttgggttgtttgcgcatatctatcgtggcgttgggcatgtacgtccgaacgttgatttgcatgcaagccgaaattaaatcattgcgattagtgcgattaaaacgttgtacatcctcgcttttaatcatgccgtcgattaaatcgcgcaatcgagtcaagtgatcaaagtgtggaataatgttttctttgtattcccgagtcaagcgcagcgcgtattttaacaaactagccatcttgtaagttagtttcatttaatgcaactttatccaataatatattatgtatcgcacgtcaagaattaacaatgcgcccgttgtcgcatctcaacacgactatgatagagatcaaataaagcgcgaattaaatagcttgcgacgcaacgtgcacgatctgtgcacgcgttccggcacgagctttgattgtaataagtttttacgaagcgatgacatgacccccgtagtgacaacgatcacgcccaaaagaactgccgactacaaaattaccgagtatgtcggtgacgttaaaactattaagccatccaatcgaccgttagtcgaatcaggaccgctggtgcgagaagccgcgaagtatggcgaatgcatcgtataacgtgtggagtccgctcattagagcgtcatgtttagacaagaaagctacatatttaattgatcccgatgattttattgataaattgaccctaactccatacacggtattctacaatggcggggttttggtcaaaatttccggactgcgattgtacatgctgttaacggctccgcccactattaatgaaattaaaaattccaattttaaaaaacgcagcaagagaaacatttgtatgaaagaatgcgtagaaggaaagaaaaatgtcgtcgacatgctgaacaacaagattaatatgcctccgtgtataaaaaaaatattgaacgatttgaaagaaaacaatgtaccgcgcggcggtatgtacaggaagaggtttatactaaactgttacattgcaaacgtggtttcgtgtgccaagtgtgaaaaccgatgtttaatcaaggctctgacgcatttctacaaccacgactccaagtgtgtgggtgaagtcatgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgctaaatttgttttttattaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcctcataaaaattaacatagttattatcgtatccatatatgtatctatcgtatagagtaaattttttgttgtcataaatatatatgtcttttttaatggggtgtatagtaccgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccattgtaatgagacgcacaaactaatatcacaaactggaaatgtctatcaatatatagttgctgatatcatggagataattaaaatgataaccatctcgcaaataaataagtattttactgttttcgtaacagttttgtaataaaaaaacctataaatattccggattattcataccgtcccaccatcgggcgcggatcagatctgcagcggccgctccagaattctagaaggtacccatggagccccacagaccgaatgtgaagacagcagtgccattgtctttggaaagctatcacatatctgaagagtatggctttcttcttccagattctctgaaagaacttccagatcattataggccttggatggaaattgccaacaaacttcctcaattgattgatgctcaccagcttcaagctcatgtggacaagatgcccctgctgagctgccagttcctgaagggtcaccgggagcagcgcctggcccacctggtcctgagcttcctcaccatgggttatgtctggcaggaaggagaggcgcagcctgcagaggtcctgccaaggaatcttgcccttccatttgtcgaagtctccaggaacttggggctccctcctatcctggtccactcagacttggtgctgacgaactggaccaaaaaagatccagacgggttcctggaaattgggaacctggagaccatcatctcatttcctgggggagagagcctgcatggttttatactggtgactgctttggtagagaaagaagcagtgcctgggataaaggctcttgttcaggccacgaatgctatcttgcagcccaaccaggaggccctgctccaagccctgcagcgactgagactgtctattcaggacatcaccaaaaccttaggacagatgcatgattatgtagatccagacatattttatgcaggcatccggatctttctctctgggtggaaagacaacccagcaatgcctgcagggctgatgtatgaaggagtttcccaagagcccctgaaatactccggcgggagtgcagctcagagcacagtgcttcatgcctttgatgagttcttaggcattcgtcatagcaaggaaagtggtgactttctgtacagaatgagggattacatgcctccttcccataaggccttcatagaagacatccactcagcaccttccctgagggactacatcctgtcatctggacaggaccacttgctgacagcttataaccagtgtgtgcaggccctggcagagctgcggagctatcacatcaccatggtcaccaaatacctcatcacagctgcagccaaggcaaagcatgggaagccaaaccatctcccagggcctcctcaggctttaaaagacaggggcacaggtggaaccgcagttatgagctttcttaagagtgtcagggataagaccttggagtcaatccttcacccacgtggttaacgctcgagtctagagggcccgcggttcgaaggtaagcctatccctaaccctctcctcggtctcgattctacgcgtaccggtcatcatcaccatcaccattgagtttgctagccactagtaccgactctgctgaagaggaggaaattctccttgaagtttccctggtgttcaaagtaaaggagtttgcaccagacgcacctctgttcactggtccggcgtattaaaacacgatacattgttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaataaagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattatttctgattgcgggcgtttttgggcgggtttcaatctaactgtgcccgattttaattcagacaacacgttagaaagcgatggtgcaggcggtggtaacatttcagacggcaaatctactaatggcggcggtggtggagctgatgataaatctaccatcggtggaggcgcaggcggggctggcggcggaggcggaggcggaggtggtggcggtgatgcagacggcggtttaggctcaaatgtctctttaggcaacacagtcggcacctcaactattgtactggtttcgggcgccgtttttggtttgaccggtctgagacgagtgcgatttttttcgtttctaatagcttccaacaattgttgtctgtcgtctaaaggtgcagcgggttgaggttccgtcggcattggtggagcgggcggcaattcagacatcgatggtggtggtggtggtggaggcgctggaatgttaggcacgggagaaggtggtggcggcggtgccgccggtataatttgttctggtttagtttgttcgcgcacgattgtgggcaccggcgcaggcgccgctggctgcacaacggaaggtcgtctgcttcgaggcagcgcttggggtggtggcaattcaatattataattggaatacaaatcgtaaaaatctgctataagcattgtaatttcgctatcgtttaccgtgccgatatttaacaaccgctcaatgtaagcaattgtattgtaaagagattgtctcaagctcgccgcacgccgataacaagccttttcatttttactacagcattgtagtggcgagacacttcgctgtcgtcgacgtacatgtatgctttgttgtcaaaaacgtcgttggcaagctttaaaatatttaaaagaacatctctgttcagcaccactgtgttgtcgtaaatgttgtttttgataatttgcgcttccgcagtatcgacacgttcaaaaaattgatgcgcatcaattttgttgttcctattattgaataaataagattgtacagattcatatctacgattcgtcatggccaccacaaatgctacgctgcaaacgctggtacaattttacgaaaactgcaaaaacgtcaaaactcggtataaaataatcaacgggcgctttggcaaaatatctattttatcgcacaagcccactagcaaattgtatttgcagaaaacaatttcggcgcacaattttaacgctgacgaaataaaagttcaccagttaatgagcgaccacccaaattttataaaaatctattttaatcacggttccatcaacaaccaagtgatcgtgatggactacattgactgtcccgatttatttgaaacactacaaattaaaggcgagctttcgtaccaacttgttagcaatattattagacagctgtgtgaagcgctcaacgatttgcacaagcacaatttcatacacaacgacataaaactcgaaaatgtcttatatttcgaagcacttgatcgcgtgtatgtttgcgattacggattgtgcaaacacgaaaactcacttagcgtgcacgacggcacgttggagtattttagtccggaaaaaattcgacacacaactatgcacgtttcgtttgactggtacgcggcgtgttaacatacaagttgctaacgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcc